(19) United States
(12) Patent Application Publication
(10) Pub. No.: US 2024/0344131 A1
(43) Pub. Date: Oct. 17, 2024

(54) GENOMIC SIGNATURES OF FIBROMYALGIA AND CHRONIC PAIN AND USES THEREOF

(71) Applicant: Center for Immunology Science, Los Angeles, CA (US)

(72) Inventor: Bruce S. GILLIS, Beverly Hills, CA (US)

(21) Appl. No.: 18/603,757

(22) Filed: Mar. 13, 2024

Related U.S. Application Data
(60) Provisional application No. 63/489,881, filed on Mar. 13, 2023.

(51) Int. Cl. C12Q 1/6883

(52) U.S. Cl. CPC C12Q 1/6883; C12Q 2600/158

(57) ABSTRACT

The invention provides methods for detecting or diagnosing fibromyalgia (FM) in an individual by determining the expression of biomarkers at the nucleic acid level. Also provided herein are methods for detecting or diagnosing FM in an individual and using said detection or diagnosis to guide administration of treatments for said conditions.

### CROSS REFERENCE TO RELATED APPLICATIONS

This application claims priority from U.S. Provisional Application No. 63/489,881 filed Mar. 13, 2023, which is incorporated by reference herein in its entirety for all purposes.

### FIELD

This invention provides methods for detecting or diagnosing immune deficiency diseases (e.g., fibromyalgia) by analyzing genetic markers associated with said diseases.

### BACKGROUND

Fibromyalgia (FM) is a chronic pain syndrome that for decades has been questioned as a medical disease or merely a collection of symptoms. Those symptoms include chronic, non-remitting pain, body area tenderness, persistent fatigue, recurrent headaches, “brain fog,” generalized anxiety, chronic depression, poor sleep, leg cramps, numbness and tingling, difficulty concentrating and restless legs while sleeping (Sarzi-Puttini, P., Giorgi, V., Marotto, D. & Atzeni, F. Fibromyalgia: an update on clinical characteristics, aetiopathogenesis and treatment. Nat Rev Rheumatol 16, 645-660 (2020). “Fibrositis” was the first name assigned to this collection of medical complaints. The first proposed criteria for identifying FM patients were put forth in 1990 by the American College of Rheumatology for the purpose of identifying patients for research (Goldenberg, D. L. Fibromyalgia syndrome. An emerging but controversial condition. JAMA 257, 2782-2787 (1987)). The proposed criteria included a history of widespread pain and eighteen designated tender points on physical examination. In 2016, the American College of Rheumatology put forth provisional criteria for FM and revised these in 2016 (Wolfe, F., et al. 2016 Revisions to the 2010/2011 fibromyalgia diagnostic criteria. Semin Arthritis Rheum 46, 319-329 (2016); Wolfe, F., et al. 2016 Revisions to the 2010/2011 fibromyalgia diagnostic criteria. Semin Arthritis Rheum 46, 319-329 (2016); Nicholas, M., et al. The IASP classification of chronic pain for ICD-11: chronic primary pain. Pain 160, 28-37 (2019)). However, these criteria are difficult to assess in the clinical realm and are not widely accepted such as evidenced by another diagnostic system proposed by the American Pain Society in 2019 (Nicholas, M., et al. The IASP classification of chronic pain for ICD-11: chronic primary pain. Pain 160, 28-37 (2019)).

Currently, there are no individual biomarkers available for accurate diagnosis of FM. Using a multiplex cytokine assay, cytokine profiles were reported for FM patients (Behm, F. G., et al. Unique immunologic patterns in fibromyalgia. BMC Clin Pathol 12, 25 (2012)). This study showed that the pro-inflammatory cytokines such as IL6, IL8, MIP-1α (CCL3) and MIP-1β (CCL4) were consistently under expressed in FM patients as compared to a control group of healthy individuals. Subsequently, a second independent study demonstrated that these biomarkers for FM were unique and did not occur in rheumatoid arthritis or systemic lupus erythematosus (Wallace, D. J., Gavin, I. M., Karpenko, O., Barkhordar, F. & Gillis, B. S. Cytokine and chemokine profiles in fibromyalgia, rheumatoid arthritis and systemic lupus erythematosus: a potentially useful tool in differential diagnosis. Rheumatol Int 35, 991-996 (2015)).

Current investigations into the pathophysiology of FM have focused on the immune system (e.g., inflammatory and anti-inflammatory cytokines) (Sarzi-Puttini, P., Giorgi, V., Marotto, D. & Atzeni, F. Fibromyalgia: an update on clinical characteristics, aetiopathogenesis and treatment. Nat Rev Rheumatol 16, 645-660 (2020)), the nervous system (e.g., neuroimmune axis, pain processing, neurotransmitters, autonomic nervous system) (Meade, E. & Garvey, M. The Role of Neuro-Immune Interaction in Chronic Pain

Conditions; Functional Somatic Syndrome, Neurogenic Inflammation, and Peripheral Neuropathy. Int J Mol Sci 23(2022)), the digestive system (a gut-brain axis and the gut microbiome) (Clos-Garcia, M., et al. Gut microbiome and serum metabolome analyses identify molecular biomarkers and altered glutamate metabolism in fibromyalgia. EBioMedicine 46, 499-511 (2019); Minerbi, A. & Fitzcharles, M. A. Gut microbiome: pertinence in fibromyalgia. Clin Exp Rheumatol 38 Suppl 123, 99-104 (2020)), and genetics (e.g., genome-wide linkage analysis, twin studies, pain and neurotransmitter gene abnormalities) (Arnold, L. M., et al. The fibromyalgia family study: a genome-wide linkage scan study. Arthritis Rheum 65, 1122-1128 (2013); Arnold, L. M., et al. Family study of fibromyalgia. Arthritis Rheum 50, 944-952 (2004); Kato, K., Sullivan, P. F., Evengard, B. & Pedersen, N. L. A population-based twin study of functional somatic syndromes. Psychol Med 39, 497-505 (2009); Armitage, R., et al. Power spectral analysis of sleep EEG in twins discordant for chronic fatigue syndrome. J Psychosom Res 66, 51-57 (2009)). However, the findings of many of these studies are often contradictory and many have not been independently confirmed.

Thus, there is a need in the art for a precise method of analysis for definitive diagnostic purposes with respect to fibromyalgia. Provided herein are methods, kits and compositions for addressing this need.

### SUMMARY

In one aspect, provided herein is a method of assaying a sample obtained from a subject, the method comprising measuring a nucleic acid expression level of a plurality of biomarkers from either Table 3 or Table 4 in the sample obtained from the subject, wherein the subject suffers from or is suspected of suffering from fibromyalgia (FM). In some cases, the method further comprises comparing the detected levels of nucleic acid expression of the plurality of biomarkers selected from Table 3 or Table 4 to the expression of the plurality of biomarkers selected from Table 3 or Table 4 in a control; and classifying the subject as having FM based on the results of the comparing step. In some cases, the comparing step comprises applying a statistical algorithm which comprises determining a correlation between the expression data obtained from the sample and the expression data from the control. In some cases, the nucleic acid expression level is RNA or cDNA. In some cases, the detecting the nucleic acid expression level comprises performing quantitative real time reverse transcriptase polymerase chain reaction (qRT-PCR), RNAseq, or whole transcriptome analysis. In some cases, the nucleic acid expression level is detected by performing RNA-seq. In some cases, the detection of the nucleic acid expression level comprises using at least one pair of oligonucleotide primers specific for each biomarker from the plurality of biomarkers selected from Table 3 or Table 4. In some cases, the sample is a bodily fluid obtained from the subject. In some cases, the bodily fluid is blood or fractions thereof, urine, saliva, or sputum. In some cases, the plurality of biomarkers selected from Table 3 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers or at least 74 biomarkers from Table 3. In some cases, the plurality of biomarkers selected from Table 3 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 3. In some cases, the plurality of biomarkers selected from Table 3 comprise biomarkers associated with the GP6 signaling pathway, the wound healing signaling pathway, RHOGDI signaling, phagosome formation, the intrinsic prothrombin activation pathway, the pulmonary fibrosis idiopathic signaling pathway, synaptic long term depression, the oxytocin signaling pathway, sperm motility or cell cycle GS/M DNA damage checkpoint regulation. In some cases, the plurality of biomarkers selected from Table 3 comprises all the biomarkers from Table 3. In some cases, the plurality of biomarkers selected from Table 4 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers, at least 84 biomarkers, at least 86 biomarkers, at least 88 biomarkers, at least 90 biomarkers, at least 92 biomarkers, at least 94 biomarkers, at least 96 biomarkers, at least 98 biomarkers or at least 99 biomarkers from Table 4. In some cases, the plurality of biomarkers selected from Table 4 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 4. In some cases, the plurality of biomarkers selected from Table 4 comprise biomarkers associated with phagosome formation, the CLEAR signaling pathway, the pyroptosis signaling pathway, TREM1 signaling, the neuroinflammation signaling pathway, Th1 pathway, LPS/Il-1 mediated inhibition of RXR function, crosstalk between dendritic cells and natural killer cells, Fcgamma Receptor-mediated phagocytosis in macrophages and monocytes, production of nitric oxide and reactive oxygen species in macrophages, Toll-Like receptor signaling, inflammasome pathway, G-protein coupled receptor signaling, Th2 pathway, LXR/RXR activation, role of pattern recognition receptors in recognition of bacteria and viruses, glycolysis I or the necroptosis signaling pathway. In some cases, the plurality of biomarkers selected from Table 4 comprises all the biomarkers from Table 4.

In another aspect, provided herein is a method of detecting a biomarker in a sample obtained from a subject suffering from or suspected of suffering from fibromyalgia (FM), the method comprising, consisting essentially of or consisting of measuring the nucleic acid expression level of a plurality of biomarkers selected from Table 3 or 4 using an amplification, hybridization and/or sequencing assay. In some cases, the sample was previously diagnosed as being FM. In some cases, the amplification, hybridization and/or sequencing assay comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, or Northern blotting. In some cases, the nucleic acid expression level is detected by performing qRT-PCR. In some cases, the detection of the nucleic acid expression level comprises using at least one pair of oligonucleotide primers per each of the plurality of biomarker selected from Table 3 or 4. In some cases, the sample is a bodily fluid obtained from the subject. In some cases, the bodily fluid is blood or fractions thereof, urine, saliva, or sputum. In some cases, the plurality of biomarkers selected from Table 3 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers or at least 74 biomarkers from Table 3. In some cases, the plurality of biomarkers selected from Table 3 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 3. In some cases, the plurality of biomarkers selected from Table 3 comprise biomarkers associated with the GP6 signaling pathway, the wound healing signaling pathway, RHOGDI signaling, phagosome formation, the intrinsic prothrombin activation pathway, the pulmonary fibrosis idiopathic signaling pathway, synaptic long term depression, the oxytocin signaling pathway, sperm motility or cell cycle GS/M DNA damage checkpoint regulation. In some cases, the plurality of biomarkers selected from Table 3 comprises all the biomarkers from Table 3. In some cases, the plurality of biomarkers selected from Table 4 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers, at least 84 biomarkers, at least 86 biomarkers, at least 88 biomarkers, at least 90 biomarkers, at least 92 biomarkers, at least 94 biomarkers, at least 96 biomarkers, at least 98 biomarkers or at least 99 biomarkers from Table 4. In some cases, the plurality of biomarkers selected from Table 4 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 4. In some cases, the plurality of biomarkers selected from Table 4 comprise biomarkers associated with phagosome formation, the CLEAR signaling pathway, the pyroptosis signaling pathway, TREM1 signaling, the neuroinflammation signaling pathway, Th1 pathway, LPS/Il-1 mediated inhibition of RXR function, crosstalk between dendritic cells and natural killer cells, Fcgamma Receptor-mediated phagocytosis in macrophages and monocytes, production of nitric oxide and reactive oxygen species in macrophages, Toll-Like receptor signaling, inflammasome pathway, G-protein coupled receptor signaling, Th2 pathway, LXR/RXR activation, role of pattern recognition receptors in recognition of bacteria and viruses, glycolysis I or the necroptosis signaling pathway. In some cases, the plurality of biomarkers selected from Table 4 comprises all the biomarkers from Table 4.

In another aspect, provided herein is a method of treating fibromyalgia (FM) in a subject, the method comprising: measuring a nucleic acid expression level of a plurality of biomarkers in a sample obtained from a subject suspected of suffering from FM, wherein the plurality of biomarkers is selected from Table 3 or 4, wherein the nucleic acid expression level of the plurality of biomarkers indicates that the subject has FM; and administering a standard of care for FM to the subject, wherein the standard of care for FM is selected from the group consisting of duloxetine, pregabalin, gabapentin and milnacipran. In some cases, the determining step further comprises comparing the nucleic acid expression levels of the plurality of biomarkers from Table 3 to the nucleic acid expression levels of the plurality of biomarkers from Table 3 in at least one sample training set(s), wherein the at least one sample training set comprises nucleic acid expression level data of the plurality of biomarkers from Table 3 from a reference FM sample, nucleic acid expression level data of the plurality of biomarkers from Table 3 from a reference non-FM sample or a combination thereof; and classifying the subject as having FM based on the results of the comparing step. In some cases, the non-FM sample is obtained from an individual not known to have FM. In some cases, the non-FM sample is obtained from an individual diagnosed with a disease of widespread pain selected from the group consisting of rheumatoid arthritis, systemic lupus erythematosus (SLE), Sjogren Syndrome and multiple sclerosis. In some cases, the comparing step comprises applying a statistical algorithm which comprises determining a correlation between the expression data obtained from the sample obtained from the subject and the expression data from the at least one training set(s); and classifying the subject as having FM based on the results of the statistical algorithm. In some cases, the plurality of biomarkers selected from Table 3 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers or at least 74 biomarkers from Table 3. In some cases, the plurality of biomarkers selected from Table 3 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 3. In some cases, the plurality of biomarkers selected from Table 3 comprise biomarkers associated with the GP6 signaling pathway, the wound healing signaling pathway, RHOGDI signaling, phagosome formation, the intrinsic prothrombin activation pathway, the pulmonary fibrosis idiopathic signaling pathway, synaptic long term depression, the oxytocin signaling pathway, sperm motility or cell cycle GS/M DNA damage checkpoint regulation. In some cases, the plurality of biomarkers selected from Table 3 comprises all the biomarkers from Table 3. In some cases, the plurality of biomarkers selected from Table 4 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers, at least 84 biomarkers, at least 86 biomarkers, at least 88 biomarkers, at least 90 biomarkers, at least 92 biomarkers, at least 94 biomarkers, at least 96 biomarkers, at least 98 biomarkers or at least 99 biomarkers from Table 4. In some cases, the plurality of biomarkers selected from Table 4 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 4. In some cases, the plurality of biomarkers selected from Table 4 comprise biomarkers associated with phagosome formation, the CLEAR signaling pathway, the pyroptosis signaling pathway, TREM1 signaling, the neuroinflammation signaling pathway, Th1 pathway, LPS/Il-1 mediated inhibition of RXR function, crosstalk between dendritic cells and natural killer cells, Fcgamma Receptor-mediated phagocytosis in macrophages and monocytes, production of nitric oxide and reactive oxygen species in macrophages, Toll-Like receptor signaling, inflammasome pathway, G-protein coupled receptor signaling, Th2 pathway, LXR/RXR activation, role of pattern recognition receptors in recognition of bacteria and viruses, glycolysis I or the necroptosis signaling pathway. In some cases, the plurality of biomarkers selected from Table 4 comprises all the biomarkers from Table 4. In some cases, the measuring the nucleic acid expression level is conducted using an amplification, hybridization and/or sequencing assay. In some cases, the amplification, hybridization and/or sequencing assay comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays or Northern blotting. In some cases, the expression level is detected by performing qRT-PCR. In some cases, the sample is a bodily fluid obtained from the subject. In some cases, the bodily fluid is blood or fractions thereof, urine, saliva, or sputum.

In still another aspect, provided herein is a system for diagnosing fibromyalgia (FM) from a sample obtained from a subject suspected of suffering from FM, the system comprising: (a) one or more processors; and (b) one or more memories operatively coupled to at least one of the one or more processors and having instructions stored thereon that, when executed by at least one of the one or more processors, cause the system to: (i) detect an expression level of each of a plurality of biomarkers from Table 3 or 4; (ii) compare the expression levels of each of the plurality of biomarkers from Table 3 to the expression levels of each of the plurality of biomarkers from Table 3 in a control or compare the expression levels of each of the plurality of biomarkers from Table 4 to the expression levels of each of the plurality of biomarkers from Table 4 in a control; and (iii) classifying the subject as having FM based on the results of the comparing step. In some cases, the control comprises at least one sample training set(s), wherein the at least one sample training set comprises expression levels of each of the plurality of biomarkers from Table 3 or 4 from a reference FM sample, expression levels of each of the plurality of biomarkers from Table 3 or 4 from a reference non-FM sample or a combination thereof. In some cases, the non-FM sample is obtained from an individual not known to have FM. In some cases, the non-FM sample is obtained from an individual diagnosed with a disease of widespread pain selected from the group consisting of rheumatoid arthritis, systemic lupus erythematosus (SLE), Sjogren Syndrome and multiple sclerosis. In some cases, the comparing step comprises applying a statistical algorithm which comprises determining a correlation between the nucleic acid expression data obtained from the sample and the expression data from the at least one training set(s); and classifying the subject as having FM based on the results of the statistical algorithm. In some cases, the nucleic acid expression level is RNA or cDNA. In some cases, the detecting the expression level comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays or Northern blotting. In some cases, the expression level is detected by performing qRT-PCR. In some cases, the detecting the expression level is performed using a device that is part of the system or in communication with at least one of the one or more processors, wherein upon receipt of instructions sent by the at least one of the one or more processors, perform the detection of the expression levels. In some cases, the plurality of biomarkers selected from Table 3 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers or at least 74 biomarkers from Table 3. In some cases, the plurality of biomarkers selected from Table 3 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 3. In some cases, the plurality of biomarkers selected from Table 3 comprise biomarkers associated with the GP6 signaling pathway, the wound healing signaling pathway, RHOGDI signaling, phagosome formation, the intrinsic prothrombin activation pathway, the pulmonary fibrosis idiopathic signaling pathway, synaptic long term depression, the oxytocin signaling pathway, sperm motility or cell cycle GS/M DNA damage checkpoint regulation. In some cases, the plurality of biomarkers selected from Table 3 comprises all the biomarkers from Table 3. In some cases, the plurality of biomarkers selected from Table 4 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers, at least 84 biomarkers, at least 86 biomarkers, at least 88 biomarkers, at least 90 biomarkers, at least 92 biomarkers, at least 94 biomarkers, at least 96 biomarkers, at least 98 biomarkers or at least 99 biomarkers from Table 4. In some cases, the plurality of biomarkers selected from Table 4 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 4. In some cases, the plurality of biomarkers selected from Table 4 comprise biomarkers associated with phagosome formation, the CLEAR signaling pathway, the pyroptosis signaling pathway, TREM1 signaling, the neuroinflammation signaling pathway, Th1 pathway, LPS/Il-1 mediated inhibition of RXR function, crosstalk between dendritic cells and natural killer cells, Fcgamma Receptor-mediated phagocytosis in macrophages and monocytes, production of nitric oxide and reactive oxygen species in macrophages, Toll-Like receptor signaling, inflammasome pathway, G-protein coupled receptor signaling, Th2 pathway, LXR/RXR activation, role of pattern recognition receptors in recognition of bacteria and viruses, glycolysis I or the necroptosis signaling pathway. In some cases, the plurality of biomarkers selected from Table 4 comprises all the biomarkers from Table 4.

In one aspect, provided herein is a method of assaying a sample obtained from a subject, the method comprising measuring a nucleic acid expression level of a plurality of biomarkers from Table 5 in the sample obtained from the subject, wherein the subject suffers from or is suspected of suffering from fibromyalgia (FM). In some cases, the method further comprises comparing the detected levels of nucleic acid expression of the plurality of biomarkers selected from Table 5 to the expression of the plurality of biomarkers selected from Table 5 in a control; and classifying the subject as having FM based on the results of the comparing step. In some cases, the comparing step comprises applying a statistical algorithm which comprises determining a correlation between the expression data obtained from the sample and the expression data from the control. In some cases, the nucleic acid expression level is RNA or cDNA. In some cases, the detecting the nucleic acid expression level comprises performing quantitative real time reverse transcriptase polymerase chain reaction (qRT-PCR), RNAseq or whole transcriptome analysis. In some cases, the nucleic acid expression level is detected by performing RNA-seq. In some cases, the detection of the nucleic acid expression level comprises using at least one pair of oligonucleotide primers specific for each biomarker from the plurality of biomarkers selected from Table 5. In some cases, the sample is a bodily fluid obtained from the subject. In some cases, the bodily fluid is blood or fractions thereof, urine, saliva, or sputum. In some cases, the plurality of biomarkers selected from Table 5 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers or at least 83 biomarkers from Table 5. In some cases, the plurality of biomarkers selected from Table 5 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 5. In some cases, the plurality of biomarkers selected from Table 5 comprise biomarkers associated with Processing of capped intron-containing pre-mRNA, the Interferon alpha/beta signaling, the Necroptosis signaling pathway, Death receptor signaling, DDX58/IFIH1-mediated induction of interferon-alpha/beta, Role of PKR in interferon induction and antiviral response, the Pyroptosis signaling pathway, ISG15 antiviral mechanism, Interferon gamma signaling, Natural killer cell signaling, Role of hyperchemokinemia in the pathogenesis of influenza, ISGylation signaling pathway, JAK/STAT signaling, Activation of IRF by cytosolic pattern recognition receptors, Interleukin-2 family signaling, RIPK1-mediated regulated necrosis or Salvage pathways of pyrimidine ribonucleotides. In some cases, the plurality of biomarkers selected from Table 5 comprises all the biomarkers from Table 5.

In one aspect, provided herein is a method of detecting a biomarker in a sample obtained from a subject suffering from or suspected of suffering from fibromyalgia (FM), the method comprising, consisting essentially of or consisting of measuring the nucleic acid expression level of a plurality of biomarkers selected from Table 5 using an amplification, hybridization and/or sequencing assay. In some cases, the sample was previously diagnosed as being FM. In some cases, the amplification, hybridization and/or sequencing assay comprises performing quantitative real time reverse transcriptase polymerase chain reaction (qRT-PCR), RNAseq, microarrays, gene chips, Serial Analysis of Gene Expression (SAGE), Rapid Analysis of Gene Expression (RAGE), nuclease protection assays, or Northern blotting. In some cases, the nucleic acid expression level is detected by performing qRT-PCR. In some cases, the detection of the nucleic acid expression level comprises using at least one pair of oligonucleotide primers per each of the plurality of biomarker selected from Table 5. In some cases, the sample is a bodily fluid obtained from the subject. In some cases, the bodily fluid is blood or fractions thereof, urine, saliva, or sputum. In some cases, the plurality of biomarkers selected from Table 5 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers, or at least 83 biomarkers from Table 5. In some cases, the plurality of biomarkers selected from Table 5 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 5. In some cases, the plurality of biomarkers selected from Table 5 comprise biomarkers associated with Processing of capped intron-containing pre-mRNA, the Interferon alpha/beta signaling, the Necroptosis signaling pathway, Death receptor signaling, DDX58/IFIH1-mediated induction of interferon-alpha/beta, Role of PKR in interferon induction and antiviral response, the Pyroptosis signaling pathway, ISG15 antiviral mechanism, Interferon gamma signaling, Natural killer cell signaling, Role of hyperchemokinemia in the pathogenesis of influenza, ISGylation signaling pathway, JAK/STAT signaling, Activation of IRF by cytosolic pattern recognition receptors, Interleukin-2 family signaling, RIPK1-mediated regulated necrosis or Salvage pathways of pyrimidine ribonucleotides. In some cases, the plurality of biomarkers selected from Table 5 comprises all the biomarkers from Table 5.

In one aspect, provided herein is a method of treating fibromyalgia (FM) in a subject, the method comprising: measuring a nucleic acid expression level of a plurality of biomarkers in a sample obtained from a subject suspected of suffering from FM, wherein the plurality of biomarkers is selected from Table 5, wherein the nucleic acid expression level of the plurality of biomarkers indicates that the subject has FM; and administering a standard of care for FM to the subject, wherein the standard of care for FM is selected from the group consisting of duloxetine, pregabalin, gabapentin and milnacipran. In some cases, the determining step further comprises comparing the nucleic acid expression levels of the plurality of biomarkers from Table 5 to the nucleic acid expression levels of the plurality of biomarkers from Table 5 in at least one sample training set(s), wherein the at least one sample training set comprises nucleic acid expression level data of the plurality of biomarkers from Table 5 from a reference FM sample, nucleic acid expression level data of the plurality of biomarkers from Table 5 from a reference non-FM sample or a combination thereof; and classifying the subject as having FM based on the results of the comparing step. In some cases, the non-FM sample is obtained from an individual not known to have FM. In some cases, the non-FM sample is obtained from an individual diagnosed with a disease of widespread pain selected from the group consisting of rheumatoid arthritis, systemic lupus erythematosus (SLE), Sjogren Syndrome and multiple sclerosis. In some cases, the comparing step comprises applying a statistical algorithm which comprises determining a correlation between the expression data obtained from the sample obtained from the subject and the expression data from the at least one training set(s); and classifying the subject as having FM based on the results of the statistical algorithm. In some cases, the plurality of biomarkers selected from Table 5 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers or at least 83 biomarkers from Table 5. In some cases, the plurality of biomarkers selected from Table 5 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 5. In some cases, the plurality of biomarkers selected from Table comprise biomarkers associated with Processing of capped intron-containing pre-mRNA, the Interferon alpha/beta signaling, the Necroptosis signaling pathway, Death receptor signaling, DDX58/IFIH1-mediated induction of interferon-alpha/beta, Role of PKR in interferon induction and antiviral response, the Pyroptosis signaling pathway, ISG15 antiviral mechanism, Interferon gamma signaling, Natural killer cell signaling, Role of hyperchemokinemia in the pathogenesis of influenza, ISGylation signaling pathway, JAK/STAT signaling, Activation of IRF by cytosolic pattern recognition receptors, Interleukin-2 family signaling, RIPK1-mediated regulated necrosis or Salvage pathways of pyrimidine ribonucleotides. In some cases, the plurality of biomarkers selected from Table 5 comprises all the biomarkers from Table 5. In some cases, the measuring the nucleic acid expression level is conducted using an amplification, hybridization and/or sequencing assay. In some cases, the amplification, hybridization and/or sequencing assay comprises performing quantitative real time reverse transcriptase polymerase chain reaction (qRT-PCR), RNAseq, microarrays, gene chips, Serial Analysis of Gene Expression (SAGE), Rapid Analysis of Gene Expression (RAGE), nuclease protection assays or Northern blotting. In some cases, the expression level is detected by performing qRT-PCR. In some cases, the sample is a bodily fluid obtained from the subject. In some cases, the bodily fluid is blood or fractions thereof, urine, saliva, or sputum.

In another aspect, provided herein is a system for diagnosing fibromyalgia (FM) from a sample obtained from a subject suspected of suffering from FM, the system comprising: (a) one or more processors; and (b) one or more memories operatively coupled to at least one of the one or more processors and having instructions stored thereon that, when executed by at least one of the one or more processors, cause the system to (i) detect an expression level of each of a plurality of biomarkers from Table 5; (ii) compare the expression levels of each of the plurality of biomarkers from Table 5 to the expression levels of each of the plurality of biomarkers from Table 5 in a control or compare the expression levels of each of the plurality of biomarkers from Table 5 to the expression levels of each of the plurality of biomarkers from Table 5 in a control; and (iii) classifying the subject as having FM based on the results of the comparing step. In some cases, the control comprises at least one sample training set(s), wherein the at least one sample training set comprises expression levels of each of the plurality of biomarkers from Table 5 from a reference FM sample, expression levels of each of the plurality of biomarkers from Table 5 from a reference non-FM sample or a combination thereof. In some cases, the non-FM sample is obtained from an individual not known to have FM. In some cases, the non-FM sample is obtained from an individual diagnosed with a disease of widespread pain selected from the group consisting of rheumatoid arthritis, systemic lupus erythematosus (SLE), Sjogren Syndrome and multiple sclerosis. In some cases, the comparing step comprises applying a statistical algorithm which comprises determining a correlation between the nucleic acid expression data obtained from the sample and the expression data from the at least one training set(s); and classifying the subject as having FM based on the results of the statistical algorithm. In some cases, the expression level is a nucleic acid expression level, wherein the nucleic acid expression level is RNA or cDNA. In some cases, the detecting the nucleic acid expression level comprises performing quantitative real time reverse transcriptase polymerase chain reaction (qRT-PCR), RNAseq, microarrays, gene chips, Serial Analysis of Gene Expression (SAGE), Rapid Analysis of Gene Expression (RAGE), nuclease protection assays or Northern blotting. In some cases, the nucleic acid expression level is detected by performing qRT-PCR. In some cases, the detecting the expression level is performed using a device that is part of the system or in communication with at least one of the one or more processors, wherein upon receipt of instructions sent by the at least one of the one or more processors, perform the detection of the expression levels. In some cases, the plurality of biomarkers selected from Table 5 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers, or at least 83 biomarkers from Table 5. In some cases, the plurality of biomarkers selected from Table 5 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 5. In some cases, the plurality of biomarkers selected from Table 5 comprise biomarkers associated with Processing of capped intron-containing pre-mRNA, the Interferon alpha/beta signaling, the Necroptosis signaling pathway, Death receptor signaling, DDX58/IFIH1-mediated induction of interferon-alpha/beta, Role of PKR in interferon induction and antiviral response, the Pyroptosis signaling pathway, ISG15 antiviral mechanism, Interferon gamma signaling, Natural killer cell signaling, Role of hyperchemokinemia in the pathogenesis of influenza, ISGylation signaling pathway, JAK/STAT signaling, Activation of IRF by cytosolic pattern recognition receptors, Interleukin-2 family signaling, RIPK1-mediated regulated necrosis or Salvage pathways of pyrimidine ribonucleotides. In some cases, the plurality of biomarkers selected from Table 5 comprises all the biomarkers from Table 5.

### BRIEF DESCRIPTION OF THE FIGURES

FIG. 1A illustrates hierarchical clustering of patient symptoms indicates a near perfect separation into two groups. A minority of patients were misclassified: three control subjects #286, #332, #335 were assigned to the FM group while two FM patients #028, #078 were assigned to the control group. Average linkage using Euclidean metrics with k=2 classes, control patients represented in blue, FM patients represented in red, misclassified patients are identified by an asterisk.

FIG. 1B illustrates clustering of FM patient symptoms. The symptoms were grouped by hierarchical clustering using average linking of Pearson correlation metric. The vertical bars represent the three groups of correlated symptoms that are present. The star ‘*’ or ‘0’ represents the significance of the cluster. ‘0’: No significant correlation, ‘*’: adjusted p-value ≤0.01.

FIG. 1C illustrates a principal component analysis (PCA) of the symptoms in FM patients. Patient 078 (outlier) is indicated by arrow.

FIGS. 2A-2B depicts a summary of DEGs identified in FM subgroups. FIG. 2A illustrates a heatmap showing RNA expression in the controls and FM samples. A total of 1300 differentially expressed transcripts were used to draw the heatmap using the algorithm of TPMs. Control patients (n=71) are compared to FM1 (n=44), FM2 (n=31) and FM 3-5 samples (n=21). In blue: low expression, in pink high expression. Samples are verticals and genes are horizontal. The 2 boxed inserts are zoomed in areas of the heatmap indicating the genes that showed the most significant differential expression for FM1 (left box) and FM2 (Right box). The label ‘3’, ‘4’ and ‘5’ correspond to the FM3, FM4 and FM5 respectively. FIG. 2B depicts a representation of the proportion of controls and FMs patients based on their gene expression. 71 patients were called homogeneous because this group of patients showed similar gene expression and clustered together. The 22 control patients that did not have the similar gene expression profile were not included for downstream analysis. Among the FM patients, different groups that had different gene expression profiles were found. The major group called ‘FM1’ was formed by 44 patients, a second group called ‘FM2’ was formed by 31 patients. The other FM patients could be separated into three clusters but were not analyzed further due to small sample size (n=8, 9 and 4) that did not give us enough statistical power compared to the FM1 and FM2 (n=44 and 31 respectively).

FIG. 3A-3B illustrates PCA of the FM patients and controls. FIG. 3A shows PCA of the entire cohort of 94 FM and 90 controls was performed using 1169 DEGs that showed the most significant differential expression. The first component axis shows 46.1% while the second component shows 8.8% of the information. No other components than the first and second components were found useful. Ellipses show 80% confidence interval of each group, the supersized dot corresponds to the centroid of the group. Control patients are represented by a dark blue dot, control patients classified as outliers are represented by a cyan dot, fibromyalgia patients FM1, FM2, FM3, FM4 are represented by a red, green, magenta, orange dot respectively. The 4 FM patients that did not group together are represented by brown dots. After removing the 2 clusters of patients from FM1 and FM2, the PCA separation of the patients from groups FM 3, 4 and 5 on three clusters based on the first component (48%; FIG. 3B).

FIG. 4A-4E illustrates analysis of differentially expressed genes (DEGs) in the FM1 Subgroup. FIG. 4A depicts a PCA of the 90 control patients and the 43 patients of FM1 subgroup using 480 DEGs that showed the most significant differential expression. The near perfect separation shows that most of the variation is represented by the first component (82%). Blue dots: control patients. Red triangle: FM patients. The ellipses correspond to the threshold at 80% confidence. Cyan dots how the outlier controls. FIG. 4B illustrates an interactome analysis of 43 FM1 patients showed a group of 338 DEGs (magenta color) and a small cluster of 24 DEGs (green color). Each dot represent a DEG, the gray dots represent DEGs that did not reach significance. FIG. 4C illustrates pathway analysis of the DEGs contained in the major cluster (magenta) and the minor cluster (green) using a threshold of −log(p value) ≥2. FIG. 4D shows a summary of biological functions related to the pathways identified in FM1 patients (FIG. 5C). FIG. 4E illustrates a small cluster of 24 genes (green) including coding and lncRNA from the interactome analysis, are associated with cell cycle regulation. Blue: Downregulation, Orange: Upregulation.

FIGS. 5A-5B show the results of GSEA analysis of the whole transcriptome for FM1 group. FIG. 5A shows enrichment in olfactory receptor biological process (Normalized Enrichment Score=2.1, p-value <0.05), while FIG. 5B shows enrichment of the genes expressing nine proteins associated with the extra cellular matrix (Normalized Enrichment Score=2.0, p-value <1E-4).

FIGS. 6A-6D depicts a PCA and pathway analysis of FM2 subgroup. FIG. 6A illustrates the PCA of the 90 control patients and the 30 patients of FM type 2 using 361 DEGs. PCA could separate the majority of Control and FM type 2 patients. Blue dots: control patients, green dots: FM2 patients, the ellipses correspond to the threshold at 80% confidence. FIG. 6B illustrates an interactome analysis of 361 DEGs that separated the FM2 using the 90 control patients as reference into two distinct clusters of up and down regulated DEGs. Blue (light grey): down regulated genes, Red (or dark grey/black): up regulated genes. FIG. 6C shows the IPA analysis and the most significant pathways found in FM2 until −log (p value) ≥3. The gene expression indicated numerous pathways downregulated and only one pathway upregulated (CLEAR signaling pathway). FIG. 6D shows a summary of biological functions related to the pathways identified in FM2 patients. Blue: downregulation, Orange (*): upregulation.

FIG. 7 shows a PCA of the control patients using the DEGs. In blue the 70 homogeneous control patients and in red the 20 patients identified as outliers.

FIGS. 8A-8B show the PCA and pathway analysis of FM3 and FM4 subgroups. FIG. 8A shows the first component axis is 29.5% and was used for separation of control patients from FM3 and FM4. Ellipses show 80% confidence interval of each group, and the supersized dots correspond to the centroid of the group. Control patients are represented by a dark blue dot, control outliers are represented by a cyan dot, FM3 are represented by a magenta dot while FM 4 are represented by an orange dot. The 4 outlier FM cases were represented by brown dots. FIG. 8B shows the most significant pathway from the IPA analysis found in FM3&4 until −log (p value) ≥2. The gene expression indicated numerous pathways associated with acute inflammatory processes are upregulated and the processing of premRNA pathway is downregulated.

### DETAILED DESCRIPTION OF THE INVENTION

### Definitions

Unless otherwise defined, all technical terms used herein have the same meaning as commonly understood by one of ordinary skill in the art to which this invention belongs.

While the following terms are believed to be well understood by one of ordinary skill in the art, the following definitions are set forth to facilitate explanation of the presently disclosed subject matter.

The term “a” or “an” refers to one or more of that entity, i.e. can refer to a plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, “protein” and “polypeptide” are used synonymously to mean any peptide-linked chain of amino acids, regardless of length or post-translational modification, e.g., glycosylation or phosphorylation.

As used herein, the term “nucleotide change” refers to, e.g., nucleotide substitution, deletion, and/or insertion, as is well understood in the art. For example, mutations contain alterations that produce silent substitutions, additions, or deletions, but do not alter the properties or activities of the encoded protein or how the proteins are made.

As used herein, the term “at least a portion” or “fragment” of a nucleic acid or polypeptide means a portion having the minimal size characteristics of such sequences, or any larger fragment of the full length molecule, up to and including the full length molecule. A fragment of a polynucleotide of the disclosure may encode a biologically active portion of a genetic regulatory element. A biologically active portion of a genetic regulatory element can be prepared by isolating a portion of one of the polynucleotides of the disclosure that comprises the genetic regulatory element and assessing activity as described herein. Similarly, a portion of a polypeptide may be 4 amino acids, 5 amino acids, 6 amino acids, 7 amino acids, and so on, going up to the full length polypeptide. The length of the portion to be used will depend on the particular application. A portion of a nucleic acid useful as a hybridization probe may be as short as 12 nucleotides; in some embodiments, it is 20 nucleotides. A portion of a polypeptide useful as an epitope may be as short as 4 amino acids. A portion of a polypeptide that performs the function of the full-length polypeptide would generally be longer than 4 amino acids.

Variant polynucleotides also encompass sequences derived from a mutagenic and recombinogenic procedure such as DNA shuffling. Strategies for such DNA shuffling are known in the art. See, for example, Stemmer (1994) PNAS 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) PNAS 94:4504-4509; Crameri et al. (1998) Nature 391:288-291; and U.S. Pat. Nos. 5,605,793 and 5,837,458.

For PCR amplifications of the polynucleotides disclosed herein, oligonucleotide primers can be designed for use in PCR reactions to amplify corresponding DNA sequences from cDNA or genomic DNA extracted from any organism of interest. Methods for designing PCR primers and PCR cloning are generally known in the art and are disclosed in Sambrook et al. (2001) Molecular Cloning: A Laboratory Manual (3^{rd} ed., Cold Spring Harbor Laboratory Press, Plainview, New York). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of primer. A pair of bi-directional primers consists of one forward and one reverse primer as commonly used in the art of DNA amplification such as in PCR amplification.

The term “cytokine” as used herein refers to small proteins that are secreted by specific cells of the immune system and glial cells, and include lymphokines, interleukins, and chemokines and their corresponding receptors, such as but not limited to IL-1, IL-2, IL-4, IL-5, IL-6, IL-7, IL-8, IL-10, IL-12, IL-13, IL-15, IL-17, IL-21, IFN-γ, IFN-α, TNF-α, IP-10, MCP-1, MIG, MIP-1α, MIP-10, GM-CSF, Eotaxin, RANTES, etc. In another aspect, the invention further includes determining the levels of one or more of IL-1RA, IL2R, IL-7, IL-12 (p40/p70), IL-13, IL-15, IL-17, IFN-α, IP-10, MIG, VEGF, G-CSF, EGF, FGF-basic and HGF. In yet another aspect, the invention also includes determining the levels of IL-9 and PDGF-BB or a combination thereof. The cytokine may be inflammatory or anti-inflammatory. In one embodiment, the cytokine to be assayed may be a full length polypeptide, protein, a glycoprotein or a fragment thereof. Other proteins that can be assayed include hormones, heat-shock proteins, antibodies such as but not limited to anti-nuclear antibody (ANA), thyroid antibodies, anti-extractable nuclear antibodies (ENA), IgG subclasses, anti-nuclear factors (FAN), rheumatoid factor (RF), receptor proteins and ligands, etc. In other embodiment, the level of cytokine assayed maybe a mRNA, miRNA, or DNA.

As used herein, the term “treatment” is defined as the application or administration of a therapeutic agent described herein, or identified by a method described herein, to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

The terms “patient”, “subject” and “individual” are used interchangeably herein, and mean any animal subject to be treated, with human patients being preferred. In some cases, the methods of the invention find use in experimental, pet or farm animals, in veterinary applications, and in the development of animal models for disease, including, but not limited to, rodents including mice, rats, canines, and hamsters, as well as primates.

By the phrases “therapeutically effective amount” and “effective dosage” is meant an amount sufficient to produce a therapeutically (e.g., clinically) desirable result; the exact nature of the result will vary depending on the nature of the disorder being treated. For example, where the disorder to be treated is fibromyalgia (FM), the result can be an alleviation of one or more symptoms of FM such as, for example, widespread pain. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the invention can include a single treatment or a series of treatments.

The term “post-biotic” as used herein can refer to microorganisms (e.g., Mycobacterium) that have been killed by heat or inactivated by other means and are delivered to an individual as inactivated, non-replicating and/or dead cells. The ideal post-biotic is one that retains all or substantially all of the properties of the live microorganism but has completely and irrevocably lost the ability to replicate itself. In some cases, the term “post-biotic” as used herein can refer to products of a microorganisms (e.g., Mycobacterium). The term “post-biotic” as used herein can refer to the waste left behind after your body digests both prebiotics and probiotics (e.g., Mycobacterium). Postbiotics as used herein can include nutrients such as vitamins B and K, amino acids, and substances called antimicrobial peptides that help to slow down the growth of harmful bacteria (e.g., Mycobacterium). Other postbiotic substances for use herein can be called short-chain fatty acids help healthy bacteria flourish.

The term “pre-biotic” as used herein can refer to materials that can act as food for probiotics (e.g., Mycobacterium) or microorganisms. Prebiotics can act as food for the probiotics. Foods with healthy amounts of fiber, such as beans, whole grains, and certain vegetables, can break down in your body to create substances that help probiotics to grow and thrive within your gut.

The term “Mycobacterium” can be used interchangeably with the term “Mycolicibacterium”. For example, “Mycobacterium smegmatis” can be referred to as “Mycolicibacterium smegmatis” interchangeably throughout.

### Overview

The present invention provides systems and methods for detecting or diagnosing fibromyalgia (FM) in an individual. In some cases, any system or method provided herein can be used to diagnose any disease that, like FM, results from an immune deficiency. Exemplary diseases that are like FM in sharing an immune deficiency can include chronic fatigue, interstitial cystitis, brain fog, sleeplessness, chronic pain, mental depression, chronic anxiety and Long COVID. In one embodiment, the detection or diagnosis of FM in the individual is used to inform treatment of FM in the individual. The treatment can be any known treatment for FM known in the art, such as, for example, known standard of care treatments for FM.

In one aspect, provided herein is a method of assaying a sample obtained from a subject or individual, the method comprising measuring a nucleic acid expression level of a plurality of biomarkers from either Table 3, Table 4, Table 5 or Table 6 in the sample obtained from the subject, wherein the subject suffers from or is suspected of suffering from fibromyalgia (FM). The method can further comprise comparing the detected levels of nucleic acid expression of the plurality of biomarkers selected from Table 3, Table 4, Table 5 or Table 6 to the expression of the plurality of biomarkers selected from Table 3, Table 4, Table 5 or Table 6 in a control and classifying the subject as having FM based on the results of the comparing step. The comparing step can comprise applying a statistical algorithm which comprises determining a correlation between the expression data obtained from the sample and the expression data from the control. In one embodiment, the nucleic acid expression level is the RNA or cDNA expression level. In some cases, the control is a reference FM sample obtained from an individual known to have FM. In some cases, the control is a non-FM sample obtained from an individual not known to have FM. The non-FM sample can be obtained from an individual diagnosed with a disease of widespread pain selected from the group consisting of rheumatoid arthritis, systemic lupus erythematosus (SLE), Sjogren Syndrome and multiple sclerosis.

In another aspect, provided herein is a method of detecting a biomarker in a sample obtained from a subject suffering from or suspected of suffering from fibromyalgia (FM), the method comprising, consisting essentially of or consisting of measuring the nucleic acid expression level of a plurality of biomarkers selected from Table 3, Table 4, Table 5 or Table 6 using an amplification, hybridization and/or sequencing assay. In one embodiment, the nucleic acid expression level is the RNA or cDNA expression level

In any of the systems or methods provided herein, the detecting the nucleic acid expression level can comprise performing quantitative real time reverse transcriptase polymerase chain reaction (qRT-PCR), RNAseq, or whole transcriptome analysis. In some cases, the nucleic acid expression level is detected by performing RNA-seq. The detection of the nucleic acid expression level can comprise using at least one pair of oligonucleotide primers specific for each biomarker from the plurality of biomarkers selected from Table 3, Table 4, Table 5 or Table 6.

In any of the systems or methods provided herein, the sample can be a bodily fluid obtained from the subject. In some cases, the bodily fluid is blood or fractions thereof, urine, saliva, or sputum.

In any of the systems or methods provided herein, the plurality of biomarkers are selected from Table 3. In some cases, the plurality of biomarkers comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers or at least 74 biomarkers from Table 3. In some cases, the plurality of biomarkers comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 3. In some cases, the plurality of biomarkers selected from Table 3 comprises biomarkers associated with the GP6 signaling pathway, the wound healing signaling pathway, RHOGDI signaling, phagosome formation, the intrinsic prothrombin activation pathway, the pulmonary fibrosis idiopathic signaling pathway, synaptic long term depression, the oxytocin signaling pathway, sperm motility or cell cycle GS/M DNA damage checkpoint regulation. In some cases, the plurality of biomarkers comprises all the biomarkers from Table 3. In some cases, a subject or subjects suffering from or suspected of suffering from fibromyalgia (FM) are those subjects whose gene expression signature as measured using a system or method provided herein reveals enrichment for gene expression of extracellular matrix (ECM) genes associated with connective tissue disorders and down regulation of genes in the Rho GDP Dissociation Inhibitor (RhoGDI) signaling pathway. In some cases, subjects whose gene signatures as determined using a method or system provided herein that show enrichment for gene expression of extracellular matrix (ECM) genes associated with connective tissue disorders and down regulation of genes in the Rho GDP Dissociation Inhibitor (RhoGDI) signaling pathway are part of the FM1 subgroup as described herein.

In any of the systems or methods provided herein, the plurality of biomarkers are selected from Table 4. In some cases, the plurality of biomarkers comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers, at least 84 biomarkers, at least 86 biomarkers, at least 88 biomarkers, at least 90 biomarkers, at least 92 biomarkers, at least 94 biomarkers, at least 96 biomarkers, at least 98 biomarkers or at least 99 biomarkers from Table 4. In some cases, the plurality of biomarkers comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 4. In some cases, the plurality of biomarkers selected from Table 4 comprise biomarkers associated with phagosome formation, the CLEAR signaling pathway, the pyroptosis signaling pathway, TREM1 signaling, the neuroinflammation signaling pathway, Th1 pathway, LPS/Il-1 mediated inhibition of RXR function, crosstalk between dendritic cells and natural killer cells, Fcgamma Receptor-mediated phagocytosis in macrophages and monocytes, production of nitric oxide and reactive oxygen species in macrophages, Toll-Like receptor signaling, inflammasome pathway, G-protein coupled receptor signaling, Th2 pathway, LXR/RXR activation, role of pattern recognition receptors in recognition of bacteria and viruses, glycolysis I or the necroptosis signaling pathway. In some cases, the plurality of biomarkers comprises all the biomarkers from Table 4. In some cases, a subject or subjects suffering from or suspected of suffering from fibromyalgia (FM) are those subjects whose gene expression signature as measured using a system or method provided herein reveals a profound reduction in the expression of inflammatory mediators and increased expression of genes involved in the Coordinated Lysosomal Expression And Regulation (CLEAR) signaling pathway. In some cases, subjects whose gene signatures as determined using a method or system provided herein reveals a profound reduction in the expression of inflammatory mediators and increased expression of genes involved in the Coordinated Lysosomal Expression And Regulation (CLEAR) signaling pathway are part of the FM2 subgroup as described herein.

In any of the systems or methods provided herein, the plurality of biomarkers are selected from Table 5. In some cases, the plurality of biomarkers comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers or at least 83 biomarkers from Table 5. In some cases, the plurality of biomarkers selected from Table 5 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 5. In some cases, the plurality of biomarkers selected from Table 5 comprise biomarkers associated with Processing of capped intron-containing pre-mRNA, the Interferon alpha/beta signaling, the Necroptosis signaling pathway, Death receptor signaling, DDX58/IFIH1-mediated induction of interferon-alpha/beta, Role of PKR in interferon induction and antiviral response, the Pyroptosis signaling pathway, ISG15 antiviral mechanism, Interferon gamma signaling, Natural killer cell signaling, Role of hyperchemokinemia in the pathogenesis of influenza, ISGylation signaling pathway, JAK/STAT signaling, Activation of IRF by cytosolic pattern recognition receptors, Interleukin-2 family signaling, RIPK1-mediated regulated necrosis or Salvage pathways of pyrimidine ribonucleotides. In some cases, the plurality of biomarkers selected from Table 5 comprises all the biomarkers from Table 5. In some cases, a subject or subjects suffering from or suspected of suffering from fibromyalgia (FM) are those subjects whose gene expression signature as measured using a system or method provided herein reveals overexpression of interferon alpha/beta and JAK/STAT pathways and downregulation of the processing of capped intron containing pre mRNA pathway. In some cases, subjects whose gene signatures as determined using a method or system provided herein reveals overexpression of interferon alpha/beta and JAK/STAT pathways and downregulation of the processing of capped intron containing pre mRNA pathway are part of the FM 3/4 subgroup as described herein.

In any of the systems or methods provided herein, the sample may have been previously diagnosed as being FM. The previous diagnosis may have been performed using any method known in the art. In one embodiment, the previous diagnosis was determined by measuring the expression level of one or more cytokines as described herein.

### Systems

In one aspect, provided herein is a system for diagnosing fibromyalgia (FM) from a sample obtained from a subject suspected of suffering from FM, the system comprising: (a) one or more processors; and (b) one or more memories operatively coupled to at least one of the one or more processors and having instructions stored thereon that, when executed by at least one of the one or more processors, cause the system to (i) detect an expression level of each of a plurality of biomarkers from Table 3, Table 4, Table 5 or Table 6; (ii) compare the expression levels of each of the plurality of biomarkers from Table 3 to the expression levels of each of the plurality of biomarkers from Table 3 in a control or compare the expression levels of each of the plurality of biomarkers from Table 4 to the expression levels of each of the plurality of biomarkers from Table 4 in a control or compare the expression levels of each of the plurality of biomarkers from Table 5 to the expression levels of each of the plurality of biomarkers from Table 5 in a control or compare the expression levels of each of the plurality of biomarkers from Table 6 to the expression levels of each of the plurality of biomarkers from Table 6 in a control; and (iii) classifying the subject as having FM based on the results of the comparing step. In some cases, the control comprises at least one sample training set(s). The at least one sample training set can comprise expression levels of each of the plurality of biomarkers from Table 3, Table 4, Table 5 or Table 6 from a reference FM sample, expression levels of each of the plurality of biomarkers from Table 3, Table 4, Table 5 or Table 6 from a reference non-FM sample or a combination thereof. The non-FM sample can be a sample obtained from an individual not known to have FM. The non-FM sample can be obtained from an individual diagnosed with a disease of widespread pain selected from the group consisting of rheumatoid arthritis, systemic lupus erythematosus (SLE), Sjogren Syndrome and multiple sclerosis. In one embodiment, the comparing step comprises applying a statistical algorithm which comprises determining a correlation between the nucleic acid expression data obtained from the sample and the expression data from the at least one training set(s); and classifying the subject as having FM based on the results of the statistical algorithm. The statistical algorithm can be any known algorithm in the art that can be used to determine correlations. In some cases, the detecting the expression level is performed using a device that is part of the system or in communication with at least one of the one or more processors, wherein upon receipt of instructions sent by the at least one of the one or more processors, perform the detection of the expression levels. In some cases, any system provided herein can be used to diagnose any disease that, like FM, results from an immune deficiency. Exemplary diseases that are like FM in sharing an immune deficiency can include chronic fatigue, interstitial cystitis, brain fog, sleeplessness, chronic pain, mental depression, chronic anxiety and Long COVID.

In some cases, the nucleic acid expression level is RNA or cDNA. Moreover, the detecting the expression level can comprise performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays or Northern blotting. In one embodiment, the expression level is detected by performing qRT-PCR.

The sample can be a bodily fluid obtained from the subject. In some cases, the bodily fluid is blood or fractions thereof, urine, saliva, or sputum.

In some cases, the plurality of biomarkers are selected from Table 3. In some cases, the plurality of biomarkers comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers or at least 74 biomarkers from Table 3. In some cases, the plurality of biomarkers comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 3. In some cases, the plurality of biomarkers selected from Table 3 comprises biomarkers associated with the GP6 signaling pathway, the wound healing signaling pathway, RHOGDI signaling, phagosome formation, the intrinsic prothrombin activation pathway, the pulmonary fibrosis idiopathic signaling pathway, synaptic long term depression, the oxytocin signaling pathway, sperm motility or cell cycle GS/M DNA damage checkpoint regulation. In some cases, the plurality of biomarkers comprises all the biomarkers from Table 3. In some cases, a subject or subjects suffering from or suspected of suffering from fibromyalgia (FM) are those subjects whose gene expression signature as measured using a system provided herein reveals enrichment for gene expression of extracellular matrix (ECM) genes associated with connective tissue disorders and down regulation of genes in the Rho GDP Dissociation Inhibitor (RhoGDI) signaling pathway. In some cases, subjects whose gene signatures as determined using a system provided herein that show enrichment for gene expression of extracellular matrix (ECM) genes associated with connective tissue disorders and down regulation of genes in the Rho GDP Dissociation Inhibitor (RhoGDI) signaling pathway are part of the FM1 subgroup as described herein.

In some cases, the plurality of biomarkers are selected from Table 4. In some cases, the plurality of biomarkers comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers, at least 84 biomarkers, at least 86 biomarkers, at least 88 biomarkers, at least 90 biomarkers, at least 92 biomarkers, at least 94 biomarkers, at least 96 biomarkers, at least 98 biomarkers or at least 99 biomarkers from Table 4. In some cases, the plurality of biomarkers comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 4. In some cases, the plurality of biomarkers selected from Table 4 comprise biomarkers associated with phagosome formation, the CLEAR signaling pathway, the pyroptosis signaling pathway, TREM1 signaling, the neuroinflammation signaling pathway, Th1 pathway, LPS/Il-1 mediated inhibition of RXR function, crosstalk between dendritic cells and natural killer cells, Fcgamma Receptor-mediated phagocytosis in macrophages and monocytes, production of nitric oxide and reactive oxygen species in macrophages, Toll-Like receptor signaling, inflammasome pathway, G-protein coupled receptor signaling, Th2 pathway, LXR/RXR activation, role of pattern recognition receptors in recognition of bacteria and viruses, glycolysis I or the necroptosis signaling pathway. In some cases, the plurality of biomarkers comprises all the biomarkers from Table 4. In some cases, a subject or subjects suffering from or suspected of suffering from fibromyalgia (FM) are those subjects whose gene expression signature as measured using a system provided herein reveals a profound reduction in the expression of inflammatory mediators and increased expression of genes involved in the Coordinated Lysosomal Expression And Regulation (CLEAR) signaling pathway. In some cases, subjects whose gene signatures as determined using a system provided herein reveals a profound reduction in the expression of inflammatory mediators and increased expression of genes involved in the Coordinated Lysosomal Expression And Regulation (CLEAR) signaling pathway are part of the FM2 subgroup as described herein.

In some cases, the plurality of biomarkers are selected from Table 5. In some cases, the plurality of biomarkers comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers or at least 83 biomarkers from Table 5. In some cases, the plurality of biomarkers selected from Table 5 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 5. In some cases, the plurality of biomarkers selected from Table 5 comprise biomarkers associated with Processing of capped intron-containing pre-mRNA, the Interferon alpha/beta signaling, the Necroptosis signaling pathway, Death receptor signaling, DDX58/IFIH1-mediated induction of interferon-alpha/beta, Role of PKR in interferon induction and antiviral response, the Pyroptosis signaling pathway, ISG15 antiviral mechanism, Interferon gamma signaling, Natural killer cell signaling, Role of hyperchemokinemia in the pathogenesis of influenza, ISGylation signaling pathway, JAK/STAT signaling, Activation of IRF by cytosolic pattern recognition receptors, Interleukin-2 family signaling, RIPK1-mediated regulated necrosis or Salvage pathways of pyrimidine ribonucleotides. In some cases, the plurality of biomarkers selected from Table 5 comprises all the biomarkers from Table 5. In some cases, a subject or subjects suffering from or suspected of suffering from fibromyalgia (FM) are those subjects whose gene expression signature as measured using a system provided herein reveals overexpression of interferon alpha/beta and JAK/STAT pathways and downregulation of the processing of capped intron containing pre mRNA pathway. In some cases, subjects whose gene signatures as determined using a system provided herein reveals overexpression of interferon alpha/beta and JAK/STAT pathways and downregulation of the processing of capped intron containing pre mRNA pathway are part of the FM 3/4 subgroup as described herein.

### Clinical Uses

In another aspect, provided herein is a method of treating fibromyalgia (FM) in a subject, the method comprising: measuring a nucleic acid expression level of a plurality of biomarkers in a sample obtained from a subject suspected of suffering from FM or any disease that, like FM, results from an immune deficiency, wherein the plurality of biomarkers is selected from Table 3, Table 4, Table 5 or Table 6, wherein the nucleic acid expression level of the plurality of biomarkers indicates that the subject has FM; and administering a standard of care for FM to the subject. Exemplary diseases that are like FM in sharing an immune deficiency can include chronic fatigue, interstitial cystitis, brain fog, sleeplessness, chronic pain, mental depression, chronic anxiety and Long COVID. The determining step can further comprise comparing the nucleic acid expression levels of the plurality of biomarkers from Table 3, Table 4, Table 5 or Table 6 to the nucleic acid expression levels of the plurality of biomarkers from Table 3, Table 4, Table 5 or Table 6 in at least one sample training set(s). The at least one sample training set can comprise nucleic acid expression level data of the plurality of biomarkers from Table 3, Table 4, Table 5 or Table 6 from a reference FM sample, nucleic acid expression level data of the plurality of biomarkers from Table 3, Table 4, Table 5 or Table 6 from a reference non-FM sample or a combination thereof. The classifying the subject as having FM can be based on the results of the comparing step. In some cases, the non-FM sample can be obtained from an individual not known to have FM. In some cases, the non-FM sample can be obtained from an individual diagnosed with a disease of widespread pain selected from the group consisting of rheumatoid arthritis, systemic lupus erythematosus (SLE), Sjogren Syndrome and multiple sclerosis. In one embodiment, the comparing step comprises applying a statistical algorithm which comprises determining a correlation between the expression data obtained from the sample obtained from the subject and the expression data from the at least one training set(s). The classifying the subject as having FM can then be based on the results of the statistical algorithm.

In some cases, the nucleic acid expression level is RNA or cDNA. Moreover, the detecting the expression level can comprise performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays or Northern blotting. In one embodiment, the expression level is detected by performing qRT-PCR.

The sample can be a bodily fluid obtained from the subject. In some cases, the bodily fluid is blood or fractions thereof, urine, saliva, or sputum.

In some cases, the plurality of biomarkers are selected from Table 3. In some cases, the plurality of biomarkers comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers or at least 74 biomarkers from Table 3. In some cases, the plurality of biomarkers comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 3. In some cases, the plurality of biomarkers selected from Table 3 comprises biomarkers associated with the GP6 signaling pathway, the wound healing signaling pathway, RHOGDI signaling, phagosome formation, the intrinsic prothrombin activation pathway, the pulmonary fibrosis idiopathic signaling pathway, synaptic long term depression, the oxytocin signaling pathway, sperm motility or cell cycle GS/M DNA damage checkpoint regulation. In some cases, the plurality of biomarkers comprises all the biomarkers from Table 3. In some cases, a subject or subjects suffering from or suspected of suffering from fibromyalgia (FM) are those subjects whose gene expression signature as measured using a method or system provided herein reveals enrichment for gene expression of extracellular matrix (ECM) genes associated with connective tissue disorders and down regulation of genes in the Rho GDP Dissociation Inhibitor (RhoGDI) signaling pathway. In some cases, subjects whose gene signatures as determined using a method or system provided herein that show enrichment for gene expression of extracellular matrix (ECM) genes associated with connective tissue disorders and down regulation of genes in the Rho GDP Dissociation Inhibitor (RhoGDI) signaling pathway are part of the FM1 subgroup as described herein.

In some cases, the plurality of biomarkers are selected from Table 4. In some cases, the plurality of biomarkers comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers, at least 84 biomarkers, at least 86 biomarkers, at least 88 biomarkers, at least 90 biomarkers, at least 92 biomarkers, at least 94 biomarkers, at least 96 biomarkers, at least 98 biomarkers or at least 99 biomarkers from Table 4. In some cases, the plurality of biomarkers comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 4. In some cases, the plurality of biomarkers selected from Table 4 comprise biomarkers associated with phagosome formation, the CLEAR signaling pathway, the pyroptosis signaling pathway, TREM1 signaling, the neuroinflammation signaling pathway, Th1 pathway, LPS/Il-1 mediated inhibition of RXR function, crosstalk between dendritic cells and natural killer cells, Fcgamma Receptor-mediated phagocytosis in macrophages and monocytes, production of nitric oxide and reactive oxygen species in macrophages, Toll-Like receptor signaling, inflammasome pathway, G-protein coupled receptor signaling, Th2 pathway, LXR/RXR activation, role of pattern recognition receptors in recognition of bacteria and viruses, glycolysis I or the necroptosis signaling pathway. In some cases, the plurality of biomarkers comprises all the biomarkers from Table 4. In some cases, a subject or subjects suffering from or suspected of suffering from fibromyalgia (FM) are those subjects whose gene expression signature as measured using a method or system provided herein reveals a profound reduction in the expression of inflammatory mediators and increased expression of genes involved in the Coordinated Lysosomal Expression And Regulation (CLEAR) signaling pathway. In some cases, subjects whose gene signatures as determined using a method or system provided herein reveals a profound reduction in the expression of inflammatory mediators and increased expression of genes involved in the Coordinated Lysosomal Expression And Regulation (CLEAR) signaling pathway are part of the FM2 subgroup as described herein.

In some cases, the plurality of biomarkers are selected from Table 5. In some cases, the plurality of biomarkers comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers or at least 83 biomarkers from Table 5. In some cases, the plurality of biomarkers selected from Table 5 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 5. In some cases, the plurality of biomarkers selected from Table 5 comprise biomarkers associated with Processing of capped intron-containing pre-mRNA, the Interferon alpha/beta signaling, the Necroptosis signaling pathway, Death receptor signaling, DDX58/IFIH1-mediated induction of interferon-alpha/beta, Role of PKR in interferon induction and antiviral response, the Pyroptosis signaling pathway, ISG15 antiviral mechanism, Interferon gamma signaling, Natural killer cell signaling, Role of hyperchemokinemia in the pathogenesis of influenza, ISGylation signaling pathway, JAK/STAT signaling, Activation of IRF by cytosolic pattern recognition receptors, Interleukin-2 family signaling, RIPK1-mediated regulated necrosis or Salvage pathways of pyrimidine ribonucleotides. In some cases, the plurality of biomarkers selected from Table 5 comprises all the biomarkers from Table 5. In some cases, a subject or subjects suffering from or suspected of suffering from fibromyalgia (FM) are those subjects whose gene expression signature as measured using a method or system provided herein reveals overexpression of interferon alpha/beta and JAK/STAT pathways and downregulation of the processing of capped intron containing pre mRNA pathway. In some cases, subjects whose gene signatures as determined using a method or system provided herein reveals overexpression of interferon alpha/beta and JAK/STAT pathways and downregulation of the processing of capped intron containing pre mRNA pathway are part of the FM 3/4 subgroup as described herein.

In some cases, the standard of care for FM is selected from the group consisting of duloxetine, pregabalin, gabapentin and milnacipran.

In some cases, the treatment can be a composition that can modulate the immune system. The modulation can be increasing the functioning of the immune system of the individual. The modulation can be decreasing the function of the immune system of the individual. The modulation can be increasing the expression of some cytokines or chemokines, while also decreasing the expression of other cytokines or chemokines. In one embodiment, provided herein is a composition comprising one or more Mycobacterial cells, wherein administration of said composition to an individual serves to modulate the functioning of the immune system of said individual. In another embodiment, provided herein is a method for improving the functioning of an individual's immune system by administering a composition provided herein, wherein the composition comprises one or more Mycobacterial cells. The one or more Mycobacterial cells present in a composition provided herein can be live-attenuated or heat-killed. In another embodiment, the one or more Mycobacterial cells present in a composition provided herein is a non-pathogenic Mycobacterial species. The non-pathogenic Mycobacterium can be live-attenuated or heat-killed. In one embodiment, the composition can be administered to a subject determined to have a FM diagnosis (e.g., FM subgroups FM1, FM2, FM3 or FM4) using a method or system provided herein. The administration, as defined herein, can include the administration of a composition provided herein in multiple aliquots and/or doses and/or on separate occasions. For example, administration can be one or times a day, semi-weekly, weekly, bi-monthly, monthly, semi-annually or annually.

In some cases case, the treatment can a composition that is formulated as a nutritional supplement, pre-biotic, probiotic and/or post-biotic. The nutritional supplement, pre-biotic, probiotic and/or post-biotic for use in treating a subject diagnosed with fibromyalgia using a method provided herein can be a nutritional supplement, pre-biotic, probiotic and/or post-biotic as described in WO2024020436A1, which is herein incorporated by reference in its entirety for all purposes. In some cases, the treatment can be a composition comprising a non-pathogenic strain of Mycobacterium selected from the group consisting of M. agri, M. brumae, M. vaccae, M. thermoresistibile, M. flavescens, M. duvalii, M. phlei, M. obuense, M. parafortuitum, M. sphagni, M. aichiense, M. rhodesiae, M. neoaurum, M. chubuense, M tokaiense, M. komossense, M. aurum, M. indicus pranii, M. tuberculosis, M. microti; M. africanum; M. kansasii, M. marinum; M. simiae; M. gastri; M. nonchromogenicum; M. terrae; M. triviale; M. gordonae; M. scrofulaceum; M. paraffinicum; M. intracellulare; M. avium; M. xenopi; M. ulcerans; M. diemhoferi, M. smegmatis; M. thamnopheos; M. flavescens; M. fortuitum; M. peregrinum; M. chelonei; M. paratuberculosis; M leprae; M. lepraemurium; M. bovis and combinations thereof that is formulated as a nutritional supplement, probiotic and/or post-biotic. In some cases, the treatment can be a composition comprising a prebiotic for a non-pathogenic strain of Mycobacterium selected from the group consisting of M. agri, M. brumae, M. vaccae, M. thermoresistibile, M. flavescens, M. duvalii, M. phlei, M. obuense, M. parafortuitum, M. sphagni, M. aichiense, M. rhodesiae, M. neoaurum, M. chubuense, M. tokaiense, M. komossense, M. aurum, M. indicus pranii, M. tuberculosis, M. microti; M. africanum; M. kansasii, M. marinum; M. simiae; M. gastri; M. nonchromogenicum; M. terrae; M. triviale; M. gordonae; M. scrofulaceum; M. paraffinicum; M. intracellulare; M. avium; M. xenopi; M. ulcerans; M. diemhoferi, M. smegmatis; M. thamnopheos; M. flavescens; M. fortuitum; M. peregrinum; M. chelonei; M. paratuberculosis; M. leprae; M. lepraemurium; M. bovis and combinations thereof. In some cases, the treatment can be a composition comprising a post-biotic obtained from a non-pathogenic strain of Mycobacterium selected from the group consisting of M. agri, M. brumae, M. vaccae, M. thermoresistibile, M. flavescens, M. duvalii, M. phlei, M. obuense, M. parafortuitum, M. sphagni, M. aichiense, M. rhodesiae, M. neoaurum, M. chubuense, M. tokaiense, M. komossense, M. aurum, M. indicus pranii, M. tuberculosis, M. microti; M. africanum; M. kansasii, M. marinum; M. simiae; M. gastri; M. nonchromogenicum; M. terrae; M. triviale; M. gordonae; M. scrofulaceum; M. paraffinicum; M. intracellulare; M. avium; M. xenopi; M. ulcerans; M. diemhoferi, M. smegmatis; M. thamnopheos; M. flavescens; M. fortuitum; M. peregrinum; M. chelonei; M. paratuberculosis; M. leprae; M. lepraemurium; M. bovis and combinations thereof. In one embodiment, the non-pathogenic strain is M. smegmatis. The non-pathogenic Mycobacterium can be live-attenuated or heat-killed. In some cases, the nutritional supplement, prebiotic, probiotic and/or post-biotic is incorporated into a food or beverage. In some cases, the food or beverage is selected from the group consisting of gums, yogurts, ice creams, cheeses, baked products, dairy and dairy substitute foods, soy-based food products, grain-based food products, starch-based food products, confectionery products, edible oil compositions, spreads, breakfast cereals, infant formulas, juices and power drinks. In some cases, the composition comprises from 2×10⁷ to 5×10⁹ colony forming units (CFUs) of the Mycobacterium. In some cases, the composition comprises at least about 5×10⁶ CFU of the Mycobacterium In some cases, the composition comprises 1 mg, 10 mg, 100 mg or 250 mg of the Mycobacterium. In one embodiment, the composition can be administered to a subject determined to have a FM diagnosis (e.g., FM subgroups FM1, FM2, FM3 or FM4) using a method or system provided herein. In one embodiment, the compositions provided herein can be formulated as probiotics. In one embodiment, the compositions provided herein can be formulated as postbiotics. In one embodiment, the compositions provided herein can be formulated as a food additive and/or food product. In one embodiment, the compositions provided herein can be formulated as a nutritional supplement or dietary supplement. In another embodiment, the compositions provided herein can be formulated as nutritional supplement, prebiotic, probiotic and/or post-biotic. The food additive or food product may be a food or beverage, The food or beverage may be selected from the group consisting of gums, yogurts, ice creams, cheeses, baked products, dairy and dairy substitute foods, soy-based food products, grain-based food products, starch-based food products, confectionery products, edible oil compositions, spreads, breakfast cereals, infant formulas, juices and power drinks. In some cases, the treatment can be a vaccine. The vaccine can be any vaccine that can induce epigenetic changes in an individual administered said vaccine. The epigenetic changes can serve to provide the individual with life-long immunity against re-occurrence of FM. The epigenetic changes can be cis-acting or trans-acting. The epigenetic changes can include changes in DNA methylation and/or histone protein modification. The vaccine can induce a chemokine and/or cytokine response in the individual. The epigenetic changes or modifications can impact the chemokine and/or cytokine responses in the individual. The vaccine composition can comprise a Mycobacterium or an antigenic fragment thereof. The Mycobacterium can be an isolated Mycobacterium or an antigenic fragment thereof. In one embodiment, the Mycobacterium is a Bacille Calmette-Guerin (BCG) strain of Mycobacterium bovis (M. bovis). Further to this embodiment, the composition can be a BCG vaccine. The BCG vaccine can be any BCG vaccine known in the art and/or commercially available. In a preferred embodiment, the BCG vaccine comprises the Tokyo 172 strain of BCG (e.g., Type I or Type II). The BCG vaccine can be live-attenuated or heat-killed. In another embodiment, the Mycobacterium is a non-pathogenic Mycobacterium species such as, for example Mycobacterium vaccae or Mycobacterium obtuense. The non-pathogenic Mycobacterium can be live-attenuated or heat-killed. The individual may have been previously diagnosed with FM or may be suspected of suffering from or being afflicted with FM.

In some of the methods provided herein, the sample obtained from a subject may have been previously diagnosed as being FM. In one embodiment, the FM diagnosis can be determined by measuring the gene expression of genes found to be associated with FM as described herein (e.g., Tables 3-6). In one embodiment, the FM diagnosis can be determined by detecting the levels of one or more cytokines in a sample obtained from the individual to see if the levels of the one or more cytokines are altered. Further to this example, to determine whether cytokine levels are altered, the cytokine levels in the individual are compared to control cytokine levels, for example, cytokine levels from a healthy patient, or cytokine levels reported for a patient without fibromyalgia (for example, levels reported in a database). In one embodiment, a positive diagnosis of fibromyalgia is provided if a majority of the cytokines tested have altered expression. In a further embodiment, a positive diagnosis of fibromyalgia is provided if at least about 67% of the cytokines tested have altered expression, or at least about 67% or more of the cytokines tested have altered expression. In a further embodiment, a positive diagnosis of fibromyalgia is provided if at least about 75%, or at least about 75% or more of the cytokines tested have altered expression. In even a further embodiment, a positive diagnosis of fibromyalgia is provided if the expression level of every cytokine tested, or about every cytokine tested in the patient is altered. In one embodiment, altered expression is determined by comparing the cytokine levels of the individual's sample to control levels. Control levels, in one embodiment, are determined by testing a sample of an individual known to not have FM. In another embodiment, control levels are known, for example, from a database. The altered level(s) of the cytokines measured in the affected individual compared to the level from control group is predictive/indicative of FM in the individual. The cytokine levels in an individual with FM, for example, cytokine levels in a FM patient's blood, in one embodiment, are higher than the cytokine levels of a healthy patient, for each cytokine tested. In another embodiment, the cytokine levels in a FM patient's blood are lower than the cytokine levels of a healthy patient, for each cytokine tested. In yet another embodiment, the cytokine levels measured in a patient with FM may be higher or lower, depending on the panel of cytokines measured in the individual.

In one embodiment, the FM diagnosis can be determined by evaluating an individual suspected of suffering from FM with the FibroFatigue scale. The FibroFatigue scale is an observer's rating scale with 12 items measuring pain, muscular tension, fatigue, concentration difficulties, failing memory, irritability, sadness, sleep disturbances, and autonomic disturbances (items derived from the CPRS) and irritable bowel, headache, and subjective experience of infection as described in Zachrisson et al., J Psychosom Res. 2002 June; 52(6):501-9, which is herein incorporated by reference in its entirety. The FibroFatigue scale can be conducted by a trained administrator. In another embodiment, the FM diagnosis can be determined by evaluating an individual suspected of suffering from FM with the FibroFatigue Scale in combination with detecting the levels of one or more cytokines in a sample obtained from the individual to see if the levels of the one or more cytokines are altered as described herein.

The present invention is not limited by any particular combination of cytokines and/or chemokines. For example, the expression levels of cytokines included in commercial cytokine/chemokine panels (or cytokine subsets thereof) can be evaluated by the methods provided herein. Various combinations of cytokines for use in the present invention are provided in Table 1 below. Subsets of these combinations may also be used in the methods provided herein. It should be understood that these combinations are representative, and should not be construed as limiting the invention.

TABLE 1
Non-limiting cytokine panels for use with the present invention.

| Panel 1 | Panel 2 | Panel 3 | Panel 4 | Panel 5 |
|---|---|---|---|---|
| IL5 | IFN-γ | TranSignal | Bio-Plex Pro | five or more |
| IL6 | IL-1β | Human Cytokine | magnetic | chemokines |
| IL8 | IL-2 | Antibody Array | Cytokine Assay | |
| IL 10 | IL-4 | 3.0 | (any of these | |
| IFN-γ | IL-5 | (or a subset of | assays may be | |
| MCP-1 | IL-6 | cytokines | used, i.e., the 8- | |
| MIP-1α | IL-8 | provided in this | plex, 17-plex, 21- | |
| MIP-1β | IL-10 | assay) | plex, 27-plex | |
| | TNF-α | | | |
| | MIP-1β | | | |
| | MCP-1 | | | |
| | MIP-1α | | | |
| | Rantes | | | |
| Panel 6 | Panel 7 | Panel 8 | Panel 9 | Panel 10 |
| IL5 | IL-6 | IL-2 | IL-8 | IFN-γ |
| IL6 | IL-8 | IL-4 | IL-10 | IL-1β |
| IL8 | IL-10 | IL-5 | TNF-α | IL-2 |
| IL 10 | TNF-α | IL-6 | MIP-1β | |
| IFN-γ | MIP-1β | | MCP-1 | |
| | MCP-1 | | Rantes | |
| | MIP-1α | | | |

The present invention also provides compositions and methods for treating an individual suffering from or suspected of suffering from conditions that share many clinical illness features with FM or any comorbid illnesses. Examples of conditions that shares clinical illness features of fibromyalgia can include any disease that results from an immune deficiency such as, for example, chronic fatigue, interstitial cystitis, brain fog, sleeplessness, chronic pain, mental depression, chronic anxiety and Long COVID. Other conditions that share many clinical illness features with FM or any comorbid illnesses can include fatigue, sleep disturbances and impairment in ability to perform activities of daily living is temporomandibular disorder (TMD). Examples of illnesses comorbid with FM can be irritable bowel syndrome (IBS), chronic tension-type headache and interstitial cystitis. The treatment can be a vaccine as provided herein.

In one embodiment, administration, as defined herein, includes the administration in multiple aliquots and/or doses and/or on separate occasions of a vaccine (e.g., Mycobacterium or an antigenic fragment thereof) or nutritional supplement, prebiotic for, probiotic and/or post-biotic obtained from a Mycobacterium as provided herein. The vaccine can be a Mycobacterium vaccine. The Mycobacterium vaccine can comprise a Mycobacterium or an antigenic fragment thereof.

In one aspect of the present invention, the Mycobacterium in a vaccine, nutritional supplement, probiotic or post-biotic as provided herein comprises a live-attenuated strain of a Mycobacterial species or an antigenic fragment thereof. In another aspect of the present invention the Mycobacterium comprises heat-killed strain of a Mycobacterial species or an antigenic fragment thereof.

Mycobacterial species for use in the present invention include, but are not limited to M. agri, M. brumae, M. vaccae, M. thermoresistibile, M. flavescens, M. duvalii, M. phlei, M. obuense, M. parafortuitum, M. sphagni, M. aichiense, M. rhodesiae, M. neoaurum, M. chubuense, M. tokaiense, M. komossense, M. aurum, M. indicus pranii, M. tuberculosis, M. microti; M. africanum; M. kansasii, M. marinum; M. simiae; M. gastri; M. nonchromogenicum; M. terrae; M. triviale; M. gordonae; M. scrofulaceum; M. paraffinicum; M. intracellulare; M. avium; M. xenopi; M. ulcerans; M. diemhoferi, M. smegmatis; M. thamnopheos; M. flavescens; M. fortuitum; M. peregrinum; M. chelonei; M. paratuberculosis; M. leprae; M. lepraemurium; M. bovis and combinations thereof. In one embodiment, the Mycobacterium is non-pathogenic. The non-pathogenic Mycobacterium can be heat-killed. The non-pathogenic Mycobacterium can be selected from M. agri, M. phlei, M. tokaiense, M. smegmatis, M. brumae, M. aurum, M. obuense and combinations thereof. In one embodiment, the non-pathogenic Mycobacterium is a rough variant. In one embodiment, the non-pathogenic Mycobacterium is a smooth variant. In one embodiment, the methods provided herein comprise administering a composition comprising M. obuense. Further to this embodiment, the M. obuense can be heat-inactivated. Further still to this embodiment, the M Obuense can be strain NCTC13365. Even further still to this embodiment, the composition comprising M. obuense is IMM-101. IMM-101 is a suspension of heat-killed whole cell Mycobacterium obuense. In one embodiment, the M. obuense can be strain ATCC27023. In one embodiment, the methods provided herein comprise administering a composition comprising M. agri. Further to this embodiment, the M. agri can be heat-inactivated. Further still to this embodiment, the M. agri can be strain ATCC27406. In one embodiment, the methods provided herein comprise administering a composition comprising M. phlei. Further to this embodiment, the M. phlei can be heat-inactivated. Further still to this embodiment, the M. phlei can be strain ATCC11758.

In one embodiment, the compositions provided herein can be used as vaccines and can accordingly be formulated as pharmaceutical compositions.

In one embodiment, administration of any vaccine, nutritional supplement, probiotic, prebiotic or post-biotic composition provided herein reduces, eliminates or alleviates one or more symptoms of FM. The one or more symptoms can be selected from chronic muscle pain, muscle spasms, or tightness, moderate or severe fatigue and decreased energy, insomnia or feeling of exhaustion, stiffness upon waking or after staying in one position for too long, difficulty remembering, concentrating, and performing simple mental tasks (“fibro fog”), abdominal pain, bloating, nausea, and constipation alternating with diarrhea (irritable bowel syndrome), tension or migraine headaches, jaw and facial tenderness, sensitivity to one or more of odors, noise, bright lights, medications, certain foods, and cold, feeling anxious or depressed, numbness or tingling in the face, arms, hands, legs, or feet, increase in urinary urgency or frequency (irritable bladder), reduced tolerance for exercise and muscle pain after exercise. A feeling of swelling (without actual swelling) in the hands and feet or a combination thereof. The reduction, elimination or alleviation can be as compared to a control. The control can be the individual prior to administration of the vaccine, nutritional supplement, probiotic, prebiotic or post-biotic as provided herein composition or a separate individual suffering from FM. In one embodiment, the reduction, elimination or alleviation of the one or more symptoms can be determined by evaluating the individual according to the Fibro-fatigue scale. In one embodiment, the reduction, elimination or alleviation of the one or more symptoms can be determined by utilizing the methods provided herein for measuring the expression levels of a plurality of biomarkers selected from Tables 3, 4, 5 and 6.

In another embodiment, administration of any vaccine, nutritional supplement, probiotic, prebiotic or post-biotic as provided herein composition provided herein increases immune system functioning. The increase in immune system functioning can be evidenced by the production or elevation thereof of T_{H}1 cytokines, upregulation of granzyme B or both. The T_{H}1 cytokines that are elevated or produced in response to administration of the compositions provided herein can include IFN-γ, IL-2, or TNF-β or a combination thereof. The increase immune system function can be evidenced by an elevation or increase in the production of one or more cytokines provided herein such as, for example, the cytokines listed in Table 1. Assessment of the alteration in immune function can be ascertained using the methods and/or kits provided herein and/or as described in US20150301062.

In one embodiment, the compositions provided herein for use in treating a disease state (e.g., fibromyalgia) diagnosed using a method provided can be formulated as pharmaceutical compositions. The pharmaceutical compositions can be formulated as vaccines. The vaccines can comprises the one or more attenuated or inactivated Mycobacterial strains or antigenic fragments thereof as well as one or more adjuvants. The adjuvants can be any adjuvants known in the art. In another embodiment, the compositions provided herein can be formulated as probiotics. In still another embodiment, the compositions provided herein can be formulated as postbiotics. In another embodiment, the compositions provided herein can be formulated as a food additive and/or food product. In still another embodiment, the compositions provided herein can be formulated as a nutritional supplement or dietary supplement. In yet another embodiment, the compositions provided herein can be formulated as a nutritional supplement, probiotic, prebiotic and post-biotic. The food additive or food product may be a food or beverage, The food or beverage may be selected from the group consisting of gums, yogurts, ice creams, cheeses, baked products, dairy and dairy substitute foods, soy-based food products, grain-based food products, starch-based food products, confectionery products, edible oil compositions, spreads, breakfast cereals, infant formulas, juices and power drinks.

Probiotic compositions as provided herein may comprise one or more Mycobacterial strains provided herein and may be formulated such that administration of the probiotic (e.g., orally, rectally, by inhalation, etc.) results in population of the individual by the Mycobacterial strains. In some embodiments, probiotic compositions are combined and/or formulated for administration to a subject. In some embodiments, probiotics contain Mycobacterial strains at known concentrations (colony forming units or CFUs). Probiotic compositions may be in the form of a pharmaceutical-type composition (e.g., capsule, tables, liquid, aerosol, etc.) or in the form of a food supplement, additive or ingredient.

In some embodiments, probiotic microbes (e.g., Mycobacterial strains provided herein) are formulated in a pharmaceutically acceptable composition for delivery to an individual. The individual may have been diagnosed as suffering from or suspected or suffering from any malady or condition provided herein. In some embodiments, probiotics are formulated with a pharmaceutically acceptable carrier suitable for a solid or semi-solid formulation. In some embodiments, probiotic microbes are formulated with a pharmaceutically acceptable carrier suitable for a liquid or gel formulation. Probiotic formulations may be formulated for enteral delivery, e.g., oral delivery, or delivery as a suppository, but can also be formulated for parenteral delivery, e.g., vaginal delivery, inhalational delivery (e.g., oral delivery, nasal delivery, and intrapulmonary delivery), and the like.

The probiotic compositions that find use in embodiments described herein may be formulated in a wide variety of oral administration dosage forms, with one or more pharmaceutically acceptable carriers. The pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material. In powders, the carrier is a finely divided solid which is a mixture with the probiotic microbes. In tablets, the microbes are mixed with the carrier having the necessary binding capacity in suitable proportions and compacted in the shape and size desired. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Other forms suitable for oral administration include liquid form preparations such as emulsions, syrups, elixirs, aqueous solutions, aqueous suspensions, or solid form preparations which are intended to be converted shortly before use to liquid form preparations. Aqueous suspensions can be prepared by dispersing the probiotic microbes in water with viscous material, such as natural or synthetic gums, resins, methylcellulose, sodium carboxymethylcellulose, and other well-known suspending agents.

The probiotic compositions (e.g., Mycobacterial strains provided herein) may be formulated for administration as suppositories. A low melting wax, such as a mixture of fatty acid glycerides or cocoa butter is first melted and the probiotic microbes are dispersed homogeneously, for example, by stirring. The molten homogeneous mixture is then poured into conveniently sized molds, allowed to cool, and to solidify.

The probiotic compositions (e.g., Mycobacterial strains provided herein) may be formulated for vaginal administration. Pessaries, tampons, creams, gels, pastes, foams or sprays, may contain agents in addition to the bacteria, such carriers, known in the art to be appropriate.

In some embodiments, probiotic compositions (e.g., Mycobacterial strains provided herein) may be formulated for delivery by inhalation. As used herein, the term “aerosol” is used in its conventional sense as referring to very fine liquid or solid particles carries by a propellant gas under pressure to a site of therapeutic application. The term “liquid formulation for delivery to respiratory tissue” and the like, as used herein, describe compositions comprising probiotic microbes with a pharmaceutically acceptable carrier in flowable liquid form. Such formulations, when used for delivery to a respiratory tissue, are generally solutions, e.g. aqueous solutions, ethanolic solutions, aqueous/ethanolic solutions, saline solutions and colloidal suspensions.

In some embodiments, postbiotic microbes (e.g., Mycobacterial strains provided herein that have been heat-killed or inactivated or live-attenuated) are formulated in a pharmaceutically acceptable composition for delivery to an individual. The individual may have been diagnosed as suffering from or suspected or suffering from any malady or condition provided herein. In some embodiments, postbiotics are formulated with a pharmaceutically acceptable carrier suitable for a solid or semi-solid formulation. In some embodiments, postbiotic microbes are formulated with a pharmaceutically acceptable carrier suitable for a liquid or gel formulation. Postbiotic formulations may be formulated for enteral delivery, e.g., oral delivery, or delivery as a suppository, but can also be formulated for parenteral delivery, e.g., vaginal delivery, inhalational delivery (e.g., oral delivery, nasal delivery, and intrapulmonary delivery), and the like.

The postbiotic compositions that find use in embodiments described herein may be formulated in a wide variety of oral administration dosage forms, with one or more pharmaceutically acceptable carriers. The pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material. In powders, the carrier is a finely divided solid which is a mixture with the postbiotic microbes. In tablets, the microbes are mixed with the carrier having the necessary binding capacity in suitable proportions and compacted in the shape and size desired. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Other forms suitable for oral administration include liquid form preparations such as emulsions, syrups, elixirs, aqueous solutions, aqueous suspensions, or solid form preparations which are intended to be converted shortly before use to liquid form preparations. Aqueous suspensions can be prepared by dispersing the postbiotic microbes in water with viscous material, such as natural or synthetic gums, resins, methylcellulose, sodium carboxymethylcellulose, and other well-known suspending agents.

The postbiotic compositions (e.g., killed or inactivated or attenuated Mycobacterial strains provided herein) may be formulated for administration as suppositories. A low melting wax, such as a mixture of fatty acid glycerides or cocoa butter is first melted and the probiotic microbes are dispersed homogeneously, for example, by stirring. The molten homogeneous mixture is then poured into conveniently sized molds, allowed to cool, and to solidify.

The postbiotic compositions (e.g., killed or inactivated or attenuated Mycobacterial strains provided herein) may be formulated for vaginal administration. Pessaries, tampons, creams, gels, pastes, foams or sprays, may contain agents in addition to the bacteria, such carriers, known in the art to be appropriate.

In some embodiments, postbiotic compositions (e.g., killed or inactivated or attenuated Mycobacterial strains provided herein) may be formulated for delivery by inhalation. As used herein, the term “aerosol” is used in its conventional sense as referring to very fine liquid or solid particles carries by a propellant gas under pressure to a site of therapeutic application. The term “liquid formulation for delivery to respiratory tissue” and the like, as used herein, describe compositions comprising probiotic microbes with a pharmaceutically acceptable carrier in flowable liquid form. Such formulations, when used for delivery to a respiratory tissue, are generally solutions, e.g. aqueous solutions, ethanolic solutions, aqueous/ethanolic solutions, saline solutions and colloidal suspensions.

In some embodiments, a postbiotic can be or can be incorporated into a comestible food or beverage or ingredient thereof. The food or beverage may be selected from the group consisting of gums, yogurts, ice creams, cheeses, baked products, dairy and dairy substitute foods, soy-based food products, grain-based food products, starch-based food products, confectionery products, edible oil compositions, spreads, breakfast cereals, infant formulas, juices and power drinks. In some embodiments, a postbiotic provided herein may be a nutritional supplement. In some embodiments, a postbiotic provided herein may be a dietary supplement. In some embodiments, a postbiotic may be a selectively fermented ingredient. Postbiotics may include complex carbohydrates, amino acids, peptides, minerals, or other essential nutritional components for the survival of the bacterial composition.

Rather than pharmaceutical-type formulation, compositions provided herein may be formulated as food additive and/or food product and incorporated into a variety of foods and beverages. In one embodiment, the one or more Mycobacterium present in the composition are all non-pathogenic strains and thus the composition can be considered as safe and non-toxic for consumption by an individual. Suitable foods and beverages include, but are not limited to gums, yogurts, ice creams, cheeses, baked products such as bread, biscuits and cakes, dairy and dairy substitute foods, soy-based food products, grain-based food products, starch-based food products, confectionery products, edible oil compositions, spreads, breakfast cereals, infant formulas, juices, power drinks, and the like.

In another embodiment, administration of any composition provided herein (e.g., a composition comprising one or more Mycobacterium provided herein such as a post-biotic composition) is immunomodulatory. In other words, the one or more Mycobacterium within the compositions provided herein can have an immunomodulatory effect on an individual administered or using said composition. In this way, the one or more Mycobacterium are immunomodulators. The immunomodulatory Mycobacterium can act to alter the immune activity of a subject directly or indirectly. For example, the immunomodulatory Mycobacterium can act directly on immune cells through receptors for bacterial components (e.g. Toll-like receptors) or by producing metabolites such as immunomodulatory short chain fatty acids (SCFAs), glutathione or gamma-glutamylcysteine.

In another embodiment, administration of any composition provided herein for treating a subject diagnosed with fibromyalgia using a method or system provided herein improves or modulates the microbiome, the Gut-Brain Axis and/or functioning of brain receptors as compared to a control. The control can be a subjected diagnosed with fibromyalgia that is not administered a composition as provided herein.

### Administration of Compositions

In certain embodiments, the compositions described herein (e.g., the immunogenic vaccine compositions) comprise, or are administered in combination with, an adjuvant. The adjuvant for administration in combination with a composition described herein may be administered before, concomitantly with, or after administration of said composition. In some embodiments, the term “adjuvant” can refer to a compound that when administered in conjunction with or as part of a composition described herein augments, enhances and/or boosts the immune response to an antigen present in the composition (e.g., the isolated Mycobacterium or antigenic fragment derived therefrom) but when the compound is administered alone does not generate an immune response to the antigen present in the composition (e.g., the isolated Mycobacterium or antigenic fragment derived therefrom). In some embodiments, the adjuvant generates an immune response to the antigen present in the composition (e.g., an isolated Mycobacterium or antigenic fragment derived therefrom) and does not produce an allergy or other adverse reaction. Adjuvants can enhance an immune response by several mechanisms including, e.g., lymphocyte recruitment, stimulation of B and/or T cells, and stimulation of macrophages. When a vaccine or immunogenic composition of the invention comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, and immunostimulatory adjuvants. Examples of adjuvants for use in the methods and compositions provided herein can include, but are not limited to, cytokines (e.g., IL-12), heat-shock proteins, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (GlaxoSmithKline), ASO4 (GlaxoSmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., in Vaccine Design: The Subunit and Adjuvant Approach (eds. Powell & Newman, Plenum Press, N Y, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al., N. Engl. J. Med. 336, 86-91 (1997)).

The compositions provided herein can comprise an antigen (e.g., an isolated Mycobacterium or antigenic fragment derived therefrom) alone or, preferably, together with a pharmaceutically acceptable carrier. Suspensions or dispersions of an antigen (e.g., an isolated Mycobacterium or antigenic fragments derived therefrom), especially isotonic aqueous suspensions or dispersions, can be used. The pharmaceutical compositions may be sterilized and/or may comprise excipients, e.g., preservatives, stabilizers, wetting agents and/or emulsifiers, solubilizers, salts for regulating osmotic pressure and/or buffers and are prepared in a manner known per se, for example by means of conventional dispersing and suspending processes. The said dispersions or suspensions may comprise viscosity-regulating agents. The suspensions or dispersions are kept at temperatures around 2-4° C., or preferentially for longer storage may be frozen and then thawed shortly before use. For injection, the vaccine or immunogenic preparations may be formulated in aqueous solutions, preferably in physiologically compatible buffers such as Hanks's solution, Ringer's solution, or physiological saline buffer. The solution may contain formulatory agents such as suspending, stabilizing and/or dispersing agents.

In certain embodiments, the compositions described herein additionally comprise a preservative, e.g., the mercury derivative thimerosal. In a specific embodiment, the pharmaceutical compositions described herein comprises 0.001% to 0.01% thimerosal. In other embodiments, the pharmaceutical compositions described herein do not comprise a preservative.

The compositions of the invention may be administered to mammals (e.g., rodents, humans) in any suitable formulation. For example, isolated Mycobacterium or antigenic fragments thereof may be formulated in pharmaceutically acceptable carriers or diluents such as physiological saline or a buffered salt solution. Suitable carriers and diluents as provided herein can be selected on the basis of mode and route of administration and standard pharmaceutical practice.

The compositions of the invention may be administered to mammals by any conventional technique. Typically, such administration will be oral, sublingual, nasal, pulmonary or parenteral (e.g., intravenous, subcutaneous, intravesicular, intramuscular, intraperitoneal, intradermal, subdermal, or intrathecal introduction). The compositions may also be administered directly to a target site by, for example, surgical delivery to an internal or external target site, or by catheter to a site accessible by a blood vessel. The compositions may be administered in a single bolus, multiple injections, or by continuous infusion (e.g., intravenously, by peritoneal dialysis, pump infusion). For parenteral administration, the compositions are preferably formulated in a sterilized pyrogen-free form.

### Dosing

The compositions (e.g., vaccine compositions, nutritional supplemental, food additives, probiotics and/or postbiotics as provided herein) described above are preferably administered to a mammal (e.g., a human) in an effective amount, that is, an amount capable of producing a desirable result in a treated individual (e.g., activating or boosting the immune response). Such a therapeutically effective amount can be determined as described below. The dosing can be as described in WO2024020436A1, which is herein incorporated by reference in its entirety for all purposes.

Toxicity and therapeutic efficacy of the compositions utilized in methods of the invention can be determined by standard pharmaceutical procedures, using either cells in culture or experimental animals to determine the LD₅₀ (the dose lethal to 50% of the population). The dose ratio between toxic and therapeutic effects is the therapeutic index and it can be expressed as the ratio LD₅₀/ED₅₀. Those compositions that exhibit large therapeutic indices are preferred. While those that exhibit toxic side effects may be used, care should be taken to design a delivery system that minimizes the potential damage of such side effects. The dosage of preferred compositions lies preferably within a range that includes an ED₅₀ with little or no toxicity. The dosage may vary within this range depending upon the dosage form employed and the route of administration utilized.

As is well known in the medical and veterinary arts, dosage for any one subject depends on many factors, including the subject's size, body surface area, age, the particular composition to be administered, time and route of administration, general health, and other drugs being administered concurrently.

In certain embodiments, a particular dosage of a composition provided herein (e.g., vaccine compositions, nutritional supplemental, food additives, probiotics and/or postbiotics as provided herein) is administered to a subject. In certain embodiments of the invention, there is provided a composition comprising a live-attenuated or heat-killed bacteria for use in the present invention, which typically may be from 10³ to 10¹¹ cells or colony forming units (CFUs), from 10⁴ to 10¹⁰ cells or CFUs, from 10⁶ to 10¹⁰ cells or CFUs, or 10⁶ to 10⁹ cells or CFUs per unit dose. The effective amount of live-attenuated or heat-killed Mycobacterium for use in the methods or compositions provided herein can be from 10³ to 10¹¹ cells or CFUs, from 10⁴ to 10¹⁰ cells or CFUs, from 10⁶ to 10¹⁰ cells or CFUs, and from 10⁶ to 10⁹ cells or CFUs per unit dose. The unit dose can be 5 ul, 10 ul, 20 ul, 30 ul, 40 ul, 50 ul, 60 ul, 70 ul, 80 ul, 90 ul, 100 ul, 125 ul, 150 ul, 175 ul, 200 ul, 250 ul, 300 ul, 350 ul, 400 ul, 450 ul, 500 ul, 600 ul, 650 ul, 700 ul, 750 ul, 800 ul, 850 ul, 900 ul, 950 ul, 1000 ul or 1500 ul. In one embodiment, the composition comprises a therapeutically effective amount of live-attenuated or heat-killed Mycobacterium (e.g., strain of BCG such as Tokyo Strain) is from 1.8×10⁶ to 3.9×10⁶ colony forming units per unit dose, wherein the unit dose is 0.1 ml. Alternatively, the dose of a vaccine composition provided herein can be from 0.01 mg to 1 mg, 0.1 mg to 0.5 mg, 0.5 mg to 1 mg, 1 mg to 1.5 mg or 1.5 mg to 2.0 mg. In one embodiment, the dose is 1 mg. In one embodiment, the dose is 0.5 mg. In one embodiment, the dose is 0.1 mg. The organisms or antigenic fragments derived therefrom can be presented as either a suspension or dry preparation. Further to the above embodiments, the route of administration can be intradermal (ID) administration.

In some embodiments, a composition (e.g., postbiotic composition and/or food product or additive or supplement) provided herein is administered over a dosing time period (e.g., <1 minute, <1 hour, <2 hours, <4 hours, <6 hours, <12 hours, <24 hours, etc.) in an amount that is sufficient to provide a desired therapeutic benefit (e.g., modulation of immune system functioning of an individual administered or using the composition). In some embodiments, the dose of the composition provided herein (e.g., post-biotic and/or nutritional supplements) is administered for the dosing time period in a concentration of from about 10 to about 1×10¹⁴ colony forming units (cfus) of the one or Mycobacterial strain(s) (e.g., 10 cfu, 100 cfu, 10¹³ cfu, 10¹³ cfu, 10¹³ cfu, 10¹³ cfu, 10¹³ cfu, 10¹³ cfu, 10¹³ cfu, 10¹³ cfu, 10¹³ cfu, 10¹³ cfu, 10¹³ cfu, 10¹³ cfu, or any suitable ranges therein (e.g., from about 10² cfu to about 10¹³ cfu, about 1×10⁴ to about 1×10¹¹ cfu, about 1×10⁶ to about 1×10⁹ cfu, about 1×10¹⁰ to about 1×10¹² cfu, etc.), etc.). In some embodiments, the dose of the composition provided herein (e.g., post-biotic and/or nutritional supplements) is administered for the dosing time period in a concentration of about 1×10¹⁰ cfus, 5×10⁹ cfus, 2.5×10⁹ cfus, 2×10⁹ cfus, 1×10⁹ cfus, 5×10⁸ cfus, 2.5×10⁸ cfus, 2×10⁸ cfus, 1×10⁸ cfus, 5×10⁷ cfus, 2.5 10⁷ cfus, 2×10⁷ cfus, 1×10⁷ cfus, 5×10⁶ cfus, 2.5×10⁶ cfus, 2×10⁶ cfus, 1×10⁶ cfus, 5×10⁵ cfus, 2.5×10⁵ cfus, 2×10⁵ cfus, 1×10⁵ cfus, 5×10⁴ cfus, 2.5×10⁴ cfus, 2×10⁴ cfus or 1×10⁴ cfus of the one or Mycobacterial strain(s). In some embodiments, the dose of the composition provided herein (e.g., post-biotic and/or nutritional supplements) is administered for the dosing time period in a concentration of about 250 mg, 100 mg, 10 mg or 1 mg of the one or Mycobacterial strain(s). In some cases, there are 5 billion (i.e., 5×10⁹ cfus) in a 250 mg dose. In some cases, there are 2 billion (i.e., 2×10⁹ cfus) in a 100 mg dose. In some cases, there are 2×10⁸ cfus in a 10 mg dose. In some cases, there are 2×10⁷ cfus in a 1 mg dose.

In certain embodiments, a particular dosage of a composition provided herein is administered to or consumed by a subject. In certain embodiments of the invention, there is provided a composition comprising a live-attenuated or heat-killed bacteria for use in the present invention, which typically may be from 10³ to 10¹¹ cells or colony forming units (CFUs), from 10⁴ to 10¹⁰ cells or CFUs, from 10⁶ to 10¹⁰ cells or CFUs, or 10⁶ to 10⁹ cells or CFUs per unit dose. The effective amount of live-attenuated or heat-killed Mycobacterium for use in the methods or compositions provided herein can be from 10³ to 10¹¹ cells or CFUs, from 10⁴ to 10¹⁰ cells or CFUs, from 10⁶ to 10¹⁰ cells or CFUs, and from 10⁶ to 10⁹ cells or CFUs per unit dose. The unit dose can be 5 ul, 10 ul, 20 ul, 30 ul, 40 ul, 50 ul, 60 ul, 70 ul, 80 ul, 90 ul, 100 ul, 125 ul, 150 ul, 175 ul, 200 ul, 250 ul, 300 ul, 350 ul, 400 ul, 450 ul, 500 ul, 600 ul, 650 ul, 700 ul, 750 ul, 800 ul, 850 ul, 900 ul, 950 ul, 1000 ul or 1500 ul. In one embodiment, the composition comprises an effective amount of live-attenuated or heat-killed Mycobacterium is from 1.8×10⁶ to 3.9×10⁶ colony forming units per unit dose. In one embodiment, the composition comprises an effective amount of live-attenuated or heat-killed Mycobacterium is from 1×10⁹ to 10×10⁹ colony forming units per unit dose. In one embodiment, the composition comprises an effective amount of live-attenuated or heat-killed Mycobacterium is from 2×10⁹ to 5×10⁹ colony forming units per unit dose. In one embodiment, the composition comprises an effective amount of live-attenuated or heat-killed Mycobacterium is from 2×10⁸ to 5×10⁹ colony forming units per unit dose. In one embodiment, the composition comprises an effective amount of live-attenuated or heat-killed Mycobacterium is from 2×10⁷ to 5×10⁹ colony forming units per unit dose. In one embodiment, the composition comprises an effective amount of live-attenuated or heat-killed Mycobacterium is from 1×10⁶ to 5×10⁶ colony forming units per unit dose. In one embodiment, the composition comprises an effective amount of live-attenuated or heat-killed Mycobacterium is from 1×10⁵ to 1×10⁶ colony forming units per unit dose. In one embodiment, the composition comprises an effective amount of live-attenuated or heat-killed Mycobacterium is from 1×10⁴ to 1×10⁵ colony forming units per unit dose. Alternatively, the dose of a composition provided herein can be from 0.01 mg to 1 mg, 0.1 mg to 0.5 mg, 0.5 mg to 1 mg, 1 mg to 1.5 mg, 1.5 mg to 2.0 mg, 2 mg to 5 mg, 5 mg to 10 mg, 10 mg to 20 mg, 20 mg to 30 mg, 30 mg to 40 mg, 40 mg to 50 mg, 50 mg to 60 mg, 60 mg to 70 mg, 70 mg to 80 mg, 80 mg to 90 mg, 90 mg to 100 mg, 100 mg to 150 mg, 150 mg to 200 mg or 200 mg to 250 mg. In one embodiment, the dose is 1 mg. In one embodiment, the dose is 0.5 mg. In one embodiment, the dose is 0.1 mg. In one embodiment, the dose is 1 mg. In one embodiment, the dose is 10 mg. In one embodiment, the dose is 100 mg. In one embodiment, the dose is 250 mg. In one embodiment, the dose is 250 mg and comprises 5×10⁹ cfus. In one embodiment, the dose is 100 mg and comprises 2×10⁹ cfus. In one embodiment, the dose is 10 mg and comprises 2×10⁸ cfus. In one embodiment, the dose is 1 mg and comprises 2×10⁷ cfus. The organisms or antigenic fragments derived therefrom can be presented as either a suspension or dry preparation.

The composition may advantageously further comprise vitamin B₁₂ and/or folacin. It has been found that a subgroup of patients suffering from fibromyalgia or chronic fatigue syndrome, may also have levels of vitamin B₁₂ in their cerebrospinal fluid that are lower than normal, and levels of homocysteine that are higher than normal.

The composition according to the invention may also comprise, such as pharmaceutically acceptable additives, e.g. solvents, adjuvants, carriers and/or preservatives as provided herein.

The methods of treatment for FM, CFS and/or related conditions as provided herein can be conducted as a series of administrations with increasing doses during a specific period. In one example, the vaccine composition can be administered in 8-10 increasing doses during 4-12 weeks, preferably 8-10 weeks. The reason for the increasing doses can be that during the first week or weeks the patient may suffer from side effects, and it is therefore advantageously to start with a low dose. The side effects may diminish after some time. In one embodiment, the vaccine composition can be administered in 2 doses spaced 4 weeks apart.

In order to obtain the desired effect for a prolonged period of time the vaccine preparation (e.g., vaccine comprising BCG) may be administered at several occasions. For example, a first series of administrations may be followed by repeated administrations given at specified intervals. The specified intervals can be approximately once a week for 5-15 weeks, preferably for 10 weeks.

To prevent recurrence, the repeated administrations may then be followed by a maintenance treatment with administrations at specified intervals. The specified intervals can be approximately once a month. The specified intervals may be continued for several years, such as 1-10 years, or approximately 5 years. In one embodiment, the maintenance treatment entails one injection of the vaccine composition per year for a specified interval of 4 years.

The doses in the repeated administrations of the maintenance treatment can be constant. In some cases, the doses in the maintenance treatment can be the dose used in the last administration in the first series.

These repeated administrations can result in an unspecific or specific activation of the immune system over a long period of time.

The administrations can be made in any way known in the art, such as, for example, injections.

Additional agents or substances can be administered simultaneously or in parallel with the vaccine compositions of the present invention. The additional agents or substance can be, for example, vitamin B12 and/or folacin.

### Assessing Treatment Efficacy

In another aspect, the invention provides methods for evaluating the efficacy of treatment in an individual diagnosed with FM. In one embodiment, the method for evaluating treatment efficacy in an individual diagnosed with FM entails subjecting the individual to the FibroFatigue scale following treatment or at various points during treatment. In one embodiment, the method for evaluating treatment efficacy in an individual diagnosed with FM entails utilizing the methods provided herein for measuring the expression levels of a plurality of biomarkers selected from Table 3 or 4. Alone or in combination with the previous embodiments, the method for evaluating the efficacy of treatment in an individual diagnosed with FM involves determining or detecting as a baseline the level of one or more cytokines expressed in the individual diagnosed with or suspecting of having FM prior to treatment. Following treatment (e.g., vaccination), subsequent measurements of one or more cytokine levels are carried out to determine the levels or patterns of expression of the one or more cytokines. The altered levels and/or patterns of expression of one or more of the cytokines measured in the individual afflicted with FM or at risk for developing FM and undergoing treatment are compared to the levels or patterns of expression of cytokines in a control. In one embodiment, the control is the levels and/or patterns of expression of the one or more cytokines in the individual before treatment. In another embodiment, the control is the levels and/or expression levels of the one or more cytokines from a healthy patient, or cytokine levels reported for a patient without fibromyalgia.

In one embodiment, the methods for assessing treatment efficacy involve determining or assaying the levels of at least one, at least two, at least three, at least four, at least five, at least six, at least seven, at least eight, at least nine, or at least ten or more cytokines in the plasma of blood samples obtained from individuals suspected of being afflicted with FM or at risk for FM after treatment with the compositions (e.g., the compositions comprising isolated Mycobacterium or antigenic fragments thereof) and comparing the levels of the assayed cytokines to a control. The control can be any control as provided herein. In a further embodiment, the method involves determining or assaying the levels of at least one, at least two, at least three, at least four, at least five, at least six, at least seven, at least eight, at least nine, or at least ten or more cytokines in the peripheral blood mononuclear cells (PBMCs) that have been separated from the plasma of blood samples obtained from the individuals after treatment and comparing the levels of the assayed cytokines to a control. The control can be any control as provided herein. These levels are then analyzed to determine if the levels are altered due to the treatment. For example, the levels in the individual's sample during and/or after treatment, in one embodiment, are compared to levels in a control sample, for example, a sample known to not have FM. In another embodiment, control levels are known, for example, from a database. In one embodiment, a change in expression in a majority of the cytokines tested toward the levels in the control is determinative/indicative of the treatment for FM being efficacious. In another embodiment, a change in expression of at least about 33% or at least about 67% of the cytokines tested is determinative/indicative of an effective treatment for FM. In a further embodiment, a treatment with a composition as provided herein is deemed to be efficacious for treating fibromyalgia if at least about 75%, or at least about 75% or more of the cytokines tested have altered expression due to the treatment. In even a further embodiment, a treatment with a composition as provided herein is deemed to be efficacious for treating fibromyalgia if the expression level of every cytokine tested, or about every cytokine tested in the patient is altered. The altered expression of one or more cytokines during or following treatment can be modifying the level or expression of the one or more cytokines to be substantially the same expression level of the one or more cytokines in a control (e.g., the levels in a healthy patient who does not have FM). As used herein, the term “substantially the same expression level” can be about 55%, about 60%, about 65%, about 70%, about 75%, about 80%, about 85%, about 90%, about 95%, about 99% or about 99% of the expression level of a particular cytokine in a control as provided herein.

The present invention is not limited by any particular combination of cytokines. For example, the cytokines to whose expression can be evaluated in order to determine treatment efficacy can be selected from IL-1, IL-2, IL-4, IL-5, IL-6, IL-7, IL-8, IL-10, IL-12, IL-13, IL-15, IL-17, IL-21, IFN-γ, IFN-α, TNF-α, IP-10, MCP-1, MIG, MIP-1α, MIP-10, GM-CSF, Eotaxin, RANTES, etc. or a combination thereof. In another aspect, the invention further includes determining the levels of one or more of IL-1RA, IL2R, IL-7, IL-12 (p40/p70), IL-13, IL-15, IL-17, IFN-α, IP-10, MIG, VEGF, G-CSF, EGF, granzyme B, FGF-basic and HGF or a combination thereof. In yet another aspect, the invention also includes determining the levels of IL-9 and PDGF-BB or a combination thereof. The cytokine may be inflammatory or anti-inflammatory. In one embodiment, the cytokine to be assayed may be a full length polypeptide, protein, a glycoprotein or a fragment thereof. Other proteins that can be assayed include hormones, heat-shock proteins, antibodies such as but not limited to anti-nuclear antibody (ANA), thyroid antibodies, anti-extractable nuclear antibodies (ENA), IgG subclasses, anti-nuclear factors (FAN), rheumatoid factor (RF), receptor proteins and ligands, etc. In other embodiment, the level of cytokine assayed maybe a mRNA, miRNA, or DNA. In another example, the expression levels of cytokines included in commercial cytokine panels (or cytokine subsets thereof) can be evaluated by the methods provided herein. Various combinations of cytokines for use in the present invention are provided in the Table 1 as provided herein. Subsets of these combinations may also be used in the methods provided herein. It should be understood that these combinations are representative, and should not be construed as limiting the invention.

### Measurement/Detection of Cytokine Levels

In one embodiment, cytokine levels in methods entailing previously diagnosing and/or assessing treatment efficacy as provided herein are tested on the protein level. In another embodiment, cytokine levels in methods entailing diagnosing and/or assessing treatment efficacy as provided herein are determined at the mRNA level. In yet another embodiment, both mRNA and protein levels for the cytokines are examined in the methods provided herein. Methods for assaying cytokines at the protein or mRNA levels are well known in the art and can be employed in the methods provided herein.

Measuring cytokine levels in methods entailing diagnosing and/or assessing treatment efficacy as provided herein can be from blood or a plasma sample that may be stimulated or un-stimulated. That is, cell proliferation may be induced prior to assaying the cytokine levels. In one embodiment, the PBMCs are un-stimulated. In another embodiment, the PBMCs are stimulated to cause proliferation of the cells prior to assaying for cytokines. Methods for stimulating PBMCs are known in the art, and include, but are not limited to, the addition of mitogens to the cells. Non-limiting examples of mitogens include lipopolysaccharide (LPS), phytohemagglutinin (PHA), or phorbol ester, such as phorbol myristate acetate (PMA) with or without ionomycin, pokeweed mitogen (PWM), concavalin A (Con-A), or combinations thereof.

In one embodiment, cytokine expression is measured at the mRNA level, for example, by quantitative RT-PCR (also known as real time RT-PCR). mRNA expression levels can also be measured by Northern blot assay, array hybridization, sequencing, etc. For example, multiplex quantitative RT-PCR, in one embodiment, is carried out to determine the mRNA expression levels of a cytokine panel. Cytokine RT-PCR kits are commercially available, for example, from Roche.

In another embodiment, secreted cytokine levels are determined (i.e., at the protein level). In one embodiment, secreted cytokine levels are determined by using an antibody array, for example, the TranSignal Human Cytokine Antibody Array 3.0, available from Panomics. The Panomics array includes antibodies directed to the following cytokines: Apol/Fas, Leptin, Rantes, ICAM-1, IL-2, IL-7, CTLA, MIP-1α, MIP-10, TGFβ, VCAM-1, IL-3, IL-8, IL-4, IL-10, IL-5, IL-12, IL-6, IL-15, IL-6R, IL-17, IL-1Rα, IL-1β, IL-1α, VEGF, IFNγ, TNFα, TNFRI, TNFRII, MIP-5, MIP-4, MMP3, Eotaxin, GM-CSF, EGF, IP-10. In this embodiment, not all cytokines in the array need be probed for. For example, the expression levels of a subset of five cytokines, or five or more cytokines, or six cytokines, or six or more cytokines, or seven cytokines, or seven or more cytokines, or ten cytokines, or ten or more cytokines, or twelve cytokines, or twelve or more cytokines may be determined when carrying out the methods of the invention.

Secreted cytokine levels, in one embodiment, are determined with a multiplex immunoassay built on magnetic beads. For example, in one embodiment, the Bio-Plex Pro magnetic Cytokine Assay is used (Bio-Rad). In this embodiment, the Assay is commercially available as a ready to use kit, for example, for the detection of eight cytokines, seventeen cytokines, 21 cytokines or 27 cytokines. The full number or a subset of the cytokines may be detected in the methods of the invention. Alternatively, expression levels of cytokines can be tested in a sample by doing multiple assays on the sample, for example, in “singleplex” format. In one embodiment, the Bio-Rad singleplex cytokine assays are used.

Another antibody based bead assay is available from Invitrogen, and is also amenable to be used in the methods of the present invention. Specifically, the Human Cytokine Thirty-Plex antibody bead kit may be employed to detect the levels of a panel of cytokines in an individual. Although the assay can detect the levels of thirty cytokines, not all thirty need to be detected in order to carry out the methods provided herein. For example, as provided above, five, six, seven, eight, nine, ten, eleven or twelve cytokines can be assayed for their expression levels. The Invitrogen kit comprises analyte specific components for the measurement of human IL-1β, IL-1RA, IL-2, IL-2R, IL-4, IL-5, IL-6, IL-7, IL-8, IL-10, IL-12p40/p70, IL-13, IL-15, IL-17, TNF-α, IFN-α, IFN-γ, GM-CSF, MIP-1α, MIP-10, IP-10, MIG, Eotaxin, RANTES, MCP-1, VEGF, G-CSF, EGF, FGF-basic, and HGF. These reagents, in one embodiment, are used in the Luminex® 100™ or 200™ System.

Methods for assaying cytokines at the protein or mRNA levels are well known in the art. Besides the assays provided above, other non-limiting examples of methods for assaying cytokines at the protein level include enzyme-linked immunoassay (ELISA), Tetramer assay, ELISPOT assay, Fluorospot assay, etc. The cytokines concentration in the plasma, culture supernatant, or cell lysate derived from PBMC can be measured, for example, by multiplex immunoassay based on Luminex xMAP bead array technology, or Bio-Plex 200 fluorescence bead reader (BioRad Laboratories, Hercules, CA). In one embodiment, the level of one or more cytokine mRNA can be detected (measured) by real time PCR, RT-PCR, Northern blot assay, array hybridization, sequencing, etc. The altered level(s) of the cytokines measured in the affected individual compared to the level from control group is predictive/indicative of FM in the individual. The cytokine levels in an individual with FM, for example, cytokine levels in a FM patient's blood, in one embodiment, are higher than the cytokine levels of a healthy patient, for each cytokine tested. In another embodiment, the cytokine levels in a FM patient's blood are lower than the cytokine levels of a healthy patient, for each cytokine tested. In yet another embodiment, the cytokine levels measured in a patient with FM may be higher or lower, depending on the panel of cytokines measured in the individual.

In one aspect, the methods provided herein further include evaluation of the individual's (i.e., individuals diagnosed with or suspected of suffering from FM) clinical and physical symptoms. For example, the method can include evaluation of physical and mental functioning as well as tender points in the individual. The physical and mental functioning as well as the pain threshold can be calculated and assigned a score on a subjective basis. The level of daily activity as well as the physical and mental functioning can be calculated and assigned a score on a subjective basis. The scores derived from the assessment of the clinical and physical symptoms may be included in the statistical analysis for the cytokines. In a further embodiment, the method includes determining the levels of various factors or markers, such as but not limited to Rheumatoid Factor (RF), or a specific marker of inflammation such as the erythrocyte sedimentation rate (ESR).

As it relates to the diagnostic and/or assessment of treatment efficacy methods provided herein, cytokine and/or gene expression can be “altered” or “differentially expressed”, in an individual, in one embodiment, if expression in the individual's sample is at least about 1.5 times higher or lower than the expression of the same cytokine or gene at a control level. In another embodiment, cytokine or gene expression is “altered” if cytokine or gene expression in the individual's sample is at least about 2 times higher or lower than the expression of the same cytokine or gene at a control or baseline level (i.e., levels reported for a healthy patient). In another embodiment, cytokine or gene expression is “altered” if cytokine or gene expression in the individual is at least about 2.5 times higher or lower (or at least about 2.5 times or more higher or lower) than the control expression level of the same cytokine or gene. In yet another embodiment, cytokine or gene expression is “altered” if cytokine or gene expression in the individual is at least about 3 times higher or lower (or at least about 3 times or more higher or lower) than the control expression level of the same cytokine or gene. In another embodiment, cytokine or gene expression is “altered” if cytokine or gene expression in the individual is at least about 5 times higher or lower (or at least about 5 times or more higher or lower) than the control gene expression level of the same cytokine. In even another embodiment, cytokine or gene expression is “altered” if cytokine or gene expression in the individual is at least about 10 times higher or lower than the control expression level of the same cytokine or gene in a control sample. In yet another embodiment, cytokine or gene expression is altered if cytokine or gene expression in the individual is at least about 10 times or more, higher or lower, than the control expression level of the same cytokine or gene. As provided above, control expression level may be determined from values in a database, from a non-disease sample (e.g., FM) or individual.

Altered expression of the cytokine or gene may be the same or different for each individual cytokine or gene that is differentially expressed. For example, the expression of one cytokine (mRNA or protein) may be 2× lower, or about 2× lower, than the expression of the same cytokine in a control sample, while the expression of a second cytokine may be 1.5× lower, or about 1.5× lower, than the expression of the same cytokine in a control sample. As discussed above, altered expression includes both higher and lower expression of the cytokine, compared to a control level.

### Statistical Methods

Various statistical methods can be used to aid in the comparison of the biomarker levels obtained from the patient and reference biomarker levels, for example, from at least one sample training set as provided herein.

In one embodiment, a supervised pattern recognition method is employed. Examples of supervised pattern recognition methods can include, but are not limited to, the nearest centroid methods (Dabney (2005) Bioinformatics 21(22):4148-4154 and Tibshirani et al. (2002) Proc. Natl. Acad. Sci. USA 99(10):6576-6572); soft independent modeling of class analysis (SIMCA) (see, for example, Wold, 1976); partial least squares analysis (PLS) (see, for example, Wold, 1966; Joreskog, 1982; Frank, 1984; Bro, R., 1997); linear descriminant analysis (LDA) (see, for example, Nillson, 1965); K-nearest neighbour analysis (KNN) (sec, for example, Brown et al., 1996); artificial neural networks (ANN) (see, for example, Wasserman, 1989; Anker et al., 1992; Hare, 1994); probabilistic neural networks (PNNs) (see, for example, Parzen, 1962; Bishop, 1995; Speckt, 1990; Broomhead et al., 1988; Patterson, 1996); rule induction (RI) (see, for example, Quinlan, 1986); and, Bayesian methods (see, for example, Bretthorst, 1990a, 1990b, 1988).

In other embodiments, an unsupervised training approach is employed, and therefore, no training set is used.

Referring to sample training sets for supervised learning approaches again, in some embodiments, a sample training set(s) can include expression data of a plurality or all of the biomarkers (e.g., the biomarkers of Table 3, Table 4, Table 5 or Table 6) as measured in a sample obtained from a a subject suspected of suffering from FM or previously diagnosed with FM. In some embodiments, the sample training set(s) are normalized to remove sample-to-sample variation. The normalization can be done using any housekeeping gene known in the art, such as, for example, GAPDH and/or beta-actin.

In some embodiments, comparing can include applying a statistical algorithm, such as, for example, any suitable multivariate statistical analysis model, which can be parametric or non-parametric. In some embodiments, applying the statistical algorithm can include determining a correlation between the expression data obtained from the sample obtained from the subject and the expression data from the training set(s). In some embodiments, cross-validation is performed, such as (for example), leave-one-out cross-validation (LOOCV). In some embodiments, integrative correlation is performed. In some embodiments, a Spearman correlation is performed. In some embodiments, a centroid based method is employed for the statistical algorithm as described in Mullins et al. (2007) Clin Chem. 53(7):1273-9, and based on gene expression data, which is herein incorporated by reference in its entirety.

Results of the gene expression performed on a sample from a subject (test sample) may be compared to a biological sample(s) or data derived from a biological sample(s) that is known or suspected to be normal (“reference sample” or “normal sample”, e.g., non-FM sample). In some embodiments, a reference sample or reference gene expression data is obtained or derived from an individual known to have FM.

The reference sample may be assayed at the same time, or at a different time from the test sample. Alternatively, the biomarker level information from a reference sample may be stored in a database or other means for access at a later date.

The biomarker level results of an assay on the test sample may be compared to the results of the same assay on a reference sample. In some cases, the results of the assay on the reference sample are from a database, or a reference value(s). In some cases, the results of the assay on the reference sample are a known or generally accepted value or range of values by those skilled in the art. In some cases, the comparison is qualitative. In other cases, the comparison is quantitative. In some cases, qualitative or quantitative comparisons may involve but are not limited to one or more of the following: comparing fluorescence values, spot intensities, absorbance values, chemiluminescent signals, histograms, critical threshold values, statistical significance values, expression levels of the genes described herein, mRNA copy numbers.

In one embodiment, an odds ratio (OR) is calculated for each biomarker level panel measurement. Here, the OR is a measure of association between the measured biomarker values for the patient and an outcome, e.g., FM diagnosis. For example, see, J. Can. Acad. Child Adolesc. Psychiatry 2010; 19(3): 227-229, which is incorporated by reference in its entirety for all purposes.

In one embodiment, a specified statistical confidence level may be determined in order to provide a confidence level regarding the FM diagnosis. For example, it may be determined that a confidence level of greater than 90% may be a useful predictor of the FM diagnosis. In other embodiments, more or less stringent confidence levels may be chosen. For example, a confidence level of about or at least about 50%, 60%, 70%, 75%, 80%, 85%, 90%, 95%, 97.5%, 99%, 99.5%, or 99.9% may be chosen. The confidence level provided may in some cases be related to the quality of the sample, the quality of the data, the quality of the analysis, the specific methods used, and/or the number of gene expression values (i.e., the number of genes) analyzed. The specified confidence level for providing the likelihood of response may be chosen on the basis of the expected number of false positives or false negatives. Methods for choosing parameters for achieving a specified confidence level or for identifying markers with diagnostic power include but are not limited to Receiver Operating Characteristic (ROC) curve analysis, binormal ROC, principal component analysis, odds ratio analysis, partial least squares analysis, singular value decomposition, least absolute shrinkage and selection operator analysis, least angle regression, and the threshold gradient directed regularization method.

Determining the FM diagnosis in some cases can be improved through the application of algorithms designed to normalize and/or improve the reliability of the gene expression data. In some embodiments of the present invention, the data analysis utilizes a computer or other device, machine or apparatus for application of the various algorithms described herein due to the large number of individual data points that are processed. A “machine learning algorithm” refers to a computational-based prediction methodology, also known to persons skilled in the art as a “classifier,” employed for characterizing a gene expression profile or profiles, e.g., to determine the FM diagnosis. The biomarker levels, determined by, e.g., microarray-based hybridization assays, sequencing assays, NanoString assays, etc., are in one embodiment subjected to the algorithm in order to classify the profile. Supervised learning generally involves “training” a classifier to recognize the distinctions among FM positive and FM negative samples, and then “testing” the accuracy of the classifier on an independent test set. Therefore, for new, unknown samples the classifier can be used to predict, for example, the class (e.g., FM positive or FM negative) in which the samples belong.

In some embodiments, a robust multi-array average (RMA) method may be used to normalize raw data. The RMA method begins by computing background-corrected intensities for each matched cell on a number of microarrays. In one embodiment, the background corrected values are restricted to positive values as described by Irizarry et al. (2003). Biostatistics April 4 (2): 249-64, incorporated by reference in its entirety for all purposes. After background correction, the base-2 logarithm of each background corrected matched-cell intensity is then obtained. The background corrected, log-transformed, matched intensity on each microarray is then normalized using the quantile normalization method in which for each input array and each probe value, the array percentile probe value is replaced with the average of all array percentile points, this method is more completely described by Bolstad et al. Bioinformatics 2003, incorporated by reference in its entirety. Following quantile normalization, the normalized data may then be fit to a linear model to obtain an intensity measure for each probe on each microarray. Tukey's median polish algorithm (Tukey, J. W., Exploratory Data Analysis. 1977, incorporated by reference in its entirety for all purposes) may then be used to determine the log-scale intensity level for the normalized probe set data.

Various other software programs may be implemented. In certain methods, feature selection and model estimation may be performed by logistic regression with lasso penalty using glmnet (Friedman et al. (2010). Journal of statistical software 33(1): 1-22, incorporated by reference in its entirety). Raw reads may be aligned using TopHat (Trapnell et al. (2009). Bioinformatics 25(9): 1105-11, incorporated by reference in its entirety). In methods, top features (N ranging from 10 to 200) are used to train a linear support vector machine (SVM) (Suykens J A K, Vandewalle J. Least Squares Support Vector Machine Classifiers. Neural Processing Letters 1999; 9(3): 293-300, incorporated by reference in its entirety) using the e1071 library (Meyer D. Support vector machines: the interface to libsvm in package e1071. 2014, incorporated by reference in its entirety). Confidence intervals, in one embodiment, are computed using the pROC package (Robin X, Turck N, Hainard A, et al. pROC: an open-source package for R and S+ to analyze and compare ROC curves. BMC bioinformatics 2011; 12: 77, incorporated by reference in its entirety).

In addition, data may be filtered to remove data that may be considered suspect. In one embodiment, data derived from microarray probes that have fewer than about 4, 5, 6, 7 or 8 guanosine+cytosine nucleotides may be considered to be unreliable due to their aberrant hybridization propensity or secondary structure issues. Similarly, data deriving from microarray probes that have more than about 12, 13, 14, 15, 16, 17, 18, 19, 20, 21, or 22 guanosine+cytosine nucleotides may in one embodiment be considered unreliable due to their aberrant hybridization propensity or secondary structure issues.

In some embodiments of the present invention, data from probe-sets may be excluded from analysis if they are not identified at a detectable level (above background).

In some embodiments of the present disclosure, probe-sets that exhibit no, or low variance may be excluded from further analysis. Low-variance probe-sets are excluded from the analysis via a Chi-Square test. In one embodiment, a probe-set is considered to be low-variance if its transformed variance is to the left of the 99 percent confidence interval of the Chi-Squared distribution with (N−1) degrees of freedom. (N−1)*Probe-set Variance/(Gene Probe-set Variance). Chi-Sq(N−1) where N is the number of input CEL files, (N−1) is the degrees of freedom for the Chi-Squared distribution, and the “probe-set variance for the gene” is the average of probe-set variances across the gene. In some embodiments of the present invention, probe-sets for a given mRNA or group of mRNAs may be excluded from further analysis if they contain less than a minimum number of probes that pass through the previously described filter steps for GC content, reliability, variance and the like. For example, in some embodiments, probe-sets for a given gene or transcript cluster may be excluded from further analysis if they contain less than about 1, 2, 3, 4, 5, 6, 7, 8, 9, 10, 11, 12, 13, 14, 15, or less than about 20 probes.

Methods of biomarker level data analysis in one embodiment, further include the use of a feature selection algorithm as provided herein. In some embodiments of the present invention, feature selection is provided by use of the LIMMA software package (Smyth, G. K. (2005). Limma: linear models for microarray data. In: Bioinformatics and Computational Biology Solutions using R and Bioconductor, R. Gentleman, V. Carey, S. Dudoit, R. Irizarry, W. Huber (eds.), Springer, New York, pages 397-420, incorporated by reference in its entirety for all purposes).

Methods of biomarker level data analysis, in one embodiment, include the use of a pre-classifier algorithm. For example, an algorithm may use a specific molecular fingerprint to pre-classify the samples according to their composition and then apply a correction/normalization factor. This data/information may then be fed into a final classification algorithm which would incorporate that information to aid in the final diagnosis.

Methods of biomarker level data analysis, in one embodiment, further include the use of a classifier algorithm as provided herein. In one embodiment of the present invention, a diagonal linear discriminant analysis, k-nearest neighbor algorithm, support vector machine (SVM) algorithm, linear support vector machine, random forest algorithm, or a probabilistic model-based method or a combination thereof is provided for classification of microarray data. In some embodiments, identified markers that distinguish samples are selected based on statistical significance of the difference in biomarker levels between classes of interest. In some cases, the statistical significance is adjusted by applying a Benjamin Hochberg or another correction for false discovery rate (FDR).

In some cases, the classifier algorithm may be supplemented with a meta-analysis approach such as that described by Fishel and Kaufman et al. 2007 Bioinformatics 23(13): 1599-606, incorporated by reference in its entirety for all purposes. In some cases, the classifier algorithm may be supplemented with a meta-analysis approach such as a repeatability analysis.

Methods for deriving and applying posterior probabilities to the analysis of biomarker level data are known in the art and have been described for example in Smyth, G. K. 2004 Stat. Appi. Genet. Mol. Biol. 3: Article 3, incorporated by reference in its entirety for all purposes. In some cases, the posterior probabilities may be used in the methods of the present invention to rank the markers provided by the classifier algorithm.

A statistical evaluation of the results of the biomarker level profiling may provide a quantitative value or values indicative of one or more of the following: FM diagnosis; the likelihood of the success of a particular therapeutic intervention. In one embodiment, the data is presented directly to the physician in its most useful form to guide patient care or is used to define patient populations in clinical trials or a patient population for a given medication. In this way, the biomarker level profiling methods provided herein serve as a therapeutic response signature. The results of the molecular profiling can be statistically evaluated using a number of methods known to the art including, but not limited to: the students T test, the two sided T test, Pearson rank sum analysis, hidden Markov model analysis, analysis of q-q plots, principal component analysis, one way ANOVA, two way ANOVA, LIMMA and the like.

In some cases, accuracy may be determined by tracking the subject over time to determine the accuracy of the original diagnosis. In other cases, accuracy may be established in a deterministic manner or using statistical methods. For example, receiver operator characteristic (ROC) analysis may be used to determine the optimal assay parameters to achieve a specific level of accuracy, specificity, positive predictive value, negative predictive value, and/or false discovery rate.

### EXAMPLES

The present invention is further illustrated by reference to the following Examples. However, it should be noted that these Examples, like the embodiments described above, are illustrative and are not to be construed as restricting the scope of the invention in any way.

### Example 1: Identification of Genomic Signatures in FM Patients

### Introduction and Objective

For more than 100 years a debate has raged over whether individuals who have had chronic, non-remitting pain, body area tenderness, persistent fatigue, recurrent headaches, “brain fog,” generalized anxiety, chronic depression, poor sleep, leg cramps, numbness and tingling, difficulty concentrating and restless legs while sleeping define an actual medical disease or merely a collection of symptoms. The first name assigned to this collection of medical complaints was that of fibrositis, a description which persisted until the late 20^{th} century. It was at that time when the American College of Rheumatology began labeling patients as experiencing fibromyalgia and claiming that it was nothing more than a “syndrome,” which by definition is only a collection of symptoms. It was not until 2013, that fibromyalgia was commonly accepted to be an actual disease. This arose after Behm, F. G. et al. (6) demonstrated in a large study of fibromyalgia patients versus matched healthy controls that fibromyalgia actually was an immune deficiency disorder wherein peripheral blood mononuclear cells are incapable of producing normal quantities of certain chemokine and cytokine proteins. Their findings led to a related blood test for diagnosing fibromyalgia. Subsequent research by Wallace, D. et al: (7) documented and proved in a second and separate laboratory the accuracy of this diagnostic methodology which confirmed that the diagnostic biomarkers in fibromyalgia (FM) were indeed unique and they did not occur in the most common rheumatic diseases of rheumatoid arthritis and systemic lupus erythematosus.

Nevertheless, there was and has been a persistent refusal to acknowledge the existence and accuracy of the aforementioned diagnostic criteria despite the extremely high levels of sensitivity and specificity that was demonstrated via the aforementioned peer-reviewed and published diagnostic methodology that revealed the underlying immune system deficiencies that define fibromyalgia.

In 2021, Gillis, et al (50), showed that the specific immune deficiencies in fibromyalgia patients as pertained to the cytokines of IL-6 and IL-8 documented that these patients were not susceptible to the cytokine storm effects induced by a COVID-19 infection. Their recommendation to therefore utilize IL-6 antagonists for the treatment of COVID-19 was thereafter acknowledged by the United States Food and Drug Administration.

Presently, there remains a persistent refusal to recognize the uniqueness of fibromyalgia and only begrudgingly do healthcare professionals show a partial acceptance of the existence of this medical disease. Therefore, it was the goal of this Example to determine the existence of a genomic definition of FM in order unequivocally demonstrate and define this disease, as well as serve as a basis to develop targeted therapies. To accomplish this goal, the diagnostic assay developed by Behm, et al. (6), was used to identify which patients met the definition of fibromyalgia, followed by comparing them via age, gender, ethnic and medical histories against a matched set of healthy individuals.

### Materials and Methods

### Study Participants

This study was performed with the approval of the institutional review board of the University of Illinois at Chicago (Office for the Protection of Research Subjects, OPRS) and all methods were performed in accordance with the relevant guidelines and regulations. The study groups consisted of 96 FM patients (91 females and 5 males) and 93 control cases (41 females and 52 males). The participants (FM and controls) included in this study did not overlap with the participants from the previous study published in 2012 (6). All participants provided written informed consent. The inclusion criteria for FM followed the 2016 criteria of the American College of Rheumatology (3-5) and had a positive fibromyalgia assay (FM/a) (3,6). The control group did not fulfill the 2016 criteria of the College of Rheumatology and had a negative fibromyalgia assay (FM/a). The FM/a included expression analysis of four cytokines, IL6, IL8, Mip1-α/CCL3 and Mip1-β/CCL4 and relied on the functioning of viable PBMC as previously described(6). Exclusion criteria, both for patients and controls, were the presence of any other chronic disease (diabetes, heart disease or cancer). A questionnaire was used for the collection of demographic and clinical data from participants. None of the patients were treated with anti-inflammatory drugs at the time and 3 months before the start of the study. The median age of patients participating in this study was 48 years of age with overall ages ranging from 28 years to 77 years. The median age of the onset of fibromyalgia was 36 years of age. The median age of the control patients was 39, with ages ranging from 20 years to 69 years. The patients were recruited based on their symptoms related to chronic body pain and tenderness as well as chronic fatigue, a sleep disorder, anxiety, joint aches, headaches, leg cramps during sleep, areas of numbness or tingling, headaches, trouble concentrating, “brain fog” and depression. The clinical characteristics of the entire cohort is presented in Table 2.

TABLE 2
Clinical characteristics of FM and control groups.

| | | |
|---|---|---|
| Clinical Features | FM group (96) | Control group (93) |

| | | |
|---|---|---|
| Gender | F- 91, M-5 | F-43, M-50 |
| Age | Median-48 | Median-45 |
| | (range 28-77) | (range 20-69) |
| Age of onset/diagnosis | Median-36 | N/A |
| | (range 12-66) | |

| Muscle/Body pain | 93 | (97%) | 0 | |
|---|---|---|---|---|
| Tender areas | 91 | (95%) | 0 | |
| Chronic fatigue | 92 | (96%) | 4 | (4%) |
| Sleep disorder | 85 | (88.5%) | 4 | (4%) |
| Anxiety | 77 | (80%) | 11 | (12%) |
| Joint aches | 84 | (87.5%) | 6 | (6.5%) |
| Frequent headaches | 56 | (58%) | 2 | (2%) |
| Restless legs/Leg cramps | 69 | (72%) | 5 | (5.4%) |
| Numbness or tingling | 74 | (77%) | 6 | (6.5%) |
| Trouble remembering | 87 | (90.6%) | 0 | |
| Trouble concentrating | 90 | (94%) | 8 | (8.6%) |
| Depression | 64 | (67%) | 10 | (10.8%) |

Samples from participants were selected based on the expression analysis of four cytokines including IL-6, IL-8, MIP-1 alpha (CCL3) and MIP-1 beta (CCL4) with all participants being evaluated using a cytokine assay (i.e., FM/a test; EpicGenetics), which confirmed the diagnosis of FM (6). Since the cytokine assay relied upon an analysis of the functioning of peripheral blood mononuclear cells (PBMCs), said cells were harvested, put through centrifugation, suspended in a specific medium, cultured in that medium and then the related supernatant was removed. Thereafter, cytokine and chemokine concentrations in the plasma, as well as in culture supernatants, were determined using a multiplex immunoassay based upon the Luminex xMAP Bead Array technology.

### Sample Collection, RNTA Extraction, Library Construction and Sequencing

For genomic analyses, blood samples (9-10 mL) from FM and control individuals were collected in Streck tubes (Streck, La Vista, NE). These samples were centrifuged at 2500 rpm to separate the plasma, PBMC and RBC layers. After removing the plasma, the RBCs were lysed using Qiagen RBC lysis buffer and centrifuged for 20 minutes at 2500 rpm at room temperature. Cell pellets were washed in PBS twice to remove any trace of RBC, then homogenized using a QIAshredder homogenizer in 600 μL RLT buffer containing β-mercaptoethanol. Total RNA was extracted using a QIAamp RNA blood mini kit (Qiagen, Germantown, MD). RNA quality was assessed using Agilent RNA screen tape and TapeStation 4200 (Agilent Technologies, Santa Clara, CA) and a nanodrop spectrophotometer was used to estimate the concentration and purity of RNA (Nano-Drop Technologies, Wilmington, DE).

RNA libraries were prepared using the Agilent SureSelectXT RNA Direct workflow following manufacturer's instructions. Briefly, 200 ng of RNA was transferred into a strip tube and the volume was adjusted using nuclease-free water. Samples were completely dried at 30° C. in a vacufuge (Eppendorf) and RNA-seq fragmentation mix was then added to each sample, mixed by vortexing gently at 2000 rpm for 10 second. Fragmentation was carried out on a thermal cycler using the following program with a heated lid on at −94° C. (8 min), 4° C. (1 min) and kept at 4° C. (∞). RNA-seq first strand master mix was added to each sample. The first strand was synthesized using the following pathway: thermal cycling condition at 25° C. (10 min), 37° C. (40 min) and then maintained at 4° C. (∞) with a heated lid on. The first stand was purified using Agencourt AMPure XP beads (Beckman Coulter Genomics). Second-strand cDNA was synthesized and end repaired following a thermal cycling condition at 16° C. (1 h) and maintained at 4° C. (∞) without a heated lid. The second-strand cDNA was purified using Agencourt AMPure beads. The 3′ ends of cDNA were dA-tailed following a thermal cycling condition at 37° C. (30 min) and maintained at 4° C. (∞) without a heated lid. Adaptors were ligated to each dA-tailed cDNA following a thermal cycling condition of 20° C. (15 min) and maintained at 4° C. (∞) without a heated lid. The adaptor ligated cDNA was purified using Agencourt AMPure beads and was amplified using pre-capture thermal cycling conditions and purified using Agencourt AMPure beads. The quality of a Pre-captured library was assessed using D1000 ScreenTape on a 4200 Tapestation system. The region between 150-400 bp was used for quantification. Hybridization was carried out overnight at 65° C. on a thermal cycler using 200 ng of pre-captured library. For capturing the targets, SureSelectXT Human All Exon V6 baits were used. The captured libraries were then amplified to add the index tags and were purified using Agencourt AMPure beads and finally eluted in a low TE buffer. The quality and quantity (region of 150-500 bp) of the libraries were assessed using high sensitivity D1000 screen tape on a 4200 Tapestation system. Paired end sequencing of the libraries were sequenced at a 2 ng/μL concentration on a NovaSeq 6000 system at 25 libraries/S4 flow cell (Illumina, San Diego, CA), with an average coverage of 136 million reads per exome and a read length of 2×150 bp. Raw data have been submitted to NCBI and GSE221921.

### Data Analysis

FASTQ files corresponding to the forward and reverse reads for 189 samples in total, 96 FM and 93 control were obtained from Basespace and used for analysis. The files were processed using the Trim Galore (Babraham Bioinformatics) and Cutadapt (DOI:10.14806/ej.17.1.200) tools to perform a quality trimming by removing short and low quality reads and to remove the adapters. RNA-seq reads were mapped to the reference genome (Gencode.v38) and aligned using a STAR aligner (15). Duplicate reads were removed and uniquely mapped transcripts were selected using Samtools (16; Danecek, Bonfield et al. 2021). The TPMs (Transcripts per million) were computed using StringTie (17) corrected by an IsoformSwitchAnalyzeR (18) and normalized using TMM (Trimmed Means of M value) (19). Differential expression analysis was performed using DESeq2 (20), using all annotated genes. Ontological analysis of gene expression was carried out using the Qiagen Ingenuity Pathway Analysis (IPA), and Gene Set Enrichment Analysis (GSEA) (21). The Interactome analysis was carried out by Pearson Correlation clustering using the 941 most differentially expressed genes for FM1 and the 361 most differentially expressed genes for FM2 using Cytoscape (22), the clusters were determined by AlegroMcode (AllegroViva Corporation, 2011) using default parameters.

### Results and Discussion

### Clinical Characteristics of FM Patients

The median age of patients participating in this study was 48 years of age with overall ages ranging from 28 to 77 years. The median age of the onset of FM was 36 years of age. The median age of control patients was 39 years, with ages ranging from 20 to 69 years. The clinical characteristics of the entire cohort are presented in Table 2.

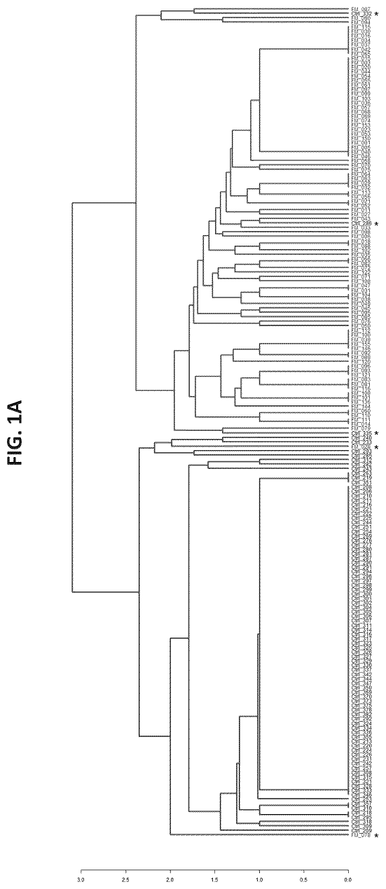

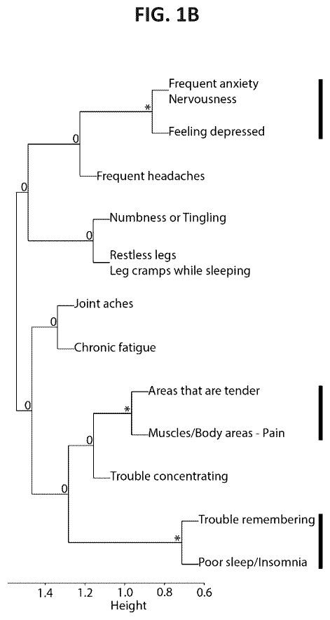

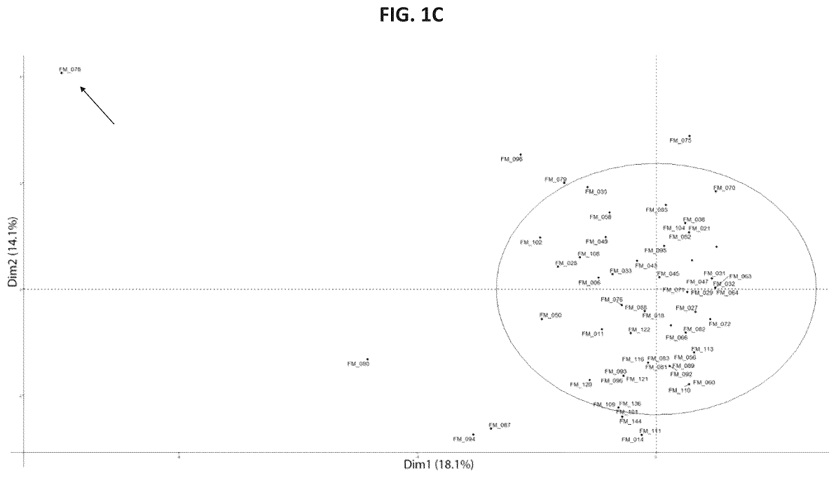

Hierarchical clustering was performed to test whether using clinical characteristics alone, the FM and control cases can be sub-grouped. A near perfect separation of two groups was observed (FIG. 1A) based on clinical symptoms. Only a minority of cases were misclassified: three control subjects: #286, #332, #335 were assigned to the FM group while two FM patients: #028, #078 were assigned to the control group. These five cases were not included in the downstream analysis.

Hierarchical clustering of the patient symptoms in the FM cohort indicated that there were three questions that were redundant: 1) patients with poor sleep and insomnia have also memory impairment, 2) depression was related to anxiety and nervousness, 3) patients who have body pain were more susceptible to have tender points (FIG. 1B). Principal Component Analysis (PCA) of the symptoms in FM patients indicate that the non-random explanation of variance is represented only on the first component and account for 18% of the observed variance (FIG. 1C). This suggests that globally the symptoms of FM patients were homogeneous, with the exception of patient ‘078’ who appeared to be the only outlier marked by the absence of body pain and tender areas and instead had the presence of depression and chronic fatigue.

### Cluster Analysis of RNA-Seq Data

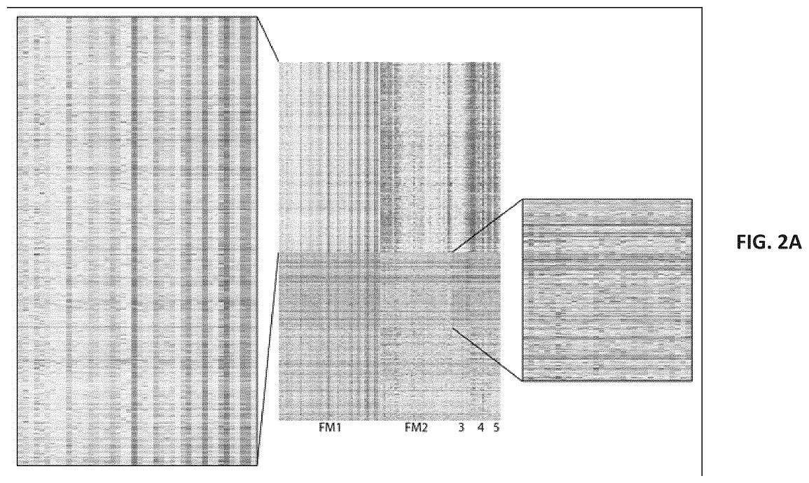

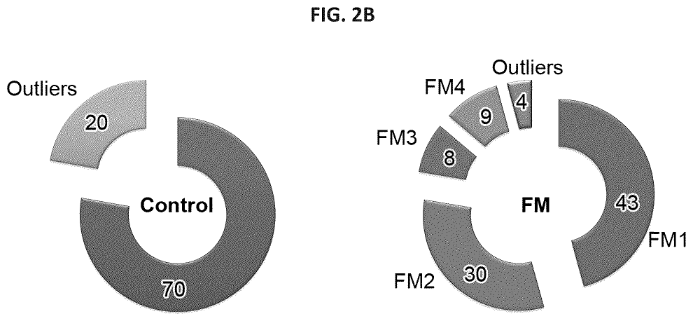

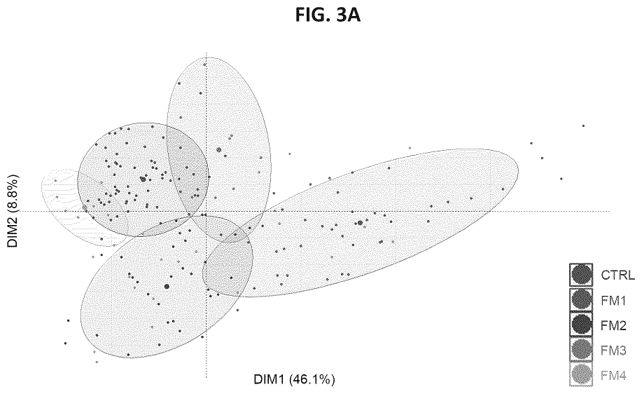

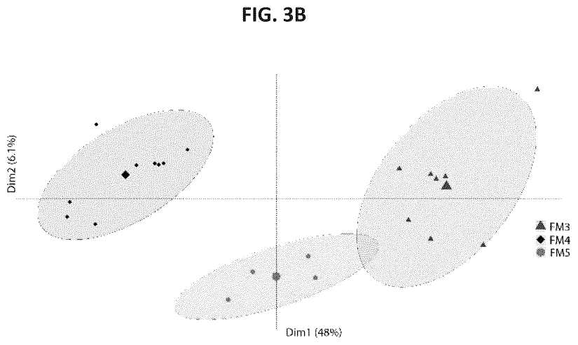

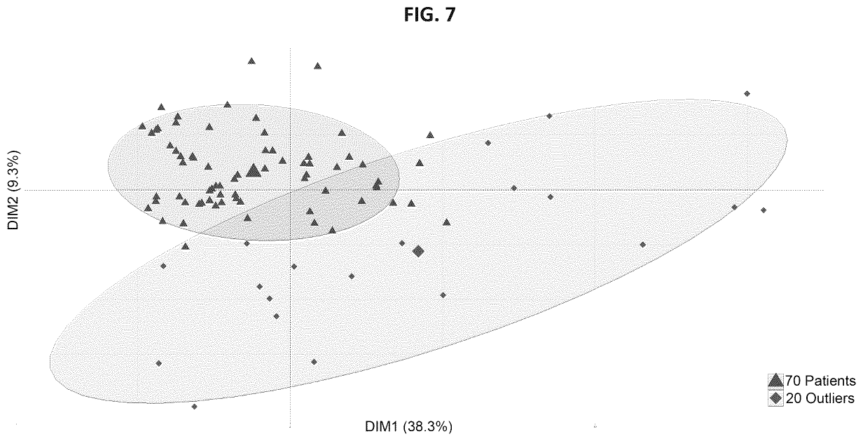

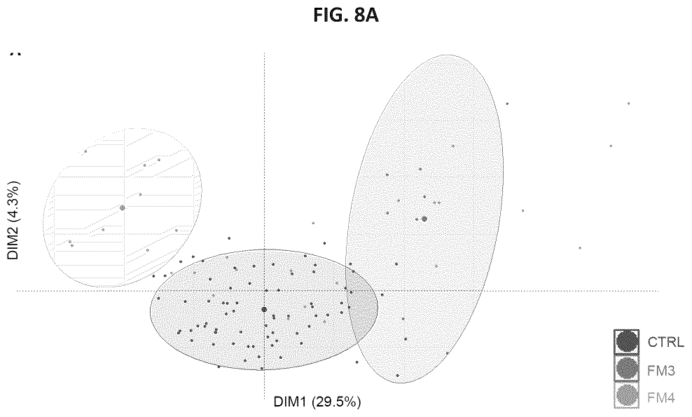

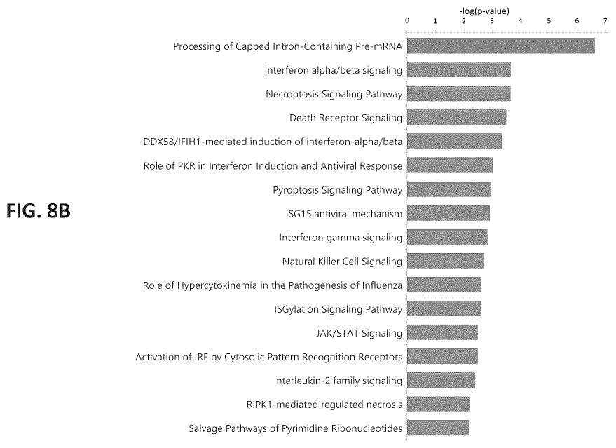

Differentially expressed genes were obtained using a linear model with the software ‘R’ (www.R-project.org/). Unsupervised clustering of differentially expressed genes was then performed by principal component analysis (PCA). To visualize the results of unsupervised clustering, the logarithm of the TPMs (transcript per million) was plotted using the heatmap function of ‘R’ (FIG. 2A). A total of 1720 differentially expressed transcripts were used to draw the heatmap using the algorithm of TPMs. Of the 90 control cases, 70 formed a homogenous tight cluster and 20 outliers (FIG. 2B and FIG. 7). The 20 outliers from the control group dispersed among FM1-3 patients. However, the entire cohort of 94 FM and 90 controls were used for all downstream analyses. The PCA indicated a homogeneous group of 43 patients which we labeled as FM1 and another group of 30 patients labeled as FM2 with unrelated underlying gene expression. The remaining group of 21 patients could be separated into two clusters of 8 and 9 patients each (FM3 and FM4) and 4 outliers (FIGS. 3, 8A). FM1, FM2 and combined FM3 and FM4 as a group were focused on for further analysis (see Tables 3-5).

A total of 1720 differentially expressed transcripts genes (DEGs) were identified in the entire FM cohort with FM1 having 914 DEGs, FM2 having 361 DEGs, FM3 having 255 DEGs, FM4 having 147 DEGs or 402 DEGs for FM3 and FM4 as a group. Of the 1720 DEGs, 1695 were protein coding, 24 lncRNA and 1 miRNA (Table 6).

### Analysis of FM1 Subgroup

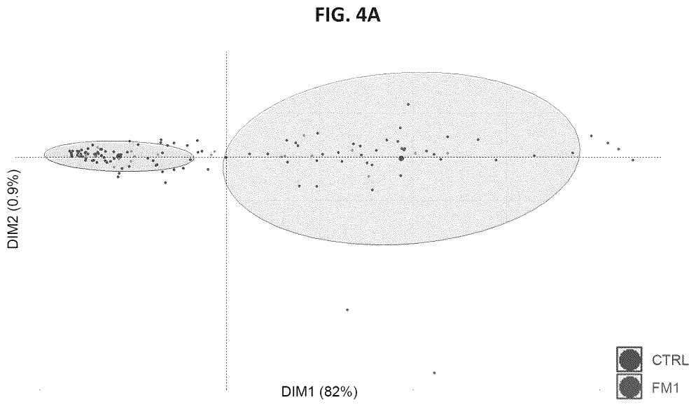

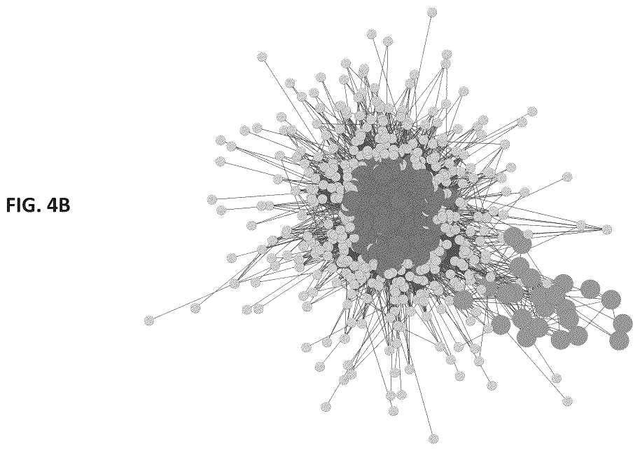

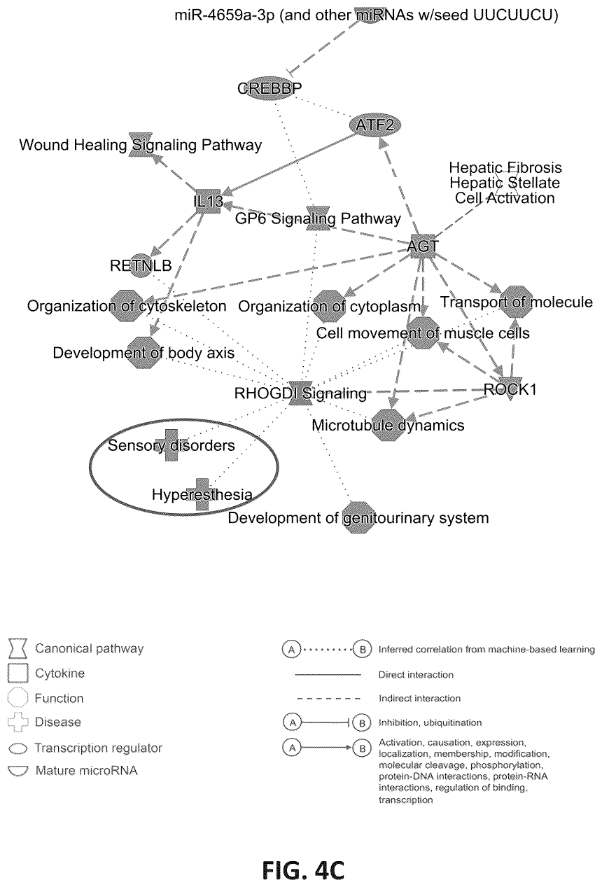

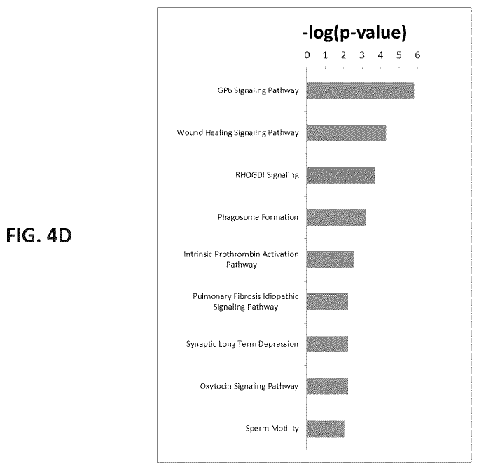

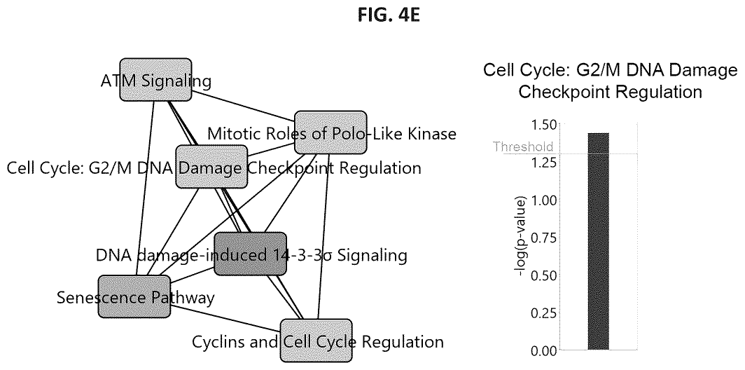

The FM1 group consisted of 43 patients with similar gene expression. PCA analysis of the 480 most differentially expressed genes (DEGs) from the 90 control patients and the 43 FM1 patients showed a clear separation of the patients between controls and FM1 (FIG. 4A). The first component of the PCA encompassed 82% of the total variation indicating that the disease state (Control vs FM1) is the major causes of gene expression difference between these two groups of patients. To understand biological pathways that are specific to the group of 43 FM1 patients, an interactome analysis was performed to pinpoint the differentially expressed genes (DEGs) that are most susceptible to be expressed in the same cells. Then an IPA was employed on these DEGs to identify the pathways that are associated with these genes.

The interactome analysis identified a major cluster composed of 338 DEGs represented in magenta and a smaller cluster of 24 DEGs represented in green (FIG. 4B). The DEGs represented in magenta indicate the presence of a cell (or group of cells) with coordinated gene expression across patients of the FM1 subgroup, while the DEGs represented in green appeared to represent a biological process. The genes included in each of these clusters were used for IPA analysis and found that the significant pathways represented by the major magenta DEGs belonged to extra-cellular matrix genes involved in connective tissue disorders (pulmonary fibrosis, wound healing, cytoskeletal organization, etc.) (see FIG. 4C, Table 3). Also these DEGs pinpoint to the presence of up regulated GP6 pathway and downregulation of Rho GDP-Dissociation Inhibitors (RHODGI) signaling (FIG. 4D). The minor cluster was composed of 24 DEGs that corresponded to cell-cycle associated genes (see FIG. 4E).

TABLE 3
Top-ranked canonical pathways in FM1 group.

| Ingenuity Canonical | −log(p- | | |
|---|---|---|---|
| Pathways | value) | zScore | Genes |
| GP6 Signaling | 5.82 | 3.32 | COL12A1, COL1A2, COL24A1, |
| Pathway | | | COL3A1, COL6A6, |
| | | | COL9A1, FGG, LAMA1, LAMA2, |
| | | | LAMA3, PIK3CA |
| Wound Healing | 4.29 | 1.94 | COL12A1, COL1A2, COL24A1, |
| Signaling Pathway | | | COL3A1, COL6A6, COL9A1, FN1, |
| | | | IL1RAPL2, LAMA1, LAMA2, |
| | | | LAMA3, MMP10, TRAP1 |
| RHOGDI Signaling | 3.69 | −2.24 | CDH10, CDH12, CDH6, CDH8, CDH9, |
| | | | GRIP1, ITGB6, MYH1, MYH2, |
| | | | MYH4, MYH7 |
| Phagosome | 3.2 | 4.58 | ADGRA3, ADGRB3, ADGRG6, FN1, |
| Formation | | | GPR156, GPRC6A, GRM1, HTR4, |
| | | | ITGB6, LGR5, MYH1, MYH2, MYH4, |
| | | | MYH7, OPRM1, PIK3CA, PLA2G4F, |
| | | | PLA2R1, RAPGEF4, RXFP2, TACR3 |
| Intrinsic Prothrombin | 2.58 | 2.00 | COL1A2, COL3A1, F11, FGG |
| Activation Pathway | | | |
| Pulmonary Fibrosis | 2.24 | 3.32 | COL12A1, COL1A2, COL24A1, |
| Idiopathic Signaling | | | COL3A1, COL6A6, COL9A1, FN1, |
| Pathway | | | ITGB6, MMP10, MMP20, PIK3CA |
| Synaptic Long Term | 2.24 | 2.83 | GRID2, GRM1, GUCY2C, PLA2G4F, |
| Depression | | | PLA2R1, PLCH1, PRKG2, RYR3 |
| Oxytocin Signaling | 2.23 | 2.53 | ABCC9, GUCY2C, KCNT2, MYH1, |
| Pathway | | | MYH2, MYH4, MYH7, PIK3CA, |
| | | | PLA2G4F, PRKG2 |
| Sperm Motility | 2.04 | 2.24 | ALK, EPHA3, PDE1C, PLA2G4F, |
| | | | PLA2R1, PLCH1, PRKG2, ROS1, TEK |
| Cell Cycle: G2/M | 1.44 | n/a | AC005578.3, CAB39L, CCNB2, |
| DNA Damage | | | CDKN3, CETN3, DNAJC27, EARS2, |
| Checkpoint | | | ELP4, FAM83D, FBXO24, ING1, |
| Regulation | | | LINC00167, LYPD3, PPP5D1, RPA2, |
| | | | SARNP, SCOC, SPATA24, SUCO, |
| | | | SYP |

### Gene Set Enrichment Analysis (GSEA) of FM1 Subgroup

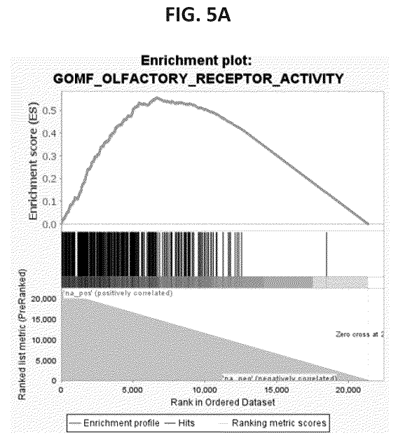

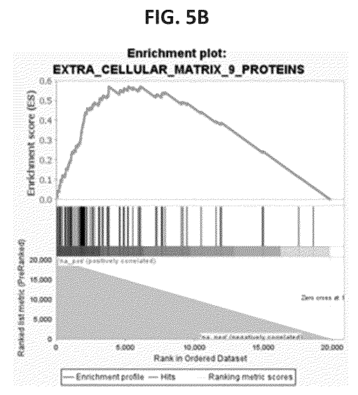

Independent of the IPA results that were searched in a pathway inside a private database, the whole gene set was analyzed using GSEA (Gene Set Enrichment Analysis, (21). This type of analysis ranks the genes from the most differentially expressed to the least differentially expressed. A search among all the known gene sets present in the public database identified the most enriched gene set. A custom gene set was analyzed that codes for nine proteins composed of the extra-cellular matrix extracted from (Reactome.org): Collagen, Fibrinogen, Elastin, Fibrillin, Fibronectin, Fibulin, Laminin, Matrilin and Tenascin. GSEA of the FM1 dataset revealed that the expression pattern most prominently correlates with olfactory receptor activity (NES=2.1, p-value <0.05) and extracellular matrix 9 proteins (NES=2.0, p-value <0.0001; FIGS. 5A-5B).

### Analysis of FM2 Subgroup

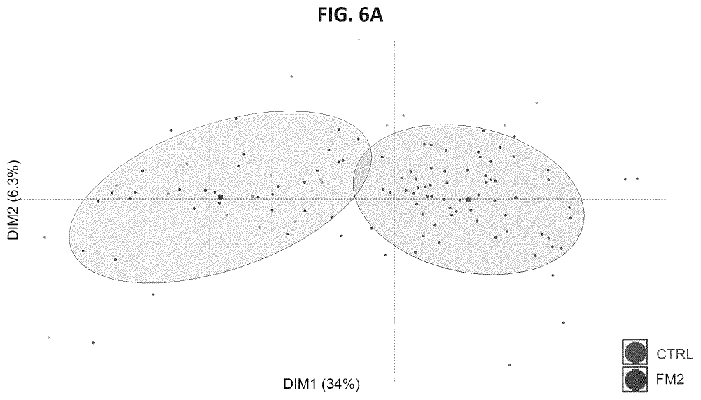

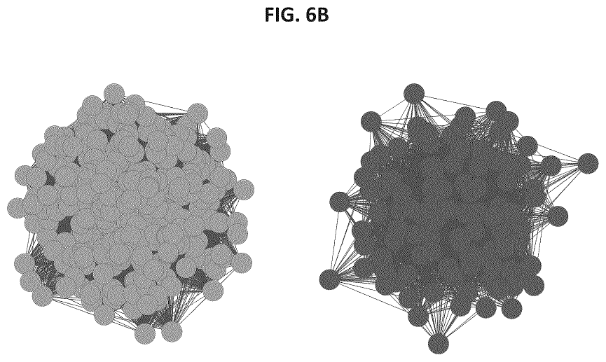

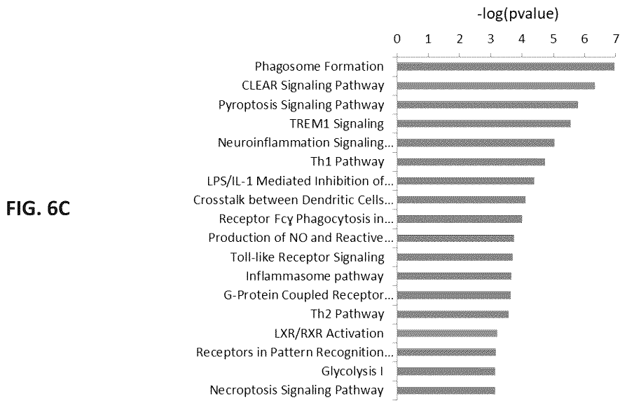

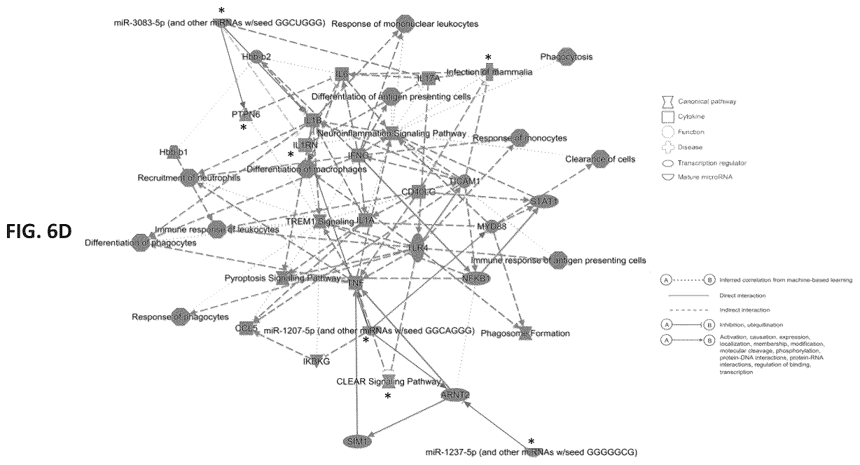

The FM2 group of patients was formed by 30 patients who possessed a similar type of gene expression. The principal component analysis of 361 DEGs from the 90 control patients and the 30 FM2 patients showed a separation of patients between the controls and the FM2 patients on the first component (FIG. 6A). The first component of the PCA encompassed 34% of the total variation, while the second encompassed a 6.3% variation documenting that the disease state (Control vs FM2) is an important cause of the gene expression differences between these two groups of patients. Interactome analysis separated the DEGs of the FM2 patients into two distinct clusters (FIG. 6B). These 2 clusters correspond to the upregulated and down-regulated DEGs. IPA analysis of the DEGs identified the most significant results in this group was the suppression or dysregulation of inflammatory processes (FIG. 6C). The top-ranked pathways dysregulated include phagosome formation, pyroptosis signaling pathway, TREM1 signaling, neuro-inflammation signaling, Th1 pathway, IL1-mediated inhibition of RXR function, crosstalk between dendritic cells and natural killer cells, toll-like receptor signaling, inflammasome pathway, Th2 pathway (FIG. 6D, Table 4). These results indicated a lymphocyte to monocyte ratio imbalance in FM2 patients (FIG. 6C). The CLEAR signaling pathway and LXR/RXR activation pathways were upregulated (FIG. 6D).

TABLE 4
Top-ranked canonical pathways in FM2 group

| Ingenuity Canonical | −log(p- | | |
|---|---|---|---|
| Pathways | value) | zScore | Genes |
| Phagosome | 6.97E+00 | −4.85 | ADGRE1, ADGRE3, ADGRG1, AP1S2, |
| Formation | | | C5AR1, CCR1, CCR2, CD14, CD36, |
| | | | FCER1G, FCGR1A, FCGR2A, FGR, |
| | | | FPR1, FPR2, HCK, HMOX1, HRH2, |
| | | | IGHM, ITGAM, LPAR1, LYN, P2RY13, |
| | | | PAK1, PLB1, PRKCD, PTAFR, S1PR3, |
| | | | TLR2, TLR4, TLR8 |
| CLEAR Signaling | 6.33E+00 | 0.47 | ASAH1, ATP6V0B, ATP6V0C, |
| Pathway | | | ATP6V0D1, ATP6V1B2, DDIT4, GAA, |
| | | | GABARAP, GNS, IFI30, INSR, PRKCD, |
| | | | PSAP, RXRA, TLR2, TLR4, TLR8, |
| | | | TNFRSF1B |
| Pyroptosis Signaling | 5.79E+00 | −3.16 | CASP4, IL1B, MEFV, NLRC4, NLRP12, |
| Pathway | | | PYCARD, TLR2, TLR4, TLR8, |
| | | | TNFRSF1B |
| TREM1 Signaling | 5.57E+00 | −3.00 | CD86, IL1B, NLRC4, NLRP12, TLR2, |
| | | | TLR4, TLR8, TREM1, TYROBP |
| Neuroinflammation | 5.05E+00 | −3.36 | CD86, CSF1R, CYBB, FOS, GABRR3, |
| Signaling Pathway | | | GRINA, HMOX1, IFNA4, IFNGR2, IL1B, |
| | | | NCF2, PSEN1, PYCARD, TLR2, TLR4, |
| | | | TLR8, TREM1, TYROBP |
| Th1 Pathway | 4.73E+00 | −0.71 | CD247, CD86, GATA3, IFNGR2, |
| | | | KLRD1, LGALS9, NOTCH2, PSEN1, |
| | | | STAT4, TBX21 |
| LPS/IL-1 Mediated | 4.39E+00 | −1.34 | ALDH2, ALDH3B1, CD14, CHST15, |
| Inhibition of RXR | | | CYP2S1, GSTP1, IL1B, IL1RN, NDST1, |
| Function | | | RARA, RXRA, SULT1A1, TLR4, |
| | | | TNFRSF1B |
| Crosstalk between | 4.11E+00 | −0.71 | CD226, CD86, KLRD1, LTBR, PRF1, |
| Dendritic Cells and | | | TLR4, TNFRSF1B, TYROBP |
| Natural Killer Cells | | | |
| Fcγ Receptor- | 4.01E+00 | −2.83 | FCGR1A, FCGR2A, FGR, HCK, |
| mediated | | | HMOX1, LYN, PAK1, PRKCD |
| Phagocytosis in | | | |
| Macrophages and | | | |
| Monocytes | | | |
| Production of Nitric | 3.74E+00 | −3.16 | CYBB, FOS, IFNGR2, LYZ, NCF2, |
| Oxide and Reactive | | | PRKCD, SIRPA, SPI1, TLR2, TLR4, |
| Oxygen Species in | | | TNFRSF1B |
| Macrophages | | | |
| Toll-like Receptor | 3.71E+00 | −2.24 | CD14, FOS, IL1B, IL1RN, TLR2, TLR4, |
| Signaling | | | TLR8 |
| Inflammasome | 3.67E+00 | −2.00 | IL1B, NLRC4, PYCARD, TLR4 |
| pathway | | | |
| G-Protein Coupled | 3.63E+00 | −2.86 | ADGRE1, ADGRE3, ADGRG1, AMOT, |
| Receptor Signaling | | | ARRB2, C5AR1, CCR1, CCR2, DUSP1, |
| | | | DUSP6, FOS, FPR1 |
| Th2 Pathway | 3.57E+00 | −1.00 | CCR1, CD247, CD86, GATA3, NOTCH2, |
| | | | PSEN1, SPI1, STAT4, TBX21 |
| LXR/RXR | 3.21E+00 | 1.41 | CD14, CD36, IL1B, IL1RN, LYZ, RXTA, |
| Activation | | | TLR4, TNFRSF1B |
| Role of Pattern | 3.16E+00 | −1.89 | C5AR1, IFNA4, IL1B, NLRC4, OAS1, |
| Recognition | | | PRKCD, TLR2, TLR4, TLR8 |
| Receptors in | | | |
| Recognition of | | | |
| Bacteria and Viruses | | | |
| Glycolysis I | 3.15E+00 | −2.00 | ALDOA, FBP1, GAPDH, PKM |
| Necroptosis | 3.14E+00 | −1.67 | CASP10, CYBB, IFNA4, PELI1, |
| Signaling Pathway | | | PYCARD, PYGL, SLC25A5, TLR4, |
| | | | TNFRSF1B |

### Analysis of FM3 and FM4 Subgroups

The FM3 and FM4 subgroups were composed of 17 cases together. Principal component analysis of the 361 DEGs in the 90 control and 17 FM patients showed a separation of patients between the controls, FM3 and FM4 on the first component (FIG. 8A). The first component of the PCA encompassed 29.50 of the total variation while the second encompassed 4.300 indicating that the disease state (Control vs FM3 and 4) is an important cause of gene expression differences between these groups of patients. Interactome analysis did not establish any significant interactions due to the small sample size. IPA analysis of the DEGs identified the following top-ranked dysregulated pathways including interferon signaling, death receptor signaling, natural killer cell signaling, JAK/STAT signaling and the processing of capped intron-containing pre-mRNA pathway (FIG. 8B, Table 5).

TABLE 5
Top-ranked canonical pathways in FM3 group and FM4 group.

| Ingenuity Canonical | −log(p- | | |
|---|---|---|---|
| Pathways | value) | zScore | Genes |
| Processing of capped | 6.62 | −0.63 | ACIN1, BUD13, CCAR1, CSTF2, |
| intron-containing | | | CWF19L2, FYTTD1, HNRNPA3, |
| pre-mRNA | | | MAGOH, MAGOHB, |
| | | | METTL14, MTREX, NCBP1, PCF11, |
| | | | PRPF3, PRPF38A, PRPF4, PRPF40A, |
| | | | RBM39, RBM7, SRSF4, WDR70, WTAP, |
| | | | ZMAT2 |
| Interferon alpha/beta | 3.66 | 2.12 | ADAR, ISG20, MX2, PTPN6, SAMHD1, |
| signaling | | | STAT1, STAT2, XAF1 |
| Necroptosis signaling | 3.65 | 1.73 | CASP1, CASP10, CFLAR, EIF2AK2, |
| pathway | | | IKBKB, MLKL, PPP3CB, STAT1, |
| | | | STAT2, TIMM13, |
| | | | TNFSF10, ZBP1 |
| Death receptor | 3.50 | 1.00 | ACIN1, CASP10, CSFLR, IKBKB, |
| signaling | | | MAP4K4, PARP2, PARP4, PARP9, |
| | | | TNFSF10 |
| DDX58/IFIH1- | 3.34 | 0.71 | CASP10, HERC5, HSP90AA1, IKBKB, |
| mediated induction | | | NLRX1, RIG1, TAX1BP1, TRIM25 |
| of interferon- | | | |
| alpha/beta | | | |
| Role of PKR in | 3.02 | 1.27 | CASP1, EIF2AK2, HSP90AA1, HSPA5, |
| interferon induction | | | IKBKB, METAP2, RIGI, STAT1, STAT2, |
| and antiviral | | | TIRAP |
| response | | | |
| Pyroptosis signaling | 2.96 | 2.12 | CASP1, GBP1, GBP4, GBP5, MEFV, |
| pathway | | | PRKAR1A, PTGER4, TXNIP |
| ISG15 antiviral | 2.92 | 1.13 | EIF2AK2, HERC5, KPNA2, MX2, RIG1, |
| mechanism | | | STAT1, TRIM25 |
| Interferon gamma | 2.84 | 2.12 | GBP1, GBP4, GBP5, PTPN6, STAT1, |
| signaling | | | TRIM22, TRIM25, TRIM38 |
| Natural killer cell | 2.72 | 1.16 | CFL1, HSPA5, LAT, MICA, NFAT5, |
| signaling | | | PIK3C3, PIK3R1, PTK2B, PTPN6, |
| | | | RAP1B, STAT4, TNFSF10 |
| Role of | 2.61 | 2.65 | CASP1, CCL2, EIF2AK2, ISG20, RIGI, |
| hyperchemokinemia | | | STAT1, STAT2 |
| in the pathogenesis | | | |
| of influenza | | | |
| ISGylation signaling | 2.61 | 1.41 | DTX3L, EIF2AK2, HERC5, NFAT5, |
| pathway | | | RIG1, STAT1, STAT2, TRIM25 |
| JAK/STAT signaling | 2.49 | 1.13 | PIK3C3, PIK3R1, PTPN6, RAP1B, |
| | | | STAT1, STAT2, STAT4 |
| Activation of IRF by | 2.49 | 1.63 | ADAR, IKBKB, RIG1, STAT1, STAT2, |
| cytosolic pattern | | | ZBP1 |
| recognition receptors | | | |
| Interleukin-2 family | 2.40 | 2.24 | PIK3R1, PTK2B, PTPN6, STAT1, |
| signaling | | | STAT4 |
| RIPK1-mediated | 2.22 | 2.00 | CFLAR, HSP90AA1, MLKL, TNFSF10 |
| regulated necrosis | | | |
| Salvage pathways of | 2.17 | 1.89 | CSNK1A1, EIF2AK2, NME1, UCK2, |
| pyrimidine | | | UCKL1, UPP1, UPRT |
| ribonucleotides | | | |

### CONCLUSIONS

FM was previously characterized as a syndrome with widespread pain and tenderness (23). Conceptually, the definition of FM has evolved over time and is perceived as a continuum representing an increased and heightened processing of pain within the nervous system (24). In 2016, nociplastic pain was proposed as a mechanistic descriptor for FM and chronic pain. Nociplastic pain is defined as pain arising from the altered function of pain related to sensory pathways in the peripheral and central nervous system, causing increased sensitivity (5). In FM patients, nociplastic pain can occur as a comorbidity with an inflammatory, immune, endocrine, genetic and psychosocial factors; all these phenotypes leading to a sensitization phenomenon characterized by a decrease in pain tolerance to afferent nociceptive stimuli (1, 25, 26). Over the years, there has been increasing recognition that chronic pain conditions are heterogeneous with a degree of overlap of phenotypes (1, 27). However, to date, there is no clear explanation to account for this clinical heterogeneity.

The study by Behm et al. produced a clearly defined laboratory test criteria that could be used for objective diagnosis of FM patients (6). The present study aimed to precisely define these patients via identification of genomic markers or signatures specific for FM patients to aid accurate diagnosis and possibly leading to development of mechanism based targeted therapeutics rather than symptom based treatment.

To this end, high throughput RNA-sequencing was utilized for whole transcriptome analysis in 94 patients with FM and 90 healthy matched control subjects (who lacked a positive FM blood test result or, in other words, had a negative cytokine assay result) using RNA from peripheral blood. The results of these analyses identified multiple subgroups within the cohort of FM patients with distinct non-overlapping gene signatures. Of note, subgroups of FM were identified with sufficient number of patients in two subgroups (FM1 and FM2) and combined FM3 and FM4 for detailed downstream analysis. The presence of multiple subgroups within the FM patient cohort explains the inherent clinical heterogeneity associated with FM and chronic pain disorders, which explains the diagnostic difficulty often encountered in a clinical setting. The two major subgroups displayed distinct transcriptional profiles indicating two different etiologies that are grouped together under the same general diagnosis of FM. Although a specific cause of FM was not identified, identification of these subgroups will help develop additional novel diagnostic markers and therapeutics for these patients. The differences observed among the patients suggest that different treatment approaches will be required for patients with FM. However, this study did identify subgroups of FM defined by transcriptional signatures.

The first group, FM1, included individuals with a DEG signature enriched for gene expression of extracellular matrix (ECM) associated with connective tissue disorders down-regulation of the Rho GDP Dissociation Inhibitor (RHOGDI) signaling pathway (Table 3). RhoGDI signaling pathway is the regulator of Rho family of GTPase that are implicated in the formation of stress fibers and in pain perception through somatosensory neurons (28). In this group of patients, deregulation of ECM and tissue homeostasis is likely mediated by fibrocytes. Fibrocytes are bone marrow-derived mesenchymal progenitor cells that directly contribute to tissue remodeling and fibrosis of tissues throughout the body by producing ECM proteins (collagen 1 and collagen III) and by secreting matrix metalloproteinases following injury, wound healing and during fibro-proliferative disorders in response to local tissue injury (29, 30). Fibrocytes traffic to sites of injury during the earliest phase of the innate immune response and exhibit both the inflammatory features of macrophages and the tissue remodeling properties of fibroblasts. Fibrocytes are distinguished by the simultaneous expression of CD34 or CD45 and collagen (29, 30). Inhibition of Rho kinase can increase resting tissue tension which regulates actomyosin contractility, formation of stress fibers (actin-myosin filaments) and maturation of focal adhesions (28, 32). The results suggest that Rho-dependent remodeling of cell matrix is affected in the FM1 patients. At the local cellular level, matrix tension has been shown to influence a wide variety of cellular events including neurite growth and angiogenesis (33, 34). Thus, cell-mediated connective tissue tension regulation may be important to protect blood vessels, sensory and autonomic nerves from prolonged tissue loads induced by various body positions such as sitting, standing, and sleeping positions. In vivo connective tissue tension may not only impact connective tissue homeostasis but also the vascular, nervous and immune cell populations that reside within the connective tissue network as well as adjacent organ specific cell populations. The presence of cell cycle associated genes in the signature indicates persistent stimuli triggered by chronic inflammation that leads to defects in DNA repair mechanisms leading to activation of fibrocytes (35, 36).

The second group, FM2, included individuals that showed a profound reduction in the expression of inflammatory mediators and increased expression of genes involved in the Coordinated Lysosomal Expression And Regulation (CLEAR) signaling pathway. In the FM2 subgroup, there was a significant immune dysregulation as reflected by the under expression of genes involved in phagosome formation, pyroptosis signaling, TREM1 signaling, neuro-inflammation, Th1 and Th2 pathways, crosstalk between dendritic cells and natural killer cells, toll-like receptor signaling and the inflammasome pathway, among others (Table 4). One of the top-ranked pathways that showed overexpression of genes includes the CLEAR signaling pathway. CLEAR pathway is a cellular program that regulates lysosomal biogenesis and participates in macromolecule clearance (37). CLEAR network is activated by lysosomal storage. The transcription factor EB (TFEB) is a master regulator of lysosomal function (37, 38). TFEB promotes the expression of genes involved in lysosomal biogenesis, such as the mannose 6-phospate receptors, which transport newly synthesized lysosomal enzymes from Golgi to lysosomes. The activity of TFEB is regulated by multiple kinases, in particular the mechanistic target of rapamycin complex 1 (mTORC1) (39-41). When phosphorylated, TFEB is retained in the cytoplasm and inhibited. Several stress signals including nutrient deprivation, proteotoxicity, and lysosomal damage, which have been reported to promote TFEB dephosphorylation, nuclear translocation and activation, leading to an increase in the number and activity of lysosomes (42). mTORC1 is activated by nutrients and growth factors, and conversely is inhibited by starvation (43). The activation or inactivation of mTORC1 in response to nutrient availability occurs on the lysosome and is regulated by a number of lysosomal membrane associated proteins (44, 45). Thus, the lysosome not only functions as a scaffolding organelle but can also participate in the nutrient sensing process. The regulation of mTORC1 signaling by the lysosome also occurs through a transcriptional mechanism mediated by TFEB, which is activated in response to lysosomal stress. TFEB direct target genes were identified by combining ChIPseq, TFEB overexpression, promoter analysis and co-expression meta-analysis (46). These genes encode for proteins that can be grouped into several distinct categories, including lysosomal hydrolases and accessory proteins, lysosomal membrane proteins, subunits of the proton pump, proteins participating in autophagy and non-lysosomal proteins involved in lysosomal biogenesis (46). The data show differential expression of genes encoding lysosomal hydrolases and accessory proteins ASAH1, GAA, GNS, IFI30, PSAP; and genes involved in lysosomal acidification ATP6V0B, ATP6V0C, ATP6V0D1, ATP6V1B2 (Table 4) suggesting dysregulation of lysosomal homeostasis in FM2 patients. TFEB also promotes the formation of autophagosomes and their fusion with lysosomes through the upregulation of several key autophagy and lysosomal genes, a process that is initiated by nutrient starvation and executed by the inhibition of extracellular signal regulated kinase 2 (ERK2)-mediated phosphorylation of TFEB at Ser142 (41). The results show differential expression of the autophagy gene GABA type A receptor-associated protein (GABARAP) (Table 4). GABARAP is a ubiquitin-like modifier that plays a role in intracellular transport of GABA(A) receptors and its interaction with the cytoskeleton. It is involved in autophagy, while the microtubule-associated protein 1A/1B-light chain 3 (LC3) is involved in elongation of the phagophore membrane. The GABARAP subfamily is essential for a later stage in autophagosome maturation (47). Through its interaction with the reticulophagy receptor TEX264, GABARAP participates in the remodeling of subdomains of the endoplasmic reticulum into autophagosomes upon nutrient stress, which then fuse with lysosomes for endoplasmic reticulum turnover (48). Other TFEB direct targets are genes belonging to distinct families of pattern recognition molecules including membrane-anchored Toll-like receptors (TLRs), which are involved in the innate immune detection of danger signals and microbial motifs (49) and the insulin signaling pathway (46). Taken together, these results indicate defects in vesicle transport and lysosomal homeostasis in FM2 patients.

The other two, FM3 and FM4 subgroups, while distinct from the FM1 and FM2, had two few subjects to clearly define the pathways involved. A combined analysis of FM groups 3 and 4 identified pathways related to acute inflammatory associated with the Th1 responsive processes with overexpression of interferon alpha/beta, JAK/STAT pathway, IL2, pyroptosis, cell death receptor and necroptosis pathways. Strong downregulation of the processing of capped intron containing pre mRNA pathway indicates global dysregulation of the transcription machinery (Table 5).

In conclusion, whole transcriptome analysis of FM patients identified novel gene expression signatures. This appears to be the first study to report genetic heterogeneity within FM patients. The two major groups of FM patients reported here have defects in the tissue homeostasis associated with ECM and the lysosomal biogenesis pathway.

TABLE 6
Differentially expressed genes (DEGs) identified in the entire FM cohort

| | | | |
|---|---|---|---|
| gene_name | Gene description | gene_id | NCBI gene ID |

FM1

| | | | |
|---|---|---|---|
| A4GNT | alpha-1,4-N- | ENSG00000118017 | 51146 |
| | acetylglucosaminyltransferase | | |
| | [Source: HGNC | | |
| | Symbol; Acc: HGNC: 17968] | | |
| AC004069.1 | nucleoporin 43 kDa (NUP43) | ENSG00000251473 | 0 |
| | pseudogene | | |
| AC005578.3 | TEC | ENSG00000280247 | 0 |
| AC027612.4 | pseudogene similar to part of | ENSG00000223703 | 0 |
| | immunoglobulin superfamily, | | |
| | member 3 (IGSF3) | | |
| AC093107.7 | ribosomal protein L15 (RPL15) | ENSG00000223718 | 0 |
| | pseudogene | | |
| AC098826.4 | pseudogene similar to putative | ENSG00000225594 | 0 |
| | zinc finger protein | | |
| | ENSP00000344568 | | |
| AC112229.1 | zinc finger protein 532 | ENSG00000230650 | 0 |
| | (ZNF532) pseudogene | | |
| ACKR4 | atypical chemokine receptor 4 | ENSG00000129048 | 51554 |
| | [Source: HGNC | | |
| | Symbol; Acc: HGNC: 1611] | | |
| ACTN4P1 | actinin alpha 4 pseudogene 1 | ENSG00000213493 | 0 |
| | [Source: HGNC | | |
| | Symbol; Acc: HGNC: 44028] | | |
| ADAM7 | ADAM metallopeptidase | ENSG00000069206 | 8756 |
| | domain 7 [Source: HGNC | | |
| | Symbol; Acc: HGNC: 214] | | |
| ADCYAP1R1 | ADCYAP receptor type I | ENSG00000078549 | 117 |
| | [Source: HGNC | | |
| | Symbol; Acc: HGNC: 242] | | |
| ADRA1B | adrenoceptor alpha 1B | ENSG00000170214 | 147 |
| | [Source: HGNC | | |
| | Symbol; Acc: HGNC: 278] | | |
| AF213884.1 | zinc finger protein 705A | ENSG00000251572 | 0 |
| | (ZNF705A) pseudogene | | |
| AGTR1 | angiotensin II receptor type 1 | ENSG00000144891 | 185 |
| | [Source: HGNC | | |
| | Symbol; Acc: HGNC: 336] | | |
| AHSG | alpha 2-HS glycoprotein | ENSG00000145192 | 197 |
| | [Source: HGNC | | |
| | Symbol; Acc: HGNC: 349] | | |
| AIMP1P1 | aminoacyl tRNA synthetase | ENSG00000234187 | 0 |
| | complex interacting | | |
| | multifunctional protein 1 | | |
| | pseudogene 1 [Source: HGNC | | |
| | Symbol; Acc: HGNC: 16543] | | |
| AK4 | adenylate kinase 4 | ENSG00000162433 | 205 |
| | [Source: HGNC | | |
| | Symbol; Acc: HGNC: 363] | | |
| ALMS1P1 | ALMS1 pseudogene 1 | ENSG00000163016 | 200420 |
| | [Source: HGNC | | |
| | Symbol; Acc: HGNC: 29586] | | |
| ANKDD1B | ankyrin repeat and death | ENSG00000189045 | 728780 |
| | domain containing 1B | | |
| | [Source: HGNC | | |
| | Symbol; Acc: HGNC: 32525] | | |
| ANOS1 | anosmin 1 [Source: HGNC | ENSG00000011201 | 0 |
| | Symbol; Acc: HGNC: 6211] | | |
| API5P2 | apoptosis inhibitor 5 | ENSG00000213962 | 0 |
| | pseudogene 2 [Source: HGNC | | |
| | Symbol; Acc: HGNC: 39070] | | |
| ARHGAP40 | Rho GTPase activating protein | ENSG00000124143 | 343578 |
| | 40 [Source: HGNC | | |
| | Symbol; Acc: HGNC: 16226] | | |
| ARHGAP42P5 | ARHGAP42 pseudogene 5 | ENSG00000258233 | 0 |
| | [Source: HGNC | | |
| | Symbol; Acc: HGNC: 43942] | | |
| ARHGEF33 | Rho guanine nucleotide | ENSG00000214694 | 100271715 |
| | exchange factor 33 | | |
| | [Source: HGNC | | |
| | Symbol; Acc: HGNC: 37252] | | |
| ARL5C | ADP ribosylation factor like | ENSG00000141748 | 390790 |
| | GTPase 5C [Source: HGNC | | |
| | Symbol; Acc: HGNC: 31111] | | |
| ARNT2 | aryl hydrocarbon receptor | ENSG00000172379 | 0 |
| | nuclear translocator 2 | | |
| | [Source: HGNC | | |
| | Symbol; Acc: HGNC: 16876] | | |
| ARSH | arylsulfatase family member H | ENSG00000205667 | 347527 |
| | [Source: HGNC | | |
| | Symbol; Acc: HGNC: 32488] | | |
| ART4 | ADP-ribosyltransferase 4 | ENSG00000111339 | 420 |
| | (inactive) (Dombrock blood | | |
| | group) [Source: HGNC | | |
| | Symbol; Acc: HGNC: 726] | | |
| ASB11 | ankyrin repeat and SOCS box | ENSG00000165192 | 140456 |
| | containing 11 [Source: HGNC | | |
| | Symbol; Acc: HGNC: 17186] | | |
| ASB14 | ankyrin repeat and SOCS box | ENSG00000239388 | 142686 |
| | containing 14 [Source: HGNC | | |
| | Symbol; Acc: HGNC: 19766] | | |
| ASB18 | ankyrin repeat and SOCS box | ENSG00000182177 | 401036 |
| | containing 18 [Source: HGNC | | |
| | Symbol; Acc: HGNC: 19770] | | |
| ASB4 | ankyrin repeat and SOCS box | ENSG00000005981 | 51666 |
| | containing 4 [Source: HGNC | | |
| | Symbol; Acc: HGNC: 16009] | | |
| ASB9 | ankyrin repeat and SOCS box | ENSG00000102048 | 140462 |
| | containing 9 [Source: HGNC | | |
| | Symbol; Acc: HGNC: 17184] | | |
| ASPN | asporin [Source: HGNC | ENSG00000106819 | 0 |
| | Symbol; Acc: HGNC: 14872] | | |
| ATP2B2 | ATPase plasma membrane | ENSG00000157087 | 491 |
| | Ca2+ transporting 2 | | |
| | [Source: HGNC | | |
| | Symbol; Acc: HGNC: 815] | | |
| ATRNL1 | attractin like 1 [Source: HGNC | ENSG00000107518 | 26033 |
| | Symbol; Acc: HGNC: 29063] | | |
| B3GALNT1 | beta-1,3-N- | ENSG00000169255 | 8706 |
| | acetylgalactosaminyltransferase | | |
| | 1 (globoside blood group) | | |
| | [Source: HGNC | | |
| | Symbol; Acc: HGNC: 918] | | |
| B3GALT2 | beta-1,3-galactosyltransferase 2 | ENSG00000162630 | 8707 |
| | [Source: HGNC | | |
| | Symbol; Acc: HGNC: 917] | | |
| BBOX1 | gamma-butyrobetaine | ENSG00000129151 | 8424 |
| | hydroxylase 1 [Source: HGNC | | |
| | Symbol; Acc: HGNC: 964] | | |
| BCO1 | beta-carotene oxygenase 1 | ENSG00000135697 | 0 |
| | [Source: HGNC | | |
| | Symbol; Acc: HGNC: 13815] | | |
| BDNF | brain derived neurotrophic | ENSG00000176697 | 627 |
| | factor [Source: HGNC | | |
| | Symbol; Acc: HGNC: 1033] | | |
| BEND6 | BEN domain containing 6 | ENSG00000151917 | 221336 |
| | [Source: HGNC | | |
| | Symbol; Acc: HGNC: 20871] | | |
| BHLHB9P1 | basic helix-loop-helix family | ENSG00000258915 | 0 |
| | member b9 pseudogene 1 | | |
| | [Source: HGNC | | |
| | Symbol; Acc: HGNC: 51373] | | |
| BMP10 | bone morphogenetic protein 10 | ENSG00000163217 | 27302 |
| | [Source: HGNC | | |
| | Symbol; Acc: HGNC: 20869] | | |
| BRINP3 | BMP/retinoic acid inducible | ENSG00000162670 | 339479 |
| | neural specific 3 | | |
| | [Source: HGNC | | |
| | Symbol; Acc: HGNC: 22393] | | |
| BRS3 | bombesin receptor subtype 3 | ENSG00000102239 | 680 |
| | [Source: HGNC | | |
| | Symbol; Acc: HGNC: 1113] | | |
| BUB3P1 | BUB3 mitotic checkpoint | ENSG00000227968 | 0 |
| | protein pseudogene 1 | | |
| | [Source: HGNC | | |
| | Symbol; Acc: HGNC: 51559] | | |
| C12orf60 | chromosome 12 open reading | ENSG00000182993 | 144608 |
| | frame 60 [Source: HGNC | | |
| | Symbol; Acc: HGNC: 28726] | | |
| C15orf65 | chromosome 15 open reading | ENSG00000261652 | 145788 |
| | frame 65 [Source: HGNC | | |
| | Symbol; Acc: HGNC: 44654] | | |
| C17orf64 | chromosome 17 open reading | ENSG00000141371 | 124773 |
| | frame 64 [Source: HGNC | | |
| | Symbol; Acc: HGNC: 26990] | | |
| C1orf189 | chromosome 1 open reading | ENSG00000163263 | 388701 |
| | frame 189 [Source: HGNC | | |
| | Symbol; Acc: HGNC: 32305] | | |
| C2orf73 | chromosome 2 open reading | ENSG00000177994 | 129852 |
| | frame 73 [Source: HGNC | | |
| | Symbol; Acc: HGNC: 26861] | | |
| C4orf17 | chromosome 4 open reading | ENSG00000138813 | 0 |
| | frame 17 [Source: HGNC | | |
| | Symbol; Acc: HGNC: 25274] | | |
| C7 | complement C7 | ENSG00000112936 | 730 |
| | [Source: HGNC | | |
| | Symbol; Acc: HGNC: 1346] | | |
| C8orf89 | chromosome 8 open reading | ENSG00000274443 | 100130301 |
| | frame 89 [Source: HGNC | | |
| | Symbol; Acc: HGNC: 51258] | | |
| CALB1 | calbindin 1 [Source: HGNC | ENSG00000104327 | 0 |
| | Symbol; Acc: HGNC: 1434] | | |
| CALCR | calcitonin receptor | ENSG00000004948 | 799 |
| | [Source: HGNC | | |
| | Symbol; Acc: HGNC: 1440] | | |
| CAPN9 | calpain 9 [Source: HGNC | ENSG00000135773 | 10753 |
| | Symbol; Acc: HGNC: 1486] | | |
| CAPZA3 | capping actin protein of muscle | ENSG00000177938 | 93661 |
| | Z-line subunit alpha 3 | | |
| | [Source: HGNC | | |
| | Symbol; Acc: HGNC: 24205] | | |
| CAVIN4 | caveolae associated protein 4 | ENSG00000170681 | 347273 |
| | [Source: HGNC | | |
| | Symbol; Acc: HGNC: 33742] | | |
| CCDC173 | coiled-coil domain containing | ENSG00000154479 | 129881 |
| | 173 [Source: HGNC | | |
| | Symbol; Acc: HGNC: 25064] | | |
| CCDC28A-AS1 | CCDC28A antisense RNA 1 | ENSG00000279968 | 0 |
| | [Source: HGNC | | |
| | Symbol; Acc: HGNC: 51715] | | |
| CCDC63 | coiled-coil domain containing | ENSG00000173093 | 160762 |
| | 63 [Source: HGNC | | |
| | Symbol; Acc: HGNC: 26669] | | |
| CCIN | calicin [Source: HGNC | ENSG00000185972 | 881 |
| | Symbol; Acc: HGNC: 1568] | | |
| CCT5P1 | chaperonin containing TCP1 | ENSG00000250444 | 0 |
| | subunit 5 pseudogene 1 | | |
| | [Source: HGNC | | |
| | Symbol; Acc: HGNC: 35135] | | |
| CDCA3 | cell division cycle associated 3 | ENSG00000111665 | 83461 |
| | [Source: HGNC | | |
| | Symbol; Acc: HGNC: 14624] | | |
| CFAP206 | cilia and flagella associated | ENSG00000272514 | 154313 |
| | protein 206 [Source: HGNC | | |
| | Symbol; Acc: HGNC: 21405] | | |
| CHMP4C | charged multivesicular body | ENSG00000164695 | 0 |
| | protein 4C [Source: HGNC | | |
| | Symbol; Acc: HGNC: 30599] | | |
| CHORDC1P5 | CHORDC1 pseudogene 5 | ENSG00000233020 | 0 |
| | [Source: HGNC | | |
| | Symbol; Acc: HGNC: 54690] | | |
| CHRM2 | cholinergic receptor muscarinic | ENSG00000181072 | 1129 |
| | 2 [Source: HGNC | | |
| | Symbol; Acc: HGNC: 1951] | | |
| CHST4 | carbohydrate sulfotransferase 4 | ENSG00000140835 | 10164 |
| | [Source: HGNC | | |
| | Symbol; Acc: HGNC: 1972] | | |
| CLDN34 | claudin 34 [Source: HGNC | ENSG00000234469 | 100288814 |
| | Symbol; Acc: HGNC: 51259] | | |
| CLSTN2 | calsyntenin 2 [Source: HGNC | ENSG00000158258 | 64084 |
| | Symbol; Acc: HGNC: 17448] | | |
| CLVS2 | clavesin 2 [Source: HGNC | ENSG00000146352 | 0 |
| | Symbol; Acc: HGNC: 23046] | | |
| CNOT7P2 | CCR4-NOT transcription | ENSG00000232185 | 0 |
| | complex subunit 7 pseudogene | | |
| | 2 [Source: HGNC | | |
| | Symbol; Acc: HGNC: 44249] | | |
| COL6A6 | collagen type VI alpha 6 chain | ENSG00000206384 | 131873 |
| | [Source: HGNC | | |
| | Symbol; Acc: HGNC: 27023] | | |
| COL8A1 | collagen type VIII alpha 1 | ENSG00000144810 | 1295 |
| | chain [Source: HGNC | | |
| | Symbol; Acc: HGNC: 2215] | | |
| COX4I2 | cytochrome c oxidase subunit | ENSG00000131055 | 84701 |
| | 412 [Source: HGNC | | |
| | Symbol; Acc: HGNC: 16232] | | |
| CPA2 | carboxypeptidase A2 | ENSG00000158516 | 1358 |
| | [Source: HGNC | | |
| | Symbol; Acc: HGNC: 2297] | | |
| CPB2 | carboxypeptidase B2 | ENSG00000080618 | 1361 |
| | [Source: HGNC | | |
| | Symbol; Acc: HGNC: 2300] | | |
| CPN2 | carboxypeptidase N subunit 2 | ENSG00000178772 | 1370 |
| | [Source: HGNC | | |
| | Symbol; Acc: HGNC: 2313] | | |
| CRYGA | crystallin gamma A | ENSG00000168582 | 1418 |
| | [Source: HGNC | | |
| | Symbol; Acc: HGNC: 2408] | | |
| CSRP3 | cysteine and glycine rich | ENSG00000129170 | 8048 |
| | protein 3 [Source: HGNC | | |
| | Symbol; Acc: HGNC: 2472] | | |
| CST2 | cystatin SA [Source: HGNC | ENSG00000170369 | 1470 |
| | Symbol; Acc: HGNC: 2474] | | |
| CTC-507E12.1 | mitochondrial ribosomal | ENSG00000234259 | 0 |
| | protein L10 pseudogene | | |
| CTD-2285G11.1 | zinc finger protein family | ENSG00000250145 | 0 |
| | pseudogene | | |
| CTD-2532D12.4 | novel transcript | ENSG00000263574 | 0 |
| CTHRC1 | collagen triple helix repeat | ENSG00000164932 | 115908 |
| | containing 1 [Source: HGNC | | |
| | Symbol; Acc: HGNC: 18831] | | |
| CTSE | cathepsin E [Source: HGNC | ENSG00000196188 | 1510 |
| | Symbol; Acc: HGNC: 2530] | | |
| CUL1P1 | cullin 1 pseudogene 1 | ENSG00000251032 | 0 |
| | [Source: HGNC | | |
| | Symbol; Acc: HGNC: 49567] | | |
| CXCL11 | C-X-C motif chemokine ligand | ENSG00000169248 | 6373 |
| | 11 [Source: HGNC | | |
| | Symbol; Acc: HGNC: 10638] | | |
| CYP4X1 | cytochrome P450 family 4 | ENSG00000186377 | 260293 |
| | subfamily X member 1 | | |
| | [Source: HGNC | | |
| | Symbol; Acc: HGNC: 20244] | | |
| CYP4Z2P | cytochrome P450 family 4 | ENSG00000154198 | 0 |
| | subfamily Z member 2, | | |
| | pseudogene [Source: HGNC | | |
| | Symbol; Acc: HGNC: 24426] | | |
| DAOA | D-amino acid oxidase activator | ENSG00000182346 | 267012 |
| | [Source: HGNC | | |
| | Symbol; Acc: HGNC: 21191] | | |
| DDX18P6 | DEAD-box helicase 18 | ENSG00000220585 | 0 |
| | pseudogene 6 [Source: HGNC | | |
| | Symbol; Acc: HGNC: 31126] | | |
| DGKI | diacylglycerol kinase iota | ENSG00000157680 | 9162 |
| | [Source: HGNC | | |
| | Symbol; Acc: HGNC: 2855] | | |
| DLX1 | distal-less homeobox 1 | ENSG00000144355 | 1745 |
| | [Source: HGNC | | |
| | Symbol; Acc: HGNC: 2914] | | |
| DLX5 | distal-less homeobox 5 | ENSG00000105880 | 1749 |
| | [Source: HGNC | | |
| | Symbol; Acc: HGNC: 2918] | | |
| DMC1 | DNA meiotic recombinase 1 | ENSG00000100206 | 11144 |
| | [Source: HGNC | | |
| | Symbol; Acc: HGNC: 2927] | | |
| DNAH17-AS1 | DNAH17 antisense RNA 1 | ENSG00000267432 | 0 |
| | [Source: HGNC | | |
| | Symbol; Acc: HGNC: 48594] | | |
| DNAJC27 | DnaJ heat shock protein family | ENSG00000115137 | 51277 |
| | (Hsp40) member C27 | | |
| | [Source: HGNC | | |
| | Symbol; Acc: HGNC: 30290] | | |
| DUSP13 | dual specificity phosphatase 13 | ENSG00000079393 | 51207 |
| | [Source: HGNC | | |
| | Symbol; Acc: HGNC: 19681] | | |
| DUSP19 | dual specificity phosphatase 19 | ENSG00000162999 | 142679 |
| | [Source: HGNC | | |
| | Symbol; Acc: HGNC: 18894] | | |
| DUXA | double homeobox A | ENSG00000258873 | 503835 |
| | [Source: HGNC | | |
| | Symbol; Acc: HGNC: 32179] | | |
| DUXB | double homeobox B | ENSG00000282757 | 100033411 |
| | [Source: HGNC | | |
| | Symbol; Acc: HGNC: 33345] | | |
| DYRK3 | dual specificity tyrosine | ENSG00000143479 | 8444 |
| | phosphorylation regulated | | |
| | kinase 3 [Source: HGNC | | |
| | Symbol; Acc: HGNC: 3094] | | |
| EDNRB | endothelin receptor type B | ENSG00000136160 | 1910 |
| | [Source: HGNC | | |
| | Symbol; Acc: HGNC: 3180] | | |
| EEF1A1P28 | eukaryotic translation | ENSG00000237709 | 0 |
| | elongation factor 1 alpha 1 | | |
| | pseudogene 28 [Source: HGNC | | |
| | Symbol; Acc: HGNC: 37902] | | |
| EFCAB6 | EF-hand calcium binding | ENSG00000186976 | 64800 |
| | domain 6 [Source: HGNC | | |
| | Symbol; Acc: HGNC: 24204] | | |
| EIF4A2P4 | eukaryotic translation initiation | ENSG00000224781 | 0 |
| | factor 4A2 pseudogene 4 | | |
| | [Source: HGNC | | |
| | Symbol; Acc: HGNC: 45101] | | |
| EIF4BP2 | eukaryotic translation initiation | ENSG00000228753 | 0 |
| | factor 4B pseudogene 2 | | |
| | [Source: HGNC | | |
| | Symbol; Acc: HGNC: 37935] | | |
| ERVMER34-1 | endogenous retrovirus group | ENSG00000226887 | 100288413 |
| | MER34 member 1, envelope | | |
| | [Source: HGNC | | |
| | Symbol; Acc: HGNC: 42970] | | |
| FABP9 | fatty acid binding protein 9 | ENSG00000205186 | 646480 |
| | [Source: HGNC | | |
| | Symbol; Acc: HGNC: 3563] | | |
| FAIM2 | Fas apoptotic inhibitory | ENSG00000135472 | 23017 |
| | molecule 2 [Source: HGNC | | |
| | Symbol; Acc: HGNC: 17067] | | |
| FAM13C | family with sequence similarity | ENSG00000148541 | 220965 |
| | 13 member C [Source: HGNC | | |
| | Symbol; Acc: HGNC: 19371] | | |
| FAM160A1 | FHF complex subunit HOOK | ENSG00000164142 | 729830 |
| | interacting protein 1A | | |
| | [Source: HGNC | | |
| | Symbol; Acc: HGNC: 34237] | | |
| FAM221B | family with sequence similarity | ENSG00000204930 | 392307 |
| | 221 member B [Source: HGNC | | |
| | Symbol; Acc: HGNC: 30762] | | |
| FAM71B | family with sequence similarity | ENSG00000170613 | 0 |
| | 71 member B [Source: HGNC | | |
| | Symbol; Acc: HGNC: 28397] | | |
| FAM74A3 | family with sequence similarity | ENSG00000274355 | 0 |
| | 74 member A3 [Source: HGNC | | |
| | Symbol; Acc: HGNC: 32031] | | |
| FAM83B | family with sequence similarity | ENSG00000168143 | 222584 |
| | 83 member B [Source: HGNC | | |
| | Symbol; Acc: HGNC: 21357] | | |
| FCRL4 | Fc receptor like 4 | ENSG00000163518 | 83417 |
| | [Source: HGNC | | |
| | Symbol; Acc: HGNC: 18507] | | |
| FETUB | fetuin B [Source: HGNC | ENSG00000090512 | 26998 |
| | Symbol; Acc: HGNC: 3658] | | |
| FHL5 | four and a half LIM domains 5 | ENSG00000112214 | 9457 |
| | [Source: HGNC | | |
| | Symbol; Acc: HGNC: 17371] | | |
| FLRT2 | fibronectin leucine rich | ENSG00000185070 | 23768 |
| | transmembrane protein 2 | | |
| | [Source: HGNC | | |
| | Symbol; Acc: HGNC: 3761] | | |
| FRK | fyn related Src family tyrosine | ENSG00000111816 | 2444 |
| | kinase [Source: HGNC | | |
| | Symbol; Acc: HGNC: 3955] | | |
| FRMD5 | FERM domain containing 5 | ENSG00000171877 | 84978 |
| | [Source: HGNC | | |
| | Symbol; Acc: HGNC: 28214] | | |
| FSCB | fibrous sheath CABYR binding | ENSG00000189139 | 84075 |
| | protein [Source: HGNC | | |
| | Symbol; Acc: HGNC: 20494] | | |
| FTCDNL1 | formiminotransferase | ENSG00000226124 | 348751 |
| | cyclodeaminase N-terminal like | | |
| | [Source: HGNC | | |
| | Symbol; Acc: HGNC: 48661] | | |
| FZD6 | frizzled class receptor 6 | ENSG00000164930 | 8323 |
| | [Source: HGNC | | |
| | Symbol; Acc: HGNC: 4044] | | |
| GABRA3 | gamma-aminobutyric acid type | ENSG00000011677 | 0 |
| | A receptor subunit alpha3 | | |
| | [Source: HGNC | | |
| | Symbol; Acc: HGNC: 4077] | | |
| GABRB2 | gamma-aminobutyric acid type | ENSG00000145864 | 2561 |
| | A receptor subunit beta2 | | |
| | [Source: HGNC | | |
| | Symbol; Acc: HGNC: 4082] | | |
| GABRR3 | gamma-aminobutyric acid type | ENSG00000183185 | 200959 |
| | A receptor subunit rho3 | | |
| | [Source: HGNC | | |
| | Symbol; Acc: HGNC: 17969] | | |
| GAGE10 | G antigen 10 [Source: HGNC | ENSG00000215274 | 102724473 |
| | Symbol; Acc: HGNC: 30968] | | |
| GAGE2A | G antigen 2A [Source: HGNC | ENSG00000189064 | 26749 |
| | Symbol; Acc: HGNC: 4099] | | |
| GALNT15 | polypeptide N- | ENSG00000131386 | 117248 |
| | acetylgalactosaminyltransferase | | |
| | 15 [Source: HGNC | | |
| | Symbol; Acc: HGNC: 21531] | | |
| GALNTL5 | polypeptide N- | ENSG00000106648 | 168391 |
| | acetylgalactosaminyltransferase | | |
| | like 5 [Source: HGNC | | |
| | Symbol; Acc: HGNC: 21725] | | |
| GASK1A | golgi associated kinase 1A | ENSG00000144649 | 0 |
| | [Source: HGNC | | |
| | Symbol; Acc: HGNC: 24485] | | |
| GBP7 | guanylate binding protein 7 | ENSG00000213512 | 388646 |
| | [Source: HGNC | | |
| | Symbol; Acc: HGNC: 29606] | | |
| GC | GC vitamin D binding protein | ENSG00000145321 | 2638 |
| | [Source: HGNC | | |
| | Symbol; Acc: HGNC: 4187] | | |
| GDF9 | growth differentiation factor 9 | ENSG00000164404 | 2661 |
| | [Source: HGNC | | |
| | Symbol; Acc: HGNC: 4224] | | |
| GKN1 | gastrokine 1 [Source: HGNC | ENSG00000169605 | 56287 |
| | Symbol; Acc: HGNC: 23217] | | |
| GNA14 | G protein subunit alpha 14 | ENSG00000156049 | 9630 |
| | [Source: HGNC | | |
| | Symbol; Acc: HGNC: 4382] | | |
| GNAQP1 | G protein subunit alpha q | ENSG00000214077 | 0 |
| | pseudogene 1 [Source: HGNC | | |
| | Symbol; Acc: HGNC: 4391] | | |
| GNRHR | gonadotropin releasing | ENSG00000109163 | 2798 |
| | hormone receptor | | |
| | [Source: HGNC | | |
| | Symbol; Acc: HGNC: 4421] | | |
| GOLGA6C | golgin A6 family member C | ENSG00000167195 | 653641 |
| | [Source: HGNC | | |
| | Symbol; Acc: HGNC: 32206] | | |
| GOLGA6L7 | golgin A6 family like 7 | ENSG00000261649 | 728310 |
| | [Source: HGNC | | |
| | Symbol; Acc: HGNC: 37442] | | |
| GPC6 | glypican 6 [Source: HGNC | ENSG00000183098 | 10082 |
| | Symbol; Acc: HGNC: 4454] | | |
| GPR12 | G protein-coupled receptor 12 | ENSG00000132975 | 2835 |
| | [Source: HGNC | | |
| | Symbol; Acc: HGNC: 4466] | | |
| GPR148 | G protein-coupled receptor 148 | ENSG00000173302 | 344561 |
| | [Source: HGNC | | |
| | Symbol; Acc: HGNC: 23623] | | |
| GPR156 | G protein-coupled receptor 156 | ENSG00000175697 | 165829 |
| | [Source: HGNC | | |
| | Symbol; Acc: HGNC: 20844] | | |
| GPR176 | G protein-coupled receptor 176 | ENSG00000166073 | 11245 |
| | [Source: HGNC | | |
| | Symbol; Acc: HGNC: 32370] | | |
| GPR21 | G protein-coupled receptor 21 | ENSG00000188394 | 2844 |
| | [Source: HGNC | | |
| | Symbol; Acc: HGNC: 4476] | | |
| GPR63 | G protein-coupled receptor 63 | ENSG00000112218 | 81491 |
| | [Source: HGNC | | |
| | Symbol; Acc: HGNC: 13302] | | |
| GPR82 | G protein-coupled receptor 82 | ENSG00000171657 | 27197 |
| | [Source: HGNC | | |
| | Symbol; Acc: HGNC: 4533] | | |
| GPR83 | G protein-coupled receptor 83 | ENSG00000123901 | 10888 |
| | [Source: HGNC | | |
| | Symbol; Acc: HGNC: 4523] | | |
| GPRC5A | G protein-coupled receptor | ENSG00000013588 | 9052 |
| | class C group 5 member A | | |
| | [Source: HGNC | | |
| | Symbol; Acc: HGNC: 9836] | | |
| GRB7 | growth factor receptor bound | ENSG00000141738 | 2886 |
| | protein 7 [Source: HGNC | | |
| | Symbol; Acc: HGNC: 4567] | | |
| GRHL1 | grainyhead like transcription | ENSG00000134317 | 29841 |
| | factor 1 [Source: HGNC | | |
| | Symbol; Acc: HGNC: 17923] | | |
| GRID1 | glutamate ionotropic receptor | ENSG00000182771 | 2894 |
| | delta type subunit 1 | | |
| | [Source: HGNC | | |
| | Symbol; Acc: HGNC: 4575] | | |
| GRM5 | glutamate metabotropic | ENSG00000168959 | 2915 |
| | receptor 5 [Source: HGNC | | |
| | Symbol; Acc: HGNC: 4597] | | |
| GSTA3 | glutathione S-transferase alpha | ENSG00000174156 | 2940 |
| | 3 [Source: HGNC | | |
| | Symbol; Acc: HGNC: 4628] | | |
| GYG1P3 | glycogenin 1 pseudogene 3 | ENSG00000231095 | 0 |
| | [Source: HGNC | | |
| | Symbol; Acc: HGNC: 39711] | | |
| H2BC1 | H2B clustered histone 1 | ENSG00000146047 | 255626 |
| | [Source: HGNC | | |
| | Symbol; Acc: HGNC: 18730] | | |
| HABP2 | hyaluronan binding protein 2 | ENSG00000148702 | 3026 |
| | [Source: HGNC | | |
| | Symbol; Acc: HGNC: 4798] | | |
| HADHBP1 | HADHB pseudogene 1 | ENSG00000238193 | 0 |
| | [Source: HGNC | | |
| | Symbol; Acc: HGNC: 54887] | | |
| HAO2 | hydroxyacid oxidase 2 | ENSG00000116882 | 51179 |
| | [Source: HGNC | | |
| | Symbol; Acc: HGNC: 4810] | | |
| HAS2 | hyaluronan synthase 2 | ENSG00000170961 | 3037 |
| | [Source: HGNC | | |
| | Symbol; Acc: HGNC: 4819] | | |
| HHIP | hedgehog interacting protein | ENSG00000164161 | 64399 |
| | [Source: HGNC | | |
| | Symbol; Acc: HGNC: 14866] | | |
| HMGB3P6 | high mobility group box 3 | ENSG00000213070 | 0 |
| | pseudogene 6 [Source: HGNC | | |
| | Symbol; Acc: HGNC: 39283] | | |
| HMGCS2 | 3-hydroxy-3-methylglutaryl- | ENSG00000134240 | 3158 |
| | CoA synthase 2 | | |
| | [Source: HGNC | | |
| | Symbol; Acc: HGNC: 5008] | | |
| HNRNPA1P23 | heterogeneous nuclear | ENSG00000240236 | 0 |
| | ribonucleoprotein A1 | | |
| | pseudogene 23 [Source: HGNC | | |
| | Symbol; Acc: HGNC: 39541] | | |
| HNRNPA1P37 | heterogeneous nuclear | ENSG00000218574 | 0 |
| | ribonucleoprotein A1 | | |
| | pseudogene 37 [Source: HGNC | | |
| | Symbol; Acc: HGNC: 48766] | | |
| HNRNPA1P4 | heterogeneous nuclear | ENSG00000206228 | 0 |
| | ribonucleoprotein A1 | | |
| | pseudogene 4 [Source: HGNC | | |
| | Symbol; Acc: HGNC: 32234] | | |
| HNRNPA1P46 | heterogeneous nuclear | ENSG00000228020 | 0 |
| | ribonucleoprotein A1 | | |
| | pseudogene 46 [Source: HGNC | | |
| | Symbol; Acc: HGNC: 48776] | | |
| HNRNPA1P53 | heterogeneous nuclear | ENSG00000229534 | 0 |
| | ribonucleoprotein A1 | | |
| | pseudogene 53 [Source: HGNC | | |
| | Symbol; Acc: HGNC: 48783] | | |
| HNRNPCP6 | heterogeneous nuclear | ENSG00000213305 | 0 |
| | ribonucleoprotein C | | |
| | pseudogene 6 [Source: HGNC | | |
| | Symbol; Acc: HGNC: 48817] | | |
| HOXD10 | homeobox D10 [Source: HGNC | ENSG00000128710 | 3236 |
| | Symbol; Acc: HGNC: 5133] | | |
| HSD17B2 | hydroxysteroid 17-beta | ENSG00000086696 | 3294 |
| | dehydrogenase 2 | | |
| | [Source: HGNC | | |
| | Symbol; Acc: HGNC: 5211] | | |
| HSD17B7P1 | hydroxysteroid 17-beta | ENSG00000232908 | 0 |
| | dehydrogenase 7 pseudogene 1 | | |
| | [Source: HGNC | | |
| | Symbol; Acc: HGNC: 18689] | | |
| HSP90AB6P | heat shock protein 90 alpha | ENSG00000235137 | 0 |
| | family class B member 6, | | |
| | pseudogene [Source: HGNC | | |
| | Symbol; Acc: HGNC: 32540] | | |
| HSPA8P9 | heat shock protein family A | ENSG00000241478 | 0 |
| | (Hsp70) member 8 pseudogene | | |
| | 9 [Source: HGNC | | |
| | Symbol; Acc: HGNC: 44924] | | |
| HTR1A | 5-hydroxytryptamine receptor | ENSG00000178394 | 3350 |
| | 1A [Source: HGNC | | |
| | Symbol; Acc: HGNC: 5286] | | |
| HTR1B | 5-hydroxytryptamine receptor | ENSG00000135312 | 3351 |
| | 1B [Source: HGNC | | |
| | Symbol; Acc: HGNC: 5287] | | |
| HTR2C | 5-hydroxytryptamine receptor | ENSG00000147246 | 0 |
| | 2C [Source: HGNC | | |
| | Symbol; Acc: HGNC: 5295] | | |
| HTR3B | 5-hydroxytryptamine receptor | ENSG00000149305 | 9177 |
| | 3B [Source: HGNC | | |
| | Symbol; Acc: HGNC: 5298] | | |
| HYKK | hydroxylysine kinase | ENSG00000188266 | 123688 |
| | [Source: HGNC | | |
| | Symbol; Acc: HGNC: 34403] | | |
| IDO2 | indoleamine 2,3-dioxygenase 2 | ENSG00000188676 | 169355 |
| | [Source: HGNC | | |
| | Symbol; Acc: HGNC: 27269] | | |
| IFNA14 | interferon alpha 14 | ENSG00000228083 | 3448 |
| | [Source: HGNC | | |
| | Symbol; Acc: HGNC: 5420] | | |
| IFNA2 | interferon alpha 2 | ENSG00000188379 | 3440 |
| | [Source: HGNC | | |
| | Symbol; Acc: HGNC: 5423] | | |
| IFNA4 | interferon alpha 4 | ENSG00000236637 | 3441 |
| | [Source: HGNC | | |
| | Symbol; Acc: HGNC: 5425] | | |
| IGSF11 | immunoglobulin superfamily | ENSG00000144847 | 152404 |
| | member 11 [Source: HGNC | | |
| | Symbol; Acc: HGNC: 16669] | | |
| IGSF5 | immunoglobulin superfamily | ENSG00000183067 | 150084 |
| | member 5 [Source: HGNC | | |
| | Symbol; Acc: HGNC: 5952] | | |
| IL13RA2 | interleukin 13 receptor subunit | ENSG00000123496 | 3598 |
| | alpha 2 [Source: HGNC | | |
| | Symbol; Acc: HGNC: 5975] | | |
| IL1RAPL1 | interleukin 1 receptor accessory | ENSG00000169306 | 11141 |
| | protein like 1 [Source: HGNC | | |
| | Symbol; Acc: HGNC: 5996] | | |
| IL1RAPL2 | interleukin 1 receptor accessory | ENSG00000189108 | 26280 |
| | protein like 2 [Source: HGNC | | |
| | Symbol; Acc: HGNC: 5997] | | |
| IL2 | interleukin 2 [Source: HGNC | ENSG00000109471 | 3558 |
| | Symbol; Acc: HGNC: 6001] | | |
| IL36B | interleukin 36 beta | ENSG00000136696 | 27177 |
| | [Source: HGNC | | |
| | Symbol; Acc: HGNC: 15564] | | |
| IL5 | interleukin 5 [Source: HGNC | ENSG00000113525 | 3567 |
| | Symbol; Acc: HGNC: 6016] | | |
| ILRUNP1 | ILRUN pseudogene 1 | ENSG00000227513 | 0 |
| | [Source: HGNC | | |
| | Symbol; Acc: HGNC: 54748] | | |
| INHBE | inhibin subunit beta E | ENSG00000139269 | 83729 |
| | [Source: HGNC | | |
| | Symbol; Acc: HGNC: 24029] | | |
| INSC | INSC spindle orientation | ENSG00000188487 | 387755 |
| | adaptor protein [Source: HGNC | | |
| | Symbol; Acc: HGNC: 33116] | | |
| JAKMIP2-AS1 | JAKMIP2 antisense RNA 1 | ENSG00000280780 | 153469 |
| | [Source: HGNC | | |
| | Symbol; Acc: HGNC: 27203] | | |
| KANK4 | KN motif and ankyrin repeat | ENSG00000132854 | 163782 |
| | domains 4 [Source: HGNC | | |
| | Symbol; Acc: HGNC: 27263] | | |
| KBTBD12 | kelch repeat and BTB domain | ENSG00000187715 | 166348 |
| | containing 12 [Source: HGNC | | |
| | Symbol; Acc: HGNC: 25731] | | |
| KCNAB1 | potassium voltage-gated | ENSG00000169282 | 7881 |
| | channel subfamily A regulatory | | |
| | beta subunit 1 [Source: HGNC | | |
| | Symbol; Acc: HGNC: 6228] | | |
| KCNJ1 | potassium inwardly rectifying | ENSG00000151704 | 3758 |
| | channel subfamily J member 1 | | |
| | [Source: HGNC | | |
| | Symbol; Acc: HGNC: 6255] | | |
| KCNJ3 | potassium inwardly rectifying | ENSG00000162989 | 3760 |
| | channel subfamily J member 3 | | |
| | [Source: HGNC | | |
| | Symbol; Acc: HGNC: 6264] | | |
| KDM4F | lysine demethylase 4F | ENSG00000255855 | 0 |
| | [Source: HGNC | | |
| | Symbol; Acc: HGNC: 52413] | | |
| KIAA1210 | KIAA1210 [Source: HGNC | ENSG00000250423 | 57481 |
| | Symbol; Acc: HGNC: 29218] | | |
| KIAA2012 | KIAA2012 [Source: HGNC | ENSG00000182329 | 100652824 |
| | Symbol; Acc: HGNC: 51250] | | |
| KITLG | KIT ligand [Source: HGNC | ENSG00000049130 | 4254 |
| | Symbol; Acc: HGNC: 6343] | | |
| KPNA2P2 | karyopherin subunit alpha 2 | ENSG00000228928 | 0 |
| | pseudogene 2 [Source: HGNC | | |
| | Symbol; Acc: HGNC: 52872] | | |
| KRT26 | keratin 26 [Source: HGNC | ENSG00000186393 | 353288 |
| | Symbol; Acc: HGNC: 30840] | | |
| KRT4 | keratin 4 [Source: HGNC | ENSG00000170477 | 0 |
| | Symbol; Acc: HGNC: 6441] | | |
| KRT8P10 | keratin 8 pseudogene 10 | ENSG00000231203 | 0 |
| | [Source: HGNC | | |
| | Symbol; Acc: HGNC: 33364] | | |
| KRTAP19-8 | keratin associated protein 19-8 | ENSG00000206102 | 728299 |
| | [Source: HGNC | | |
| | Symbol; Acc: HGNC: 33898] | | |
| LA16c-60G3.7 | single stranded DNA binding | ENSG00000213727 | 0 |
| | protein 3 (SSBP3) pseudogene | | |
| LAD1 | ladinin 1 [Source: HGNC | ENSG00000159166 | 3898 |
| | Symbol; Acc: HGNC: 6472] | | |
| LGSN | lengsin, lens protein with | ENSG00000146166 | 51557 |
| | glutamine synthetase domain | | |
| | [Source: HGNC | | |
| | Symbol; Acc: HGNC: 21016] | | |
| LHX8 | LIM homeobox 8 | ENSG00000162624 | 431707 |
| | [Source: HGNC | | |
| | Symbol; Acc: HGNC: 28838] | | |
| LINC00293 | long intergenic non-protein | ENSG00000253314 | 497634 |
| | coding RNA 293 | | |
| | [Source: HGNC | | |
| | Symbol; Acc: HGNC: 39078] | | |
| LINC00544 | long intergenic non-protein | ENSG00000122043 | 0 |
| | coding RNA 544 | | |
| | [Source: HGNC | | |
| | Symbol; Acc: HGNC: 43679] | | |
| LINC01118 | long intergenic non-protein | ENSG00000222005 | 388948 |
| | coding RNA 1118 | | |
| | [Source: HGNC | | |
| | Symbol; Acc: HGNC: 49261] | | |
| LINC01630 | long intergenic non-protein | ENSG00000227115 | 100287225 |
| | coding RNA 1630 | | |
| | [Source: HGNC | | |
| | Symbol; Acc: HGNC: 52295] | | |
| LINGO4 | leucine rich repeat and Ig | ENSG00000213171 | 339398 |
| | domain containing 4 | | |
| | [Source: HGNC | | |
| | Symbol; Acc: HGNC: 31814] | | |
| LIPG | lipase G, endothelial type | ENSG00000101670 | 9388 |
| | [Source: HGNC | | |
| | Symbol; Acc: HGNC: 6623] | | |
| LIPK | lipase family member K | ENSG00000204021 | 643414 |
| | [Source: HGNC | | |
| | Symbol; Acc: HGNC: 23444] | | |
| LMNTD1 | lamin tail domain containing 1 | ENSG00000152936 | 160492 |
| | [Source: HGNC | | |
| | Symbol; Acc: HGNC: 26683] | | |
| LRP2BP | LRP2 binding protein | ENSG00000109771 | 55805 |
| | [Source: HGNC | | |
| | Symbol; Acc: HGNC: 25434] | | |
| LRRC17 | leucine rich repeat containing | ENSG00000128606 | 10234 |
| | 17 [Source: HGNC | | |
| | Symbol; Acc: HGNC: 16895] | | |
| LRRC30 | leucine rich repeat containing | ENSG00000206422 | 339291 |
| | 30 [Source: HGNC | | |
| | Symbol; Acc: HGNC: 30219] | | |
| LRRTM3 | leucine rich repeat | ENSG00000198739 | 0 |
| | transmembrane neuronal 3 | | |
| | [Source: HGNC | | |
| | Symbol; Acc: HGNC: 19410] | | |
| LRTM1 | leucine rich repeats and | ENSG00000144771 | 57408 |
| | transmembrane domains 1 | | |
| | [Source: HGNC | | |
| | Symbol; Acc: HGNC: 25023] | | |
| LYG2 | lysozyme g2 [Source: HGNC | ENSG00000185674 | 254773 |
| | Symbol; Acc: HGNC: 29615] | | |
| LYRM4-AS1 | LYRM4 antisense RNA 1 | ENSG00000272142 | 0 |
| | [Source: NCBI gene (formerly | | |
| | Entrezgene); Acc: 100129461] | | |
| MAB21L1 | mab-21 like 1 [Source: HGNC | ENSG00000180660 | 4081 |
| | Symbol; Acc: HGNC: 6757] | | |
| MAGEA11 | MAGE family member A11 | ENSG00000185247 | 4110 |
| | [Source: HGNC | | |
| | Symbol; Acc: HGNC: 6798] | | |
| MAGEA12 | MAGE family member A12 | ENSG00000213401 | 4111 |
| | [Source: HGNC | | |
| | Symbol; Acc: HGNC: 6799] | | |
| MAGEA3 | MAGE family member A3 | ENSG00000221867 | 4102 |
| | [Source: HGNC | | |
| | Symbol; Acc: HGNC: 6801] | | |
| MAGEA4 | MAGE family member A4 | ENSG00000147381 | 4103 |
| | [Source: HGNC | | |
| | Symbol; Acc: HGNC: 6802] | | |
| MAGEB6B | MAGE family member B6B | ENSG00000232030 | 0 |
| | [Source: HGNC | | |
| | Symbol; Acc: HGNC: 28824] | | |
| MAJIN | membrane anchored junction | ENSG00000168070 | 283129 |
| | protein [Source: HGNC | | |
| | Symbol; Acc: HGNC: 27441] | | |
| MAMDC2 | MAM domain containing 2 | ENSG00000165072 | 256691 |
| | [Source: HGNC | | |
| | Symbol; Acc: HGNC: 23673] | | |
| MAOA | monoamine oxidase A | ENSG00000189221 | 4128 |
| | [Source: HGNC | | |
| | Symbol; Acc: HGNC: 6833] | | |
| MATN3 | matrilin 3 [Source: HGNC | ENSG00000132031 | 0 |
| | Symbol; Acc: HGNC: 6909] | | |
| MLF1 | myeloid leukemia factor 1 | ENSG00000178053 | 4291 |
| | [Source: HGNC | | |
| | Symbol; Acc: HGNC: 7125] | | |
| MLPH | melanophilin [Source: HGNC | ENSG00000115648 | 79083 |
| | Symbol; Acc: HGNC: 29643] | | |
| MMP20 | matrix metallopeptidase 20 | ENSG00000137674 | 9313 |
| | [Source: HGNC | | |
| | Symbol; Acc: HGNC: 7167] | | |
| MOCOS | molybdenum cofactor sulfurase | ENSG00000075643 | 0 |
| | [Source: HGNC | | |
| | Symbol; Acc: HGNC: 18234] | | |
| MRAP2 | melanocortin 2 receptor | ENSG00000135324 | 112609 |
| | accessory protein 2 | | |
| | [Source: HGNC | | |
| | Symbol; Acc: HGNC: 21232] | | |
| MRGPRX4 | MAS related GPR family | ENSG00000179817 | 117196 |
| | member X4 [Source: HGNC | | |
| | Symbol; Acc: HGNC: 17617] | | |
| MRGPRX5P | MAS related GPR family | ENSG00000255536 | 0 |
| | member X5, pseudogene | | |
| | [Source: HGNC | | |
| | Symbol; Acc: HGNC: 54329] | | |
| MRPL3P1 | mitochondrial ribosomal | ENSG00000215349 | 0 |
| | protein L3 pseudogene 1 | | |
| | [Source: HGNC | | |
| | Symbol; Acc: HGNC: 29701] | | |
| MS4A12 | membrane spanning 4-domains | ENSG00000071203 | 54860 |
| | A12 [Source: HGNC | | |
| | Symbol; Acc: HGNC: 13370] | | |
| MSTRG | MSTRG | MSTRG | #N/A |
| MSTRG | MSTRG | MSTRG | #N/A |
| MSTRG | MSTRG | MSTRG | #N/A |
| MSTRG | MSTRG | MSTRG | #N/A |
| MSTRG | MSTRG | MSTRG | #N/A |
| MSTRG | MSTRG | MSTRG | #N/A |
| MSTRG | MSTRG | MSTRG | #N/A |
| MSTRG | MSTRG | MSTRG | #N/A |
| MSTRG | MSTRG | MSTRG | #N/A |
| MSTRG | MSTRG | MSTRG | #N/A |
| MSTRG | MSTRG | MSTRG | #N/A |
| MSTRG | MSTRG | MSTRG | #N/A |
| MSTRG | MSTRG | MSTRG | #N/A |
| MSTRG | MSTRG | MSTRG | #N/A |
| MSTRG | MSTRG | MSTRG | #N/A |
| MSTRG | MSTRG | MSTRG | #N/A |
| MSTRG | MSTRG | MSTRG | #N/A |
| MSTRG | MSTRG | MSTRG | #N/A |
| MTNR1A | melatonin receptor 1A | ENSG00000168412 | 4543 |
| | [Source: HGNC | | |
| | Symbol; Acc: HGNC: 7463] | | |
| MYOC | myocilin [Source: HGNC | ENSG00000034971 | 4653 |
| | Symbol; Acc: HGNC: 7610] | | |
| MYOT | myotilin [Source: HGNC | ENSG00000120729 | 9499 |
| | Symbol; Acc: HGNC: 12399] | | |
| NCOA4P1 | nuclear receptor coactivator 4 | ENSG00000258629 | 0 |
| | pseudogene 1 [Source: HGNC | | |
| | Symbol; Acc: HGNC: 20022] | | |
| NKAIN3 | sodium/potassium transporting | ENSG00000185942 | 286183 |
| | ATPase interacting 3 | | |
| | [Source: HGNC | | |
| | Symbol; Acc: HGNC: 26829] | | |
| NMBR | neuromedin B receptor | ENSG00000135577 | 4829 |
| | [Source: HGNC | | |
| | Symbol; Acc: HGNC: 7843] | | |
| NOX3 | NADPH oxidase 3 | ENSG00000074771 | 50508 |
| | [Source: HGNC | | |
| | Symbol; Acc: HGNC: 7890] | | |
| NPY2R | neuropeptide Y receptor Y2 | ENSG00000185149 | 4887 |
| | [Source: HGNC | | |
| | Symbol; Acc: HGNC: 7957] | | |
| NPY5R | neuropeptide Y receptor Y5 | ENSG00000164129 | 4889 |
| | [Source: HGNC | | |
| | Symbol; Acc: HGNC: 7958] | | |
| NRIP3 | nuclear receptor interacting | ENSG00000175352 | 56675 |
| | protein 3 [Source: HGNC | | |
| | Symbol; Acc: HGNC: 1167] | | |
| NTS | neurotensin [Source: HGNC | ENSG00000133636 | 4922 |
| | Symbol; Acc: HGNC: 8038] | | |
| NWD1 | NACHT and WD repeat | ENSG00000188039 | 284434 |
| | domain containing 1 | | |
| | [Source: HGNC | | |
| | Symbol; Acc: HGNC: 27619] | | |
| ODAM | odontogenic, ameloblast | ENSG00000109205 | 54959 |
| | associated [Source: HGNC | | |
| | Symbol; Acc: HGNC: 26043] | | |
| OR10A7 | olfactory receptor family 10 | ENSG00000179919 | 121364 |
| | subfamily A member 7 | | |
| | [Source: HGNC | | |
| | Symbol; Acc: HGNC: 15329] | | |
| OR10AB1P | olfactory receptor family 10 | ENSG00000176716 | 0 |
| | subfamily AB member 1 | | |
| | pseudogene [Source: HGNC | | |
| | Symbol; Acc: HGNC: 14804] | | |
| OR10G7 | olfactory receptor family 10 | ENSG00000182634 | 390265 |
| | subfamily G member 7 | | |
| | [Source: HGNC | | |
| | Symbol; Acc: HGNC: 14842] | | |
| OR10S1 | olfactory receptor family 10 | ENSG00000196248 | 219873 |
| | subfamily S member 1 | | |
| | [Source: HGNC | | |
| | Symbol; Acc: HGNC: 14807] | | |
| OR10X1 | olfactory receptor family 10 | ENSG00000279111 | 128367 |
| | subfamily X member 1 | | |
| | [Source: HGNC | | |
| | Symbol; Acc: HGNC: 14995] | | |
| OR2K2 | olfactory receptor family 2 | ENSG00000171133 | 26248 |
| | subfamily K member 2 | | |
| | [Source: HGNC | | |
| | Symbol; Acc: HGNC: 8264] | | |
| OR2M4 | olfactory receptor family 2 | ENSG00000171180 | 26245 |
| | subfamily M member 4 | | |
| | [Source: HGNC | | |
| | Symbol; Acc: HGNC: 8270] | | |
| OR4C5 | olfactory receptor family 4 | ENSG00000176540 | 79346 |
| | subfamily C member 5 | | |
| | [Source: HGNC | | |
| | Symbol; Acc: HGNC: 14702] | | |
| OR4D11 | olfactory receptor family 4 | ENSG00000176200 | 219986 |
| | subfamily D member 11 | | |
| | [Source: HGNC | | |
| | Symbol; Acc: HGNC: 15174] | | |
| OR51E1 | olfactory receptor family 51 | ENSG00000180785 | 143503 |
| | subfamily E member 1 | | |
| | [Source: HGNC | | |
| | Symbol; Acc: HGNC: 15194] | | |
| OR51S1 | olfactory receptor family 51 | ENSG00000176922 | 119692 |
| | subfamily S member 1 | | |
| | [Source: HGNC | | |
| | Symbol; Acc: HGNC: 15204] | | |
| OR52A4P | olfactory receptor family 52 | ENSG00000205494 | 0 |
| | subfamily A member 4 | | |
| | pseudogene [Source: HGNC | | |
| | Symbol; Acc: HGNC: 19579] | | |
| OR52B6 | olfactory receptor family 52 | ENSG00000187747 | 340980 |
| | subfamily B member 6 | | |
| | [Source: HGNC | | |
| | Symbol; Acc: HGNC: 15211] | | |
| OR52E5 | olfactory receptor family 52 | ENSG00000277932 | 390082 |
| | subfamily E member 5 | | |
| | [Source: HGNC | | |
| | Symbol; Acc: HGNC: 15214] | | |
| OR5F1 | olfactory receptor family 5 | ENSG00000149133 | 338674 |
| | subfamily F member 1 | | |
| | [Source: HGNC | | |
| | Symbol; Acc: HGNC: 8343] | | |
| OR5H14 | olfactory receptor family 5 | ENSG00000236032 | 403273 |
| | subfamily H member 14 | | |
| | [Source: HGNC | | |
| | Symbol; Acc: HGNC: 31286] | | |
| OR6C2 | olfactory receptor family 6 | ENSG00000179695 | 341416 |
| | subfamily C member 2 | | |
| | [Source: HGNC | | |
| | Symbol; Acc: HGNC: 15436] | | |
| OR7A17 | olfactory receptor family 7 | ENSG00000185385 | 26333 |
| | subfamily A member 17 | | |
| | [Source: HGNC | | |
| | Symbol; Acc: HGNC: 8363] | | |
| OR7E111FP | olfactory receptor family 7 | ENSG00000250804 | 0 |
| | subfamily E member 111F | | |
| | pseudogene [Source: HGNC | | |
| | Symbol; Acc: HGNC: 55115] | | |
| OR7E115P | olfactory receptor family 7 | ENSG00000182531 | 0 |
| | subfamily E member 115 | | |
| | pseudogene [Source: HGNC | | |
| | Symbol; Acc: HGNC: 15127] | | |
| OR7E121P | olfactory receptor family 7 | ENSG00000244222 | 0 |
| | subfamily E member 121 | | |
| | pseudogene [Source: HGNC | | |
| | Symbol; Acc: HGNC: 15049] | | |
| OR7E122P | olfactory receptor family 7 | ENSG00000215160 | 0 |
| | subfamily E member 122 | | |
| | pseudogene [Source: HGNC | | |
| | Symbol; Acc: HGNC: 15050] | | |
| OR7E5P | olfactory receptor family 7 | ENSG00000214880 | 0 |
| | subfamily E member 5 | | |
| | pseudogene [Source: HGNC | | |
| | Symbol; Acc: HGNC: 8435] | | |
| OR8B3 | olfactory receptor family 8 | ENSG00000284609 | 390271 |
| | subfamily B member 3 | | |
| | [Source: HGNC | | |
| | Symbol; Acc: HGNC: 8472] | | |
| OR8F1P | olfactory receptor family 8 | ENSG00000239426 | 0 |
| | subfamily F member 1 | | |
| | pseudogene [Source: HGNC | | |
| | Symbol; Acc: HGNC: 14691] | | |
| ORC6 | origin recognition complex | ENSG00000091651 | 23594 |
| | subunit 6 [Source: HGNC | | |
| | Symbol; Acc: HGNC: 17151] | | |
| OTX2 | orthodenticle homeobox 2 | ENSG00000165588 | 5015 |
| | [Source: HGNC | | |
| | Symbol; Acc: HGNC: 8522] | | |
| PAGE3 | PAGE family member 3 | ENSG00000204279 | 0 |
| | [Source: HGNC | | |
| | Symbol; Acc: HGNC: 4110] | | |
| PARP1P1 | poly(ADP-ribose) polymerase | ENSG00000227105 | 0 |
| | 1 pseudogene 1 [Source: HGNC | | |
| | Symbol; Acc: HGNC: 275] | | |
| PAXBP1-AS1 | PAXBP1 antisense RNA 1 | ENSG00000238197 | 100506215 |
| | [Source: HGNC | | |
| | Symbol; Acc: HGNC: 39603] | | |
| PBK | PDZ binding kinase | ENSG00000168078 | 55872 |
| | [Source: HGNC | | |
| | Symbol; Acc: HGNC: 18282] | | |
| PCDHB 1 | protocadherin beta 1 | ENSG00000171815 | 29930 |
| | [Source: HGNC | | |
| | Symbol; Acc: HGNC: 8680] | | |
| PCSK1 | proprotein convertase | ENSG00000175426 | 5122 |
| | subtilisin/kexin type 1 | | |
| | [Source: HGNC | | |
| | Symbol; Acc: HGNC: 8743] | | |
| PDCD1LG2 | programmed cell death 1 ligand | ENSG00000197646 | 80380 |
| | 2 [Source: HGNC | | |
| | Symbol; Acc: HGNC: 18731] | | |
| PDE11A | MSTRG | ENSG00000284741 | #N/A |
| PDGFRL | platelet derived growth factor | ENSG00000104213 | 5157 |
| | receptor like [Source: HGNC | | |
| | Symbol; Acc: HGNC: 8805] | | |
| PDLIM3 | PDZ and LIM domain 3 | ENSG00000154553 | 27295 |
| | [Source: HGNC | | |
| | Symbol; Acc: HGNC: 20767] | | |
| PGBD5 | piggyBac transposable element | ENSG00000177614 | 0 |
| | derived 5 [Source: HGNC | | |
| | Symbol; Acc: HGNC: 19405] | | |
| PGM5P2 | phosphoglucomutase 5 | ENSG00000277778 | 595135 |
| | pseudogene 2 [Source: HGNC | | |
| | Symbol; Acc: HGNC: 18965] | | |
| PHF24 | PHD finger protein 24 | ENSG00000122733 | 23349 |
| | [Source: HGNC | | |
| | Symbol; Acc: HGNC: 29180] | | |
| PHYHIP | phytanoyl-CoA 2-hydroxylase | ENSG00000168490 | 9796 |
| | interacting protein | | |
| | [Source: HGNC | | |
| | Symbol; Acc: HGNC: 16865] | | |
| PIK3R3 | phosphoinositide-3-kinase | ENSG00000117461 | 8503 |
| | regulatory subunit 3 | | |
| | [Source: HGNC | | |
| | Symbol; Acc: HGNC: 8981] | | |
| PLA2G4F | phospholipase A2 group IVF | ENSG00000168907 | 255189 |
| | [Source: HGNC | | |
| | Symbol; Acc: HGNC: 27396] | | |
| PLD5 | phospholipase D family | ENSG00000180287 | 200150 |
| | member 5 [Source: HGNC | | |
| | Symbol; Acc: HGNC: 26879] | | |
| PNLIPRP3 | pancreatic lipase related protein | ENSG00000203837 | 119548 |
| | 3 [Source: HGNC | | |
| | Symbol; Acc: HGNC: 23492] | | |
| PNPT1P1 | polyribonucleotide | ENSG00000229241 | 0 |
| | nucleotidyltransferase 1 | | |
| | pseudogene 1 [Source: HGNC | | |
| | Symbol; Acc: HGNC: 44468] | | |
| POU1F1 | POU class 1 homeobox 1 | ENSG00000064835 | 5449 |
| | [Source: HGNC | | |
| | Symbol; Acc: HGNC: 9210] | | |
| PPIAP15 | peptidylprolyl isomerase A | ENSG00000244196 | 0 |
| | pseudogene 15 [Source: HGNC | | |
| | Symbol; Acc: HGNC: 9268] | | |
| PPIAP23 | peptidylprolyl isomerase A | ENSG00000214178 | 0 |
| | pseudogene 23 [Source: HGNC | | |
| | Symbol; Acc: HGNC: 39268] | | |
| PPIAP33 | peptidylprolyl isomerase A | ENSG00000178654 | 0 |
| | pseudogene 33 [Source: HGNC | | |
| | Symbol; Acc: HGNC: 49752] | | |
| PPIAP74 | peptidylprolyl isomerase A | ENSG00000220343 | 0 |
| | pseudogene 74 [Source: HGNC | | |
| | Symbol; Acc: HGNC: 53698] | | |
| PPIAP77 | peptidylprolyl isomerase A | ENSG00000249092 | 0 |
| | pseudogene 77 [Source: HGNC | | |
| | Symbol; Acc: HGNC: 53701] | | |
| PPM1E | protein phosphatase, | ENSG00000175175 | 22843 |
| | Mg2+/Mn2+ dependent 1E | | |
| | [Source: HGNC | | |
| | Symbol; Acc: HGNC: 19322] | | |
| PPP5D1 | PPP5 tetratricopeptide repeat | ENSG00000230510 | 100506012 |
| | domain containing 1, | | |
| | pseudogene [Source: HGNC | | |
| | Symbol; Acc: HGNC: 44209] | | |
| PRELP | proline and arginine rich end | ENSG00000188783 | 5549 |
| | leucine rich repeat protein | | |
| | [Source: HGNC | | |
| | Symbol; Acc: HGNC: 9357] | | |
| PRKAA2 | protein kinase AMP-activated | ENSG00000162409 | 5563 |
| | catalytic subunit alpha 2 | | |
| | [Source: HGNC | | |
| | Symbol; Acc: HGNC: 9377] | | |
| PRPF38AP2 | PRP38 domain containing A | ENSG00000236582 | 0 |
| | pseudogene 2 [Source: HGNC | | |
| | Symbol; Acc: HGNC: 44693] | | |
| PRSS37 | serine protease 37 | ENSG00000165076 | 136242 |
| | [Source: HGNC | | |
| | Symbol; Acc: HGNC: 29211] | | |
| PSG4 | pregnancy specific beta-1- | ENSG00000243137 | 5672 |
| | glycoprotein 4 [Source: HGNC | | |
| | Symbol; Acc: HGNC: 9521] | | |
| PSG6 | pregnancy specific beta-1- | ENSG00000170848 | 5675 |
| | glycoprotein 6 [Source: HGNC | | |
| | Symbol; Acc: HGNC: 9523] | | |
| PTN | pleiotrophin [Source: HGNC | ENSG00000105894 | 5764 |
| | Symbol; Acc: HGNC: 9630] | | |
| RBBP4P4 | RBBP4 pseudogene 4 | ENSG00000214561 | 0 |
| | [Source: HGNC | | |
| | Symbol; Acc: HGNC: 42371] | | |
| RBMXP1 | RBMX pseudogene 1 | ENSG00000216835 | 0 |
| | [Source: HGNC | | |
| | Symbol; Acc: HGNC: 9911] | | |
| RFX4 | regulatory factor X4 | ENSG00000111783 | 5992 |
| | [Source: HGNC | | |
| | Symbol; Acc: HGNC: 9985] | | |
| RFX8 | regulatory factor X8 | ENSG00000196460 | 731220 |
| | [Source: HGNC | | |
| | Symbol; Acc: HGNC: 37253] | | |
| RGS17 | regulator of G protein signaling | ENSG00000091844 | 26575 |
| | 17 [Source: HGNC | | |
| | Symbol; Acc: HGNC: 14088] | | |
| RGS4 | regulator of G protein signaling | ENSG00000117152 | 5999 |
| | 4 [Source: HGNC | | |
| | Symbol; Acc: HGNC: 10000] | | |
| RGS5 | regulator of G protein signaling | ENSG00000143248 | 8490 |
| | 5 [Source: HGNC | | |
| | Symbol; Acc: HGNC: 10001] | | |
| RGS7BP | regulator of G protein signaling | ENSG00000186479 | 401190 |
| | 7 binding protein | | |
| | [Source: HGNC | | |
| | Symbol; Acc: HGNC: 23271] | | |
| RHCG | Rh family C glycoprotein | ENSG00000140519 | 51458 |
| | [Source: HGNC | | |
| | Symbol; Acc: HGNC: 18140] | | |
| RHOJ | ras homolog family member J | ENSG00000126785 | 57381 |
| | [Source: HGNC | | |
| | Symbol; Acc: HGNC: 688] | | |
| ROR1 | receptor tyrosine kinase like | ENSG00000185483 | 4919 |
| | orphan receptor 1 | | |
| | [Source: HGNC | | |
| | Symbol; Acc: HGNC: 10256] | | |
| RORB | RAR related orphan receptor B | ENSG00000198963 | 6096 |
| | [Source: HGNC | | |
| | Symbol; Acc: HGNC: 10259] | | |
| RP1-100J12.1 | novel transcript, antisense to | ENSG00000273254 | 0 |
| | USP16 | | |
| RP11-101E19.8 | similar to zinc finger protein | ENSG00000228984 | 0 |
| | 682 | | |
| RP11-119H12.4 | TAF4b RNA polymerase II, | ENSG00000234841 | 0 |
| | TATA box binding protein | | |
| | (TBP)-associated factor, | | |
| | 105 kDa (TAF4B) pseudogene | | |
| RP11-160H12.3 | suppressor of cytokine | ENSG00000254629 | 0 |
| | signaling 6 (SOCS6) | | |
| | pseudogene | | |
| RP11-163O19.16 | tripartite motif-containing | ENSG00000265973 | 0 |
| | pseudogene | | |
| RP11-23B15.5 | novel transcript, sense intronic | ENSG00000287070 | 0 |
| | to TRMO | | |
| RP11-259P15.4 | ring finger protein 13 (RNF13) | ENSG00000242068 | 0 |
| | pseudogene | | |
| RP11-274B18.3 | dermatan sulfate epimerase | ENSG00000224025 | 0 |
| | (DSE) pseudogene | | |
| RP11-313I2.5 | Cdon homolog (mouse) | ENSG00000255540 | 0 |
| | (CDON) pseudogene | | |
| RP11-317B7.2 | pseudogene similar to part of | ENSG00000249006 | 0 |
| | HBS1-like | | |
| RP1-131F15.2 | T-cell activation leucine repeat- | ENSG00000237115 | 0 |
| | rich protein (TA-LRRP) | | |
| | pseudogene | | |
| RP11-320A16.1 | capping protein (actin filament) | ENSG00000261549 | 0 |
| | muscle Z-line, alpha 2 | | |
| | (CAPZA2) pseudogene | | |
| RP11-329A14.1 | amyotrophic lateral sclerosis 2 | ENSG00000235105 | 0 |
| | (juvenile) chromosome region, | | |
| | candidate 2 (ALS2CR2) | | |
| | pseudogene | | |
| RP11-346J10.3 | POM121 transmembrane | ENSG00000273957 | 0 |
| | nucleoporin (POM121) | | |
| | pseudogene | | |
| RP11-348N17.2 | novel ankyrin repeat domain 20 | ENSG00000288694 | 0 |
| | family member pseudogene | | |
| RP11-353J17.5 | novel transcript | ENSG00000284800 | 0 |
| RP11-360F5.1 | novel transcript | ENSG00000249207 | 0 |
| RP11-365F18.3 | Male-specific lethal-3 homolog | ENSG00000239254 | 0 |
| | 1 (Msl3l1) pseudogene | | |
| RP11-377D9.1 | ribosomal protein S26 (RPS26) | ENSG00000243129 | 0 |
| | pseudogene | | |
| RP11-44N12.4 | RNA binding motif protein 4 | ENSG00000253415 | 0 |
| | family pseudogene | | |
| RP11-466J24.1 | piggyBac transposable element | ENSG00000251501 | 0 |
| | derived 3 (PGBD3) | | |
| | pseudogene | | |
| RP11-471N19.1 | piggyBac transposable element | ENSG00000257308 | 0 |
| | derived 3 (PGBD3) | | |
| | pseudogene | | |
| RP11-487E13.1 | novel transcript | ENSG00000249008 | 0 |
| RP11-512F24.1 | heterogeneous nuclear | ENSG00000232499 | 0 |
| | ribonucleoprotein A3 | | |
| | (hnRNPA3) pseudogene | | |
| RP11-529G21.3 | pseudogene similar to | ENSG00000241671 | 0 |
| | KIAA1328 | | |
| RP11-618I10.2 | UDP glucuronosyltransferase 2 | ENSG00000250612 | 0 |
| | family pseudogene | | |
| RP11-66B24.4 | ALDH1A3 antisense RNA 1 | ENSG00000259583 | 101927751 |
| | [Source: HGNC | | |
| | Symbol; Acc: HGNC: 55416] | | |
| RP11-703I16.3 | telomeric repeat binding factor | ENSG00000266995 | 0 |
| | (NIMA-interacting) 1 (TERF1) | | |
| | pseudogene | | |
| RP11-760D2.13 | zinc finger protein 195 | ENSG00000278205 | 0 |
| | (ZNF195) pseudogene | | |
| RP1-59B16.1 | interferon stimulated | ENSG00000220748 | 0 |
| | exonuclease gene 20 kDa-like 2 | | |
| | (ISG20L2) pseudogene | | |
| RP3-326L13.2 | TEC | ENSG00000279437 | 0 |
| RP3-359N14.1 | chromatin assembly factor 1, | ENSG00000219088 | 0 |
| | p60 subunit B (CHAF1B) | | |
| | pseudogene | | |
| RP3-528L19.1 | novel transcript, antisense to | ENSG00000286887 | 0 |
| | SGK1 | | |
| RSPH1 | radial spoke head component 1 | ENSG00000160188 | 89765 |
| | [Source: HGNC | | |
| | Symbol; Acc: HGNC: 12371] | | |
| RSPO3 | R-spondin 3 [Source: HGNC | ENSG00000146374 | 84870 |
| | Symbol; Acc: HGNC: 20866] | | |
| SAGE2P | sarcoma antigen 2, pseudogene | ENSG00000198022 | 0 |
| | [Source: HGNC | | |
| | Symbol; Acc: HGNC: 51337] | | |
| SAXO2 | stabilizer of axonemal | ENSG00000188659 | 283726 |
| | microtubules 2 [Source: HGNC | | |
| | Symbol; Acc: HGNC: 33727] | | |
| SCGN | secretagogin, EF-hand calcium | ENSG00000079689 | 10590 |
| | binding protein [Source: HGNC | | |
| | Symbol; Acc: HGNC: 16941] | | |
| SCIN | scinderin [Source: HGNC | ENSG00000006747 | 85477 |
| | Symbol; Acc: HGNC: 21695] | | |
| SCOC | short coiled-coil protein | ENSG00000153130 | 60592 |
| | [Source: HGNC | | |
| | Symbol; Acc: HGNC: 20335] | | |
| SDC1 | syndecan 1 [Source: HGNC | ENSG00000115884 | 6382 |
| | Symbol; Acc: HGNC: 10658] | | |
| SDR9C7 | short chain | ENSG00000170426 | 121214 |
| | dehydrogenase/reductase | | |
| | family 9C member 7 | | |
| | [Source: HGNC | | |
| | Symbol; Acc: HGNC: 29958] | | |
| SELE | selectin E [Source: HGNC | ENSG00000007908 | 6401 |
| | Symbol; Acc: HGNC: 10718] | | |
| SELENOP | selenoprotein P [Source: HGNC | ENSG00000250722 | 6414 |
| | Symbol; Acc: HGNC: 10751] | | |
| SEPHS1P4 | selenophosphate synthetase 1 | ENSG00000230146 | 0 |
| | pseudogene 4 [Source: HGNC | | |
| | Symbol; Acc: HGNC: 42169] | | |
| SERPINB12 | serpin family B member 12 | ENSG00000166634 | 89777 |
| | [Source: HGNC | | |
| | Symbol; Acc: HGNC: 14220] | | |
| SERPINB7 | serpin family B member 7 | ENSG00000166396 | 8710 |
| | [Source: HGNC | | |
| | Symbol; Acc: HGNC: 13902] | | |
| SETSIP | SET like protein | ENSG00000230667 | 0 |
| | [Source: HGNC | | |
| | Symbol; Acc: HGNC: 42937] | | |
| SFTPA2 | surfactant protein A2 | ENSG00000185303 | 729238 |
| | [Source: HGNC | | |
| | Symbol; Acc: HGNC: 10799] | | |
| SH2D6 | SH2 domain containing 6 | ENSG00000152292 | 284948 |
| | [Source: HGNC | | |
| | Symbol; Acc: HGNC: 30439] | | |
| SHC4 | SHC adaptor protein 4 | ENSG00000185634 | 0 |
| | [Source: HGNC | | |
| | Symbol; Acc: HGNC: 16743] | | |
| SLC13A1 | solute carrier family 13 | ENSG00000081800 | 6561 |
| | member 1 [Source: HGNC | | |
| | Symbol; Acc: HGNC: 10916] | | |
| SLC16A2 | solute carrier family 16 | ENSG00000147100 | 0 |
| | member 2 [Source: HGNC | | |
| | Symbol; Acc: HGNC: 10923] | | |
| SLC25A15P2 | solute carrier family 25 | ENSG00000237164 | 0 |
| | member 15 pseudogene 2 | | |
| | [Source: HGNC | | |
| | Symbol; Acc: HGNC: 39843] | | |
| SLC25A38P1 | solute carrier family 25 | ENSG00000229785 | 0 |
| | member 38 pseudogene 1 | | |
| | [Source: HGNC | | |
| | Symbol; Acc: HGNC: 43858] | | |
| SLC25A5P7 | solute carrier family 25 | ENSG00000218180 | 0 |
| | member 5 pseudogene 7 | | |
| | [Source: HGNC | | |
| | Symbol; Acc: HGNC: 513] | | |
| SLC26A5 | solute carrier family 26 | ENSG00000170615 | 375611 |
| | member 5 [Source: HGNC | | |
| | Symbol; Acc: HGNC: 9359] | | |
| SLC2A12 | solute carrier family 2 member | ENSG00000146411 | 154091 |
| | 12 [Source: HGNC | | |
| | Symbol; Acc: HGNC: 18067] | | |
| SLC36A2 | solute carrier family 36 | ENSG00000186335 | 153201 |
| | member 2 [Source: HGNC | | |
| | Symbol; Acc: HGNC: 18762] | | |
| SLC4A9 | solute carrier family 4 member | ENSG00000113073 | 83697 |
| | 9 [Source: HGNC | | |
| | Symbol; Acc: HGNC: 11035] | | |
| SLC6A2 | solute carrier family 6 member | ENSG00000103546 | 6530 |
| | 2 [Source: HGNC | | |
| | Symbol; Acc: HGNC: 11048] | | |
| SLC6A5 | solute carrier family 6 member | ENSG00000165970 | 9152 |
| | 5 [Source: HGNC | | |
| | Symbol; Acc: HGNC: 11051] | | |
| SLC7A14 | solute carrier family 7 member | ENSG00000013293 | 0 |
| | 14 [Source: HGNC | | |
| | Symbol; Acc: HGNC: 29326] | | |
| SLC7A2 | solute carrier family 7 member | ENSG00000003989 | 6542 |
| | 2 [Source: HGNC | | |
| | Symbol; Acc: HGNC: 11060] | | |
| SLC9B1 | solute carrier family 9 member | ENSG00000164037 | 150159 |
| | B1 [Source: HGNC | | |
| | Symbol; Acc: HGNC: 24244] | | |
| SLCO1A2 | solute carrier organic anion | ENSG00000084453 | 6579 |
| | transporter family member 1A2 | | |
| | [Source: HGNC | | |
| | Symbol; Acc: HGNC: 10956] | | |
| SLCO1C1 | solute carrier organic anion | ENSG00000139155 | 53919 |
| | transporter family member 1C1 | | |
| | [Source: HGNC | | |
| | Symbol; Acc: HGNC: 13819] | | |
| SLITRK1 | SLIT and NTRK like family | ENSG00000178235 | 114798 |
| | member 1 [Source: HGNC | | |
| | Symbol; Acc: HGNC: 20297] | | |
| SLITRK2 | SLIT and NTRK like family | ENSG00000185985 | 84631 |
| | member 2 [Source: HGNC | | |
| | Symbol; Acc: HGNC: 13449] | | |
| SMYD1 | SET and MYND domain | ENSG00000115593 | 150572 |
| | containing 1 [Source: HGNC | | |
| | Symbol; Acc: HGNC: 20986] | | |
| SNTG1 | syntrophin gamma 1 | ENSG00000147481 | 54212 |
| | [Source: HGNC | | |
| | Symbol; Acc: HGNC: 13740] | | |
| SNX19P2 | sorting nexin 19 pseudogene 2 | ENSG00000235500 | 0 |
| | [Source: HGNC | | |
| | Symbol; Acc: HGNC: 38115] | | |
| SNX19P3 | sorting nexin 19 pseudogene 3 | ENSG00000264570 | 0 |
| | [Source: HGNC | | |
| | Symbol; Acc: HGNC: 38116] | | |
| SPP1 | secreted phosphoprotein 1 | ENSG00000118785 | 6696 |
| | [Source: HGNC | | |
| | Symbol; Acc: HGNC: 11255] | | |
| SRPX2 | sushi repeat containing protein | ENSG00000102359 | 27286 |
| | X-linked 2 [Source: HGNC | | |
| | Symbol; Acc: HGNC: 30668] | | |
| SSX3 | SSX family member 3 | ENSG00000165584 | 10214 |
| | [Source: HGNC | | |
| | Symbol; Acc: HGNC: 11337] | | |
| SSX6P | SSX family member 6, | ENSG00000171483 | 0 |
| | pseudogene [Source: HGNC | | |
| | Symbol; Acc: HGNC: 19652] | | |
| SSXP1 | SSX family pseudogene 1 | ENSG00000197185 | 0 |
| | [Source: HGNC | | |
| | Symbol; Acc: HGNC: 30637] | | |
| ST3GAL1P1 | ST3GAL1 pseudogene 1 | ENSG00000250656 | 0 |
| | [Source: HGNC | | |
| | Symbol; Acc: HGNC: 45174] | | |
| STC1 | stanniocalcin 1 [Source: HGNC | ENSG00000159167 | 6781 |
| | Symbol; Acc: HGNC: 11373] | | |
| STEAP2 | STEAP2 metalloreductase | ENSG00000157214 | 261729 |
| | [Source: HGNC | | |
| | Symbol; Acc: HGNC: 17885] | | |
| SUGCT | succinyl-CoA: glutarate-CoA | ENSG00000175600 | 79783 |
| | transferase [Source: HGNC | | |
| | Symbol; Acc: HGNC: 16001] | | |
| SULT1C4 | sulfotransferase family 1C | ENSG00000198075 | 27233 |
| | member 4 [Source: HGNC | | |
| | Symbol; Acc: HGNC: 11457] | | |
| SV2B | synaptic vesicle glycoprotein | ENSG00000185518 | 9899 |
| | 2B [Source: HGNC | | |
| | Symbol; Acc: HGNC: 16874] | | |
| SV2C | synaptic vesicle glycoprotein | ENSG00000122012 | 22987 |
| | 2C [Source: HGNC | | |
| | Symbol; Acc: HGNC: 30670] | | |
| SYT14P1 | synaptotagmin 14 pseudogene | ENSG00000215127 | 0 |
| | 1 [Source: HGNC | | |
| | Symbol; Acc: HGNC: 33429] | | |
| SYT16 | synaptotagmin 16 | ENSG00000139973 | 83851 |
| | [Source: HGNC | | |
| | Symbol; Acc: HGNC: 23142] | | |
| TBATA | thymus, brain and testes | ENSG00000166220 | 219793 |
| | associated [Source: HGNC | | |
| | Symbol; Acc: HGNC: 23511] | | |
| TBPL2 | TATA-box binding protein like | ENSG00000182521 | 387332 |
| | 2 [Source: HGNC | | |
| | Symbol; Acc: HGNC: 19841] | | |
| TEAD1 | TEA domain transcription | ENSG00000187079 | 7003 |
| | factor 1 [Source: HGNC | | |
| | Symbol; Acc: HGNC: 11714] | | |
| TERF1P6 | TERF1 pseudogene 6 | ENSG00000223824 | 0 |
| | [Source: HGNC | | |
| | Symbol; Acc: HGNC: 54602] | | |
| TET1P1 | tet methylcytosine dioxygenase | ENSG00000232204 | 0 |
| | 1 pseudogene 1 [Source: HGNC | | |
| | Symbol; Acc: HGNC: 33586] | | |
| TKTL2 | transketolase like 2 | ENSG00000151005 | 84076 |
| | [Source: HGNC | | |
| | Symbol; Acc: HGNC: 25313] | | |
| TMEM130 | transmembrane protein 130 | ENSG00000166448 | 222865 |
| | [Source: HGNC | | |
| | Symbol; Acc: HGNC: 25429] | | |
| TMEM196 | transmembrane protein 196 | ENSG00000173452 | 256130 |
| | [Source: HGNC | | |
| | Symbol; Acc: HGNC: 22431] | | |
| TMEM207 | transmembrane protein 207 | ENSG00000198398 | 131920 |
| | [Source: HGNC | | |
| | Symbol; Acc: HGNC: 33705] | | |
| TMEM253 | transmembrane protein 253 | ENSG00000232070 | 643382 |
| | [Source: HGNC | | |
| | Symbol; Acc: HGNC: 32545] | | |
| TMEM45A | transmembrane protein 45A | ENSG00000181458 | 55076 |
| | [Source: HGNC | | |
| | Symbol; Acc: HGNC: 25480] | | |
| TMEM59L | transmembrane protein 59 like | ENSG00000105696 | 25789 |
| | [Source: HGNC | | |
| | Symbol; Acc: HGNC: 13237] | | |
| TMPRSS11D | transmembrane serine protease | ENSG00000153802 | 9407 |
| | 11D [Source: HGNC | | |
| | Symbol; Acc: HGNC: 24059] | | |
| TMPRSS12 | transmembrane serine protease | ENSG00000186452 | 0 |
| | 12 [Source: HGNC | | |
| | Symbol; Acc: HGNC: 28779] | | |
| TMPRSS2 | transmembrane serine protease | ENSG00000184012 | 7113 |
| | 2 [Source: HGNC | | |
| | Symbol; Acc: HGNC: 11876] | | |
| TMPRSS7 | transmembrane serine protease | ENSG00000176040 | 344805 |
| | 7 [Source: HGNC | | |
| | Symbol; Acc: HGNC: 30846] | | |
| TMSB15A | thymosin beta 15A | ENSG00000158164 | 0 |
| | [Source: HGNC | | |
| | Symbol; Acc: HGNC: 30744] | | |
| TPI1P1 | triosephosphate isomerase 1 | ENSG00000226415 | 0 |
| | pseudogene 1 [Source: HGNC | | |
| | Symbol; Acc: HGNC: 35449] | | |
| TPI1P3 | triosephosphate isomerase 1 | ENSG00000186743 | 0 |
| | pseudogene 3 [Source: HGNC | | |
| | Symbol; Acc: HGNC: 38070] | | |
| TPRXL | tetrapeptide repeat homeobox | ENSG00000180438 | 348825 |
| | like (pseudogene) | | |
| | [Source: HGNC | | |
| | Symbol; Acc: HGNC: 32178] | | |
| TREH | trehalase [Source: HGNC | ENSG00000118094 | 11181 |
| | Symbol; Acc: HGNC: 12266] | | |
| TRIM49C | tripartite motif containing 49C | ENSG00000204449 | 642612 |
| | [Source: HGNC | | |
| | Symbol; Acc: HGNC: 38877] | | |
| TRIM6 | tripartite motif containing 6 | ENSG00000121236 | 117854 |
| | [Source: HGNC | | |
| | Symbol; Acc: HGNC: 16277] | | |
| TRIM63 | tripartite motif containing 63 | ENSG00000158022 | 84676 |
| | [Source: HGNC | | |
| | Symbol; Acc: HGNC: 16007] | | |
| TRIM64DP | tripartite motif containing 64D, | ENSG00000254751 | 0 |
| | pseudogene [Source: HGNC | | |
| | Symbol; Acc: HGNC: 43974] | | |
| TRIM67 | tripartite motif containing 67 | ENSG00000119283 | 440730 |
| | [Source: HGNC | | |
| | Symbol; Acc: HGNC: 31859] | | |
| TTC23L | tetratricopeptide repeat domain | ENSG00000205838 | 153657 |
| | 23 like [Source: HGNC | | |
| | Symbol; Acc: HGNC: 26355] | | |
| TTC29 | tetratricopeptide repeat domain | ENSG00000137473 | 83894 |
| | 29 [Source: HGNC | | |
| | Symbol; Acc: HGNC: 29936] | | |
| TUBB3P1 | tubulin beta 3 class III | ENSG00000220418 | 0 |
| | pseudogene 1 [Source: HGNC | | |
| | Symbol; Acc: HGNC: 42339] | | |
| TUBB4AP1 | tubulin beta 4A class Iva | ENSG00000228466 | 0 |
| | pseudogene 1 [Source: HGNC | | |
| | Symbol; Acc: HGNC: 42340] | | |
| TUBB8P7 | tubulin beta 8 class VIII | ENSG00000261812 | 0 |
| | pseudogene 7 [Source: HGNC | | |
| | Symbol; Acc: HGNC: 42345] | | |
| TULP2 | TUB like protein 2 | ENSG00000104804 | 7288 |
| | [Source: HGNC | | |
| | Symbol; Acc: HGNC: 12424] | | |
| TXNDC8 | thioredoxin domain containing | ENSG00000204193 | 255220 |
| | 8 [Source: HGNC | | |
| | Symbol; Acc: HGNC: 31454] | | |
| UBE2NL | ubiquitin conjugating enzyme | ENSG00000276380 | 389898 |
| | E2 N like (gene/pseudogene) | | |
| | [Source: HGNC | | |
| | Symbol; Acc: HGNC: 31710] | | |
| UBQLN3 | ubiquilin 3 [Source: HGNC | ENSG00000175520 | 50613 |
| | Symbol; Acc: HGNC: 12510] | | |
| UCP1 | uncoupling protein 1 | ENSG00000109424 | 7350 |
| | [Source: HGNC | | |
| | Symbol; Acc: HGNC: 12517] | | |
| UGT1A3 | UDP glucuronosyltransferase | ENSG00000288702 | 54659 |
| | family 1 member A3 | | |
| | [Source: HGNC | | |
| | Symbol; Acc: HGNC: 12535] | | |
| UGT3A1 | UDP glycosyltransferase | ENSG00000145626 | 133688 |
| | family 3 member A1 | | |
| | [Source: HGNC | | |
| | Symbol; Acc: HGNC: 26625] | | |
| UGT3A2 | UDP glycosyltransferase | ENSG00000168671 | 167127 |
| | family 3 member A2 | | |
| | [Source: HGNC | | |
| | Symbol; Acc: HGNC: 27266] | | |
| ULBP1 | UL16 binding protein 1 | ENSG00000111981 | 80329 |
| | [Source: HGNC | | |
| | Symbol; Acc: HGNC: 14893] | | |
| UPK1B | uroplakin 1B [Source: HGNC | ENSG00000114638 | 7348 |
| | Symbol; Acc: HGNC: 12578] | | |
| UPP2 | uridine phosphorylase 2 | ENSG00000007001 | 151531 |
| | [Source: HGNC | | |
| | Symbol; Acc: HGNC: 23061] | | |
| USP26 | ubiquitin specific peptidase 26 | ENSG00000134588 | 83844 |
| | [Source: HGNC | | |
| | Symbol; Acc: HGNC: 13485] | | |
| VPS35P1 | VPS35 pseudogene 1 | ENSG00000260809 | 0 |
| | [Source: HGNC | | |
| | Symbol; Acc: HGNC: 51805] | | |
| VSTM4 | V-set and transmembrane | ENSG00000165633 | 196740 |
| | domain containing 4 | | |
| | [Source: HGNC | | |
| | Symbol; Acc: HGNC: 26470] | | |
| WIPF3 | WAS/WASL interacting | ENSG00000122574 | 644150 |
| | protein family member 3 | | |
| | [Source: HGNC | | |
| | Symbol; Acc: HGNC: 22004] | | |
| XRCC6P4 | X-ray repair cross | ENSG00000253517 | 0 |
| | complementing 6 pseudogene 4 | | |
| | [Source: HGNC | | |
| | Symbol; Acc: HGNC: 45186] | | |
| ZIM2 | zinc finger imprinted 2 | ENSG00000269699 | 23619 |
| | [Source: HGNC | | |
| | Symbol; Acc: HGNC: 12875] | | |
| ZNF215 | zinc finger protein 215 | ENSG00000149054 | 7762 |
| | [Source: HGNC | | |
| | Symbol; Acc: HGNC: 13007] | | |
| ZNF229 | zinc finger protein 229 | ENSG00000278318 | 7772 |
| | [Source: HGNC | | |
| | Symbol; Acc: HGNC: 13022] | | |
| ZNF652P1 | zinc finger protein 652 | ENSG00000235278 | 0 |
| | pseudogene 1 [Source: HGNC | | |
| | Symbol; Acc: HGNC: 35166] | | |
| ZNF774 | zinc finger protein 774 | ENSG00000196391 | 342132 |
| | [Source: HGNC | | |
| | Symbol; Acc: HGNC: 33108] | | |
| ZNF812P | zinc finger protein 812, | ENSG00000224689 | 0 |
| | pseudogene [Source: HGNC | | |
| | Symbol; Acc: HGNC: 33242] | | |
| ZP4 | zona pellucida glycoprotein 4 | ENSG00000116996 | 57829 |
| | [Source: HGNC | | |
| | Symbol; Acc: HGNC: 15770] | | |
| ZSCAN5B | zinc finger and SCAN domain | ENSG00000197213 | 342933 |
| | containing 5B [Source: HGNC | | |
| | Symbol; Acc: HGNC: 34246] | | |

FM2

| | | | |
|---|---|---|---|
| ACAP2 | ArfGAP with coiled-coil, | ENSG00000114331 | 23527 |
| | ankyrin repeat and PH domains | | |
| | 2 [Source: HGNC | | |
| | Symbol; Acc: HGNC: 16469] | | |
| ADGRE1 | adhesion G protein-coupled | ENSG00000174837 | 2015 |
| | receptor E1 [Source: HGNC | | |
| | Symbol; Acc: HGNC: 3336] | | |
| ADGRE3 | adhesion G protein-coupled | ENSG00000131355 | 84658 |
| | receptor E3 [Source: HGNC | | |
| | Symbol; Acc: HGNC: 23647] | | |
| ADGRG1 | adhesion G protein-coupled | ENSG00000205336 | 9289 |
| | receptor G1 [Source: HGNC | | |
| | Symbol; Acc: HGNC: 4512] | | |
| AGTRAP | angiotensin II receptor | ENSG00000177674 | 57085 |
| | associated protein | | |
| | [Source: HGNC | | |
| | Symbol; Acc: HGNC: 13539] | | |
| ALDH2 | aldehyde dehydrogenase 2 | ENSG00000111275 | 217 |
| | family member [Source: HGNC | | |
| | Symbol; Acc: HGNC: 404] | | |
| ALDH3B1 | aldehyde dehydrogenase 3 | ENSG00000006534 | 221 |
| | family member B1 | | |
| | [Source: HGNC | | |
| | Symbol; Acc: HGNC: 410] | | |
| ALDOA | aldolase, fructose-bisphosphate | ENSG00000149925 | 226 |
| | A [Source: HGNC | | |
| | Symbol; Acc: HGNC: 414] | | |
| AMOT | angiomotin [Source: HGNC | ENSG00000126016 | 0 |
| | Symbol; Acc: HGNC: 17810] | | |
| ANPEP | alanyl aminopeptidase, | ENSG00000166825 | 290 |
| | membrane [Source: HGNC | | |
| | Symbol; Acc: HGNC: 500] | | |
| AP1S2 | adaptor related protein complex | ENSG00000182287 | 8905 |
| | 1 subunit sigma 2 | | |
| | [Source: HGNC | | |
| | Symbol; Acc: HGNC: 560] | | |
| APLP2 | amyloid beta precursor like | ENSG00000084234 | 334 |
| | protein 2 [Source: HGNC | | |
| | Symbol; Acc: HGNC: 598] | | |
| AQP9 | aquaporin 9 [Source: HGNC | ENSG00000103569 | 366 |
| | Symbol; Acc: HGNC: 643] | | |
| ARHGEF10L | Rho guanine nucleotide | ENSG00000074964 | 55160 |
| | exchange factor 10 like | | |
| | [Source: HGNC | | |
| | Symbol; Acc: HGNC: 25540] | | |
| ARHGEF11 | Rho guanine nucleotide | ENSG00000132694 | 9826 |
| | exchange factor 11 | | |
| | [Source: HGNC | | |
| | Symbol; Acc: HGNC: 14580] | | |
| ARID3A | AT-rich interaction domain 3A | ENSG00000116017 | 1820 |
| | [Source: HGNC | | |
| | Symbol; Acc: HGNC: 3031] | | |
| ARRB2 | arrestin beta 2 [Source: HGNC | ENSG00000141480 | 409 |
| | Symbol; Acc: HGNC: 712] | | |
| ASAH1 | N-acylsphingosine | ENSG00000104763 | 427 |
| | amidohydrolase 1 | | |
| | [Source: HGNC | | |
| | Symbol; Acc: HGNC: 735] | | |
| ASGR1 | asialoglycoprotein receptor 1 | ENSG00000141505 | 432 |
| | [Source: HGNC | | |
| | Symbol; Acc: HGNC: 742] | | |
| ATP11A | ATPase phospholipid | ENSG00000068650 | 23250 |
| | transporting 11A | | |
| | [Source: HGNC | | |
| | Symbol; Acc: HGNC: 13552] | | |
| ATP6V0B | ATPase H+ transporting V0 | ENSG00000117410 | 533 |
| | subunit b [Source: HGNC | | |
| | Symbol; Acc: HGNC: 861] | | |
| ATP6V0C | ATPase H+ transporting V0 | ENSG00000185883 | 527 |
| | subunit c [Source: HGNC | | |
| | Symbol; Acc: HGNC: 855] | | |
| ATP6V0D1 | ATPase H+ transporting V0 | ENSG00000159720 | 9114 |
| | subunit d1 [Source: HGNC | | |
| | Symbol; Acc: HGNC: 13724] | | |
| ATP6V1B2 | ATPase H+ transporting V1 | ENSG00000147416 | 526 |
| | subunit B2 [Source: HGNC | | |
| | Symbol; Acc: HGNC: 854] | | |
| ATP9A | ATPase phospholipid | ENSG00000054793 | 10079 |
| | transporting 9A (putative) | | |
| | [Source: HGNC | | |
| | Symbol; Acc: HGNC: 13540] | | |
| B3GNT8 | UDP-GlcNAc: betaGal beta- | ENSG00000177191 | 374907 |
| | 1,3-N- | | |
| | acetylglucosaminyltransferase | | |
| | 8 [Source: HGNC | | |
| | Symbol; Acc: HGNC: 24139] | | |
| BCKDK | branched chain keto acid | ENSG00000103507 | 10295 |
| | dehydrogenase kinase | | |
| | [Source: HGNC | | |
| | Symbol; Acc: HGNC: 16902] | | |
| BCL6 | BCL6 transcription repressor | ENSG00000113916 | 604 |
| | [Source: HGNC | | |
| | Symbol; Acc: HGNC: 1001] | | |
| BCL6B | BCL6B transcription repressor | ENSG00000161940 | 255877 |
| | [Source: HGNC | | |
| | Symbol; Acc: HGNC: 1002] | | |
| BLVRB | biliverdin reductase B | ENSG00000090013 | 645 |
| | [Source: HGNC | | |
| | Symbol; Acc: HGNC: 1063] | | |
| BMP2K | BMP2 inducible kinase | ENSG00000138756 | 55589 |
| | [Source: HGNC | | |
| | Symbol; Acc: HGNC: 18041] | | |
| BRD2 | bromodomain containing 2 | ENSG00000235307 | 6046 |
| | [Source: HGNC | | |
| | Symbol; Acc: HGNC: 1103] | | |
| BRI3 | brain protein I3 [Source: HGNC | ENSG00000164713 | 0 |
| | Symbol; Acc: HGNC: 1109] | | |
| BRINP2 | BMP/retinoic acid inducible | ENSG00000198797 | 57795 |
| | neural specific 2 | | |
| | [Source: HGNC | | |
| | Symbol; Acc: HGNC: 13746] | | |
| BST1 | bone marrow stromal cell | ENSG00000109743 | 683 |
| | antigen 1 [Source: HGNC | | |
| | Symbol; Acc: HGNC: 1118] | | |
| BTF3 | basic transcription factor 3 | ENSG00000145741 | 689 |
| | [Source: HGNC | | |
| | Symbol; Acc: HGNC: 1125] | | |
| C19orf38 | chromosome 19 open reading | ENSG00000214212 | 255809 |
| | frame 38 [Source: HGNC | | |
| | Symbol; Acc: HGNC: 34073] | | |
| C1orf43 | chromosome 1 open reading | ENSG00000143612 | 25912 |
| | frame 43 [Source: HGNC | | |
| | Symbol; Acc: HGNC: 29876] | | |
| C5AR1 | complement C5a receptor 1 | ENSG00000197405 | 728 |
| | [Source: HGNC | | |
| | Symbol; Acc: HGNC: 1338] | | |
| CAPG | capping actin protein, gelsolin | ENSG00000042493 | 822 |
| | like [Source: HGNC | | |
| | Symbol; Acc: HGNC: 1474] | | |
| CASP10 | caspase 10 [Source: HGNC | ENSG00000003400 | 843 |
| | Symbol; Acc: HGNC: 1500] | | |
| CASP4 | caspase 4 [Source: HGNC | ENSG00000196954 | 837 |
| | Symbol; Acc: HGNC: 1505] | | |
| CCDC162P | coiled-coil domain containing | ENSG00000203799 | 0 |
| | 162, pseudogene | | |
| | [Source: HGNC | | |
| | Symbol; Acc: HGNC: 21565] | | |
| CCR1 | C-C motif chemokine receptor | ENSG00000163823 | 1230 |
| | 1 [Source: HGNC | | |
| | Symbol; Acc: HGNC: 1602] | | |
| CCR2 | C-C motif chemokine receptor | ENSG00000121807 | 729230 |
| | 2 [Source: HGNC | | |
| | Symbol; Acc: HGNC: 1603] | | |
| CD14 | CD14 molecule | ENSG00000170458 | 929 |
| | [Source: HGNC | | |
| | Symbol; Acc: HGNC: 1628] | | |
| CD2 | CD2 molecule [Source: HGNC | ENSG00000116824 | 914 |
| | Symbol; Acc: HGNC: 1639] | | |
| CD226 | CD226 molecule | ENSG00000150637 | 10666 |
| | [Source: HGNC | | |
| | Symbol; Acc: HGNC: 16961] | | |
| CD247 | CD247 molecule | ENSG00000198821 | 919 |
| | [Source: HGNC | | |
| | Symbol; Acc: HGNC: 1677] | | |
| CD300LF | CD300 molecule like family | ENSG00000186074 | 146722 |
| | member f [Source: HGNC | | |
| | Symbol; Acc: HGNC: 29883] | | |
| CD33 | CD33 molecule | ENSG00000105383 | 945 |
| | [Source: HGNC | | |
| | Symbol; Acc: HGNC: 1659] | | |
| CD36 | CD36 molecule | ENSG00000135218 | 948 |
| | [Source: HGNC | | |
| | Symbol; Acc: HGNC: 1663] | | |
| CD44 | CD44 molecule (Indian blood | ENSG00000026508 | 960 |
| | group) [Source: HGNC | | |
| | Symbol; Acc: HGNC: 1681] | | |
| CD68 | CD68 molecule | ENSG00000129226 | 968 |
| | [Source: HGNC | | |
| | Symbol; Acc: HGNC: 1693] | | |
| CD74 | CD74 molecule | ENSG00000019582 | 972 |
| | [Source: HGNC | | |
| | Symbol; Acc: HGNC: 1697] | | |
| CD81 | CD81 molecule | ENSG00000110651 | 975 |
| | [Source: HGNC | | |
| | Symbol; Acc: HGNC: 1701] | | |
| CD86 | CD86 molecule | ENSG00000114013 | 942 |
| | [Source: HGNC | | |
| | Symbol; Acc: HGNC: 1705] | | |
| CDC42EP1 | CDC42 effector protein 1 | ENSG00000128283 | 0 |
| | [Source: HGNC | | |
| | Symbol; Acc: HGNC: 17014] | | |
| CDH12 | cadherin 12 [Source: HGNC | ENSG00000154162 | 1010 |
| | Symbol; Acc: HGNC: 1751] | | |
| CFAP44 | cilia and flagella associated | ENSG00000206530 | 55779 |
| | protein 44 [Source: HGNC | | |
| | Symbol; Acc: HGNC: 25631] | | |
| CHST15 | carbohydrate sulfotransferase | ENSG00000182022 | 51363 |
| | 15 [Source: HGNC | | |
| | Symbol; Acc: HGNC: 18137] | | |
| CLEC10A | C-type lectin domain | ENSG00000132514 | 10462 |
| | containing 10A [Source: HGNC | | |
| | Symbol; Acc: HGNC: 16916] | | |
| COL6A6 | collagen type VI alpha 6 chain | ENSG00000206384 | 131873 |
| | [Source: HGNC | | |
| | Symbol; Acc: HGNC: 27023] | | |
| COQ7-DT | COQ7 divergent transcript | ENSG00000261465 | 0 |
| | [Source: HGNC | | |
| | Symbol; Acc: HGNC: 55362] | | |
| CORO1C | coronin 1C [Source: HGNC | ENSG00000110880 | 23603 |
| | Symbol; Acc: HGNC: 2254] | | |
| COTL1 | coactosin like F-actin binding | ENSG00000103187 | 23406 |
| | protein 1 [Source: HGNC | | |
| | Symbol; Acc: HGNC: 18304] | | |
| CPNE2 | copine 2 [Source: HGNC | ENSG00000140848 | 221184 |
| | Symbol; Acc: HGNC: 2315] | | |
| CREG1 | cellular repressor of E1A | ENSG00000143162 | 8804 |
| | stimulated genes 1 | | |
| | [Source: HGNC | | |
| | Symbol; Acc: HGNC: 2351] | | |
| CRTAP | cartilage associated protein | ENSG00000170275 | 10491 |
| | [Source: HGNC | | |
| | Symbol; Acc: HGNC: 2379] | | |
| CSF1R | colony stimulating factor 1 | ENSG00000182578 | 1436 |
| | receptor [Source: HGNC | | |
| | Symbol; Acc: HGNC: 2433] | | |
| CSF3R | colony stimulating factor 3 | ENSG00000119535 | 1441 |
| | receptor [Source: HGNC | | |
| | Symbol; Acc: HGNC: 2439] | | |
| CST3 | cystatin C [Source: HGNC | ENSG00000101439 | 1471 |
| | Symbol; Acc: HGNC: 2475] | | |
| CST7 | cystatin F [Source: HGNC | ENSG00000077984 | 8530 |
| | Symbol; Acc: HGNC: 2479] | | |
| CSTA | cystatin A [Source: HGNC | ENSG00000121552 | 1475 |
| | Symbol; Acc: HGNC: 2481] | | |
| CTSE | cathepsin E [Source: HGNC | ENSG00000196188 | 1510 |
| | Symbol; Acc: HGNC: 2530] | | |
| CTSH | cathepsin H [Source: HGNC | ENSG00000103811 | 1512 |
| | Symbol; Acc: HGNC: 2535] | | |
| CTSS | cathepsin S [Source: HGNC | ENSG00000163131 | 1520 |
| | Symbol; Acc: HGNC: 2545] | | |
| CTSW | cathepsin W [Source: HGNC | ENSG00000172543 | 1521 |
| | Symbol; Acc: HGNC: 2546] | | |
| CTSZ | cathepsin Z [Source: HGNC | ENSG00000101160 | 1522 |
| | Symbol; Acc: HGNC: 2547] | | |
| CXCL16 | C-X-C motif chemokine ligand | ENSG00000161921 | 58191 |
| | 16 [Source: HGNC | | |
| | Symbol; Acc: HGNC: 16642] | | |
| CYBB | cytochrome b-245 beta chain | ENSG00000165168 | 1536 |
| | [Source: HGNC | | |
| | Symbol; Acc: HGNC: 2578] | | |
| CYBRD1 | cytochrome b reductase 1 | ENSG00000071967 | 79901 |
| | [Source: HGNC | | |
| | Symbol; Acc: HGNC: 20797] | | |
| CYP1B1 | cytochrome P450 family 1 | ENSG00000138061 | 1545 |
| | subfamily B member 1 | | |
| | [Source: HGNC | | |
| | Symbol; Acc: HGNC: 2597] | | |
| CYP2S1 | cytochrome P450 family 2 | ENSG00000167600 | 29785 |
| | subfamily S member 1 | | |
| | [Source: HGNC | | |
| | Symbol; Acc: HGNC: 15654] | | |
| DDIT4 | DNA damage inducible | ENSG00000168209 | 54541 |
| | transcript 4 [Source: HGNC | | |
| | Symbol; Acc: HGNC: 24944] | | |
| DENND5A | DENN domain containing 5A | ENSG00000184014 | 23258 |
| | [Source: HGNC | | |
| | Symbol; Acc: HGNC: 19344] | | |
| DENND6B | DENN domain containing 6B | ENSG00000205593 | 414918 |
| | [Source: HGNC | | |
| | Symbol; Acc: HGNC: 32690] | | |
| DLX1 | distal-less homeobox 1 | ENSG00000144355 | 1745 |
| | [Source: HGNC | | |
| | Symbol; Acc: HGNC: 2914] | | |
| DMBX1 | diencephalon/mesencephalon | ENSG00000197587 | 127343 |
| | homeobox 1 [Source: HGNC | | |
| | Symbol; Acc: HGNC: 19026] | | |
| DNAJC27 | DnaJ heat shock protein family | ENSG00000115137 | 51277 |
| | (Hsp40) member C27 | | |
| | [Source: HGNC | | |
| | Symbol; Acc: HGNC: 30290] | | |
| DPYSL2 | dihydropyrimidinase like 2 | ENSG00000092964 | 1808 |
| | [Source: HGNC | | |
| | Symbol; Acc: HGNC: 3014] | | |
| DRAM2 | DNA damage regulated | ENSG00000156171 | 128338 |
| | autophagy modulator 2 | | |
| | [Source: HGNC | | |
| | Symbol; Acc: HGNC: 28769] | | |
| DUSP1 | dual specificity phosphatase 1 | ENSG00000120129 | 1843 |
| | [Source: HGNC | | |
| | Symbol; Acc: HGNC: 3064] | | |
| DUSP3 | dual specificity phosphatase 3 | ENSG00000108861 | 1845 |
| | [Source: HGNC | | |
| | Symbol; Acc: HGNC: 3069] | | |
| DUSP5 | dual specificity phosphatase 5 | ENSG00000138166 | 1847 |
| | [Source: HGNC | | |
| | Symbol; Acc: HGNC: 3071] | | |
| DUSP6 | dual specificity phosphatase 6 | ENSG00000139318 | 1848 |
| | [Source: HGNC | | |
| | Symbol; Acc: HGNC: 3072] | | |
| DYSF | dysferlin [Source: HGNC | ENSG00000135636 | 8291 |
| | Symbol; Acc: HGNC: 3097] | | |
| EDEM2 | ER degradation enhancing | ENSG00000088298 | 55741 |
| | alpha-mannosidase like protein | | |
| | 2 [Source: HGNC | | |
| | Symbol; Acc: HGNC: 15877] | | |
| EIF1 | eukaryotic translation initiation | ENSG00000173812 | 10209 |
| | factor 1 [Source: HGNC | | |
| | Symbol; Acc: HGNC: 3249] | | |
| EIF4E2 | eukaryotic translation initiation | ENSG00000135930 | 9470 |
| | factor 4E family member 2 | | |
| | [Source: HGNC | | |
| | Symbol; Acc: HGNC: 3293] | | |
| EIF4EBP1 | eukaryotic translation initiation | ENSG00000187840 | 1978 |
| | factor 4E binding protein 1 | | |
| | [Source: HGNC | | |
| | Symbol; Acc: HGNC: 3288] | | |
| EMILIN2 | elastin microfibril interfacer 2 | ENSG00000132205 | 0 |
| | [Source: HGNC | | |
| | Symbol; Acc: HGNC: 19881] | | |
| EOMES | eomesodermin [Source: HGNC | ENSG00000163508 | 8320 |
| | Symbol; Acc: HGNC: 3372] | | |
| EPB41L3 | erythrocyte membrane protein | ENSG00000082397 | 23136 |
| | band 4.1 like 3 [Source: HGNC | | |
| | Symbol; Acc: HGNC: 3380] | | |
| ERGIC1 | endoplasmic reticulum-golgi | ENSG00000113719 | 57222 |
| | intermediate compartment 1 | | |
| | [Source: HGNC | | |
| | Symbol; Acc: HGNC: 29205] | | |
| ERMAP | erythroblast membrane | ENSG00000164010 | 114625 |
| | associated protein (Scianna | | |
| | blood group) [Source: HGNC | | |
| | Symbol; Acc: HGNC: 15743] | | |
| ESD | esterase D [Source: HGNC | ENSG00000139684 | 2098 |
| | Symbol; Acc: HGNC: 3465] | | |
| ETS1 | ETS proto-oncogene 1, | ENSG00000134954 | 2113 |
| | transcription factor | | |
| | [Source: HGNC | | |
| | Symbol; Acc: HGNC: 3488] | | |
| ETS2 | ETS proto-oncogene 2, | ENSG00000157557 | 2114 |
| | transcription factor | | |
| | [Source: HGNC | | |
| | Symbol; Acc: HGNC: 3489] | | |
| FAM221B | family with sequence similarity | ENSG00000204930 | 392307 |
| | 221 member B [Source: HGNC | | |
| | Symbol; Acc: HGNC: 30762] | | |
| FBP1 | fructose-bisphosphatase 1 | ENSG00000165140 | 2203 |
| | [Source: HGNC | | |
| | Symbol; Acc: HGNC: 3606] | | |
| FCER1G | Fc fragment of IgE receptor Ig | ENSG00000158869 | 2207 |
| | [Source: HGNC | | |
| | Symbol; Acc: HGNC: 3611] | | |
| FCGR1A | Fc fragment of IgG receptor Ia | ENSG00000150337 | 2209 |
| | [Source: HGNC | | |
| | Symbol; Acc: HGNC: 3613] | | |
| FCGR2A | Fc fragment of IgG receptor Iia | ENSG00000143226 | 2212 |
| | [Source: HGNC | | |
| | Symbol; Acc: HGNC: 3616] | | |
| FCGRT | Fc fragment of IgG receptor | ENSG00000104870 | 2217 |
| | and transporter [Source: HGNC | | |
| | Symbol; Acc: HGNC: 3621] | | |
| FCN1 | ficolin 1 [Source: HGNC | ENSG00000085265 | 0 |
| | Symbol; Acc: HGNC: 3623] | | |
| FCRL2 | Fc receptor like 2 | ENSG00000132704 | 79368 |
| | [Source: HGNC | | |
| | Symbol; Acc: HGNC: 14875] | | |
| FGL2 | fibrinogen like 2 | ENSG00000127951 | 10875 |
| | [Source: HGNC | | |
| | Symbol; Acc: HGNC: 3696] | | |
| FGR | FGR proto-oncogene, Src | ENSG00000000938 | 2268 |
| | family tyrosine kinase | | |
| | [Source: HGNC | | |
| | Symbol; Acc: HGNC: 3697] | | |
| FOS | Fos proto-oncogene, AP-1 | ENSG00000170345 | 2353 |
| | transcription factor subunit | | |
| | [Source: HGNC | | |
| | Symbol; Acc: HGNC: 3796] | | |
| FPR1 | formyl peptide receptor 1 | ENSG00000171051 | 2357 |
| | [Source: HGNC | | |
| | Symbol; Acc: HGNC: 3826] | | |
| FPR2 | formyl peptide receptor 2 | ENSG00000171049 | 2358 |
| | [Source: HGNC | | |
| | Symbol; Acc: HGNC: 3827] | | |
| FRG2B | FSHD region gene 2 family | ENSG00000225899 | 441581 |
| | member B [Source: HGNC | | |
| | Symbol; Acc: HGNC: 33518] | | |
| FTH1 | ferritin heavy chain 1 | ENSG00000167996 | 2495 |
| | [Source: HGNC | | |
| | Symbol; Acc: HGNC: 3976] | | |
| FTL | ferritin light chain | ENSG00000087086 | 2512 |
| | [Source: HGNC | | |
| | Symbol; Acc: HGNC: 3999] | | |
| FTMT | ferritin mitochondrial | ENSG00000181867 | 0 |
| | [Source: HGNC | | |
| | Symbol; Acc: HGNC: 17345] | | |
| GAA | alpha glucosidase | ENSG00000171298 | 2548 |
| | [Source: HGNC | | |
| | Symbol; Acc: HGNC: 4065] | | |
| GABARAP | GABA type A receptor- | ENSG00000170296 | 11337 |
| | associated protein | | |
| | [Source: HGNC | | |
| | Symbol; Acc: HGNC: 4067] | | |
| GABRR3 | gamma-aminobutyric acid type | ENSG00000183185 | 200959 |
| | A receptor subunit rho3 | | |
| | [Source: HGNC | | |
| | Symbol; Acc: HGNC: 17969] | | |
| GAPDH | glyceraldehyde-3-phosphate | ENSG00000111640 | 2597 |
| | dehydrogenase [Source: HGNC | | |
| | Symbol; Acc: HGNC: 4141] | | |
| GAS7 | growth arrest specific 7 | ENSG00000007237 | 8522 |
| | [Source: HGNC | | |
| | Symbol; Acc: HGNC: 4169] | | |
| GASK1B | golgi associated kinase 1B | ENSG00000164125 | 51313 |
| | [Source: HGNC | | |
| | Symbol; Acc: HGNC: 25312] | | |
| GATA3 | GATA binding protein 3 | ENSG00000107485 | 2625 |
| | [Source: HGNC | | |
| | Symbol; Acc: HGNC: 4172] | | |
| GLCCI1 | glucocorticoid induced 1 | ENSG00000106415 | 113263 |
| | [Source: HGNC | | |
| | Symbol; Acc: HGNC: 18713] | | |
| GLIPR1 | GLI pathogenesis related 1 | ENSG00000139278 | 11010 |
| | [Source: HGNC | | |
| | Symbol; Acc: HGNC: 17001] | | |
| GNA15 | G protein subunit alpha 15 | ENSG00000060558 | 2769 |
| | [Source: HGNC | | |
| | Symbol; Acc: HGNC: 4383] | | |
| GNAI2 | G protein subunit alpha i2 | ENSG00000114353 | 2771 |
| | [Source: HGNC | | |
| | Symbol; Acc: HGNC: 4385] | | |
| GNLY | granulysin [Source: HGNC | ENSG00000115523 | 10578 |
| | Symbol; Acc: HGNC: 4414] | | |
| GNS | glucosamine (N-acetyl)-6- | ENSG00000135677 | 2799 |
| | sulfatase [Source: HGNC | | |
| | Symbol; Acc: HGNC: 4422] | | |
| GPBAR1 | G protein-coupled bile acid | ENSG00000179921 | 151306 |
| | receptor 1 [Source: HGNC | | |
| | Symbol; Acc: HGNC: 19680] | | |
| GPX1 | glutathione peroxidase 1 | ENSG00000233276 | 2876 |
| | [Source: HGNC | | |
| | Symbol; Acc: HGNC: 4553] | | |
| GRINA | glutamate ionotropic receptor | ENSG00000178719 | 2907 |
| | NMDA type subunit associated | | |
| | protein 1 [Source: HGNC | | |
| | Symbol; Acc: HGNC: 4589] | | |
| GRK3 | G protein-coupled receptor | ENSG00000100077 | 157 |
| | kinase 3 [Source: HGNC | | |
| | Symbol; Acc: HGNC: 290] | | |
| GRN | granulin precursor | ENSG00000030582 | 2896 |
| | [Source: HGNC | | |
| | Symbol; Acc: HGNC: 4601] | | |
| GSTP1 | glutathione S-transferase pi 1 | ENSG00000084207 | 2950 |
| | [Source: HGNC | | |
| | Symbol; Acc: HGNC: 4638] | | |
| H1-3 | H1.3 linker histone, cluster | ENSG00000124575 | 3007 |
| | member [Source: HGNC | | |
| | Symbol; Acc: HGNC: 4717] | | |
| HCK | HCK proto-oncogene, Src | ENSG00000101336 | 3055 |
| | family tyrosine kinase | | |
| | [Source: HGNC | | |
| | Symbol; Acc: HGNC: 4840] | | |
| HEG1 | heart development protein with | ENSG00000173706 | 0 |
| | EGF like domains 1 | | |
| | [Source: HGNC | | |
| | Symbol; Acc: HGNC: 29227] | | |
| HHEX | hematopoietically expressed | ENSG00000152804 | 0 |
| | homeobox [Source: HGNC | | |
| | Symbol; Acc: HGNC: 4901] | | |
| HIPK3 | homeodomain interacting | ENSG00000110422 | 10114 |
| | protein kinase 3 | | |
| | [Source: HGNC | | |
| | Symbol; Acc: HGNC: 4915] | | |
| HJV | hemojuvelin BMP co-receptor | ENSG00000168509 | 148738 |
| | [Source: HGNC | | |
| | Symbol; Acc: HGNC: 4887] | | |
| HMOX1 | heme oxygenase 1 | ENSG00000100292 | 3162 |
| | [Source: HGNC | | |
| | Symbol; Acc: HGNC: 5013] | | |
| HNMT | histamine N-methyltransferase | ENSG00000150540 | 3176 |
| | [Source: HGNC | | |
| | Symbol; Acc: HGNC: 5028] | | |
| HRH2 | histamine receptor H2 | ENSG00000113749 | 3274 |
| | [Source: HGNC | | |
| | Symbol; Acc: HGNC: 5183] | | |
| IFI30 | IFI30 lysosomal thiol reductase | ENSG00000216490 | 10437 |
| | [Source: HGNC | | |
| | Symbol; Acc: HGNC: 5398] | | |
| IFNA22P | interferon alpha 22, | ENSG00000224416 | 0 |
| | pseudogene [Source: HGNC | | |
| | Symbol; Acc: HGNC: 5431] | | |
| IFNA4 | interferon alpha 4 | ENSG00000236637 | 3441 |
| | [Source: HGNC | | |
| | Symbol; Acc: HGNC: 5425] | | |
| IFNGR2 | interferon gamma receptor 2 | ENSG00000159128 | 3460 |
| | [Source: HGNC | | |
| | Symbol; Acc: HGNC: 5440] | | |
| IGHM | immunoglobulin heavy | ENSG00000211899 | 0 |
| | constant mu [Source: HGNC | | |
| | Symbol; Acc: HGNC: 5541] | | |
| IKZF3 | IKAROS family zinc finger 3 | ENSG00000161405 | 22806 |
| | [Source: HGNC | | |
| | Symbol; Acc: HGNC: 13178] | | |
| IL1B | interleukin 1 beta | ENSG00000125538 | 3553 |
| | [Source: HGNC | | |
| | Symbol; Acc: HGNC: 5992] | | |
| IL1RN | interleukin 1 receptor | ENSG00000136689 | 3557 |
| | antagonist [Source: HGNC | | |
| | Symbol; Acc: HGNC: 6000] | | |
| ING1 | inhibitor of growth family | ENSG00000153487 | 3621 |
| | member 1 [Source: HGNC | | |
| | Symbol; Acc: HGNC: 6062] | | |
| INSR | insulin receptor [Source: HGNC | ENSG00000171105 | 3643 |
| | Symbol; Acc: HGNC: 6091] | | |
| IRF5 | interferon regulatory factor 5 | ENSG00000128604 | 3663 |
| | [Source: HGNC | | |
| | Symbol; Acc: HGNC: 6120] | | |
| ITGAM | integrin subunit alpha M | ENSG00000169896 | 3684 |
| | [Source: HGNC | | |
| | Symbol; Acc: HGNC: 6149] | | |
| ITK | IL2 inducible T cell kinase | ENSG00000113263 | 3702 |
| | [Source: HGNC | | |
| | Symbol; Acc: HGNC: 6171] | | |
| JAML | junction adhesion molecule like | ENSG00000160593 | 120425 |
| | [Source: HGNC | | |
| | Symbol; Acc: HGNC: 19084] | | |
| JUP | junction plakoglobin | ENSG00000173801 | 3728 |
| | [Source: HGNC | | |
| | Symbol; Acc: HGNC: 6207] | | |
| KCNE3 | potassium voltage-gated | ENSG00000175538 | 10008 |
| | channel subfamily E regulatory | | |
| | subunit 3 [Source: HGNC | | |
| | Symbol; Acc: HGNC: 6243] | | |
| KCNJ2 | potassium inwardly rectifying | ENSG00000123700 | 3759 |
| | channel subfamily J member 2 | | |
| | [Source: HGNC | | |
| | Symbol; Acc: HGNC: 6263] | | |
| KCNQ1 | potassium voltage-gated | ENSG00000053918 | 3784 |
| | channel subfamily Q member 1 | | |
| | [Source: HGNC | | |
| | Symbol; Acc: HGNC: 6294] | | |
| KCTD12 | potassium channel | ENSG00000178695 | 115207 |
| | tetramerization domain | | |
| | containing 12 [Source: HGNC | | |
| | Symbol; Acc: HGNC: 14678] | | |
| KDM1B | lysine demethylase 1B | ENSG00000165097 | 221656 |
| | [Source: HGNC | | |
| | Symbol; Acc: HGNC: 21577] | | |
| KLF4 | Kruppel like factor 4 | ENSG00000136826 | 9314 |
| | [Source: HGNC | | |
| | Symbol; Acc: HGNC: 6348] | | |
| KLRD1 | killer cell lectin like receptor | ENSG00000134539 | 3824 |
| | D1 [Source: HGNC | | |
| | Symbol; Acc: HGNC: 6378] | | |
| KRTAP6-3 | keratin associated protein 6-3 | ENSG00000212938 | 337968 |
| | [Source: HGNC | | |
| | Symbol; Acc: HGNC: 18933] | | |
| LAPTM5 | lysosomal protein | ENSG00000162511 | 7805 |
| | transmembrane 5 | | |
| | [Source: HGNC | | |
| | Symbol; Acc: HGNC: 29612] | | |
| LGALS2 | galectin 2 [Source: HGNC | ENSG00000100079 | 3957 |
| | Symbol; Acc: HGNC: 6562] | | |
| LGALS3 | galectin 3 [Source: HGNC | ENSG00000131981 | 3958 |
| | Symbol; Acc: HGNC: 6563] | | |
| LGALS9 | galectin 9 [Source: HGNC | ENSG00000168961 | 3965 |
| | Symbol; Acc: HGNC: 6570] | | |
| LINC00167 | long intergenic non-protein | ENSG00000233220 | 0 |
| | coding RNA 167 | | |
| | [Source: HGNC | | |
| | Symbol; Acc: HGNC: 30468] | | |
| LMO2 | LIM domain only 2 | ENSG00000135363 | 4005 |
| | [Source: HGNC | | |
| | Symbol; Acc: HGNC: 6642] | | |
| LPAR1 | lysophosphatidic acid receptor | ENSG00000198121 | 1902 |
| | 1 [Source: HGNC | | |
| | Symbol; Acc: HGNC: 3166] | | |
| LRBA | LPS responsive beige-like | ENSG00000198589 | 987 |
| | anchor protein [Source: HGNC | | |
| | Symbol; Acc: HGNC: 1742] | | |
| LRP1 | LDL receptor related protein 1 | ENSG00000123384 | 4035 |
| | [Source: HGNC | | |
| | Symbol; Acc: HGNC: 6692] | | |
| LRRC1 | leucine rich repeat containing 1 | ENSG00000137269 | 55227 |
| | [Source: HGNC | | |
| | Symbol; Acc: HGNC: 14307] | | |
| LRRC25 | leucine rich repeat containing | ENSG00000175489 | 126364 |
| | 25 [Source: HGNC | | |
| | Symbol; Acc: HGNC: 29806] | | |
| LRRK2 | leucine rich repeat kinase 2 | ENSG00000188906 | 120892 |
| | [Source: HGNC | | |
| | Symbol; Acc: HGNC: 18618] | | |
| LTA4H | leukotriene A4 hydrolase | ENSG00000111144 | 4048 |
| | [Source: HGNC | | |
| | Symbol; Acc: HGNC: 6710] | | |
| LTBR | lymphotoxin beta receptor | ENSG00000111321 | 4055 |
| | [Source: HGNC | | |
| | Symbol; Acc: HGNC: 6718] | | |
| LY86 | lymphocyte antigen 86 | ENSG00000112799 | 9450 |
| | [Source: HGNC | | |
| | Symbol; Acc: HGNC: 16837] | | |
| LYN | LYN proto-oncogene, Src | ENSG00000254087 | 4067 |
| | family tyrosine kinase | | |
| | [Source: HGNC | | |
| | Symbol; Acc: HGNC: 6735] | | |
| LYPD3 | LY6/PLAUR domain | ENSG00000124466 | 0 |
| | containing 3 [Source: HGNC | | |
| | Symbol; Acc: HGNC: 24880] | | |
| LYRM7 | LYR motif containing 7 | ENSG00000186687 | 90624 |
| | [Source: HGNC | | |
| | Symbol; Acc: HGNC: 28072] | | |
| LYZ | lysozyme [Source: HGNC | ENSG00000090382 | 4069 |
| | Symbol; Acc: HGNC: 6740] | | |
| MAFB | MAF bZIP transcription factor | ENSG00000204103 | 9935 |
| | B [Source: HGNC | | |
| | Symbol; Acc: HGNC: 6408] | | |
| MAPKAPK3 | MAPK activated protein kinase | ENSG00000114738 | 7867 |
| | 3 [Source: HGNC | | |
| | Symbol; Acc: HGNC: 6888] | | |
| MBD5 | methyl-CpG binding domain | ENSG00000204406 | 55777 |
| | protein 5 [Source: HGNC | | |
| | Symbol; Acc: HGNC: 20444] | | |
| MCM3AP-AS1 | MCM3AP antisense RNA 1 | ENSG00000215424 | 114044 |
| | [Source: HGNC | | |
| | Symbol; Acc: HGNC: 16417] | | |
| MCM8 | minichromosome maintenance | ENSG00000125885 | 84515 |
| | 8 homologous recombination | | |
| | repair factor [Source: HGNC | | |
| | Symbol; Acc: HGNC: 16147] | | |
| MED13L | mediator complex subunit 13L | ENSG00000123066 | 23389 |
| | [Source: HGNC | | |
| | Symbol; Acc: HGNC: 22962] | | |
| MEFV | MEFV innate immuity | ENSG00000103313 | 4210 |
| | regulator, pyrin [Source: HGNC | | |
| | Symbol; Acc: HGNC: 6998] | | |
| MICAL2 | microtubule associated | ENSG00000133816 | 9645 |
| | monooxygenase, calponin and | | |
| | LIM domain containing 2 | | |
| | [Source: HGNC | | |
| | Symbol; Acc: HGNC: 24693] | | |
| MIR664B | microRNA 664b | ENSG00000284450 | 100847052 |
| | [Source: HGNC | | |
| | Symbol; Acc: HGNC: 43501] | | |
| MNDA | myeloid cell nuclear | ENSG00000163563 | 0 |
| | differentiation antigen | | |
| | [Source: HGNC | | |
| | Symbol; Acc: HGNC: 7183] | | |
| MPEG1 | macrophage expressed 1 | ENSG00000197629 | 219972 |
| | [Source: HGNC | | |
| | Symbol; Acc: HGNC: 29619] | | |
| MS4A1 | membrane spanning 4-domains | ENSG00000156738 | 931 |
| | Al [Source: HGNC | | |
| | Symbol; Acc: HGNC: 7315] | | |
| MS4A6A | membrane spanning 4-domains | ENSG00000110077 | 64231 |
| | A6A [Source: HGNC | | |
| | Symbol; Acc: HGNC: 13375] | | |
| MS4A7 | membrane spanning 4-domains | ENSG00000166927 | 58475 |
| | A7 [Source: HGNC | | |
| | Symbol; Acc: HGNC: 13378] | | |
| MSRB1 | methionine sulfoxide reductase | ENSG00000198736 | 51734 |
| | B1 [Source: HGNC | | |
| | Symbol; Acc: HGNC: 14133] | | |
| MSTRG | MSTRG | ENSG00000284741 | #N/A |
| MSTRG | MSTRG | MSTRG | #N/A |
| MSTRG | MSTRG | MSTRG | #N/A |
| MSTRG | MSTRG | MSTRG | #N/A |
| MSTRG | MSTRG | MSTRG | #N/A |
| MTRNR2L12 | MT-RNR2 like 12 | ENSG00000269028 | 100462981 |
| | [Source: HGNC | | |
| | Symbol; Acc: HGNC: 37169] | | |
| MTRNR2L8 | MT-RNR2 like 8 | ENSG00000255823 | 100463486 |
| | [Source: HGNC | | |
| | Symbol; Acc: HGNC: 37165] | | |
| NAGA | alpha-N- | ENSG00000198951 | 4668 |
| | acetylgalactosaminidase | | |
| | [Source: HGNC | | |
| | Symbol; Acc: HGNC: 7631] | | |
| NAGK | N-acetylglucosamine kinase | ENSG00000124357 | 55577 |
| | [Source: HGNC | | |
| | Symbol; Acc: HGNC: 17174] | | |
| NAPSB | napsin B aspartic peptidase, | ENSG00000131401 | 256236 |
| | pseudogene [Source: HGNC | | |
| | Symbol; Acc: HGNC: 13396] | | |
| NCF1B | neutrophil cytosolic factor 1B | ENSG00000182487 | 654816 |
| | pseudogene [Source: HGNC | | |
| | Symbol; Acc: HGNC: 32522] | | |
| NCF2 | neutrophil cytosolic factor 2 | ENSG00000116701 | 4688 |
| | [Source: HGNC | | |
| | Symbol; Acc: HGNC: 7661] | | |
| NCOA4 | nuclear receptor coactivator 4 | ENSG00000266412 | 8031 |
| | [Source: HGNC | | |
| | Symbol; Acc: HGNC: 7671] | | |
| NDST1 | N-deacetylase and N- | ENSG00000070614 | 3340 |
| | sulfotransferase 1 | | |
| | [Source: HGNC | | |
| | Symbol; Acc: HGNC: 7680] | | |
| NEK6 | NIMA related kinase 6 | ENSG00000119408 | 10783 |
| | [Source: HGNC | | |
| | Symbol; Acc: HGNC: 7749] | | |
| NFAM1 | NFAT activating protein with | ENSG00000235568 | 150372 |
| | ITAM motif 1 [Source: HGNC | | |
| | Symbol; Acc: HGNC: 29872] | | |
| NFE2 | nuclear factor, erythroid 2 | ENSG00000123405 | 4778 |
| | [Source: HGNC | | |
| | Symbol; Acc: HGNC: 7780] | | |
| NIBAN2 | niban apoptosis regulator 2 | ENSG00000136830 | 64855 |
| | [Source: HGNC | | |
| | Symbol; Acc: HGNC: 25282] | | |
| NINJ1 | ninjurin 1 [Source: HGNC | ENSG00000131669 | 4814 |
| | Symbol; Acc: HGNC: 7824] | | |
| NLRC4 | NLR family CARD domain | ENSG00000091106 | 58484 |
| | containing 4 [Source: HGNC | | |
| | Symbol; Acc: HGNC: 16412] | | |
| NLRP12 | NLR family pyrin domain | ENSG00000142405 | 91662 |
| | containing 12 [Source: HGNC | | |
| | Symbol; Acc: HGNC: 22938] | | |
| NOTCH2 | notch receptor 2 | ENSG00000134250 | 4853 |
| | [Source: HGNC | | |
| | Symbol; Acc: HGNC: 7882] | | |
| Novel Gene | novel transcript, similar to YY1 | ENSG00000280614 | 0 |
| | associated myogenesis RNA 1 | | |
| | YAM1 | | |
| Novel Gene | novel transcript, similar to YY1 | ENSG00000280800 | 0 |
| | associated myogenesis RNA 1 | | |
| | YAM1 | | |
| Novel Gene | novel transcript, similar to YY1 | ENSG00000288444 | 0 |
| | associated myogenesis RNA 1 | | |
| | YAM1 | | |
| Novel Gene | novel transcript | ENSG00000278918 | 0 |
| Novel Gene | novel transcript, antisense to | ENSG00000280211 | 0 |
| | RNF40 | | |
| Novel Gene | heterogeneous nuclear | ENSG00000232499 | 0 |
| | ribonucleoprotein A3 | | |
| | (hnRNPA3) pseudogene | | |
| Novel Gene | novel transcript | ENSG00000249207 | 0 |
| Novel Gene | novel transcript | ENSG00000287234 | 0 |
| NPAT | nuclear protein, coactivator of | ENSG00000149308 | 4863 |
| | histone transcription | | |
| | [Source: HGNC | | |
| | Symbol; Acc: HGNC: 7896] | | |
| NPC2 | NPC intracellular cholesterol | ENSG00000119655 | 10577 |
| | transporter 2 [Source: HGNC | | |
| | Symbol; Acc: HGNC: 14537] | | |
| NR3C2 | nuclear receptor subfamily 3 | ENSG00000151623 | 4306 |
| | group C member 2 | | |
| | [Source: HGNC | | |
| | Symbol; Acc: HGNC: 7979] | | |
| NUMB | NUMB endocytic adaptor | ENSG00000133961 | 8650 |
| | protein [Source: HGNC | | |
| | Symbol; Acc: HGNC: 8060] | | |
| OAF | out at first homolog | ENSG00000184232 | 220323 |
| | [Source: HGNC | | |
| | Symbol; Acc: HGNC: 28752] | | |
| OAS1 | 2′-5′-oligoadenylate synthetase | ENSG00000089127 | 4938 |
| | 1 [Source: HGNC | | |
| | Symbol; Acc: HGNC: 8086] | | |
| OAZ1 | ornithine decarboxylase | ENSG00000104904 | 4946 |
| | antizyme 1 [Source: HGNC | | |
| | Symbol; Acc: HGNC: 8095] | | |
| OR2L1P | olfactory receptor family 2 | ENSG00000224227 | 26247 |
| | subfamily L member 1 | | |
| | pseudogene [Source: HGNC | | |
| | Symbol; Acc: HGNC: 8265] | | |
| P2RY13 | purinergic receptor P2Y13 | ENSG00000181631 | 0 |
| | [Source: HGNC | | |
| | Symbol; Acc: HGNC: 4537] | | |
| PADI2 | peptidyl arginine deiminase 2 | ENSG00000117115 | 11240 |
| | [Source: HGNC | | |
| | Symbol; Acc: HGNC: 18341] | | |
| PAK1 | p21 (RAC1) activated kinase 1 | ENSG00000149269 | 5058 |
| | [Source: HGNC | | |
| | Symbol; Acc: HGNC: 8590] | | |
| PCBD1 | pterin-4 alpha-carbinolamine | ENSG00000166228 | 5092 |
| | dehydratase 1 [Source: HGNC | | |
| | Symbol; Acc: HGNC: 8646] | | |
| PDCD6IP | programmed cell death 6 | ENSG00000170248 | 10015 |
| | interacting protein | | |
| | [Source: HGNC | | |
| | Symbol; Acc: HGNC: 8766] | | |
| PDPR2P | pyruvate dehydrogenase | ENSG00000214331 | 0 |
| | phosphatase regulatory subunit | | |
| | 2, pseudogene [Source: HGNC | | |
| | Symbol; Acc: HGNC: 27556] | | |
| PEA15 | proliferation and apoptosis | ENSG00000162734 | 8682 |
| | adaptor protein 15 | | |
| | [Source: HGNC | | |
| | Symbol; Acc: HGNC: 8822] | | |
| PELI1 | pellino E3 ubiquitin protein | ENSG00000197329 | 57162 |
| | ligase 1 [Source: HGNC | | |
| | Symbol; Acc: HGNC: 8827] | | |
| PGD | phosphogluconate | ENSG00000142657 | 5226 |
| | dehydrogenase [Source: HGNC | | |
| | Symbol; Acc: HGNC: 8891] | | |
| PICALM | phosphatidylinositol binding | ENSG00000073921 | 8301 |
| | clathrin assembly protein | | |
| | [Source: HGNC | | |
| | Symbol; Acc: HGNC: 15514] | | |
| PILRA | paired immunoglobin like type | ENSG00000085514 | 29992 |
| | 2 receptor alpha | | |
| | [Source: HGNC | | |
| | Symbol; Acc: HGNC: 20396] | | |
| PKM | pyruvate kinase M1/2 | ENSG00000067225 | 5315 |
| | [Source: HGNC | | |
| | Symbol; Acc: HGNC: 9021] | | |
| PLAGL2 | PLAGI like zinc finger 2 | ENSG00000126003 | 5326 |
| | [Source: HGNC | | |
| | Symbol; Acc: HGNC: 9047] | | |
| PLAUR | plasminogen activator, | ENSG00000011422 | 5329 |
| | urokinase receptor | | |
| | [Source: HGNC | | |
| | Symbol; Acc: HGNC: 9053] | | |
| PLB1 | phospholipase B1 | ENSG00000163803 | 151056 |
| | [Source: HGNC | | |
| | Symbol; Acc: HGNC: 30041] | | |
| PLBD1 | phospholipase B domain | ENSG00000121316 | 79887 |
| | containing 1 [Source: HGNC | | |
| | Symbol; Acc: HGNC: 26215] | | |
| PPP5D1P | PPP5 tetratricopeptide repeat | ENSG00000230510 | 100506012 |
| | domain containing 1, | | |
| | pseudogene [Source: HGNC | | |
| | Symbol; Acc: HGNC: 44209] | | |
| PRF1 | perforin 1 [Source: HGNC | ENSG00000180644 | 5551 |
| | Symbol; Acc: HGNC: 9360] | | |
| PRKCD | protein kinase C delta | ENSG00000163932 | 5580 |
| | [Source: HGNC | | |
| | Symbol; Acc: HGNC: 9399] | | |
| PRR5L | proline rich 5 like | ENSG00000135362 | 79899 |
| | [Source: HGNC | | |
| | Symbol; Acc: HGNC: 25878] | | |
| PRSS23 | serine protease 23 | ENSG00000150687 | 11098 |
| | [Source: HGNC | | |
| | Symbol; Acc: HGNC: 14370] | | |
| PSAP | prosaposin [Source: HGNC | ENSG00000197746 | 5660 |
| | Symbol; Acc: HGNC: 9498] | | |
| PSEN1 | presenilin 1 [Source: HGNC | ENSG00000080815 | 5663 |
| | Symbol; Acc: HGNC: 9508] | | |
| PTAFR | platelet activating factor | ENSG00000169403 | 5724 |
| | receptor [Source: HGNC | | |
| | Symbol; Acc: HGNC: 9582] | | |
| PTPRE | protein tyrosine phosphatase | ENSG00000132334 | 5791 |
| | receptor type E [Source: HGNC | | |
| | Symbol; Acc: HGNC: 9669] | | |
| PTRHD1 | peptidy1-tRNA hydrolase | ENSG00000184924 | 0 |
| | domain containing 1 | | |
| | [Source: HGNC | | |
| | Symbol; Acc: HGNC: 33782] | | |
| PYCARD | PYD and CARD domain | ENSG00000103490 | 29108 |
| | containing [Source: HGNC | | |
| | Symbol; Acc: HGNC: 16608] | | |
| PYGL | glycogen phosphorylase L | ENSG00000100504 | 5836 |
| | [Source: HGNC | | |
| | Symbol; Acc: HGNC: 9725] | | |
| QPCT | glutaminyl-peptide | ENSG00000115828 | 25797 |
| | cyclotransferase | | |
| | [Source: HGNC | | |
| | Symbol; Acc: HGNC: 9753] | | |
| RAB31 | RAB31, member RAS | ENSG00000168461 | 11031 |
| | oncogene family | | |
| | [Source: HGNC | | |
| | Symbol; Acc: HGNC: 9771] | | |
| RAB32 | RAB32, member RAS | ENSG00000118508 | 10981 |
| | oncogene family | | |
| | [Source: HGNC | | |
| | Symbol; Acc: HGNC: 9772] | | |
| RAB3D | RAB3D, member RAS | ENSG00000105514 | 0 |
| | oncogene family | | |
| | [Source: HGNC | | |
| | Symbol; Acc: HGNC: 9779] | | |
| RAB5IF | RAB5 interacting factor | ENSG00000101084 | 55969 |
| | [Source: HGNC | | |
| | Symbol; Acc: HGNC: 15870] | | |
| RARA | retinoic acid receptor alpha | ENSG00000131759 | 5914 |
| | [Source: HGNC | | |
| | Symbol; Acc: HGNC: 9864] | | |
| RASGRP4 | RAS guanyl releasing protein 4 | ENSG00000171777 | 115727 |
| | [Source: HGNC | | |
| | Symbol; Acc: HGNC: 18958] | | |
| RASSF2 | Ras association domain family | ENSG00000101265 | 9770 |
| | member 2 [Source: HGNC | | |
| | Symbol; Acc: HGNC: 9883] | | |
| RBIS | ribosomal biogenesis factor | ENSG00000176731 | 401466 |
| | [Source: HGNC | | |
| | Symbol; Acc: HGNC: 32235] | | |
| RBM26 | RNA binding motif protein 26 | ENSG00000139746 | 64062 |
| | [Source: HGNC | | |
| | Symbol; Acc: HGNC: 20327] | | |
| RBM47 | RNA binding motif protein 47 | ENSG00000163694 | 54502 |
| | [Source: HGNC | | |
| | Symbol; Acc: HGNC: 30358] | | |
| RBM5 | RNA binding motif protein 5 | ENSG00000003756 | 10181 |
| | [Source: HGNC | | |
| | Symbol; Acc: HGNC: 9902] | | |
| RBMS2 | RNA binding motif single | ENSG00000076067 | 5939 |
| | stranded interacting protein 2 | | |
| | [Source: HGNC | | |
| | Symbol; Acc: HGNC: 9909] | | |
| RBP7 | retinol binding protein 7 | ENSG00000162444 | 116362 |
| | [Source: HGNC | | |
| | Symbol; Acc: HGNC: 30316] | | |
| RGS2 | regulator of G protein signaling | ENSG00000116741 | 5997 |
| | 2 [Source: HGNC | | |
| | Symbol; Acc: HGNC: 9998] | | |
| RIN2 | Ras and Rab interactor 2 | ENSG00000132669 | 54453 |
| | [Source: HGNC | | |
| | Symbol; Acc: HGNC: 18750] | | |
| RN7SL1 | RNA component of signal | ENSG00000276168 | 6029 |
| | recognition particle 7SL1 | | |
| | [Source: HGNC | | |
| | Symbol; Acc: HGNC: 10038] | | |
| RN7SL151P | RNA, 7SL, cytoplasmic 151, | ENSG00000244230 | 0 |
| | pseudogene [Source: HGNC | | |
| | Symbol; Acc: HGNC: 46167] | | |
| RN7SL396P | RNA, 7SL, cytoplasmic 396, | ENSG00000244642 | 0 |
| | pseudogene [Source: HGNC | | |
| | Symbol; Acc: HGNC: 46412] | | |
| RN7SL4P | RNA, 7SL, cytoplasmic 4, | ENSG00000263740 | 0 |
| | pseudogene [Source: HGNC | | |
| | Symbol; Acc: HGNC: 10039] | | |
| RN7SL5P | RNA, 7SL, cytoplasmic 5, | ENSG00000265735 | 0 |
| | pseudogene [Source: HGNC | | |
| | Symbol; Acc: HGNC: 10040] | | |
| RN7SL674P | RNA, 7SL, cytoplasmic 674, | ENSG00000239899 | 0 |
| | pseudogene [Source: HGNC | | |
| | Symbol; Acc: HGNC: 46690] | | |
| RN7SL752P | RNA, 7SL, cytoplasmic 752, | ENSG00000239437 | 0 |
| | pseudogene [Source: HGNC | | |
| | Symbol; Acc: HGNC: 46768] | | |
| RNASE2 | ribonuclease A family member | ENSG00000169385 | 6036 |
| | 2 [Source: HGNC | | |
| | Symbol; Acc: HGNC: 10045] | | |
| RNASE6 | ribonuclease A family member | ENSG00000169413 | 6039 |
| | k6 [Source: HGNC | | |
| | Symbol; Acc: HGNC: 10048] | | |
| RNF130 | ring finger protein 130 | ENSG00000113269 | 55819 |
| | [Source: HGNC | | |
| | Symbol; Acc: HGNC: 18280] | | |
| RNPEP | arginyl aminopeptidase | ENSG00000176393 | 6051 |
| | [Source: HGNC | | |
| | Symbol; Acc: HGNC: 10078] | | |
| RTN1 | reticulon 1 [Source: HGNC | ENSG00000139970 | 6252 |
| | Symbol; Acc: HGNC: 10467] | | |
| RUSC2 | RUN and SH3 domain | ENSG00000198853 | 9853 |
| | containing 2 [Source: HGNC | | |
| | Symbol; Acc: HGNC: 23625] | | |
| RXRA | retinoid X receptor alpha | ENSG00000186350 | 6256 |
| | [Source: HGNC | | |
| | Symbol; Acc: HGNC: 10477] | | |
| S100A10 | S100 calcium binding protein | ENSG00000197747 | 6281 |
| | A10 [Source: HGNC | | |
| | Symbol; Acc: HGNC: 10487] | | |
| S100A12 | S100 calcium binding protein | ENSG00000163221 | 6283 |
| | A12 [Source: HGNC | | |
| | Symbol; Acc: HGNC: 10489] | | |
| S100A6 | S100 calcium binding protein | ENSG00000197956 | 6277 |
| | A6 [Source: HGNC | | |
| | Symbol; Acc: HGNC: 10496] | | |
| S100A9 | S100 calcium binding protein | ENSG00000163220 | 6280 |
| | A9 [Source: HGNC | | |
| | Symbol; Acc: HGNC: 10499] | | |
| S1PR3 | sphingosine-1-phosphate | ENSG00000213694 | 1903 |
| | receptor 3 [Source: HGNC | | |
| | Symbol; Acc: HGNC: 3167] | | |
| SARAF | store-operated calcium entry | ENSG00000133872 | 51669 |
| | associated regulatory factor | | |
| | [Source: HGNC | | |
| | Symbol; Acc: HGNC: 28789] | | |
| SATB1 | SATB homeobox 1 | ENSG00000182568 | 6304 |
| | [Source: HGNC | | |
| | Symbol; Acc: HGNC: 10541] | | |
| SEC14L1 | SEC14 like lipid binding 1 | ENSG00000129657 | 6397 |
| | [Source: HGNC | | |
| | Symbol; Acc: HGNC: 10698] | | |
| SECTM1 | secreted and transmembrane 1 | ENSG00000141574 | 6398 |
| | [Source: HGNC | | |
| | Symbol; Acc: HGNC: 10707] | | |
| SEMA4A | semaphorin 4A [Source: HGNC | ENSG00000196189 | 64218 |
| | Symbol; Acc: HGNC: 10729] | | |
| SERINC2 | serine incorporator 2 | ENSG00000168528 | 347735 |
| | [Source: HGNC | | |
| | Symbol; Acc: HGNC: 23231] | | |
| SERPINB1 | serpin family B member 1 | ENSG00000021355 | 1992 |
| | [Source: HGNC | | |
| | Symbol; Acc: HGNC: 3311] | | |
| SGK1 | serum/glucocorticoid regulated | ENSG00000118515 | 6446 |
| | kinase 1 [Source: HGNC | | |
| | Symbol; Acc: HGNC: 10810] | | |
| SIRPA | signal regulatory protein alpha | ENSG00000198053 | 140885 |
| | [Source: HGNC | | |
| | Symbol; Acc: HGNC: 9662] | | |
| SIRPB1 | signal regulatory protein beta 1 | ENSG00000101307 | 10326 |
| | [Source: HGNC | | |
| | Symbol; Acc: HGNC: 15928] | | |
| SIRPB2 | signal regulatory protein beta 2 | ENSG00000196209 | 284759 |
| | [Source: HGNC | | |
| | Symbol; Acc: HGNC: 16247] | | |
| SKA2 | spindle and kinetochore | ENSG00000182628 | 348235 |
| | associated complex subunit 2 | | |
| | [Source: HGNC | | |
| | Symbol; Acc: HGNC: 28006] | | |
| SLC15A3 | solute carrier family 15 | ENSG00000110446 | 51296 |
| | member 3 [Source: HGNC | | |
| | Symbol; Acc: HGNC: 18068] | | |
| SLC22A15 | solute carrier family 22 | ENSG00000163393 | 55356 |
| | member 15 [Source: HGNC | | |
| | Symbol; Acc: HGNC: 20301] | | |
| SLC25A5 | solute carrier family 25 | ENSG00000005022 | 292 |
| | member 5 [Source: HGNC | | |
| | Symbol; Acc: HGNC: 10991] | | |
| SLC29A1 | solute carrier family 29 | ENSG00000112759 | 2030 |
| | member 1 (Augustine blood | | |
| | group) [Source: HGNC | | |
| | Symbol; Acc: HGNC: 11003] | | |
| SLC46A2 | solute carrier family 46 | ENSG00000119457 | 57864 |
| | member 2 [Source: HGNC | | |
| | Symbol; Acc: HGNC: 16055] | | |
| SLC7A7 | solute carrier family 7 member | ENSG00000155465 | 9056 |
| | 7 [Source: HGNC | | |
| | Symbol; Acc: HGNC: 11065] | | |
| SLC9A7P1 | solute carrier family 9 member | ENSG00000227825 | 121456 |
| | 7 pseudogene 1 [Source: HGNC | | |
| | Symbol; Acc: HGNC: 32679] | | |
| SMARCD3 | SWI/SNF related, matrix | ENSG00000082014 | 6604 |
| | associated, actin dependent | | |
| | regulator of chromatin, | | |
| | subfamily d, member 3 | | |
| | [Source: HGNC | | |
| | Symbol; Acc: HGNC: 11108] | | |
| SNCAIP | synuclein alpha interacting | ENSG00000064692 | 9627 |
| | protein [Source: HGNC | | |
| | Symbol; Acc: HGNC: 11139] | | |
| SNORA22 | small nucleolar RNA, H/ACA | ENSG00000206634 | 677807 |
| | box 22 [Source: HGNC | | |
| | Symbol; Acc: HGNC: 32612] | | |
| SNORA22C | small nucleolar RNA, H/ACA | ENSG00000207344 | 109616965 |
| | box 22C [Source: HGNC | | |
| | Symbol; Acc: HGNC: 52197] | | |
| SNORA38B | small nucleolar RNA, H/ACA | ENSG00000200394 | 100124536 |
| | box 38B [Source: HGNC | | |
| | Symbol; Acc: HGNC: 33617] | | |
| SNORA3A | small nucleolar RNA, H/ACA | ENSG00000200983 | 619562 |
| | box 3A [Source: HGNC | | |
| | Symbol; Acc: HGNC: 32586] | | |
| SNORA51 | small nucleolar RNA, H/ACA | ENSG00000271798 | 677831 |
| | box 51 [Source: HGNC | | |
| | Symbol; Acc: HGNC: 32644] | | |
| SNORA60 | small nucleolar RNA, H/ACA | ENSG00000199266 | 0 |
| | box 60 [Source: HGNC | | |
| | Symbol; Acc: HGNC: 32654] | | |
| SNORA81 | small nucleolar RNA, H/ACA | ENSG00000221420 | 677847 |
| | box 81 [Source: HGNC | | |
| | Symbol; Acc: HGNC: 32667] | | |
| SORT1 | sortilin 1 [Source: HGNC | ENSG00000134243 | 6272 |
| | Symbol; Acc: HGNC: 11186] | | |
| SOWAHD | sosondowah ankyrin repeat | ENSG00000187808 | 0 |
| | domain family member D | | |
| | [Source: HGNC | | |
| | Symbol; Acc: HGNC: 32960] | | |
| SPI1 | Spi-1 proto-oncogene | ENSG00000066336 | 6688 |
| | [Source: HGNC | | |
| | Symbol; Acc: HGNC: 11241] | | |
| SPNS3 | sphingolipid transporter 3 | ENSG00000182557 | 201305 |
| | (putative) [Source: HGNC | | |
| | Symbol; Acc: HGNC: 28433] | | |
| SQOR | sulfide quinone oxidoreductase | ENSG00000137767 | 58472 |
| | [Source: HGNC | | |
| | Symbol; Acc: HGNC: 20390] | | |
| SRGN | serglycin [Source: HGNC | ENSG00000122862 | 5552 |
| | Symbol; Acc: HGNC: 9361] | | |
| ST6GALNAC2 | ST6 N-acetylgalactosaminide | ENSG00000070731 | 10610 |
| | alpha-2,6-sialyltransferase 2 | | |
| | [Source: HGNC | | |
| | Symbol; Acc: HGNC: 10867] | | |
| ST8SIA6 | ST8 alpha-N-acetyl- | ENSG00000148488 | 338596 |
| | neuraminide alpha-2,8- | | |
| | sialyltransferase 6 | | |
| | [Source: HGNC | | |
| | Symbol; Acc: HGNC: 23317] | | |
| STAT4 | signal transducer and activator | ENSG00000138378 | 6775 |
| | of transcription 4 | | |
| | [Source: HGNC | | |
| | Symbol; Acc: HGNC: 11365] | | |
| STX10 | syntaxin 10 [Source: HGNC | ENSG00000104915 | 8677 |
| | Symbol; Acc: HGNC: 11428] | | |
| STX12 | syntaxin 12 [Source: HGNC | ENSG00000117758 | 23673 |
| | Symbol; Acc: HGNC: 11430] | | |
| SULF2 | sulfatase 2 [Source: HGNC | ENSG00000196562 | 55959 |
| | Symbol; Acc: HGNC: 20392] | | |
| SULT1A1 | sulfotransferase family 1A | ENSG00000196502 | 6817 |
| | member 1 [Source: HGNC | | |
| | Symbol; Acc: HGNC: 11453] | | |
| SYNE2 | spectrin repeat containing | ENSG00000054654 | 23224 |
| | nuclear envelope protein 2 | | |
| | [Source: HGNC | | |
| | Symbol; Acc: HGNC: 17084] | | |
| TALDO1 | transaldolase 1 [Source: HGNC | ENSG00000177156 | 6888 |
| | Symbol; Acc: HGNC: 11559] | | |
| TBX21 | T-box transcription factor 21 | ENSG00000073861 | 0 |
| | [Source: HGNC | | |
| | Symbol; Acc: HGNC: 11599] | | |
| TBXAS1 | thromboxane A synthase 1 | ENSG00000059377 | 6916 |
| | [Source: HGNC | | |
| | Symbol; Acc: HGNC: 11609] | | |
| TET2 | tet methylcytosine dioxygenase | ENSG00000168769 | 54790 |
| | 2 [Source: HGNC | | |
| | Symbol; Acc: HGNC: 25941] | | |
| TGFBI | transforming growth factor beta | ENSG00000120708 | 7045 |
| | induced [Source: HGNC | | |
| | Symbol; Acc: HGNC: 11771] | | |
| TIMP2 | TIMP metallopeptidase | ENSG00000035862 | 7077 |
| | inhibitor 2 [Source: HGNC | | |
| | Symbol; Acc: HGNC: 11821] | | |
| TKT | transketolase [Source: HGNC | ENSG00000163931 | 7086 |
| | Symbol; Acc: HGNC: 11834] | | |
| TLE5 | TLE family member 5, | ENSG00000104964 | 166 |
| | transcriptional modulator | | |
| | [Source: HGNC | | |
| | Symbol; Acc: HGNC: 307] | | |
| TLR2 | toll like receptor 2 | ENSG00000137462 | 7097 |
| | [Source: HGNC | | |
| | Symbol; Acc: HGNC: 11848] | | |
| TLR4 | toll like receptor 4 | ENSG00000136869 | 7099 |
| | [Source: HGNC | | |
| | Symbol; Acc: HGNC: 11850] | | |
| TLR8 | toll like receptor 8 | ENSG00000101916 | 51311 |
| | [Source: HGNC | | |
| | Symbol; Acc: HGNC: 15632] | | |
| TMEM150B | transmembrane protein 150B | ENSG00000180061 | 284417 |
| | [Source: HGNC | | |
| | Symbol; Acc: HGNC: 34415] | | |
| TMEM170B | transmembrane protein 170B | ENSG00000205269 | 100113407 |
| | [Source: HGNC | | |
| | Symbol; Acc: HGNC: 34244] | | |
| TNFRSF1B | TNF receptor superfamily | ENSG00000028137 | 7133 |
| | member 1B [Source: HGNC | | |
| | Symbol; Acc: HGNC: 11917] | | |
| TNN | tenascin N [Source: HGNC | ENSG00000120332 | 63923 |
| | Symbol; Acc: HGNC: 22942] | | |
| TNRC6C | trinucleotide repeat containing | ENSG00000078687 | 57690 |
| | adaptor 6C [Source: HGNC | | |
| | Symbol; Acc: HGNC: 29318] | | |
| TRDC | T cell receptor delta constant | ENSG00000211829 | 0 |
| | [Source: HGNC | | |
| | Symbol; Acc: HGNC: 12253] | | |
| TREM1 | triggering receptor expressed | ENSG00000124731 | 54210 |
| | on myeloid cells 1 | | |
| | [Source: HGNC | | |
| | Symbol; Acc: HGNC: 17760] | | |
| TRIB1 | tribbles pseudokinase 1 | ENSG00000173334 | 10221 |
| | [Source: HGNC | | |
| | Symbol; Acc: HGNC: 16891] | | |
| TSHZ3 | teashirt zinc finger homeobox 3 | ENSG00000121297 | 57616 |
| | [Source: HGNC | | |
| | Symbol; Acc: HGNC: 307001 | | |
| TUBA1A | tubulin alpha 1a | ENSG00000167552 | 7846 |
| | [Source: HGNC | | |
| | Symbol; Acc: HGNC: 20766] | | |
| TYROBP | transmembrane immune | ENSG00000011600 | 7305 |
| | signaling adaptor TYROBP | | |
| | [Source: HGNC | | |
| | Symbol; Acc: HGNC: 12449] | | |
| UBBP4 | ubiquitin B pseudogene 4 | ENSG00000263563 | 0 |
| | [Source: HGNC | | |
| | Symbol; Acc: HGNC: 12467] | | |
| UBE2D1 | ubiquitin conjugating enzyme | ENSG00000072401 | 7321 |
| | E2 D1 [Source: HGNC | | |
| | Symbol; Acc: HGNC: 12474] | | |
| UBE2R2 | ubiquitin conjugating enzyme | ENSG00000107341 | 0 |
| | E2 R2 [Source: HGNC | | |
| | Symbol; Acc: HGNC: 19907] | | |
| UNC119 | unc-119 lipid binding | ENSG00000109103 | 9094 |
| | chaperone [Source: HGNC | | |
| | Symbol; Acc: HGNC: 12565] | | |
| UNC93B1 | unc-93 homolog B1, TLR | ENSG00000110057 | 81622 |
| | signaling regulator | | |
| | [Source: HGNC | | |
| | Symbol; Acc: HGNC: 13481] | | |
| USP11 | ubiquitin specific peptidase 11 | ENSG00000102226 | 8237 |
| | [Source: HGNC | | |
| | Symbol; Acc: HGNC: 12609] | | |
| VCAN | versican [Source: HGNC | ENSG00000038427 | 1462 |
| | Symbol; Acc: HGNC: 2464] | | |
| VDR | vitamin D receptor | ENSG00000111424 | 7421 |
| | [Source: HGNC | | |
| | Symbol; Acc: HGNC: 12679] | | |
| VIM | vimentin [Source: HGNC | ENSG00000026025 | 7431 |
| | Symbol; Acc: HGNC: 12692] | | |
| VNN1 | vanin 1 [Source: HGNC | ENSG00000112299 | 8876 |
| | Symbol; Acc: HGNC: 12705] | | |
| VOPP1 | VOPP1 WW domain binding | ENSG00000154978 | 81552 |
| | protein [Source: HGNC | | |
| | Symbol; Acc: HGNC: 34518] | | |
| WARS1 | tryptophanyl-tRNA synthetase | ENSG00000140105 | 7453 |
| | 1 [Source: HGNC | | |
| | Symbol; Acc: HGNC: 12729] | | |
| WDFY3 | WD repeat and FYVE domain | ENSG00000163625 | 23001 |
| | containing 3 [Source: HGNC | | |
| | Symbol; Acc: HGNC: 20751] | | |
| WLS | Wnt ligand secretion mediator | ENSG00000116729 | 79971 |
| | [Source: HGNC | | |
| | Symbol; Acc: HGNC: 30238] | | |
| XRCC6P2 | X-ray repair cross | ENSG00000234825 | 0 |
| | complementing 6 pseudogene 2 | | |
| | [Source: HGNC | | |
| | Symbol; Acc: HGNC: 45184] | | |
| YBX3 | Y-box binding protein 3 | ENSG00000060138 | 8531 |
| | [Source: HGNC | | |
| | Symbol; Acc: HGNC: 2428] | | |
| ZCCHC14 | zinc finger CCHC-type | ENSG00000140948 | 23174 |
| | containing 14 [Source: HGNC | | |
| | Symbol; Acc: HGNC: 24134] | | |
| ZDHHC7 | zinc finger DHHC-type | ENSG00000153786 | 55625 |
| | palmitoyltransferase 7 | | |
| | [Source: HGNC | | |
| | Symbol; Acc: HGNC: 184591 | | |
| ZEB2 | zinc finger E-box binding | ENSG00000169554 | 9839 |
| | homeobox 2 [Source: HGNC | | |
| | Symbol; Acc: HGNC: 14881] | | |
| ZNF106 | zinc finger protein 106 | ENSG00000103994 | 64397 |
| | [Source: HGNC | | |
| | Symbol; Acc: HGNC: 12886] | | |
| ZNF253 | zinc finger protein 253 | ENSG00000256771 | 56242 |
| | [Source: HGNC | | |
| | Symbol; Acc: HGNC: 13497] | | |
| ZNF385A | zinc finger protein 385A | ENSG00000161642 | 25946 |
| | [Source: HGNC | | |
| | Symbol; Acc: HGNC: 17521] | | |
| ZNF703 | zinc finger protein 703 | ENSG00000183779 | 80139 |
| | [Source: HGNC | | |
| | Symbol; Acc: HGNC: 25883] | | |
| ZNF724 | zinc finger protein 724 | ENSG00000196081 | 440519 |
| | [Source: HGNC | | |
| | Symbol; Acc: HGNC: 32460] | | |

FM3

| | | | |
|---|---|---|---|
| 7SK | 7SK RNA | ENSG00000202198 | 0 |
| | [Source: RFAM; Acc: RF00100] | | |
| AC005562.1 | novel transcript | ENSG00000214719 | 0 |
| ACOT1 | acyl-CoA thioesterase 1 | ENSG00000184227 | 641371 |
| | [Source: HGNC | | |
| | Symbol; Acc: HGNC: 33128] | | |
| ACOT13 | acyl-CoA thioesterase 13 | ENSG00000112304 | 55856 |
| | [Source: HGNC | | |
| | Symbol; Acc: HGNC: 20999] | | |
| ACSF2 | acyl-CoA synthetase family | ENSG00000167107 | 80221 |
| | member 2 [Source: HGNC | | |
| | Symbol; Acc: HGNC: 26101] | | |
| ACVR1B | activin A receptor type 1B | ENSG00000135503 | 91 |
| | [Source: HGNC | | |
| | Symbol; Acc: HGNC: 172] | | |
| ADAM17 | ADAM metallopeptidase | ENSG00000151694 | 6868 |
| | domain 17 [Source: HGNC | | |
| | Symbol; Acc: HGNC: 195] | | |
| ADGRE5 | adhesion G protein-coupled | ENSG00000123146 | 976 |
| | receptor E5 [Source: HGNC | | |
| | Symbol; Acc: HGNC: 1711] | | |
| AKAP13 | A-kinase anchoring protein 13 | ENSG00000170776 | 11214 |
| | [Source: HGNC | | |
| | Symbol; Acc: HGNC: 371] | | |
| AKIP1 | A-kinase interacting protein 1 | ENSG00000166452 | 56672 |
| | [Source: HGNC | | |
| | Symbol; Acc: HGNC: 1170] | | |
| AP000936.1 | ribosomal protein S27 | ENSG00000234268 | 0 |
| | (metallopanstimulin 1) | | |
| | (RPS27) pseudogene | | |
| AP3B1 | adaptor related protein complex | ENSG00000132842 | 8546 |
| | 3 subunit beta 1 | | |
| | [Source: HGNC | | |
| | Symbol; Acc: HGNC: 566] | | |
| AP5M1 | adaptor related protein complex | ENSG00000053770 | 55745 |
| | 5 subunit mu 1 [Source: HGNC | | |
| | Symbol; Acc: HGNC: 20192] | | |
| APLP2 | amyloid beta precursor like | ENSG00000084234 | 334 |
| | protein 2 [Source: HGNC | | |
| | Symbol; Acc: HGNC: 598] | | |
| APOOL | apolipoprotein O like | ENSG00000155008 | 139322 |
| | [Source: HGNC | | |
| | Symbol; Acc: HGNC: 24009] | | |
| ARFGEF1 | ADP ribosylation factor | ENSG00000066777 | 10565 |
| | guanine nucleotide exchange | | |
| | factor 1 [Source: HGNC | | |
| | Symbol; Acc: HGNC: 15772] | | |
| ARL6IP5 | ADP ribosylation factor like | ENSG00000144746 | 10550 |
| | GTPase 6 interacting protein 5 | | |
| | [Source: HGNC | | |
| | Symbol; Acc: HGNC: 16937] | | |
| ART5 | ADP-ribosyltransferase 5 | ENSG00000167311 | 116969 |
| | [Source: HGNC | | |
| | Symbol; Acc: HGNC: 24049] | | |
| ATAD1 | ATPase family AAA domain | ENSG00000138138 | 84896 |
| | containing 1 [Source: HGNC | | |
| | Symbol; Acc: HGNC: 25903] | | |
| ATP11B | ATPase phospholipid | ENSG00000058063 | 23200 |
| | transporting 11B (putative) | | |
| | [Source: HGNC | | |
| | Symbol; Acc: HGNC: 13553] | | |
| ATP2B1 | ATPase plasma membrane | ENSG00000070961 | 490 |
| | Ca2+ transporting 1 | | |
| | [Source: HGNC | | |
| | Symbol; Acc: HGNC: 814] | | |
| ATP5MK | ATP synthase membrane | ENSG00000173915 | 84833 |
| | subunit k [Source: HGNC | | |
| | Symbol; Acc: HGNC: 30889] | | |
| B4GALT4 | beta-1,4-galactosyltransferase 4 | ENSG00000121578 | 8702 |
| | [Source: HGNC | | |
| | Symbol; Acc: HGNC: 927] | | |
| BATF2 | basic leucine zipper ATF-like | ENSG00000168062 | 116071 |
| | transcription factor 2 | | |
| | [Source: HGNC | | |
| | Symbol; Acc: HGNC: 25163] | | |
| BBX | BBX high mobility group box | ENSG00000114439 | 56987 |
| | domain containing | | |
| | [Source: HGNC | | |
| | Symbol; Acc: HGNC: 14422] | | |
| BCAS3 | BCAS3 microtubule associated | ENSG00000141376 | 54828 |
| | cell migration factor | | |
| | [Source: HGNC | | |
| | Symbol; Acc: HGNC: 14347] | | |
| BCKDHA | branched chain keto acid | ENSG00000248098 | 593 |
| | dehydrogenase El subunit | | |
| | alpha [Source: HGNC | | |
| | Symbol; Acc: HGNC: 986] | | |
| BDNF | brain derived neurotrophic | ENSG00000176697 | 627 |
| | factor [Source: HGNC | | |
| | Symbol; Acc: HGNC: 1033] | | |
| BIN3 | bridging integrator 3 | ENSG00000147439 | 55909 |
| | [Source: HGNC | | |
| | Symbol; Acc: HGNC: 1054] | | |
| BTBD9 | BTB domain containing 9 | ENSG00000183826 | 114781 |
| | [Source: HGNC | | |
| | Symbol; Acc: HGNC: 21228] | | |
| BUB1 | BUB1 mitotic checkpoint | ENSG00000169679 | 699 |
| | serine/threonine kinase | | |
| | [Source: HGNC | | |
| | Symbol; Acc: HGNC: 1148] | | |
| C1orf74 | chromosome 1 open reading | ENSG00000162757 | 148304 |
| | frame 74 [Source: HGNC | | |
| | Symbol; Acc: HGNC: 26319] | | |
| C5orf24 | chromosome 5 open reading | ENSG00000181904 | 134553 |
| | frame 24 [Source: HGNC | | |
| | Symbol; Acc: HGNC: 26746] | | |
| C8orf89 | chromosome 8 open reading | ENSG00000274443 | 100130301 |
| | frame 89 [Source: HGNC | | |
| | Symbol; Acc: HGNC: 51258] | | |
| CALU | calumenin [Source: HGNC | ENSG00000128595 | 813 |
| | Symbol; Acc: HGNC: 1458] | | |
| CASP1 | caspase 1 [Source: HGNC | ENSG00000137752 | 834 |
| | Symbol; Acc: HGNC: 1499] | | |
| CASP10 | caspase 10 [Source: HGNC | ENSG00000003400 | 843 |
| | Symbol; Acc: HGNC: 1500] | | |
| CCAR1 | cell division cycle and | ENSG00000060339 | 55749 |
| | apoptosis regulator 1 | | |
| | [Source: HGNC | | |
| | Symbol; Acc: HGNC: 24236] | | |
| CCDC152 | coiled-coil domain containing | ENSG00000198865 | 100129792 |
| | 152 [Source: HGNC | | |
| | Symbol; Acc: HGNC: 34438] | | |
| CCDC82 | coiled-coil domain containing | ENSG00000149231 | 79780 |
| | 82 [Source: HGNC | | |
| | Symbol; Acc: HGNC: 26282] | | |
| CCIN | calicin [Source: HGNC | ENSG00000185972 | 881 |
| | Symbol; Acc: HGNC: 1568] | | |
| CCL2 | C-C motif chemokine ligand 2 | ENSG00000108691 | 6347 |
| | [Source: HGNC | | |
| | Symbol; Acc: HGNC: 10618] | | |
| CCNL1 | cyclin L1 [Source: HGNC | ENSG00000163660 | 57018 |
| | Symbol; Acc: HGNC: 20569] | | |
| CD164 | CD164 molecule | ENSG00000135535 | 8763 |
| | [Source: HGNC | | |
| | Symbol; Acc: HGNC: 1632] | | |
| CD200R1 | CD200 receptor 1 | ENSG00000163606 | 131450 |
| | [Source: HGNC | | |
| | Symbol; Acc: HGNC: 24235] | | |
| CD300LF | CD300 molecule like family | ENSG00000186074 | 146722 |
| | member f [Source: HGNC | | |
| | Symbol; Acc: HGNC: 29883] | | |
| CDC27 | cell division cycle 27 | ENSG00000004897 | 996 |
| | [Source: HGNC | | |
| | Symbol; Acc: HGNC: 1728] | | |
| CFL1 | cofilin 1 [Source: HGNC | ENSG00000172757 | 1072 |
| | Symbol; Acc: HGNC: 1874] | | |
| CFLAR | CASP8 and FADD like | ENSG00000003402 | 8837 |
| | apoptosis regulator | | |
| | [Source: HGNC | | |
| | Symbol; Acc: HGNC: 1876] | | |
| CHD1 | chromodomain helicase DNA | ENSG00000153922 | 1105 |
| | binding protein 1 | | |
| | [Source: HGNC | | |
| | Symbol; Acc: HGNC: 1915] | | |
| CHROMR | cholesterol induced regulator of | ENSG00000223960 | 101927027 |
| | metabolism RNA | | |
| | [Source: HGNC | | |
| | Symbol; Acc: HGNC: 54059] | | |
| CLIP1 | CAP-Gly domain containing | ENSG00000130779 | 6249 |
| | linker protein 1 [Source: HGNC | | |
| | Symbol; Acc: HGNC: 10461] | | |
| CMAHP | cytidine monophospho-N- | ENSG00000168405 | 0 |
| | acetylneuraminic acid | | |
| | hydroxylase, pseudogene | | |
| | [Source: HGNC | | |
| | Symbol; Acc: HGNC: 2098] | | |
| CNP | 2′,3′-cyclic nucleotide 3′ | ENSG00000173786 | 1267 |
| | phosphodiesterase | | |
| | [Source: HGNC | | |
| | Symbol; Acc: HGNC: 2158] | | |
| COPZ1 | COPI coat complex subunit | ENSG00000111481 | 22818 |
| | zeta 1 [Source: HGNC | | |
| | Symbol; Acc: HGNC: 2243] | | |
| CPM | carboxypeptidase M | ENSG00000135678 | 1368 |
| | [Source: HGNC | | |
| | Symbol; Acc: HGNC: 2311] | | |
| CREBRF | CREB3 regulatory factor | ENSG00000164463 | 153222 |
| | [Source: HGNC | | |
| | Symbol; Acc: HGNC: 24050] | | |
| CRYGS | crystallin gamma S | ENSG00000213139 | 0 |
| | [Source: HGNC | | |
| | Symbol; Acc: HGNC: 2417] | | |
| CSNK1A1 | casein kinase 1 alpha 1 | ENSG00000113712 | 1452 |
| | [Source: HGNC | | |
| | Symbol; Acc: HGNC: 2451] | | |
| CST2 | cystatin SA [Source: HGNC | ENSG00000170369 | 1470 |
| | Symbol; Acc: HGNC: 2474] | | |
| CTD-3099C6.13 | novel zinc finger protein | ENSG00000269825 | 0 |
| CUL4A | cullin 4A [Source: HGNC | ENSG00000139842 | 8451 |
| | Symbol; Acc: HGNC: 2554] | | |
| CYP4F12 | cytochrome P450 family 4 | ENSG00000186204 | 66002 |
| | subfamily F member 12 | | |
| | [Source: HGNC | | |
| | Symbol; Acc: HGNC: 18857] | | |
| CYTIP | cytohesin 1 interacting protein | ENSG00000115165 | 9595 |
| | [Source: HGNC | | |
| | Symbol; Acc: HGNC: 9506] | | |
| DCAF7 | DDB1 and CUL4 associated | ENSG00000136485 | 0 |
| | factor 7 [Source: HGNC | | |
| | Symbol; Acc: HGNC: 30915] | | |
| DCUN1D5 | defective in cullin neddylation | ENSG00000137692 | 84259 |
| | 1 domain containing 5 | | |
| | [Source: HGNC | | |
| | Symbol; Acc: HGNC: 28409] | | |
| DDX18P3 | DEAD-box helicase 18 | ENSG00000217414 | 0 |
| | pseudogene 3 [Source: HGNC | | |
| | Symbol; Acc: HGNC: 33966] | | |
| DDX58 | DexD/H-box helicase 58 | ENSG00000107201 | 23586 |
| | [Source: HGNC | | |
| | Symbol; Acc: HGNC: 19102] | | |
| DDX60 | DexD/H-box helicase 60 | ENSG00000137628 | 55601 |
| | [Source: HGNC | | |
| | Symbol; Acc: HGNC: 25942] | | |
| DDX60L | DexD/H-box 60 like | ENSG00000181381 | 91351 |
| | [Source: HGNC | | |
| | Symbol; Acc: HGNC: 26429] | | |
| DENND1B | DENN domain containing 1B | ENSG00000213047 | 163486 |
| | [Source: HGNC | | |
| | Symbol; Acc: HGNC: 28404] | | |
| DLST | dihydrolipoamide S- | ENSG00000119689 | 1743 |
| | succinyltransferase | | |
| | [Source: HGNC | | |
| | Symbol; Acc: HGNC: 2911] | | |
| DMTF1 | cyclin D binding myb like | ENSG00000135164 | 9988 |
| | transcription factor 1 | | |
| | [Source: HGNC | | |
| | Symbol; Acc: HGNC: 14603] | | |
| DNAJA4 | DnaJ heat shock protein family | ENSG00000140403 | 55466 |
| | (Hsp40) member A4 | | |
| | [Source: HGNC | | |
| | Symbol; Acc: HGNC: 14885] | | |
| DNAJB7 | DnaJ heat shock protein family | ENSG00000172404 | 150353 |
| | (Hsp40) member B7 | | |
| | [Source: HGNC | | |
| | Symbol; Acc: HGNC: 24986] | | |
| DNAJC13 | DnaJ heat shock protein family | ENSG00000138246 | 23317 |
| | (Hsp40) member C13 | | |
| | [Source: HGNC | | |
| | Symbol; Acc: HGNC: 30343] | | |
| DOCK2 | dedicator of cytokinesis 2 | ENSG00000134516 | 1794 |
| | [Source: HGNC | | |
| | Symbol; Acc: HGNC: 2988] | | |
| DTD2 | D-aminoacyl-tRNA deacylase | ENSG00000129480 | 112487 |
| | 2 [Source: HGNC | | |
| | Symbol; Acc: HGNC: 20277] | | |
| DTX3L | deltex E3 ubiquitin ligase 3L | ENSG00000163840 | 151636 |
| | [Source: HGNC | | |
| | Symbol; Acc: HGNC: 30323] | | |
| EIF2AK2 | eukaryotic translation initiation | ENSG00000055332 | 5610 |
| | factor 2 alpha kinase 2 | | |
| | [Source: HGNC | | |
| | Symbol; Acc: HGNC: 9437] | | |
| ELMO1 | engulfment and cell motility 1 | ENSG00000155849 | 9844 |
| | [Source: HGNC | | |
| | Symbol; Acc: HGNC: 16286] | | |
| FAM120A | family with sequence similarity | ENSG00000048828 | 23196 |
| | 120A [Source: HGNC | | |
| | Symbol; Acc: HGNC: 13247] | | |
| FAM124B | family with sequence similarity | ENSG00000124019 | 79843 |
| | 124 member B [Source: HGNC | | |
| | Symbol; Acc: HGNC: 26224] | | |
| FAM76A | family with sequence similarity | ENSG00000009780 | 199870 |
| | 76 member A [Source: HGNC | | |
| | Symbol; Acc: HGNC: 28530] | | |
| FBXO45 | F-box protein 45 | ENSG00000174013 | 200933 |
| | [Source: HGNC | | |
| | Symbol; Acc: HGNC: 29148] | | |
| FCRL4 | Fc receptor like 4 | ENSG00000163518 | 83417 |
| | [Source: HGNC | | |
| | Symbol; Acc: HGNC: 18507] | | |
| FGD2 | FYVE, RhoGEF and PH | ENSG00000146192 | 221472 |
| | domain containing 2 | | |
| | [Source: HGNC | | |
| | Symbol; Acc: HGNC: 3664] | | |
| FNBP1 | formin binding protein 1 | ENSG00000187239 | 23048 |
| | [Source: HGNC | | |
| | Symbol; Acc: HGNC: 17069] | | |
| FRG2B | FSHD region gene 2 family | ENSG00000225899 | 441581 |
| | member B [Source: HGNC | | |
| | Symbol; Acc: HGNC: 33518] | | |
| FSCN3 | fascin actin-bundling protein 3 | ENSG00000106328 | 29999 |
| | [Source: HGNC | | |
| | Symbol; Acc: HGNC: 3961] | | |
| FYTTD1 | forty-two-three domain | ENSG00000122068 | 0 |
| | containing 1 [Source: HGNC | | |
| | Symbol; Acc: HGNC: 25407] | | |
| GABPA | GA binding protein | ENSG00000154727 | 2551 |
| | transcription factor subunit | | |
| | alpha [Source: HGNC | | |
| | Symbol; Acc: HGNC: 4071] | | |
| GBP1 | guanylate binding protein 1 | ENSG00000117228 | 2633 |
| | [Source: HGNC | | |
| | Symbol; Acc: HGNC: 4182] | | |
| GBP4 | guanylate binding protein 4 | ENSG00000162654 | 115361 |
| | [Source: HGNC | | |
| | Symbol; Acc: HGNC: 20480] | | |
| GBP5 | guanylate binding protein 5 | ENSG00000154451 | 115362 |
| | [Source: HGNC | | |
| | Symbol; Acc: HGNC: 19895] | | |
| GEMIN8 | gem nuclear organelle | ENSG00000046647 | 54960 |
| | associated protein 8 | | |
| | [Source: HGNC | | |
| | Symbol; Acc: HGNC: 26044] | | |
| GFM1 | G elongation factor | ENSG00000168827 | 85476 |
| | mitochondrial 1 | | |
| | [Source: HGNC | | |
| | Symbol; Acc: HGNC: 13780] | | |
| GIMAP4 | GTPase, IMAP family member | ENSG00000133574 | 0 |
| | 4 [Source: HGNC | | |
| | Symbol; Acc: HGNC: 21872] | | |
| GNAQP1 | G protein subunit alpha q | ENSG00000214077 | 0 |
| | pseudogene 1 [Source: HGNC | | |
| | Symbol; Acc: HGNC: 4391] | | |
| GOLGA2P6 | GOLGA2 pseudogene 6 | ENSG00000241621 | 0 |
| | [Source: HGNC | | |
| | Symbol; Acc: HGNC: 44948] | | |
| GOLGA8N | golgin A8 family member N | ENSG00000283589 | 0 |
| | [Source: HGNC | | |
| | Symbol; Acc: HGNC: 44405] | | |
| GOLPH3L | golgi phosphoprotein 3 like | ENSG00000143457 | 55204 |
| | [Source: HGNC | | |
| | Symbol; Acc: HGNC: 24882] | | |
| GOSR1 | golgi SNAP receptor complex | ENSG00000108587 | 9527 |
| | member 1 [Source: HGNC | | |
| | Symbol; Acc: HGNC: 4430] | | |
| GPBP1 | GC-rich promoter binding | ENSG00000062194 | 65056 |
| | protein 1 [Source: HGNC | | |
| | Symbol; Acc: HGNC: 29520] | | |
| GSPT2 | G1 to S phase transition 2 | ENSG00000189369 | 23708 |
| | [Source: HGNC | | |
| | Symbol; Acc: HGNC: 4622] | | |
| H3P16 | H3 histone pseudogene 16 | ENSG00000178458 | 0 |
| | [Source: HGNC | | |
| | Symbol; Acc: HGNC: 42982] | | |
| HERC5 | HECT and RLD domain | ENSG00000138646 | 51191 |
| | containing E3 ubiquitin protein | | |
| | ligase 5 [Source: HGNC | | |
| | Symbol; Acc: HGNC: 24368] | | |
| HINT1P1 | histidine triad nucleotide | ENSG00000231531 | 0 |
| | binding protein 1 pseudogene 1 | | |
| | [Source: HGNC | | |
| | Symbol; Acc: HGNC: 18467] | | |
| HMGB3P20 | high mobility group box 3 | ENSG00000226059 | 0 |
| | pseudogene 20 [Source: HGNC | | |
| | Symbol; Acc: HGNC: 39312] | | |
| HNRNPDL | heterogeneous nuclear | ENSG00000152795 | 9987 |
| | ribonucleoprotein D like | | |
| | [Source: HGNC | | |
| | Symbol; Acc: HGNC: 5037] | | |
| HNRNPH3 | heterogeneous nuclear | ENSG00000096746 | 3189 |
| | ribonucleoprotein H3 | | |
| | [Source: HGNC | | |
| | Symbol; Acc: HGNC: 5043] | | |
| HSP90AA1 | heat shock protein 90 alpha | ENSG00000080824 | 3320 |
| | family class A member 1 | | |
| | [Source: HGNC | | |
| | Symbol; Acc: HGNC: 5253] | | |
| HSPA5 | heat shock protein family A | ENSG00000044574 | 3309 |
| | (Hsp70) member 5 | | |
| | [Source: HGNC | | |
| | Symbol; Acc: HGNC: 5238] | | |
| HSPA8P5 | heat shock protein family A | ENSG00000256356 | 0 |
| | (Hsp70) member 8 pseudogene | | |
| | 5 [Source: HGNC | | |
| | Symbol; Acc: HGNC: 44920] | | |
| IFI16 | interferon gamma inducible | ENSG00000163565 | 3428 |
| | protein 16 [Source: HGNC | | |
| | Symbol; Acc: HGNC: 5395] | | |
| IFI44 | interferon induced protein 44 | ENSG00000137965 | 10561 |
| | [Source: HGNC | | |
| | Symbol; Acc: HGNC: 16938] | | |
| IFNA22P | interferon alpha 22, | ENSG00000224416 | 0 |
| | pseudogene [Source: HGNC | | |
| | Symbol; Acc: HGNC: 5431] | | |
| IFT80 | intraflagellar transport 80 | ENSG00000068885 | 57560 |
| | [Source: HGNC | | |
| | Symbol; Acc: HGNC: 29262] | | |
| IGKV2D-29 | immunoglobulin kappa variable | ENSG00000243264 | 0 |
| | 2D-29 [Source: HGNC | | |
| | Symbol; Acc: HGNC: 5800] | | |
| IGLV3-16 | immunoglobulin lambda | ENSG00000211665 | 0 |
| | variable 3-16 [Source: HGNC | | |
| | Symbol; Acc: HGNC: 5901] | | |
| IGLV6-57 | immunoglobulin lambda | ENSG00000211640 | 0 |
| | variable 6-57 [Source: HGNC | | |
| | Symbol; Acc: HGNC: 5927] | | |
| IQGAP2 | IQ motif containing GTPase | ENSG00000145703 | 10788 |
| | activating protein 2 | | |
| | [Source: HGNC | | |
| | Symbol; Acc: HGNC: 6111] | | |
| ISG20 | interferon stimulated | ENSG00000172183 | 3669 |
| | exonuclease gene 20 | | |
| | [Source: HGNC | | |
| | Symbol; Acc: HGNC: 6130] | | |
| JAZF1 | JAZF zinc finger 1 | ENSG00000153814 | 221895 |
| | [Source: HGNC | | |
| | Symbol; Acc: HGNC: 28917] | | |
| KDM4C | lysine demethylase 4C | ENSG00000107077 | 23081 |
| | [Source: HGNC | | |
| | Symbol; Acc: HGNC: 17071] | | |
| KHDC4 | KH domain containing 4, pre- | ENSG00000132680 | 0 |
| | mRNA splicing factor | | |
| | [Source: HGNC | | |
| | Symbol; Acc: HGNC: 29145] | | |
| KIAA0513 | KIAA0513 [Source: HGNC | ENSG00000135709 | 9764 |
| | Symbol; Acc: HGNC: 29058] | | |
| KRTAP26-1 | keratin associated protein 26-1 | ENSG00000197683 | 388818 |
| | [Source: HGNC | | |
| | Symbol; Acc: HGNC: 33760] | | |
| LAP3 | leucine aminopeptidase 3 | ENSG00000002549 | 51056 |
| | [Source: HGNC | | |
| | Symbol; Acc: HGNC: 18449] | | |
| LAT2 | linker for activation of T cells | ENSG00000086730 | 7462 |
| | family member 2 | | |
| | [Source: HGNC | | |
| | Symbol; Acc: HGNC: 12749] | | |
| LCOR | ligand dependent nuclear | ENSG00000196233 | 84458 |
| | receptor corepressor | | |
| | [Source: HGNC | | |
| | Symbol; Acc: HGNC: 29503] | | |
| LEXM | lymphocyte expansion | ENSG00000162398 | 163747 |
| | molecule [Source: HGNC | | |
| | Symbol; Acc: HGNC: 26854] | | |
| LGALS8 | galectin 8 [Source: HGNC | ENSG00000116977 | 3964 |
| | Symbol; Acc: HGNC: 6569] | | |
| LILRB2 | leukocyte immunoglobulin like | ENSG00000274513 | 10288 |
| | receptor B2 [Source: HGNC | | |
| | Symbol; Acc: HGNC: 6606] | | |
| LIN7A | lin-7 homolog A, crumbs cell | ENSG00000111052 | 8825 |
| | polarity complex component | | |
| | [Source: HGNC | | |
| | Symbol; Acc: HGNC: 17787] | | |
| LINC01151 | long intergenic non-protein | ENSG00000253819 | 0 |
| | coding RNA 1151 | | |
| | [Source: HGNC | | |
| | Symbol; Acc: HGNC: 49471] | | |
| LINGO4 | leucine rich repeat and Ig | ENSG00000213171 | 339398 |
| | domain containing 4 | | |
| | [Source: HGNC | | |
| | Symbol; Acc: HGNC: 31814] | | |
| LRR1 | leucine rich repeat protein 1 | ENSG00000165501 | 122769 |
| | [Source: HGNC | | |
| | Symbol; Acc: HGNC: 19742] | | |
| LSG1 | large 60S subunit nuclear | ENSG00000041802 | 0 |
| | export GTPase 1 | | |
| | [Source: HGNC | | |
| | Symbol; Acc: HGNC: 25652] | | |
| LYRM2 | LYR motif containing 2 | ENSG00000083099 | 57226 |
| | [Source: HGNC | | |
| | Symbol; Acc: HGNC: 25229] | | |
| LYST | lysosomal trafficking regulator | ENSG00000143669 | 1130 |
| | [Source: HGNC | | |
| | Symbol; Acc: HGNC: 1968] | | |
| MAGOH | mago homolog, exon junction | ENSG00000162385 | 0 |
| | complex subunit | | |
| | [Source: HGNC | | |
| | Symbol; Acc: HGNC: 6815] | | |
| MAGOHB | mago homolog B, exon | ENSG00000111196 | 55110 |
| | junction complex subunit | | |
| | [Source: HGNC | | |
| | Symbol; Acc: HGNC: 25504] | | |
| MAP4K4 | mitogen-activated protein | ENSG00000071054 | 9448 |
| | kinase kinase kinase kinase 4 | | |
| | [Source: HGNC | | |
| | Symbol; Acc: HGNC: 6866] | | |
| MEFV | MEFV innate immuity | ENSG00000103313 | 4210 |
| | regulator, pyrin [Source: HGNC | | |
| | Symbol; Acc: HGNC: 6998] | | |
| MICA | MHC class I polypeptide- | ENSG00000235233 | 100507436 |
| | related sequence A | | |
| | [Source: HGNC | | |
| | Symbol; Acc: HGNC: 7090] | | |
| MIGA1 | mitoguardin 1 [Source: HGNC | ENSG00000180488 | 374986 |
| | Symbol; Acc: HGNC: 24741] | | |
| MIR1248 | microRNA 1248 | ENSG00000283958 | 100302143 |
| | [Source: HGNC | | |
| | Symbol; Acc: HGNC: 35314] | | |
| MIR181B1 | microRNA 181b-1 | ENSG00000207975 | 406955 |
| | [Source: HGNC | | |
| | Symbol; Acc: HGNC: 31550] | | |
| MIS18BP1 | MIS18 binding protein 1 | ENSG00000129534 | 55320 |
| | [Source: HGNC | | |
| | Symbol; Acc: HGNC: 20190] | | |
| MLKL | mixed lineage kinase domain | ENSG00000168404 | 197259 |
| | like pseudokinase | | |
| | [Source: HGNC | | |
| | Symbol; Acc: HGNC: 26617] | | |
| MPZL2 | myelin protein zero like 2 | ENSG00000149573 | 10205 |
| | [Source: HGNC | | |
| | Symbol; Acc: HGNC: 3496] | | |
| MRGPRX5P | MAS related GPR family | ENSG00000255536 | 0 |
| | member X5, pseudogene | | |
| | [Source: HGNC | | |
| | Symbol; Acc: HGNC: 54329] | | |
| MRPS7 | mitochondrial ribosomal | ENSG00000125445 | 51081 |
| | protein S7 [Source: HGNC | | |
| | Symbol; Acc: HGNC: 14499] | | |
| MTHFD2L | methylenetetrahydrofolate | ENSG00000163738 | 441024 |
| | dehydrogenase (NADP+ | | |
| | dependent) 2 like | | |
| | [Source: HGNC | | |
| | Symbol; Acc: HGNC: 31865] | | |
| MTREX | Mtr4 exosome RNA helicase | ENSG00000039123 | 23517 |
| | [Source: HGNC | | |
| | Symbol; Acc: HGNC: 18734] | | |
| MX2 | MX dynamin like GTPase 2 | ENSG00000183486 | 4600 |
| | [Source: HGNC | | |
| | Symbol; Acc: HGNC: 7533] | | |
| MYOF | myoferlin [Source: HGNC | ENSG00000138119 | 26509 |
| | Symbol; Acc: HGNC: 3656] | | |
| NA | #N/A | MSTRG | #N/A |
| NA | #N/A | MSTRG | #N/A |
| NA | #N/A | MSTRG | #N/A |
| NANOGP4 | Nanog homeobox pseudogene | ENSG00000237065 | 0 |
| | 4 [Source: HGNC | | |
| | Symbol; Acc: HGNC: 23102] | | |
| NECAP1 | NECAP endocytosis associated | ENSG00000089818 | 0 |
| | 1 [Source: HGNC | | |
| | Symbol; Acc: HGNC: 24539] | | |
| NEMF | nuclear export mediator factor | ENSG00000165525 | 9147 |
| | [Source: HGNC | | |
| | Symbol; Acc: HGNC: 10663] | | |
| NFAT5 | nuclear factor of activated T | ENSG00000102908 | 10725 |
| | cells 5 [Source: HGNC | | |
| | Symbol; Acc: HGNC: 7774] | | |
| NIPBL | NIPBL cohesin loading factor | ENSG00000164190 | 25836 |
| | [Source: HGNC | | |
| | Symbol; Acc: HGNC: 28862] | | |
| NME8 | NME/NM23 family member 8 | ENSG00000086288 | 51314 |
| | [Source: HGNC | | |
| | Symbol; Acc: HGNC: 16473] | | |
| NOP56P3 | NOP56 ribonucleoprotein | ENSG00000257956 | 0 |
| | pseudogene 3 [Source: HGNC | | |
| | Symbol; Acc: HGNC: 49801] | | |
| NPRL2 | NPR2 like, GATOR1 complex | ENSG00000114388 | 10641 |
| | subunit [Source: HGNC | | |
| | Symbol; Acc: HGNC: 24969] | | |
| NSMCE2 | NSE2 (MMS21) homolog, | ENSG00000156831 | 286053 |
| | SMC5-SMC6 complex SUMO | | |
| | ligase [Source: HGNC | | |
| | Symbol; Acc: HGNC: 26513] | | |
| NUB1 | negative regulator of ubiquitin | ENSG00000013374 | 51667 |
| | like proteins 1 [Source: HGNC | | |
| | Symbol; Acc: HGNC: 17623] | | |
| NUDT7 | nudix hydrolase 7 | ENSG00000140876 | 283927 |
| | [Source: HGNC | | |
| | Symbol; Acc: HGNC: 8054] | | |
| OR10AB1P | olfactory receptor family 10 | ENSG00000176716 | 0 |
| | subfamily AB member 1 | | |
| | pseudogene [Source: HGNC | | |
| | Symbol; Acc: HGNC: 14804] | | |
| OR51S1 | olfactory receptor family 51 | ENSG00000176922 | 119692 |
| | subfamily S member 1 | | |
| | [Source: HGNC | | |
| | Symbol; Acc: HGNC: 15204] | | |
| OR52B6 | olfactory receptor family 52 | ENSG00000187747 | 340980 |
| | subfamily B member 6 | | |
| | [Source: HGNC | | |
| | Symbol; Acc: HGNC: 15211] | | |
| OR5H14 | olfactory receptor family 5 | ENSG00000236032 | 403273 |
| | subfamily H member 14 | | |
| | [Source: HGNC | | |
| | Symbol; Acc: HGNC: 31286] | | |
| OR7E121P | olfactory receptor family 7 | ENSG00000244222 | 0 |
| | subfamily E member 121 | | |
| | pseudogene [Source: HGNC | | |
| | Symbol; Acc: HGNC: 15049] | | |
| OR9Q2 | olfactory receptor family 9 | ENSG00000186513 | 219957 |
| | subfamily Q member 2 | | |
| | [Source: HGNC | | |
| | Symbol; Acc: HGNC: 15328] | | |
| OTULIN | OTU deubiquitinase with linear | ENSG00000154124 | 90268 |
| | linkage specificity | | |
| | Source: HGNC | | |
| | Symbol; Acc: HGNC: 25118] | | |
| PAN3 | poly(A) specific ribonuclease | ENSG00000152520 | 255967 |
| | subunit PAN3 [Source: HGNC | | |
| | Symbol; Acc: HGNC: 29991] | | |
| PAPSS1 | 3′-phosphoadenosine 5′- | ENSG00000138801 | 9061 |
| | phosphosulfate synthase 1 | | |
| | [Source: HGNC | | |
| | Symbol; Acc: HGNC: 8603] | | |
| PAQR5 | progestin and adipoQ receptor | ENSG00000137819 | 54852 |
| | family member 5 | | |
| | [Source: HGNC | | |
| | Symbol; Acc: HGNC: 29645] | | |
| PARP2 | poly(ADP-ribose) polymerase | ENSG00000129484 | 10038 |
| | 2 [Source: HGNC | | |
| | Symbol; Acc: HGNC: 272] | | |
| PARP4 | poly(ADP-ribose) polymerase | ENSG00000102699 | 143 |
| | family member 4 | | |
| | [Source: HGNC | | |
| | Symbol; Acc: HGNC: 271] | | |
| PARP9 | poly(ADP-ribose) polymerase | ENSG00000138496 | 83666 |
| | family member 9 | | |
| | [Source: HGNC | | |
| | Symbol; Acc: HGNC: 24118] | | |
| PCF11 | PCF11 cleavage and | ENSG00000165494 | 51585 |
| | polyadenylation factor subunit | | |
| | [Source: HGNC | | |
| | Symbol; Acc: HGNC: 30097] | | |
| PCM1 | pericentriolar material 1 | ENSG00000078674 | 5108 |
| | [Source: HGNC | | |
| | Symbol; Acc: HGNC: 8727] | | |
| PDE11A | #N/A | ENSG00000284741 | #N/A |
| PIK3C3 | phosphatidylinositol 3-kinase | ENSG00000078142 | 5289 |
| | catalytic subunit type 3 | | |
| | [Source: HGNC | | |
| | Symbol; Acc: HGNC: 8974] | | |
| PIK3R1 | phosphoinositide-3-kinase | ENSG00000145675 | 5295 |
| | regulatory subunit 1 | | |
| | [Source: HGNC | | |
| | Symbol; Acc: HGNC: 8979] | | |
| PLAC8 | placenta associated 8 | ENSG00000145287 | 51316 |
| | [Source: HGNC | | |
| | Symbol; Acc: HGNC: 19254] | | |
| PLD5P1 | PLD5 pseudogene 1 | ENSG00000283930 | 0 |
| | [Source: HGNC | | |
| | Symbol; Acc: HGNC: 55072] | | |
| PRAL | p53 regulation associated | ENSG00000279296 | 0 |
| | IncRNA [Source: HGNC | | |
| | Symbol; Acc: HGNC: 52646] | | |
| PREPL | prolyl endopeptidase like | ENSG00000138078 | 9581 |
| | [Source: HGNC | | |
| | Symbol; Acc: HGNC: 30228] | | |
| PRIM2 | DNA primase subunit 2 | ENSG00000146143 | 5558 |
| | [Source: HGNC | | |
| | Symbol; Acc: HGNC: 9370] | | |
| PRPF40A | pre-mRNA processing factor | ENSG00000196504 | 55660 |
| | 40 homolog A [Source: HGNC | | |
| | Symbol; Acc: HGNC: 16463] | | |
| PRR19 | proline rich 19 [Source: HGNC | ENSG00000188368 | 284338 |
| | Symbol; Acc: HGNC: 33728] | | |
| PRUNE1 | prune exopolyphosphatase 1 | ENSG00000143363 | 58497 |
| | [Source: HGNC | | |
| | Symbol; Acc: HGNC: 13420] | | |
| PTGER4 | prostaglandin E receptor 4 | ENSG00000171522 | 0 |
| | [Source: HGNC | | |
| | Symbol; Acc: HGNC: 9596] | | |
| PTK2B | protein tyrosine kinase 2 beta | ENSG00000120899 | 2185 |
| | [Source: HGNC | | |
| | Symbol; Acc: HGNC: 9612] | | |
| PTPN6 | protein tyrosine phosphatase | ENSG00000111679 | 5777 |
| | non-receptor type 6 | | |
| | [Source: HGNC | | |
| | Symbol; Acc: HGNC: 9658] | | |
| PTPRC | protein tyrosine phosphatase | ENSG00000081237 | 5788 |
| | receptor type C [Source: HGNC | | |
| | Symbol; Acc: HGNC: 9666] | | |
| RAI2 | retinoic acid induced 2 | ENSG00000131831 | 10742 |
| | [Source: HGNC | | |
| | Symbol; Acc: HGNC: 9835] | | |
| RAP1B | RAP1B, member of RAS | ENSG00000127314 | 5908 |
| | oncogene family | | |
| | [Source: HGNC | | |
| | Symbol; Acc: HGNC: 9857] | | |
| RBM39 | RNA binding motif protein 39 | ENSG00000131051 | 9584 |
| | [Source: HGNC | | |
| | Symbol; Acc: HGNC: 15923] | | |
| RBM7P1 | RBM7 pseudogene 1 | ENSG00000223427 | 0 |
| | [Source: HGNC | | |
| | Symbol; Acc: HGNC: 54704] | | |
| REPS1 | RALBP1 associated Eps | ENSG00000135597 | 85021 |
| | domain containing 1 | | |
| | [Source: HGNC | | |
| | Symbol; Acc: HGNC: 15578] | | |
| RER1 | retention in endoplasmic | ENSG00000157916 | 11079 |
| | reticulum sorting receptor 1 | | |
| | [Source: HGNC | | |
| | Symbol; Acc: HGNC: 30309] | | |
| RLN2 | relaxin 2 [Source: HGNC | ENSG00000107014 | 6019 |
| | Symbol; Acc: HGNC: 10027] | | |
| RP11-163O19.16 | tripartite motif-containing | ENSG00000265973 | 0 |
| | pseudogene | | |
| RP11-169K16.8 | ribosomal protein S16 (RPS16) | ENSG00000178715 | 0 |
| | pseudogene | | |
| RP11-20P5.2 | novel pseudogene | ENSG00000229060 | 0 |
| RP11-35G9.3 | novel transcript, antisense to | ENSG00000267040 | 0 |
| | ATP8B1 | | |
| RP11-365F18.3 | Male-specific lethal-3 homolog | ENSG00000239254 | 0 |
| | 1 (Msl3l1) pseudogene | | |
| RP11-674E16.5 | novel transcript | ENSG00000284952 | 0 |
| RPL23AP58 | ribosomal protein L23a | ENSG00000228657 | 0 |
| | pseudogene 58 [Source: HGNC | | |
| | Symbol; Acc: HGNC: 36138] | | |
| RPL26P15 | ribosomal protein L26 | ENSG00000233829 | 0 |
| | pseudogene 15 [Source: HGNC | | |
| | Symbol; Acc: HGNC: 35512] | | |
| RPS10P7 | ribosomal protein S10 | ENSG00000223396 | 0 |
| | pseudogene 7 [Source: HGNC | | |
| | Symbol; Acc: HGNC: 36423] | | |
| RPS27AP11 | RPS27A pseudogene 11 | ENSG00000218208 | 0 |
| | [Source: HGNC | | |
| | Symbol; Acc: HGNC: 36126] | | |
| RPSAP29 | ribosomal protein SA | ENSG00000244722 | 0 |
| | pseudogene 29 [Source: HGNC | | |
| | Symbol; Acc: HGNC: 36680] | | |
| RPSAP53 | ribosomal protein SA | ENSG00000214263 | 0 |
| | pseudogene 53 [Source: HGNC | | |
| | Symbol; Acc: HGNC: 36641] | | |
| RTP4 | receptor transporter protein 4 | ENSG00000136514 | 0 |
| | [Source: HGNC | | |
| | Symbol; Acc: HGNC: 23992] | | |
| SAMD9 | sterile alpha motif domain | ENSG00000205413 | 54809 |
| | containing 9 [Source: HGNC | | |
| | Symbol; Acc: HGNC: 1348] | | |
| SAMD9L | sterile alpha motif domain | ENSG00000177409 | 219285 |
| | containing 9 like | | |
| | [Source: HGNC | | |
| | Symbol; Acc: HGNC: 1349] | | |
| SAMHD1 | SAM and HD domain | ENSG00000101347 | 25939 |
| | containing deoxynucleoside | | |
| | triphosphate | | |
| | triphosphohydrolase 1 | | |
| | [Source: HGNC | | |
| | Symbol; Acc: HGNC: 15925] | | |
| SAT1 | spermidine/spermine N1- | ENSG00000130066 | 0 |
| | acetyltransferase 1 | | |
| | [Source: HGNC | | |
| | Symbol; Acc: HGNC: 10540] | | |
| SECISBP2 | SECIS binding protein 2 | ENSG00000187742 | 79048 |
| | [Source: HGNC | | |
| | Symbol; Acc: HGNC: 30972] | | |
| SENP6 | SUMO specific peptidase 6 | ENSG00000112701 | 26054 |
| | [Source: HGNC | | |
| | Symbol; Acc: HGNC: 20944] | | |
| SEPTIN7P14 | septin 7 pseudogene 14 | ENSG00000245958 | 0 |
| | [Source: HGNC | | |
| | Symbol; Acc: HGNC: 44219] | | |
| SERBP1 | SERPINE1 mRNA binding | ENSG00000142864 | 26135 |
| | protein 1 [Source: HGNC | | |
| | Symbol; Acc: HGNC: 17860] | | |
| SERPINA11 | serpin family A member 11 | ENSG00000186910 | 256394 |
| | [Source: HGNC | | |
| | Symbol; Acc: HGNC: 19193] | | |
| SERPINB9 | serpin family B member 9 | ENSG00000170542 | 5272 |
| | [Source: HGNC | | |
| | Symbol; Acc: HGNC: 8955] | | |
| SLC10A7 | solute carrier family 10 | ENSG00000120519 | 84068 |
| | member 7 [Source: HGNC | | |
| | Symbol; Acc: HGNC: 23088] | | |
| SLC25A14P1 | solute carrier family 25 | ENSG00000213608 | 0 |
| | member 14 pseudogene 1 | | |
| | [Source: HGNC | | |
| | Symbol; Acc: HGNC: 43856] | | |
| SLC25A38P1 | solute carrier family 25 | ENSG00000229785 | 0 |
| | member 38 pseudogene 1 | | |
| | [Source: HGNC | | |
| | Symbol; Acc: HGNC: 43858] | | |
| SLC30A7 | solute carrier family 30 | ENSG00000162695 | 148867 |
| | member 7 [Source: HGNC | | |
| | Symbol; Acc: HGNC: 19306] | | |
| SLC9A9 | solute carrier family 9 member | ENSG00000181804 | 0 |
| | A9 [Source: HGNC | | |
| | Symbol; Acc: HGNC: 20653] | | |
| SMARCC1 | SWI/SNF related, matrix | ENSG00000173473 | 0 |
| | associated, actin dependent | | |
| | regulator of chromatin | | |
| | subfamily c member 1 | | |
| | [Source: HGNC | | |
| | Symbol; Acc: HGNC: 11104] | | |
| SNHG10 | small nucleolar RNA host gene | ENSG00000247092 | 283596 |
| | 10 [Source: HGNC | | |
| | Symbol; Acc: HGNC: 27510] | | |
| SNHG12 | small nucleolar RNA host gene | ENSG00000197989 | 85028 |
| | 12 [Source: HGNC | | |
| | Symbol; Acc: HGNC: 30062] | | |
| SNORA70G | small nucleolar RNA, H/ACA | ENSG00000206650 | 100379132 |
| | box 70G [Source: HGNC | | |
| | Symbol; Acc: HGNC: 34359] | | |
| SNTG2 | syntrophin gamma 2 | ENSG00000172554 | 54221 |
| | [Source: HGNC | | |
| | Symbol; Acc: HGNC: 13741] | | |
| SNX27 | sorting nexin 27 | ENSG00000143376 | 81609 |
| | [Source: HGNC | | |
| | Symbol; Acc: HGNC: 20073] | | |
| SP110 | SP110 nuclear body protein | ENSG00000135899 | 3431 |
| | [Source: HGNC | | |
| | Symbol; Acc: HGNC: 5401] | | |
| SPRED1 | sprouty related EVH1 domain | ENSG00000166068 | 161742 |
| | containing 1 [Source: HGNC | | |
| | Symbol; Acc: HGNC: 20249] | | |
| SRGAP2B | SLIT-ROBO Rho GTPase | ENSG00000196369 | 647135 |
| | activating protein 2B | | |
| | [Source: HGNC | | |
| | Symbol; Acc: HGNC: 35237] | | |
| SRSF4 | serine and arginine rich | ENSG00000116350 | 6429 |
| | splicing factor 4 | | |
| | [Source: HGNC | | |
| | Symbol; Acc: HGNC: 10786] | | |
| STAT1 | signal transducer and activator | ENSG00000115415 | 6772 |
| | of transcription 1 | | |
| | [Source: HGNC | | |
| | Symbol; Acc: HGNC: 11362] | | |
| STAT2 | signal transducer and activator | ENSG00000170581 | 6773 |
| | of transcription 2 | | |
| | [Source: HGNC | | |
| | Symbol; Acc: HGNC: 11363] | | |
| STAT4 | signal transducer and activator | ENSG00000138378 | 6775 |
| | of transcription 4 | | |
| | [Source: HGNC | | |
| | Symbol; Acc: HGNC: 11365] | | |
| SWAP70 | switching B cell complex | ENSG00000133789 | 23075 |
| | subunit SWAP70 | | |
| | [Source: HGNC | | |
| | Symbol; Acc: HGNC: 17070] | | |
| TAS2R3 | taste 2 receptor member 3 | ENSG00000127362 | 50831 |
| | [Source: HGNC | | |
| | Symbol; Acc: HGNC: 14910] | | |
| TAX1BP1 | Tax1 binding protein 1 | ENSG00000106052 | 8887 |
| | [Source: HGNC | | |
| | Symbol; Acc: HGNC: 11575] | | |
| TBPL2 | TATA-box binding protein like | ENSG00000182521 | 387332 |
| | 2 [Source: HGNC | | |
| | Symbol; Acc: HGNC: 19841] | | |
| TBX19 | T-box transcription factor 19 | ENSG00000143178 | 9095 |
| | [Source: HGNC | | |
| | Symbol; Acc: HGNC: 11596] | | |
| TDRD3 | tudor domain containing 3 | ENSG00000083544 | 81550 |
| | [Source: HGNC | | |
| | Symbol; Acc: HGNC: 20612] | | |
| TENT2 | terminal nucleotidyltransferase | ENSG00000164329 | 167153 |
| | 2 [Source: HGNC | | |
| | Symbol; Acc: HGNC: 26776] | | |
| TES | testin LIM domain protein | ENSG00000135269 | 26136 |
| | [Source: HGNC | | |
| | Symbol; Acc: HGNC: 14620] | | |
| TIA1 | TIA1 cytotoxic granule | ENSG00000116001 | 7072 |
| | associated RNA binding | | |
| | protein [Source: HGNC | | |
| | Symbol; Acc: HGNC: 11802] | | |
| TIRAP | TIR domain containing adaptor | ENSG00000150455 | 114609 |
| | protein [Source: HGNC | | |
| | Symbol; Acc: HGNC: 17192] | | |
| TKT | transketolase [Source: HGNC | ENSG00000163931 | 7086 |
| | Symbol; Acc: HGNC: 11834] | | |
| TMEM59L | transmembrane protein 59 like | ENSG00000105696 | 25789 |
| | [Source: HGNC | | |
| | Symbol; Acc: HGNC: 13237] | | |
| TNFSF10 | TNF superfamily member 10 | ENSG00000121858 | 8743 |
| | [Source: HGNC | | |
| | Symbol; Acc: HGNC: 11925] | | |
| TPRKB | TP53RK binding protein | ENSG00000144034 | 51002 |
| | [Source: HGNC | | |
| | Symbol; Acc: HGNC: 24259] | | |
| TRAP1 | TNF receptor associated | ENSG00000126602 | 10131 |
| | protein 1 [Source: HGNC | | |
| | Symbol; Acc: HGNC: 16264] | | |
| TRAV16 | T cell receptor alpha variable | ENSG00000211796 | 0 |
| | 16 [Source: HGNC | | |
| | Symbol; Acc: HGNC: 12112] | | |
| TRIM22 | tripartite motif containing 22 | ENSG00000132274 | 10346 |
| | [Source: HGNC | | |
| | Symbol; Acc: HGNC: 16379] | | |
| TRIM38 | tripartite motif containing 38 | ENSG00000112343 | 10475 |
| | [Source: HGNC | | |
| | Symbol; Acc: HGNC: 10059] | | |
| TXNIP | thioredoxin interacting protein | ENSG00000265972 | 10628 |
| | [Source: HGNC | | |
| | Symbol; Acc: HGNC: 16952] | | |
| UBA3 | ubiquitin like modifier | ENSG00000144744 | 9039 |
| | activating enzyme 3 | | |
| | [Source: HGNC | | |
| | Symbol; Acc: HGNC: 12470] | | |
| UBE2Q2P1 | ubiquitin conjugating enzyme | ENSG00000189136 | 388165 |
| | E2 Q2 pseudogene 1 | | |
| | [Source: HGNC | | |
| | Symbol; Acc: HGNC: 37439] | | |
| UBE2V1 | ubiquitin conjugating enzyme | ENSG00000244687 | 7335 |
| | E2 V1 [Source: HGNC | | |
| | Symbol; Acc: HGNC: 12494] | | |
| UCK2 | uridine-cytidine kinase 2 | ENSG00000143179 | 7371 |
| | [Source: HGNC | | |
| | Symbol; Acc: HGNC: 12562] | | |
| UCKL1 | uridine-cytidine kinase 1 like 1 | ENSG00000198276 | 54963 |
| | [Source: HGNC | | |
| | Symbol; Acc: HGNC: 15938] | | |
| UEVLD | UEV and lactate/malate | ENSG00000151116 | 55293 |
| | dehyrogenase domains | | |
| | [Source: HGNC | | |
| | Symbol; Acc: HGNC: 30866] | | |
| UIMC1 | ubiquitin interaction motif | ENSG00000087206 | 51720 |
| | containing 1 [Source: HGNC | | |
| | Symbol; Acc: HGNC: 30298] | | |
| UNC50 | unc-50 inner nuclear membrane | ENSG00000115446 | 25972 |
| | RNA binding protein | | |
| | [Source: HGNC | | |
| | Symbol; Acc: HGNC: 16046] | | |
| UPP1 | uridine phosphorylase 1 | ENSG00000183696 | 7378 |
| | [Source: HGNC | | |
| | Symbol; Acc: HGNC: 12576] | | |
| UPRT | uracil | ENSG00000094841 | 139596 |
| | phosphoribosyltransferase | | |
| | homolog [Source: HGNC | | |
| | Symbol; Acc: HGNC: 28334] | | |
| VPS13C | vacuolar protein sorting 13 | ENSG00000129003 | 54832 |
| | homolog C [Source: HGNC | | |
| | Symbol; Acc: HGNC: 23594] | | |
| VPS29 | VPS29 retromer complex | ENSG00000111237 | 51699 |
| | component [Source: HGNC | | |
| | Symbol; Acc: HGNC: 14340] | | |
| WAPL | WAPL cohesin release factor | ENSG00000062650 | 23063 |
| | [Source: HGNC | | |
| | Symbol; Acc: HGNC: 23293] | | |
| WASHC4 | WASH complex subunit 4 | ENSG00000136051 | 23325 |
| | [Source: HGNC | | |
| | Symbol; Acc: HGNC: 29174] | | |
| WDFY2 | WD repeat and FYVE domain | ENSG00000139668 | 115825 |
| | containing 2 [Source: HGNC | | |
| | Symbol; Acc: HGNC: 20482] | | |
| WDR70 | WD repeat domain 70 | ENSG00000082068 | 55100 |
| | [Source: HGNC | | |
| | Symbol; Acc: HGNC: 25495] | | |
| WSB1 | WD repeat and SOCS box | ENSG00000109046 | 26118 |
| | containing 1 [Source: HGNC | | |
| | Symbol; Acc: HGNC: 19221] | | |
| WTAP | WT1 associated protein | ENSG00000146457 | 9589 |
| | [Source: HGNC | | |
| | Symbol; Acc: HGNC: 16846] | | |
| XAF1 | XIAP associated factor 1 | ENSG00000132530 | 54739 |
| | [Source: HGNC | | |
| | Symbol; Acc: HGNC: 30932] | | |
| XRN1 | 5′-3′ exoribonuclease 1 | ENSG00000114127 | 54464 |
| | [Source: HGNC | | |
| | Symbol; Acc: HGNC: 30654] | | |
| ZBP1 | Z-DNA binding protein 1 | ENSG00000124256 | 81030 |
| | [Source: HGNC | | |
| | Symbol; Acc: HGNC: 16176] | | |
| ZDHHC21 | zinc finger DHHC-type | ENSG00000175893 | 340481 |
| | palmitoyltransferase 21 | | |
| | [Source: HGNC | | |
| | Symbol; Acc: HGNC: 20750] | | |
| ZNF239 | zinc finger protein 239 | ENSG00000196793 | 8187 |
| | [Source: HGNC | | |
| | Symbol; Acc: HGNC: 13031] | | |
| ZNF281 | zinc finger protein 281 | ENSG00000162702 | 23528 |
| | [Source: HGNC | | |
| | Symbol; Acc: HGNC: 13075] | | |
| ZNF365 | zinc finger protein 365 | ENSG00000138311 | 22891 |
| | [Source: HGNC | | |
| | Symbol; Acc: HGNC: 18194] | | |
| ZNF473 | zinc finger protein 473 | ENSG00000142528 | 25888 |
| | [Source: HGNC | | |
| | Symbol; Acc: HGNC: 23239] | | |
| ZNF483 | zinc finger protein 483 | ENSG00000173258 | 158399 |
| | [Source: HGNC | | |
| | Symbol; Acc: HGNC: 23384] | | |
| ZNF493 | zinc finger protein 493 | ENSG00000196268 | 284443 |
| | [Source: HGNC | | |
| | Symbol; Acc: HGNC: 23708] | | |
| ZNF569 | zinc finger protein 569 | ENSG00000196437 | 148266 |
| | [Source: HGNC | | |
| | Symbol; Acc: HGNC: 24737] | | |
| ZNF778 | zinc finger protein 778 | ENSG00000170100 | 197320 |
| | [Source: HGNC | | |
| | Symbol; Acc: HGNC: 26479] | | |
| ZSWIM6 | zinc finger SWIM-type | ENSG00000130449 | 57688 |
| | containing 6 [Source: HGNC | | |
| | Symbol; Acc: HGNC: 29316] | | |

FM4

| | | | |
|---|---|---|---|
| AC018755.16 | putative ATP-binding domain- | ENSG00000269388 | 0 |
| | containing protein 3-like | | |
| | protein (ABP3L) pseudogene | | |
| AGMAT | agmatinase [Source: HGNC | ENSG00000116771 | 0 |
| | Symbol; Acc: HGNC: 18407] | | |
| AMMECR1L | AMMECR1 like | ENSG00000144233 | 83607 |
| | [Source: HGNC | | |
| | Symbol; Acc: HGNC: 28658] | | |
| ANAPC4 | anaphase promoting complex | ENSG00000053900 | 29945 |
| | subunit 4 [Source: HGNC | | |
| | Symbol; Acc: HGNC: 19990] | | |
| ANKRD33B | ankyrin repeat domain 33B | ENSG00000164236 | 651746 |
| | [Source: HGNC | | |
| | Symbol; Acc: HGNC: 35240] | | |
| ANKRD49 | ankyrin repeat domain 49 | ENSG00000168876 | 54851 |
| | [Source: HGNC | | |
| | Symbol; Acc: HGNC: 25970] | | |
| AOC3 | amine oxidase copper | ENSG00000131471 | 8639 |
| | containing 3 [Source: HGNC | | |
| | Symbol; Acc: HGNC: 550] | | |
| AP1S1 | adaptor related protein complex | ENSG00000106367 | 1174 |
| | 1 subunit sigma 1 | | |
| | [Source: HGNC | | |
| | Symbol; Acc: HGNC: 559] | | |
| AP5S1 | adaptor related protein complex | ENSG00000125843 | 55317 |
| | 5 subunit sigma 1 | | |
| | [Source: HGNC | | |
| | Symbol; Acc: HGNC: 15875] | | |
| ATF7IP2 | activating transcription factor 7 | ENSG00000166669 | 80063 |
| | interacting protein 2 | | |
| | [Source: HGNC | | |
| | Symbol; Acc: HGNC: 20397] | | |
| ATG9A | autophagy related 9A | ENSG00000198925 | 79065 |
| | [Source: HGNC | | |
| | Symbol; Acc: HGNC: 22408] | | |
| AVIL | advillin [Source: HGNC | ENSG00000135407 | 10677 |
| | Symbol; Acc: HGNC: 14188] | | |
| BCKDHB | branched chain keto acid | ENSG00000083123 | 594 |
| | dehydrogenase E1 subunit beta | | |
| | [Source: HGNC | | |
| | Symbol; Acc: HGNC: 987] | | |
| BLOC1S6 | biogenesis of lysosomal | ENSG00000104164 | 26258 |
| | organelles complex 1 subunit 6 | | |
| | [Source: HGNC | | |
| | Symbol; Acc: HGNC: 8549] | | |
| BPGM | bisphosphoglycerate mutase | ENSG00000172331 | 669 |
| | [Source: HGNC | | |
| | Symbol; Acc: HGNC: 1093] | | |
| C10orf105 | chromosome 10 open reading | ENSG00000214688 | 414152 |
| | frame 105 [Source: HGNC | | |
| | Symbol; Acc: HGNC: 20304] | | |
| C12orf65 | mitochondrial translation | ENSG00000130921 | 91574 |
| | release factor in rescue | | |
| | [Source: HGNC | | |
| | Symbol; Acc: HGNC: 26784] | | |
| C18orf25 | chromosome 18 open reading | ENSG00000152242 | 147339 |
| | frame 25 [Source: HGNC | | |
| | Symbol; Acc: HGNC: 28172] | | |
| C1orf198 | chromosome 1 open reading | ENSG00000119280 | 84886 |
| | frame 198 [Source: HGNC | | |
| | Symbol; Acc: HGNC: 25900] | | |
| C20orf96 | chromosome 20 open reading | ENSG00000196476 | 140680 |
| | frame 96 [Source: HGNC | | |
| | Symbol; Acc: HGNC: 16227] | | |
| C4orf3 | chromosome 4 open reading | ENSG00000164096 | 401152 |
| | frame 3 [Source: HGNC | | |
| | Symbol; Acc: HGNC: 19225] | | |
| C4orf50 | chromosome 4 open reading | ENSG00000181215 | 0 |
| | frame 50 [Source: HGNC | | |
| | Symbol; Acc: HGNC: 33766] | | |
| C9orf139 | long intergenic non-protein | ENSG00000180539 | 401563 |
| | coding RNA 2908 | | |
| | [Source: HGNC | | |
| | Symbol; Acc: HGNC: 31426] | | |
| CASP3 | caspase 3 [Source: HGNC | ENSG00000164305 | 836 |
| | Symbol; Acc: HGNC: 1504] | | |
| CCDC112 | coiled-coil domain containing | ENSG00000164221 | 153733 |
| | 112 [Source: HGNC | | |
| | Symbol; Acc: HGNC: 28599] | | |
| CCDC117 | coiled-coil domain containing | ENSG00000159873 | 150275 |
| | 117 [Source: HGNC | | |
| | Symbol; Acc: HGNC: 26599] | | |
| CCDC137 | coiled-coil domain containing | ENSG00000185298 | 339230 |
| | 137 [Source: HGNC | | |
| | Symbol; Acc: HGNC: 33451] | | |
| CCDC51 | coiled-coil domain containing | ENSG00000164051 | 79714 |
| | 51 [Source: HGNC | | |
| | Symbol; Acc: HGNC: 25714] | | |
| CDC42SE1 | CDC42 small effector 1 | ENSG00000197622 | 56882 |
| | [Source: HGNC | | |
| | Symbol; Acc: HGNC: 17719] | | |
| CDKN3 | cyclin dependent kinase | ENSG00000100526 | 1033 |
| | inhibitor 3 [Source: HGNC | | |
| | Symbol; Acc: HGNC: 1791] | | |
| CEP89 | centrosomal protein 89 | ENSG00000121289 | 84902 |
| | [Source: HGNC | | |
| | Symbol; Acc: HGNC: 25907] | | |
| CH17-60O17.5 | novel immunoglobulin heavy | ENSG00000278082 | 0 |
| | variable gene | | |
| CHRNA10 | cholinergic receptor nicotinic | ENSG00000129749 | 57053 |
| | alpha 10 subunit | | |
| | [Source: HGNC | | |
| | Symbol; Acc: HGNC: 13800] | | |
| CLK4 | CDC like kinase 4 | ENSG00000113240 | 0 |
| | [Source: HGNC | | |
| | Symbol; Acc: HGNC: 13659] | | |
| CLPX | caseinolytic mitochondrial | ENSG00000166855 | 10845 |
| | matrix peptidase chaperone | | |
| | subunit X [Source: HGNC | | |
| | Symbol; Acc: HGNC: 2088] | | |
| CNNM2 | cyclin and CBS domain | ENSG00000148842 | 54805 |
| | divalent metal cation transport | | |
| | mediator 2 [Source: HGNC | | |
| | Symbol; Acc: HGNC: 103] | | |
| COA3 | cytochrome c oxidase assembly | ENSG00000183978 | 0 |
| | factor 3 [Source: HGNC | | |
| | Symbol; Acc: HGNC: 24990] | | |
| COQ9 | coenzyme Q9 [Source: HGNC | ENSG00000088682 | 0 |
| | Symbol; Acc: HGNC: 25302] | | |
| COX14 | cytochrome c oxidase assembly | ENSG00000178449 | 84987 |
| | factor COX14 [Source: HGNC | | |
| | Symbol; Acc: HGNC: 28216] | | |
| CSTF2 | cleavage stimulation factor | ENSG00000101811 | 1478 |
| | subunit 2 [Source: HGNC | | |
| | Symbol; Acc: HGNC: 2484] | | |
| CTB-109A12.2 | Novel protein | ENSG00000285868 | 0 |
| CZIB | CXXC motif containing zinc | ENSG00000162384 | 54987 |
| | binding protein [Source: HGNC | | |
| | Symbol; Acc: HGNC: 26059] | | |
| DACT1 | dishevelled binding antagonist | ENSG00000165617 | 51339 |
| | of beta catenin 1 | | |
| | [Source: HGNC | | |
| | Symbol; Acc: HGNC: 17748] | | |
| DPCD | deleted in primary ciliary | ENSG00000166171 | 25911 |
| | dyskinesia homolog (mouse) | | |
| | [Source: HGNC | | |
| | Symbol; Acc: HGNC: 24542] | | |
| DTYMK | deoxythymidylate kinase | ENSG00000168393 | 1841 |
| | [Source: HGNC | | |
| | Symbol; Acc: HGNC: 3061] | | |
| DUSP4 | dual specificity phosphatase 4 | ENSG00000120875 | 1846 |
| | [Source: HGNC | | |
| | Symbol; Acc: HGNC: 3070] | | |
| EMC4 | ER membrane protein complex | ENSG00000128463 | 51234 |
| | subunit 4 [Source: HGNC | | |
| | Symbol; Acc: HGNC: 28032] | | |
| ERH | ERH mRNA splicing and | ENSG00000100632 | 2079 |
| | mitosis factor [Source: HGNC | | |
| | Symbol; Acc: HGNC: 3447] | | |
| FAM162A | family with sequence similarity | ENSG00000114023 | 0 |
| | 162 member A [Source: HGNC | | |
| | Symbol; Acc: HGNC: 17865] | | |
| FAM219A | family with sequence similarity | ENSG00000164970 | 203259 |
| | 219 member A [Source: HGNC | | |
| | Symbol; Acc: HGNC: 19920] | | |
| FDPS | farnesyl diphosphate synthase | ENSG00000160752 | 2224 |
| | [Source: HGNC | | |
| | Symbol; Acc: HGNC: 3631] | | |
| FIGNL2 | fidgetin like 2 [Source: HGNC | ENSG00000261308 | 401720 |
| | Symbol; Acc: HGNC: 13287] | | |
| FITM2 | fat storage inducing | ENSG00000197296 | 128486 |
| | transmembrane protein 2 | | |
| | [Source: HGNC | | |
| | Symbol; Acc: HGNC: 16135] | | |
| GLS | glutaminase [Source: HGNC | ENSG00000115419 | 2744 |
| | Symbol; Acc: HGNC: 4331] | | |
| HMGN4 | high mobility group | ENSG00000182952 | 10473 |
| | nucleosomal binding domain 4 | | |
| | [Source: HGNC | | |
| | Symbol; Acc: HGNC: 4989] | | |
| HNRNPA3 | heterogeneous nuclear | ENSG00000170144 | 220988 |
| | ribonucleoprotein A3 | | |
| | [Source: HGNC | | |
| | Symbol; Acc: HGNC: 24941] | | |
| HSDL1 | hydroxysteroid dehydrogenase | ENSG00000103160 | 83693 |
| | like 1 [Source: HGNC | | |
| | Symbol; Acc: HGNC: 16475] | | |
| IMPA1 | inositol monophosphatase 1 | ENSG00000133731 | 3612 |
| | [Source: HGNC | | |
| | Symbol; Acc: HGNC: 6050] | | |
| INTS4 | integrator complex subunit 4 | ENSG00000149262 | 92105 |
| | [Source: HGNC | | |
| | Symbol; Acc: HGNC: 25048] | | |
| KIF3A | kinesin family member 3A | ENSG00000131437 | 11127 |
| | [Source: HGNC | | |
| | Symbol; Acc: HGNC: 6319] | | |
| KMT5A | lysine methyltransferase 5A | ENSG00000183955 | 387893 |
| | [Source: HGNC | | |
| | Symbol; Acc: HGNC: 29489] | | |
| KMT5B | lysine methyltransferase 5B | ENSG00000110066 | 51111 |
| | [Source: HGNC | | |
| | Symbol; Acc: HGNC: 24283] | | |
| KRBOX4 | KRAB box domain containing | ENSG00000147121 | 55634 |
| | 4 [Source: HGNC | | |
| | Symbol; Acc: HGNC: 26007] | | |
| L3MBTL1 | L3MBTL histone methyl- | ENSG00000185513 | 26013 |
| | lysine binding protein 1 | | |
| | [Source: HGNC | | |
| | Symbol; Acc: HGNC: 15905] | | |
| LRRFIP2 | LRR binding FLII interacting | ENSG00000093167 | 9209 |
| | protein 2 [Source: HGNC | | |
| | Symbol; Acc: HGNC: 6703] | | |
| ME1 | malic enzyme 1 | ENSG00000065833 | 4199 |
| | [Source: HGNC | | |
| | Symbol; Acc: HGNC: 6983] | | |
| METTL14 | methyltransferase 14, N6- | ENSG00000145388 | 57721 |
| | adenosine-methyltransferase | | |
| | subunit [Source: HGNC | | |
| | Symbol; Acc: HGNC: 29330] | | |
| MFSD6L | major facilitator superfamily | ENSG00000185156 | 162387 |
| | domain containing 6 like | | |
| | [Source: HGNC | | |
| | Symbol; Acc: HGNC: 26656] | | |
| MIR3913-1 | microRNA 3913-1 | ENSG00000264405 | 100500903 |
| | [Source: HGNC | | |
| | Symbol; Acc: HGNC: 38884] | | |
| MRPL1 | mitochondrial ribosomal | ENSG00000169288 | 65008 |
| | protein L1 [Source: HGNC | | |
| | Symbol; Acc: HGNC: 14275] | | |
| MRPS31 | mitochondrial ribosomal | ENSG00000102738 | 10240 |
| | protein S31 [Source: HGNC | | |
| | Symbol; Acc: HGNC: 16632] | | |
| MSRB2 | methionine sulfoxide reductase | ENSG00000148450 | 0 |
| | B2 [Source: HGNC | | |
| | Symbol; Acc: HGNC: 17061] | | |
| MTND2P28 | MT-ND2 pseudogene 28 | ENSG00000225630 | 0 |
| | [Source: HGNC | | |
| | Symbol; Acc: HGNC: 42129] | | |
| MYL6B | myosin light chain 6B | ENSG00000196465 | 140465 |
| | [Source: HGNC | | |
| | Symbol; Acc: HGNC: 29823] | | |
| MYO7A | myosin VIIA [Source: HGNC | ENSG00000137474 | 4647 |
| | Symbol; Acc: HGNC: 7606] | | |
| NA | #N/A | MSTRG | #N/A |
| NA | #N/A | MSTRG | #N/A |
| NA | #N/A | MSTRG | #N/A |
| NATD1 | N-acetyltransferase domain | ENSG00000274180 | 256302 |
| | containing 1 [Source: HGNC | | |
| | Symbol; Acc: HGNC: 30770] | | |
| NFU1 | NFU1 iron-sulfur cluster | ENSG00000169599 | 27247 |
| | scaffold [Source: HGNC | | |
| | Symbol; Acc: HGNC: 16287] | | |
| NHLRC4 | NHL repeat containing 4 | ENSG00000257108 | 283948 |
| | [Source: HGNC | | |
| | Symbol; Acc: HGNC: 26700] | | |
| NKRF | NFKB repressing factor | ENSG00000186416 | 55922 |
| | [Source: HGNC | | |
| | Symbol; Acc: HGNC: 19374] | | |
| NME1 | NME/NM23 nucleoside | ENSG00000239672 | 4830 |
| | diphosphate kinase 1 | | |
| | [Source: HGNC | | |
| | Symbol; Acc: HGNC: 7849] | | |
| NSRP1P1 | nuclear speckle splicing | ENSG00000235613 | 0 |
| | regulatory protein 1 | | |
| | pseudogene 1 [Source: HGNC | | |
| | Symbol; Acc: HGNC: 38087] | | |
| OARD1 | O-acyl-ADP-ribose deacylase 1 | ENSG00000124596 | 221443 |
| | [Source: HGNC | | |
| | Symbol; Acc: HGNC: 21257] | | |
| ODR4 | odr-4 GPCR localization factor | ENSG00000157181 | 54953 |
| | homolog [Source: HGNC | | |
| | Symbol; Acc: HGNC: 24299] | | |
| OVCA2 | OVCA2 serine hydrolase | ENSG00000262664 | 124641 |
| | domain containing | | |
| | [Source: HGNC | | |
| | Symbol; Acc: HGNC: 24203] | | |
| OXNAD1 | oxidoreductase NAD binding | ENSG00000154814 | 92106 |
| | domain containing 1 | | |
| | [Source: HGNC | | |
| | Symbol; Acc: HGNC: 25128] | | |
| OXSM | 3-oxoacyl-ACP synthase, | ENSG00000151093 | 54995 |
| | mitochondrial [Source: HGNC | | |
| | Symbol; Acc: HGNC: 26063] | | |
| PACSIN2 | protein kinase C and casein | ENSG00000100266 | 11252 |
| | kinase substrate in neurons 2 | | |
| | [Source: HGNC | | |
| | Symbol; Acc: HGNC: 8571] | | |
| PAPSS2 | 3′-phosphoadenosine 5′- | ENSG00000198682 | 9060 |
| | phosphosulfate synthase 2 | | |
| | [Source: HGNC | | |
| | Symbol; Acc: HGNC: 8604] | | |
| PATJ | PATJ crumbs cell polarity | ENSG00000132849 | 10207 |
| | complex component | | |
| | [Source: HGNC | | |
| | Symbol; Acc: HGNC: 28881] | | |
| PDCD1LG2 | programmed cell death 1 ligand | ENSG00000197646 | 80380 |
| | 2 [Source: HGNC | | |
| | Symbol; Acc: HGNC: 18731] | | |
| PIGB | phosphatidylinositol glycan | ENSG00000069943 | 9488 |
| | anchor biosynthesis class B | | |
| | [Source: HGNC | | |
| | Symbol; Acc: HGNC: 8959] | | |
| PIGV | phosphatidylinositol glycan | ENSG00000060642 | 55650 |
| | anchor biosynthesis class V | | |
| | [Source: HGNC | | |
| | Symbol; Acc: HGNC: 26031] | | |
| PMM1 | phosphomannomutase 1 | ENSG00000100417 | 5372 |
| | [Source: HGNC | | |
| | Symbol; Acc: HGNC: 9114] | | |
| POP5 | POP5 homolog, ribonuclease | ENSG00000167272 | 51367 |
| | P/MRP subunit [Source: HGNC | | |
| | Symbol; Acc: HGNC: 17689] | | |
| POPDC2 | popeye domain containing 2 | ENSG00000121577 | 64091 |
| | [Source: HGNC | | |
| | Symbol; Acc: HGNC: 17648] | | |
| PPID | peptidylprolyl isomerase D | ENSG00000171497 | 5481 |
| | [Source: HGNC | | |
| | Symbol; Acc: HGNC: 9257] | | |
| PPP3CB | protein phosphatase 3 catalytic | ENSG00000107758 | 5532 |
| | subunit beta [Source: HGNC | | |
| | Symbol; Acc: HGNC: 9315] | | |
| PRPF38A | pre-mRNA processing factor | ENSG00000134748 | 84950 |
| | 38A [Source: HGNC | | |
| | Symbol; Acc: HGNC: 25930] | | |
| PRPS1 | phosphoribosyl pyrophosphate | ENSG00000147224 | 5631 |
| | synthetase 1 [Source: HGNC | | |
| | Symbol; Acc: HGNC: 9462] | | |
| PUS3 | pseudouridine synthase 3 | ENSG00000110060 | 83480 |
| | [Source: HGNC | | |
| | Symbol; Acc: HGNC: 25461] | | |
| QPCTL | glutaminyl-peptide | ENSG00000011478 | 54814 |
| | cyclotransferase like | | |
| | [Source: HGNC | | |
| | Symbol; Acc: HGNC: 25952] | | |
| RAB40C | RAB40C, member RAS | ENSG00000197562 | 57799 |
| | oncogene family | | |
| | [Source: HGNC | | |
| | Symbol; Acc: HGNC: 18285] | | |
| RAD51C | RAD51 paralog C | ENSG00000108384 | 5889 |
| | [Source: HGNC | | |
| | Symbol; Acc: HGNC: 9820] | | |
| RALGPS1 | Ral GEF with PH domain and | ENSG00000136828 | 9649 |
| | SH3 binding motif 1 | | |
| | [Source: HGNC | | |
| | Symbol; Acc: HGNC: 16851] | | |
| RAVER1 | ribonucleoprotein, PTB binding | ENSG00000161847 | 0 |
| | 1 [Source: HGNC | | |
| | Symbol; Acc: HGNC: 30296] | | |
| RCN3 | reticulocalbin 3 [Source: HGNC | ENSG00000142552 | 57333 |
| | Symbol; Acc: HGNC: 21145] | | |
| RGS18 | regulator of G protein signaling | ENSG00000150681 | 64407 |
| | 18 [Source: HGNC | | |
| | Symbol; Acc: HGNC: 14261] | | |
| RP11-108K14.8 | novel protein | ENSG00000254536 | 0 |
| RP11-182N22.9 | TEC | ENSG00000279608 | 0 |
| RP11-203F10.6 | ribosomal protein L21 (RPL21) | ENSG00000219133 | 0 |
| | pseudogene | | |
| RP11-346I3.5 | novel transcript | ENSG00000285791 | 0 |
| RP11-631M6.2 | family with sequence similarity | ENSG00000250461 | 0 |
| | 133, member B (FAM133B) | | |
| | pseudogene | | |
| RP11-972P1.10 | novel transcript, sense intronic | ENSG00000274427 | 0 |
| | to RILPL1 | | |
| RP5-854E16.2 | CDK5 regulatory subunit | ENSG00000224628 | 0 |
| | associated protein 3 | | |
| | (CDK5RAP3) pseudogene | | |
| RPP25 | ribonuclease P and MRP | ENSG00000178718 | 54913 |
| | subunit p25 [Source: HGNC | | |
| | Symbol; Acc: HGNC: 30361] | | |
| SEMA4G | semaphorin 4G [Source: HGNC | ENSG00000095539 | 57715 |
| | Symbol; Acc: HGNC: 10735] | | |
| SGTB | small glutamine rich | ENSG00000197860 | 54557 |
| | tetratricopeptide repeat co- | | |
| | chaperone beta [Source: HGNC | | |
| | Symbol; Acc: HGNC: 23567] | | |
| SLC16A11 | solute carrier family 16 | ENSG00000174326 | 162515 |
| | member 11 [Source: HGNC | | |
| | Symbol; Acc: HGNC: 23093] | | |
| SLC16A7 | solute carrier family 16 | ENSG00000118596 | 9194 |
| | member 7 [Source: HGNC | | |
| | Symbol; Acc: HGNC: 10928] | | |
| SLC33A1 | solute carrier family 33 | ENSG00000169359 | 9197 |
| | member 1 [Source: HGNC | | |
| | Symbol; Acc: HGNC: 95] | | |
| SMTN | smoothelin [Source: HGNC | ENSG00000183963 | 6525 |
| | Symbol; Acc: HGNC: 11126] | | |
| TBCA | tubulin folding cofactor A | ENSG00000171530 | 6902 |
| | [Source: HGNC | | |
| | Symbol; Acc: HGNC: 11579] | | |
| TEN1-CDK3 | TEN1-CDK3 readthrough | ENSG00000261408 | 100529145 |
| | (NMD candidate) | | |
| | [Source: HGNC | | |
| | Symbol; Acc: HGNC: 44420] | | |
| THAP6 | THAP domain containing 6 | ENSG00000174796 | 152815 |
| | [Source: HGNC | | |
| | Symbol; Acc: HGNC: 23189] | | |
| THYN1 | thymocyte nuclear protein 1 | ENSG00000151500 | 29087 |
| | [Source: HGNC | | |
| | Symbol; Acc: HGNC: 29560] | | |
| TIMM13 | translocase of inner | ENSG00000099800 | 26517 |
| | mitochondrial membrane 13 | | |
| | [Source: HGNC | | |
| | Symbol; Acc: HGNC: 11816] | | |
| TMA16 | translation machinery | ENSG00000198498 | 0 |
| | associated 16 homolog | | |
| | [Source: HGNC | | |
| | Symbol; Acc: HGNC: 25638] | | |
| TRIM37 | tripartite motif containing 37 | ENSG00000108395 | 4591 |
| | [Source: HGNC | | |
| | Symbol; Acc: HGNC: 7523] | | |
| TRMT6 | tRNA methyltransferase 6 non- | ENSG00000089195 | 51605 |
| | catalytic subunit | | |
| | [Source: HGNC | | |
| | Symbol; Acc: HGNC: 20900] | | |
| TRNAU1AP | tRNA selenocysteine 1 | ENSG00000180098 | 0 |
| | associated protein 1 | | |
| | [Source: HGNC | | |
| | Symbol; Acc: HGNC: 30813] | | |
| TXLNG | taxilin gamma [Source: HGNC | ENSG00000086712 | 55787 |
| | Symbol; Acc: HGNC: 18578] | | |
| UBTD1 | ubiquitin domain containing 1 | ENSG00000165886 | 80019 |
| | [Source: HGNC | | |
| | Symbol; Acc: HGNC: 25683] | | |
| USP6NL | USP6 N-terminal like | ENSG00000148429 | 9712 |
| | [Source: HGNC | | |
| | Symbol; Acc: HGNC: 16858] | | |
| WEE1 | WEE1 G2 checkpoint kinase | ENSG00000166483 | 7465 |
| | [Source: HGNC | | |
| | Symbol; Acc: HGNC: 12761] | | |
| ZC3H8 | zinc finger CCCH-type | ENSG00000144161 | 84524 |
| | containing 8 [Source: HGNC | | |
| | Symbol; Acc: HGNC: 30941] | | |
| ZCRB1 | zinc finger CCHC-type and | ENSG00000139168 | 0 |
| | RNA binding motif containing | | |
| | 1 [Source: HGNC | | |
| | Symbol; Acc: HGNC: 29620] | | |
| ZDHHC23 | zinc finger DHHC-type | ENSG00000184307 | 254887 |
| | palmitoyltransferase 23 | | |
| | [Source: HGNC | | |
| | Symbol; Acc: HGNC: 28654] | | |
| ZMAT2 | zinc finger matrin-type 2 | ENSG00000146007 | 153527 |
| | [Source: HGNC | | |
| | Symbol; Acc: HGNC: 26433] | | |
| ZMYND15 | zinc finger MYND-type | ENSG00000141497 | 84225 |
| | containing 15 [Source: HGNC | | |
| | Symbol; Acc: HGNC: 20997] | | |
| ZNF133 | zinc finger protein 133 | ENSG00000125846 | 7692 |
| | [Source: HGNC | | |
| | Symbol; Acc: HGNC: 12917] | | |
| ZNF146 | zinc finger protein 146 | ENSG00000167635 | 7705 |
| | [Source: HGNC | | |
| | Symbol; Acc: HGNC: 12931] | | |
| ZNF280C | zinc finger protein 280C | ENSG00000056277 | 0 |
| | [Source: HGNC | | |
| | Symbol; Acc: HGNC: 25955] | | |
| ZNF43 | zinc finger protein 43 | ENSG00000198521 | 7594 |
| | [Source: HGNC | | |
| | Symbol; Acc: HGNC: 13109] | | |
| ZNF439 | zinc finger protein 439 | ENSG00000171291 | 90594 |
| | [Source: HGNC | | |
| | Symbol; Acc: HGNC: 20873] | | |
| ZNF530 | zinc finger protein 530 | ENSG00000183647 | 348327 |
| | [Source: HGNC | | |
| | Symbol; Acc: HGNC: 29297] | | |
| ZNF554 | zinc finger protein 554 | ENSG00000172006 | 115196 |
| | [Source: HGNC | | |
| | Symbol; Acc: HGNC: 26629] | | |
| ZNF608 | zinc finger protein 608 | ENSG00000168916 | 57507 |
| | [Source: HGNC | | |
| | Symbol; Acc: HGNC: 29238] | | |

### REFERENCES

1. Sarzi-Puttini P, Giorgi V, Marotto D, Atzeni F. Fibromyalgia: an update on clinical characteristics, aetiopathogenesis and treatment. Nat Rev Rheumatol. 2020; 16(11):645-60.
2. Goldenberg D L. Fibromyalgia syndrome. An emerging but controversial condition. JAMA. 1987; 257(20):2782-7.
3. Wolfe F, Clauw D J, Fitzcharles M A, Goldenberg D L, Hauser W, Katz R L, et al. 2016 Revisions to the 2010/2011 fibromyalgia diagnostic criteria. Semin Arthritis Rheum. 2016; 46(3):319-29.
4. Chinn S, Caldwell W, Gritsenko K. Fibromyalgia Pathogenesis and Treatment Options Update. Curr Pain Headache Rep. 2016; 20(4):25.
5. Nicholas M, Vlaeyen J W S, Rief W, Barke A, Aziz Q, Benoliel R, et al. The IASP classification of chronic pain for ICD-11: chronic primary pain. Pain. 2019; 160(1):28-37.
6. Behm F G, Gavin I M, Karpenko O, Lindgren V, Gaitonde S, Gashkoff P A, et al. Unique immunologic patterns in fibromyalgia. BMC Clin Pathol. 2012; 12:25.
7. Wallace D J, Gavin I M, Karpenko O, Barkhordar F, Gillis B S. Cytokine and chemokine profiles in fibromyalgia, rheumatoid arthritis and systemic lupus erythematosus: a potentially useful tool in differential diagnosis. Rheumatol Int. 2015; 35(6):991-6.
8. Meade E, Garvey M. The Role of Neuro-Immune Interaction in Chronic Pain Conditions; Functional Somatic Syndrome, Neurogenic Inflammation, and Peripheral Neuropathy. Int J Mol Sci. 2022; 23(15).
9. Clos-Garcia M, Andres-Marin N, Fernandez-Eulate G, Abecia L, Lavin J L, van Liempd S, et al. Gut microbiome and serum metabolome analyses identify molecular biomarkers and altered glutamate metabolism in fibromyalgia. EbioMedicine. 2019; 46:499-511.
10. Minerbi A, Fitzcharles M A. Gut microbiome: pertinence in fibromyalgia. Clin Exp Rheumatol. 2020; 38 Suppl 123(1):99-104.
11. Arnold L M, Fan J, Russell I J, Yunus M B, Khan M A, Kushner I, et al. The fibromyalgia family study: a genome-wide linkage scan study. Arthritis Rheum. 2013; 65(4):1122-8.
12. Arnold L M, Hudson J I, Hess E V, Ware A E, Fritz D A, Auchenbach M B, et al. Family study of fibromyalgia. Arthritis Rheum. 2004; 50(3):944-52.
13. Kato K, Sullivan P F, Evengard B, Pedersen N L. A population-based twin study of functional somatic syndromes. Psychol Med. 2009; 39(3):497-505.
14. Armitage R, Landis C, Hoffmann R, Lentz M, Watson N, Goldberg J, et al. Power spectral analysis of sleep EEG in twins discordant for chronic fatigue syndrome. J Psychosom Res. 2009; 66(1):51-7.
15. Dobin A, Davis C A, Schlesinger F, Drenkow J, Zaleski C, Jha S, et al. STAR: ultrafast universal RNA-seq aligner. Bioinformatics. 2013; 29(1):15-21.
16. Danecek P, Bonfield J K, Liddle J, Marshall J, Ohan V, Pollard M O, et al. Twelve years of SAMtools and BCFtools. Gigascience. 2021; 10(2).
17. Pertea M, Pertea G M, Antonescu C M, Chang T C, Mendell J T, Salzberg S L. StringTie enables improved reconstruction of a transcriptome from RNA-seq reads. Nat Biotechnol. 2015; 33(3):290-5.
18. Vitting-Seerup K, Sandelin A. The Landscape of Isoform Switches in Human Cancers. Mol Cancer Res. 2017; 15(9):1206-20.
19. Smid M, Coebergh van den Braak R R J, van de Werken H J G, van Riet J, van Galen A, de Weerd V, et al. Gene length corrected trimmed mean of M-values (GeTMM) processing of RNA-seq data performs similarly in intersample analyses while improving intrasample comparisons. BMC Bioinformatics. 2018; 19(1):236.
20. Love M I, Huber W, Anders S. Moderated estimation of fold change and dispersion for RNA-seq data with DESeq2. Genome Biol. 2014; 15(12):550.
21. Subramanian A, Tamayo P, Mootha V K, Mukherjee S, Ebert B L, Gillette M A, et al. Gene set enrichment analysis: a knowledge-based approach for interpreting genome-wide expression profiles. Proc Natl Acad Sci USA. 2005; 102(43):15545-50.
22. Smoot M E, Ono K, Ruscheinski J, Wang P L, Ideker T. Cytoscape 2.8: new features for data integration and network visualization. Bioinformatics. 2011; 27(3):431-2.
23. Wolfe F, Smythe H A, Yunus M B, Bennett R M, Bombardier C, Goldenberg D L, et al. The American College of Rheumatology 1990 Criteria for the Classification of Fibromyalgia. Report of the Multicenter Criteria Committee. Arthritis Rheum. 1990; 33(2):160-72.
24. Ablin K, Clauw D J. From fibrositis to functional somatic syndromes to a bell-shaped curve of pain and sensory sensitivity: evolution of a clinical construct. Rheum Dis Clin North Am. 2009; 35(2):233-51.
25. Siracusa R, Paola R D, Cuzzocrea S, Impellizzeri D. Fibromyalgia: Pathogenesis, Mechanisms, Diagnosis and Treatment Options Update. Int J Mol Sci. 2021; 22(8).
26. Galvez-Sanchez C M, Reyes Del Paso G A. Diagnostic Criteria for Fibromyalgia: Critical Review and Future Perspectives. J Clin Med. 2020; 9(4).
27. Maixner W, Fillingim R B, Williams D A, Smith S B, Slade G D. Overlapping Chronic Pain Conditions: Implications for Diagnosis and Classification. J Pain. 2016; 17(9 Suppl):T93-T107.
28. Kalpachidou T, Spiecker L, Kress M, Quarta S. Rho GTPases in the Physiology and Pathophysiology of Peripheral Sensory Neurons. Cells. 2019; 8(6).
29. Reinhardt J W, Breuer C K. Fibrocytes: A Critical Review and Practical Guide. Front Immunol. 2021; 12:784401.
30. Bucala R. Fibrocytes at 20 Years. Mol Med. 2015; 21 Suppl 1:S3-5.
31. Quan T E, Cowper S, Wu S P, Bockenstedt L K, Bucala R. Circulating fibrocytes: collagen-secreting cells of the peripheral blood. Int J Biochem Cell Biol. 2004; 36(4):598-606.
32. Heasman S J, Ridley A J. Mammalian Rho GTPases: new insights into their functions from in vivo studies. Nat Rev Mol Cell Biol. 2008; 9(9):690-701.
33. Langevin H M, Cornbrooks C J, Taatjes D J. Fibroblasts form a body-wide cellular network. Histochem Cell Biol. 2004; 122(1):7-15.
34. Kniazeva E, Putnam A J. Endothelial cell traction and ECM density influence both capillary morphogenesis and maintenance in 3-D. Am J Physiol Cell Physiol. 2009; 297(1):C179-87.
35. Lemos D R, Duffield J S. Tissue-resident mesenchymal stromal cells: Implications for tissue-specific antifibrotic therapies. Sci Transl Med. 2018; 10(426).
36. Koliaraki V, Prados A, Armaka M, Kollias G. The mesenchymal context in inflammation, immunity and cancer. Nat Immunol. 2020; 21(9):974-82.
37. Sardiello M, Palmieri M, di Ronza A, Medina D L, Valenza M, Gennarino V A, et al. A gene network regulating lysosomal biogenesis and function. Science. 2009; 325(5939):473-7.
38. Sardiello M. Transcription factor EB: from master coordinator of lysosomal pathways to candidate therapeutic target in degenerative storage diseases. Ann N Y Acad Sci. 2016; 1371(1):3-14.
39. Settembre C, Zoncu R, Medina D L, Vetrini F, Erdin S, Erdin S, et al. A lysosome-tonucleus signalling mechanism senses and regulates the lysosome via mTOR and TFEB. EMBO J. 2012; 31(5):1095-108.
40. Vega-Rubin-de-Celis S, Pena-Llopis S, Konda M, Brugarolas J. Multistep regulation of TFEB by MTORC1. Autophagy. 2017; 13(3):464-72.
41. Settembre C, Di Malta C, Polito V A, Garcia Arencibia M, Vetrini F, Erdin S, et al. TFEB links autophagy to lysosomal biogenesis. Science. 2011; 332(6036):1429-33.
42. Napolitano G, Ballabio A. TFEB at a glance. J Cell Sci. 2016; 129(13):2475-81.
43. Saxton R A, Sabatini D M. mTOR Signaling in Growth, Metabolism, and Disease. Cell. 2017; 168(6):960-76.
44. Lim C Y, Zoncu R. The lysosome as a command-and-control center for cellular metabolism. J Cell Biol. 2016; 214(6):653-64.
45. Perera R M, Zoncu R. The Lysosome as a Regulatory Hub. Annu Rev Cell Dev Biol. 2016; 32:223-53.
46. Palmieri M, Impey S, Kang H, di Ronza A, Pelz C, Sardiello M, et al. Characterization of the CLEAR network reveals an integrated control of cellular clearance pathways. Hum Mol Genet. 2011; 20(19):3852-66.
47. Schaaf M B, Keulers T G, Vooijs M A, Rouschop K M. LC3/GABARAP family proteins: autophagy-(un)related functions. FASEB J. 2016; 30(12):3961-78.
48. Chino H, Hatta T, Natsume T, Mizushima N. Intrinsically Disordered Protein TEX264 Mediates ER-phagy. Mol Cell. 2019; 74(5):909-21 e6.
49. Kanneganti T D, Lamkanfi M, Nunez G. Intracellular NOD-like receptors in host defense and disease. Immunity. 2007; 27(4):549-59.
50. Gillis, et al., Interleukin Deficiency Disorder Patient Responses to COVID-19 Infections. Journal of Advances in Medicine and Medical Research. 2021. Pages 40-45.
51. Molina, F. et al. Are patients with fibromyalgia in a prothrombotic state?. Biol. Res. Nurs. 21(2), 224-230 (2019).
52. Rus, A. et al. Nitric oxide, inflammation, lipid profile, and cortisol in normal- and overweight women with fibromyalgia. Biol. Res. Nurs. 18(2), 138-146 (2016).
53. Dolcino, M., Tinazzi, E., Puccetti, A. & Lunardi, C. Gene expression profiling in fibromyalgia indicates an autoimmune origin of the disease and opens new avenues for targeted therapy. J. Clin. Med. 9(6), 1814 (2020).
54. Han, C. L., Sheng, Y. C., Wang, S. Y., Chen, Y. H. & Kang, J. H. Serum proteome profiles revealed dysregulated proteins and mechanisms associated with fibromyalgia syndrome in women. Sci. Rep. 10(1), 12347 (2020).
55. Wahlen, K. et al. Significant correlation between plasma proteome profile and pain intensity, sensitivity, and psychological distress in women with fibromyalgia. Sci. Rep. 10(1), 12508 (2020).
56. De Ciampi Andrade, D. et al. Epigenetics insights into chronic pain: DNA hypomethylation in fibromyalgia—a controlled pilot-study. Pain 158(8), 1473-1480 (2017).
57. Polli, A., Nijs, J., Ickmans, K., Velkeniers, B. & Godderis, L. Linking lifestyle factors to complex pain states: 3 reasons why understanding epigenetics may improve the delivery of patient-centered care. J. Orthop. Sports Phys. Ther. 49(10), 683-687 (2019).

It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

All publications, patents and patent applications herein are incorporated by reference to the same extent as if each individual publication or patent application was specifically and individually indicated to be incorporated by reference. It is not an admission that any of the information provided herein is prior art or relevant to the presently claimed inventions, or that any publication specifically or implicitly referenced is prior art.

While the invention has been described in connection with specific embodiments thereof, the foregoing description has been given for clearness of understanding only and no unnecessary limitations should be understood therefrom. It will be understood that the description is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims. Those skilled in the art will recognize, or be able to ascertain using no more than routine experimentation, many equivalents to the specific embodiments of the invention described herein. Such equivalents are intended to be encompassed by the following claims.

### Further Numbered Embodiments of the Disclosure

Other subject matter contemplated by the present disclosure is set out in the following numbered embodiments:

1. A method of assaying a sample obtained from a subject, the method comprising measuring a nucleic acid expression level of a plurality of biomarkers from either Table 3 or Table 4 in the sample obtained from the subject, wherein the subject suffers from or is suspected of suffering from fibromyalgia (FM).

2. The method of embodiment 1, wherein the method further comprises comparing the detected levels of nucleic acid expression of the plurality of biomarkers selected from Table 3 or Table 4 to the expression of the plurality of biomarkers selected from Table 3 or Table 4 in a control; and classifying the subject as having FM based on the results of the comparing step.

3. The method of embodiment 2, wherein the comparing step comprises applying a statistical algorithm which comprises determining a correlation between the expression data obtained from the sample and the expression data from the control.

4. The method of any one of the above embodiments, wherein the nucleic acid expression level is RNA or cDNA.

5. The method any one of the above embodiments, wherein the detecting the nucleic acid expression level comprises performing quantitative real time reverse transcriptase polymerase chain reaction (qRT-PCR), RNAseq or whole transcriptome analysis.

6. The method of embodiment 5, wherein the nucleic acid expression level is detected by performing RNA-seq.

7. The method of embodiment 6, wherein the detection of the nucleic acid expression level comprises using at least one pair of oligonucleotide primers specific for each biomarker from the plurality of biomarkers selected from Table 3 or Table 4.

8. The method of any one of the above embodiments, wherein the sample is a bodily fluid obtained from the subject.

9. The method of embodiment 8, wherein the bodily fluid is blood or fractions thereof, urine, saliva, or sputum.

10. The method of any one of the above embodiments, wherein the plurality of biomarkers selected from Table 3 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers or at least 74 biomarkers from Table 3.

11. The method of any one of the above embodiments, wherein the plurality of biomarkers selected from Table 3 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 3.

12. The method of any one of embodiments 1-9, wherein the plurality of biomarkers selected from Table 3 comprise biomarkers associated with the GP6 signaling pathway, the wound healing signaling pathway, RHOGDI signaling, phagosome formation, the intrinsic prothrombin activation pathway, the pulmonary fibrosis idiopathic signaling pathway, synaptic long term depression, the oxytocin signaling pathway, sperm motility or cell cycle GS/M DNA damage checkpoint regulation.

13. The method of any one of embodiments 1-9, wherein the plurality of biomarkers selected from Table 3 comprises all the biomarkers from Table 3.

14. The method of any one of embodiments 1-9, wherein the plurality of biomarkers selected from Table 4 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers, at least 84 biomarkers, at least 86 biomarkers, at least 88 biomarkers, at least 90 biomarkers, at least 92 biomarkers, at least 94 biomarkers, at least 96 biomarkers, at least 98 biomarkers or at least 99 biomarkers from Table 4.

15. The method of any one of embodiments 1-9, wherein the plurality of biomarkers selected from Table 4 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 4.

16. The method of any one of embodiments 1-9, wherein the plurality of biomarkers selected from Table 4 comprise biomarkers associated with phagosome formation, the CLEAR signaling pathway, the pyroptosis signaling pathway, TREM1 signaling, the neuroinflammation signaling pathway, Th1 pathway, LPS/Il-1 mediated inhibition of RXR function, crosstalk between dendritic cells and natural killer cells, Fcgamma Receptor-mediated phagocytosis in macrophages and monocytes, production of nitric oxide and reactive oxygen species in macrophages, Toll-Like receptor signaling, inflammasome pathway, G-protein coupled receptor signaling, Th2 pathway, LXR/RXR activation, role of pattern recognition receptors in recognition of bacteria and viruses, glycolysis I or the necroptosis signaling pathway.

17. The method of any one of embodiments 1-9, wherein the plurality of biomarkers selected from Table 4 comprises all the biomarkers from Table 4.

18. A method of detecting a biomarker in a sample obtained from a subject suffering from or suspected of suffering from fibromyalgia (FM), the method comprising, consisting essentially of or consisting of measuring the nucleic acid expression level of a plurality of biomarkers selected from Table 3 or 4 using an amplification, hybridization and/or sequencing assay.

19. The method of embodiment 18, wherein the sample was previously diagnosed as being FM.

20. The method of embodiment 18 or 19, wherein the amplification, hybridization and/or sequencing assay comprises performing quantitative real time reverse transcriptase polymerase chain reaction (qRT-PCR), RNAseq, microarrays, gene chips, Serial Analysis of Gene Expression (SAGE), Rapid Analysis of Gene Expression (RAGE), nuclease protection assays, or Northern blotting.

21. The method of embodiment 20, wherein the nucleic acid expression level is detected by performing qRT-PCR.

22. The method of embodiment 18, wherein the detection of the nucleic acid expression level comprises using at least one pair of oligonucleotide primers per each of the plurality of biomarker selected from Table 3 or 4.

23. The method of any one of embodiments 18-22, wherein the sample is a bodily fluid obtained from the subject.

24. The method of embodiment 23, wherein the bodily fluid is blood or fractions thereof, urine, saliva, or sputum.

25. The method of any one embodiments 18-24, wherein the plurality of biomarkers selected from Table 3 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers or at least 74 biomarkers from Table 3.

26. The method of any one of embodiments 18-24, wherein the plurality of biomarkers selected from Table 3 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 3.

27. The method of any one of embodiments 18-24, wherein the plurality of biomarkers selected from Table 3 comprise biomarkers associated with the GP6 signaling pathway, the wound healing signaling pathway, RHOGDI signaling, phagosome formation, the intrinsic prothrombin activation pathway, the pulmonary fibrosis idiopathic signaling pathway, synaptic long term depression, the oxytocin signaling pathway, sperm motility or cell cycle GS/M DNA damage checkpoint regulation.

28. The method of any one of embodiments 18-24, wherein the plurality of biomarkers selected from Table 3 comprises all the biomarkers from Table 3.

29. The method of any one of embodiments 18-24, wherein the plurality of biomarkers selected from Table 4 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers, at least 84 biomarkers, at least 86 biomarkers, at least 88 biomarkers, at least 90 biomarkers, at least 92 biomarkers, at least 94 biomarkers, at least 96 biomarkers, at least 98 biomarkers or at least 99 biomarkers from Table 4.

30. The method of any one of embodiments 18-24, wherein the plurality of biomarkers selected from Table 4 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 4.

31. The method of any one of embodiments 18-24, wherein the plurality of biomarkers selected from Table 4 comprise biomarkers associated with phagosome formation, the CLEAR signaling pathway, the pyroptosis signaling pathway, TREM1 signaling, the neuroinflammation signaling pathway, Th1 pathway, LPS/Il-1 mediated inhibition of RXR function, crosstalk between dendritic cells and natural killer cells, Fcgamma Receptor-mediated phagocytosis in macrophages and monocytes, production of nitric oxide and reactive oxygen species in macrophages, Toll-Like receptor signaling, inflammasome pathway, G-protein coupled receptor signaling, Th2 pathway, LXR/RXR activation, role of pattern recognition receptors in recognition of bacteria and viruses, glycolysis I or the necroptosis signaling pathway.

32. The method of any one of embodiments 18-24, wherein the plurality of biomarkers selected from Table 4 comprises all the biomarkers from Table 4.

33. A method of treating fibromyalgia (FM) in a subject, the method comprising:
measuring a nucleic acid expression level of a plurality of biomarkers in a sample obtained from a subject suspected of suffering from FM, wherein the plurality of biomarkers is selected from Table 3 or 4, wherein the nucleic acid expression level of the plurality of biomarkers indicates that the subject has FM; and
administering a standard of care for FM to the subject, wherein the standard of care for FM is selected from the group consisting of duloxetine, pregabalin, gabapentin and milnacipran.

34. The method of embodiment 33, wherein the determining step further comprises comparing the nucleic acid expression levels of the plurality of biomarkers from Table 3 to the nucleic acid expression levels of the plurality of biomarkers from Table 3 in at least one sample training set(s), wherein the at least one sample training set comprises nucleic acid expression level data of the plurality of biomarkers from Table 3 from a reference FM sample, nucleic acid expression level data of the plurality of biomarkers from Table 3 from a reference non-FM sample or a combination thereof, and classifying the subject as having FM based on the results of the comparing step.

35. The method of embodiment 34, wherein the non-FM sample is obtained from an individual not known to have FM.

36. The method of embodiment 34, wherein the non-FM sample is obtained from an individual diagnosed with a disease of widespread pain selected from the group consisting of rheumatoid arthritis, systemic lupus erythematosus (SLE), Sjogren Syndrome and multiple sclerosis.

37. The method of any one of embodiments 34-36, wherein the comparing step comprises applying a statistical algorithm which comprises determining a correlation between the expression data obtained from the sample obtained from the subject and the expression data from the at least one training set(s); and classifying the subject as having FM based on the results of the statistical algorithm.

38. The method of any one of embodiments 33-37, wherein the plurality of biomarkers selected from Table 3 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers or at least 74 biomarkers from Table 3.

39. The method of any one of embodiments 33-37, wherein the plurality of biomarkers selected from Table 3 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 3.

40. The method of any one of embodiments 33-37, wherein the plurality of biomarkers selected from Table 3 comprise biomarkers associated with the GP6 signaling pathway, the wound healing signaling pathway, RHOGDI signaling, phagosome formation, the intrinsic prothrombin activation pathway, the pulmonary fibrosis idiopathic signaling pathway, synaptic long term depression, the oxytocin signaling pathway, sperm motility or cell cycle GS/M DNA damage checkpoint regulation.

41. The method of any one of embodiments 33-37, wherein the plurality of biomarkers selected from Table 3 comprises all the biomarkers from Table 3.

42. The method of any one of embodiments 33-37, wherein the plurality of biomarkers selected from Table 4 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers, at least 84 biomarkers, at least 86 biomarkers, at least 88 biomarkers, at least 90 biomarkers, at least 92 biomarkers, at least 94 biomarkers, at least 96 biomarkers, at least 98 biomarkers or at least 99 biomarkers from Table 4.

43. The method of any one of embodiments 33-37, wherein the plurality of biomarkers selected from Table 4 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 4.

44. The method of any one of embodiments 33-37, wherein the plurality of biomarkers selected from Table 4 comprise biomarkers associated with phagosome formation, the CLEAR signaling pathway, the pyroptosis signaling pathway, TREM1 signaling, the neuroinflammation signaling pathway, Th1 pathway, LPS/Il-1 mediated inhibition of RXR function, crosstalk between dendritic cells and natural killer cells, Fcgamma Receptor-mediated phagocytosis in macrophages and monocytes, production of nitric oxide and reactive oxygen species in macrophages, Toll-Like receptor signaling, inflammasome pathway, G-protein coupled receptor signaling, Th2 pathway, LXR/RXR activation, role of pattern recognition receptors in recognition of bacteria and viruses, glycolysis I or the necroptosis signaling pathway.

45. The method of any one of embodiments 33-37, wherein the plurality of biomarkers selected from Table 4 comprises all the biomarkers from Table 4.

46. The method of any one of embodiments 33-45, wherein the measuring the nucleic acid expression level is conducted using an amplification, hybridization and/or sequencing assay.

47. The method of embodiment 46, wherein the amplification, hybridization and/or sequencing assay comprises performing quantitative real time reverse transcriptase polymerase chain reaction (qRT-PCR), RNAseq, microarrays, gene chips, Serial Analysis of Gene Expression (SAGE), Rapid Analysis of Gene Expression (RAGE), nuclease protection assays or Northern blotting.

48. The method of embodiment 47, wherein the expression level is detected by performing qRT-PCR.

49. The method of any one of embodiments 33-48, wherein the sample is a bodily fluid obtained from the subject.

50. The method of embodiment 49, wherein the bodily fluid is blood or fractions thereof, urine, saliva, or sputum.

51. A system for diagnosing fibromyalgia (FM) from a sample obtained from a subject suspected of suffering from FM, the system comprising:
(a) one or more processors; and
(b) one or more memories operatively coupled to at least one of the one or more processors and having instructions stored thereon that, when executed by at least one of the one or more processors, cause the system to
   (i) detect an expression level of each of a plurality of biomarkers from Table 3 or 4;
   (ii) compare the expression levels of each of the plurality of biomarkers from Table 3 to the expression levels of each of the plurality of biomarkers from Table 3 in a control or compare the expression levels of each of the plurality of biomarkers from Table 4 to the expression levels of each of the plurality of biomarkers from Table 4 in a control; and
   (iii) classifying the subject as having FM based on the results of the comparing step.

52. The system of embodiment 51, wherein the control comprises at least one sample training set(s), wherein the at least one sample training set comprises expression levels of each of the plurality of biomarkers from Table 3 or 4 from a reference FM sample, expression levels of each of the plurality of biomarkers from Table 3 or 4 from a reference non-FM sample or a combination thereof.

53. The system of embodiment 52, wherein the non-FM sample is obtained from an individual not known to have FM.

54. The system of embodiment 53, wherein the non-FM sample is obtained from an individual diagnosed with a disease of widespread pain selected from the group consisting of rheumatoid arthritis, systemic lupus erythematosus (SLE), Sjogren Syndrome and multiple sclerosis.

55. The system of any one of embodiments 51-54, wherein the comparing step comprises applying a statistical algorithm which comprises determining a correlation between the nucleic acid expression data obtained from the sample and the expression data from the at least one training set(s); and classifying the subject as having FM based on the results of the statistical algorithm.

56. The system of any one of embodiments 51-55, wherein the expression level is a nucleic acid expression level, wherein the nucleic acid expression level is RNA or cDNA.

57. The system of embodiment 56, wherein the detecting the expression level comprises performing quantitative real time reverse transcriptase polymerase chain reaction (qRT-PCR), RNAseq, microarrays, gene chips, Serial Analysis of Gene Expression (SAGE), Rapid Analysis of Gene Expression (RAGE), nuclease protection assays or Northern blotting.

58. The system of embodiment 57, wherein the expression level is detected by performing qRT-PCR.

59. The system of any one of embodiments 51-58, wherein the detecting the expression level is performed using a device that is part of the system or in communication with at least one of the one or more processors, wherein upon receipt of instructions sent by the at least one of the one or more processors, perform the detection of the expression levels.

60. The system of any one of embodiments 51-59, wherein the plurality of biomarkers selected from Table 3 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers or at least 74 biomarkers from Table 3.

61. The system of any one of embodiments 51-59, wherein the plurality of biomarkers selected from Table 3 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 3.

62. The system of any one of embodiments 51-59, wherein the plurality of biomarkers selected from Table 3 comprise biomarkers associated with the GP6 signaling pathway, the wound healing signaling pathway, RHOGDI signaling, phagosome formation, the intrinsic prothrombin activation pathway, the pulmonary fibrosis idiopathic signaling pathway, synaptic long term depression, the oxytocin signaling pathway, sperm motility or cell cycle GS/M DNA damage checkpoint regulation.

63. The system of any one of embodiments 51-59, wherein the plurality of biomarkers selected from Table 3 comprises all the biomarkers from Table 3.

64. The system of any one of embodiments 51-59, wherein the plurality of biomarkers selected from Table 4 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers, at least 84 biomarkers, at least 86 biomarkers, at least 88 biomarkers, at least 90 biomarkers, at least 92 biomarkers, at least 94 biomarkers, at least 96 biomarkers, at least 98 biomarkers or at least 99 biomarkers from Table 4.

65. The system of any one of embodiments 51-59, wherein the plurality of biomarkers selected from Table 4 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 4.

66. The system of any one of embodiments 51-59, wherein the plurality of biomarkers selected from Table 4 comprise biomarkers associated with phagosome formation, the CLEAR signaling pathway, the pyroptosis signaling pathway, TREM1 signaling, the neuroinflammation signaling pathway, Th1 pathway, LPS/Il-1 mediated inhibition of RXR function, crosstalk between dendritic cells and natural killer cells, Fcgamma Receptor-mediated phagocytosis in macrophages and monocytes, production of nitric oxide and reactive oxygen species in macrophages, Toll-Like receptor signaling, inflammasome pathway, G-protein coupled receptor signaling, Th2 pathway, LXR/RXR activation, role of pattern recognition receptors in recognition of bacteria and viruses, glycolysis I or the necroptosis signaling pathway.

67. The system of any one of embodiments 51-59, wherein the plurality of biomarkers selected from Table 4 comprises all the biomarkers from Table 4.

68. A method of assaying a sample obtained from a subject, the method comprising measuring a nucleic acid expression level of a plurality of biomarkers from Table 5 in the sample obtained from the subject, wherein the subject suffers from or is suspected of suffering from fibromyalgia (FM).

69. The method of embodiment 68, wherein the method further comprises comparing the detected levels of nucleic acid expression of the plurality of biomarkers selected from Table 5 to the expression of the plurality of biomarkers selected from Table 5 in a control; and classifying the subject as having FM based on the results of the comparing step.

70. The method of embodiment 68, wherein the comparing step comprises applying a statistical algorithm which comprises determining a correlation between the expression data obtained from the sample and the expression data from the control.

71. The method of any one of embodiments 68-70, wherein the nucleic acid expression level is RNA or cDNA.

72. The method any one of embodiments 68-71, wherein the detecting the nucleic acid expression level comprises performing quantitative real time reverse transcriptase polymerase chain reaction (qRT-PCR), RNAseq or whole transcriptome analysis.

73. The method of embodiment 72, wherein the nucleic acid expression level is detected by performing RNA-seq.

74. The method of embodiment 73, wherein the detection of the nucleic acid expression level comprises using at least one pair of oligonucleotide primers specific for each biomarker from the plurality of biomarkers selected from Table 5.

75. The method of any one of embodiments 68-74, wherein the sample is a bodily fluid obtained from the subject.

76. The method of embodiment 75, wherein the bodily fluid is blood or fractions thereof, urine, saliva, or sputum.

77. The method of any one of embodiments 68-76, wherein the plurality of biomarkers selected from Table 5 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers or at least 83 biomarkers from Table 5.

78. The method of any one of embodiments 68-76, wherein the plurality of biomarkers selected from Table 5 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 5.

79. The method of any one of embodiments 68-76, wherein the plurality of biomarkers selected from Table 5 comprise biomarkers associated with Processing of capped intron-containing pre-mRNA, the Interferon alpha/beta signaling, the Necroptosis signaling pathway, Death receptor signaling, DDX58/IFIH1-mediated induction of interferon-alpha/beta, Role of PKR in interferon induction and antiviral response, the Pyroptosis signaling pathway, ISG15 antiviral mechanism, Interferon gamma signaling, Natural killer cell signaling, Role of hyperchemokinemia in the pathogenesis of influenza, ISGylation signaling pathway, JAK/STAT signaling, Activation of IRF by cytosolic pattern recognition receptors, Interleukin-2 family signaling, RIPK1-mediated regulated necrosis or Salvage pathways of pyrimidine ribonucleotides.

80. The method of any one of embodiments 68-76, wherein the plurality of biomarkers selected from Table 5 comprises all the biomarkers from Table 5.

81. A method of detecting a biomarker in a sample obtained from a subject suffering from or suspected of suffering from fibromyalgia (FM), the method comprising, consisting essentially of or consisting of measuring the nucleic acid expression level of a plurality of biomarkers selected from Table 5 using an amplification, hybridization and/or sequencing assay.

82. The method of embodiment 81, wherein the sample was previously diagnosed as being FM.

83. The method of embodiment 81 or 82, wherein the amplification, hybridization and/or sequencing assay comprises performing quantitative real time reverse transcriptase polymerase chain reaction (qRT-PCR), RNAseq, microarrays, gene chips, Serial Analysis of Gene Expression (SAGE), Rapid Analysis of Gene Expression (RAGE), nuclease protection assays, or Northern blotting.

84. The method of embodiment 83, wherein the nucleic acid expression level is measured by performing qRT-PCR.

85. The method of embodiment 81, wherein the measuring of the nucleic acid expression level comprises using at least one pair of oligonucleotide primers per each of the plurality of biomarker selected from Table 5.

86. The method of any one of embodiments 81-85, wherein the sample is a bodily fluid obtained from the subject.

87. The method of embodiment 86, wherein the bodily fluid is blood or fractions thereof, urine, saliva, or sputum.

88. The method of any one of embodiments 81-87, wherein the plurality of biomarkers selected from Table 5 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers, or at least 83 biomarkers from Table 5.

89. The method of any one of embodiments 81-87, wherein the plurality of biomarkers selected from Table 5 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 5.

90. The method of any one of embodiments 81-87, wherein the plurality of biomarkers selected from Table 5 comprise biomarkers associated with Processing of capped intron-containing pre-mRNA, the Interferon alpha/beta signaling, the Necroptosis signaling pathway, Death receptor signaling, DDX58/IFIH1-mediated induction of interferon-alpha/beta, Role of PKR in interferon induction and antiviral response, the Pyroptosis signaling pathway, ISG15 antiviral mechanism, Interferon gamma signaling, Natural killer cell signaling, Role of hyperchemokinemia in the pathogenesis of influenza, ISGylation signaling pathway, JAK/STAT signaling, Activation of IRF by cytosolic pattern recognition receptors, Interleukin-2 family signaling, RIPK1-mediated regulated necrosis or Salvage pathways of pyrimidine ribonucleotides.

91. The method of any one of embodiments 81-87, wherein the plurality of biomarkers selected from Table 5 comprises all the biomarkers from Table 5.

92. A method of treating fibromyalgia (FM) in a subject, the method comprising:
measuring a nucleic acid expression level of a plurality of biomarkers in a sample obtained from a subject suspected of suffering from FM, wherein the plurality of biomarkers is selected from Table 5, wherein the nucleic acid expression level of the plurality of biomarkers indicates that the subject has FM; and
administering a standard of care for FM to the subject, wherein the standard of care for FM is selected from the group consisting of duloxetine, pregabalin, gabapentin and milnacipran.

93. The method of embodiment 92, wherein the determining step further comprises comparing the nucleic acid expression levels of the plurality of biomarkers from Table 5 to the nucleic acid expression levels of the plurality of biomarkers from Table 5 in at least one sample training set(s), wherein the at least one sample training set comprises nucleic acid expression level data of the plurality of biomarkers from Table 5 from a reference FM sample, nucleic acid expression level data of the plurality of biomarkers from Table 5 from a reference non-FM sample or a combination thereof, and classifying the subject as having FM based on the results of the comparing step.

94. The method of embodiment 93, wherein the non-FM sample is obtained from an individual not known to have FM.

95. The method of embodiment 93, wherein the non-FM sample is obtained from an individual diagnosed with a disease of widespread pain selected from the group consisting of rheumatoid arthritis, systemic lupus erythematosus (SLE), Sjogren Syndrome and multiple sclerosis.

96. The method of any one of embodiments 93-95, wherein the comparing step comprises applying a statistical algorithm which comprises determining a correlation between the expression data obtained from the sample obtained from the subject and the expression data from the at least one training set(s); and classifying the subject as having FM based on the results of the statistical algorithm.

97. The method of any one of embodiments 92-95, wherein the plurality of biomarkers selected from Table 5 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers or at least 83 biomarkers from Table 5.

98. The method of any one of embodiments 92-95, wherein the plurality of biomarkers selected from Table 5 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 5.

99. The method of any one of embodiments 92-95, wherein the plurality of biomarkers selected from Table comprise biomarkers associated with Processing of capped intron-containing pre-mRNA, the Interferon alpha/beta signaling, the Necroptosis signaling pathway, Death receptor signaling, DDX58/IFIH1-mediated induction of interferon-alpha/beta, Role of PKR in interferon induction and antiviral response, the Pyroptosis signaling pathway, ISG15 antiviral mechanism, Interferon gamma signaling, Natural killer cell signaling, Role of hyperchemokinemia in the pathogenesis of influenza, ISGylation signaling pathway, JAK/STAT signaling, Activation of IRF by cytosolic pattern recognition receptors, Interleukin-2 family signaling, RIPK1-mediated regulated necrosis or Salvage pathways of pyrimidine ribonucleotides.

100. The method of any one of embodiments 92-95, wherein the plurality of biomarkers selected from Table 5 comprises all the biomarkers from Table 5.

101. The method of any one of embodiments 92-100, wherein the measuring the nucleic acid expression level is conducted using an amplification, hybridization and/or sequencing assay.

102. The method of embodiment 101, wherein the amplification, hybridization and/or sequencing assay comprises performing quantitative real time reverse transcriptase polymerase chain reaction (qRT-PCR), RNAseq, microarrays, gene chips, Serial Analysis of Gene Expression (SAGE), Rapid Analysis of Gene Expression (RAGE), nuclease protection assays or Northern blotting.

103. The method of embodiment 102, wherein the expression level is detected by performing qRT-PCR.

104. The method of any one of embodiments 92-103, wherein the sample is a bodily fluid obtained from the subject.

105. The method of embodiment 104, wherein the bodily fluid is blood or fractions thereof, urine, saliva, or sputum.

106. A system for diagnosing fibromyalgia (FM) from a sample obtained from a subject suspected of suffering from FM, the system comprising:
(a) one or more processors; and
(b) one or more memories operatively coupled to at least one of the one or more processors and having instructions stored thereon that, when executed by at least one of the one or more processors, cause the system to
   (i) detect an expression level of each of a plurality of biomarkers from Table 5;
   (ii) compare the expression levels of each of the plurality of biomarkers from Table 5 to the expression levels of each of the plurality of biomarkers from Table 5 in a control or compare the expression levels of each of the plurality of biomarkers from Table 5 to the expression levels of each of the plurality of biomarkers from Table 5 in a control; and
   (iii) classifying the subject as having FM based on the results of the comparing step.

107. The system of embodiment 106, wherein the control comprises at least one sample training set(s), wherein the at least one sample training set comprises expression levels of each of the plurality of biomarkers from Table 5 from a reference FM sample, expression levels of each of the plurality of biomarkers from Table 5 from a reference non-FM sample or a combination thereof.

108. The system of embodiment 107, wherein the non-FM sample is obtained from an individual not known to have FM.

109. The system of embodiment 107, wherein the non-FM sample is obtained from an individual diagnosed with a disease of widespread pain selected from the group consisting of rheumatoid arthritis, systemic lupus erythematosus (SLE), Sjogren Syndrome and multiple sclerosis.

110. The system of any one of embodiments 106-109, wherein the comparing step comprises applying a statistical algorithm which comprises determining a correlation between the nucleic acid expression data obtained from the sample and the expression data from the at least one training set(s); and classifying the subject as having FM based on the results of the statistical algorithm.

111. The system of any one of embodiments 106-110, wherein the expression level is a nucleic acid expression level, wherein the nucleic acid expression level is RNA or cDNA.

112. The system of embodiment 111, wherein the detecting the nucleic acid expression level comprises performing quantitative real time reverse transcriptase polymerase chain reaction (qRT-PCR), RNAseq, microarrays, gene chips, Serial Analysis of Gene Expression (SAGE), Rapid Analysis of Gene Expression (RAGE), nuclease protection assays or Northern blotting.

113. The system of embodiment 111 or 112, wherein the nucleic acid expression level is detected by performing qRT-PCR.

114. The system of any one of embodiments 106-113, wherein the detecting the expression level is performed using a device that is part of the system or in communication with at least one of the one or more processors, wherein upon receipt of instructions sent by the at least one of the one or more processors, perform the detection of the expression levels.

115. The system of any one of embodiments 106-114, wherein the plurality of biomarkers selected from Table 5 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers, or at least 83 biomarkers from Table 5.

116. The system of any one of embodiments 106-114, wherein the plurality of biomarkers selected from Table 5 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 5.

117. The system of any one of embodiments 106-114, wherein the plurality of biomarkers selected from Table 5 comprise biomarkers associated with Processing of capped intron-containing pre-mRNA, the Interferon alpha/beta signaling, the Necroptosis signaling pathway, Death receptor signaling, DDX58/IFIH1-mediated induction of interferon-alpha/beta, Role of PKR in interferon induction and antiviral response, the Pyroptosis signaling pathway, ISG15 antiviral mechanism, Interferon gamma signaling, Natural killer cell signaling, Role of hyperchemokinemia in the pathogenesis of influenza, ISGylation signaling pathway, JAK/STAT signaling, Activation of IRF by cytosolic pattern recognition receptors, Interleukin-2 family signaling, RIPK1-mediated regulated necrosis or Salvage pathways of pyrimidine ribonucleotides.

118. The system of any one of embodiments 106-114, wherein the plurality of biomarkers selected from Table 5 comprises all the biomarkers from Table 5.

1. A method of assaying a sample obtained from a subject, the method comprising measuring a nucleic acid expression level of a plurality of biomarkers from either Table 3, Table 4 or Table 5 in the sample obtained from the subject, wherein the subject suffers from or is suspected of suffering from a disease that results from an immune deficiency selected from the group consisting of fibromyalgia (FM), chronic fatigue, interstitial cystitis, brain fog, sleeplessness, chronic pain, mental depression, chronic anxiety, irritable bowel syndrome (IBS) and Long COVID.

2.-9. (canceled)

10. The method of claim 1, wherein the plurality of biomarkers selected from Table 3 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers or all the biomarkers from Table 3.

11. (canceled)

12. The method of claim 1, wherein the plurality of biomarkers selected from Table 3 comprise biomarkers associated with the GP6 signaling pathway, the wound healing signaling pathway, RHOGDI signaling, phagosome formation, the intrinsic prothrombin activation pathway, the pulmonary fibrosis idiopathic signaling pathway, synaptic long-term depression, the oxytocin signaling pathway, sperm motility or cell cycle GS/M DNA damage checkpoint regulation.

13. (canceled)

14. The method of claim 1, wherein the plurality of biomarkers selected from Table 4 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers, at least 84 biomarkers, at least 86 biomarkers, at least 88 biomarkers, at least 90 biomarkers, at least 92 biomarkers, at least 94 biomarkers, at least 96 biomarkers, at least 98 biomarkers, at least 99 biomarkers or all the biomarkers from Table 4.

15. The method of claim 1, wherein the plurality of biomarkers selected from Table 4 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 3, Table 4 or Table 5.

16. The method of claim 1, wherein the plurality of biomarkers selected from Table 4 comprise biomarkers associated with phagosome formation, the CLEAR signaling pathway, the pyroptosis signaling pathway, TREM1 signaling, the neuroinflammation signaling pathway, Th1 pathway, LPS/Il-1 mediated inhibition of RXR function, crosstalk between dendritic cells and natural killer cells, Fcgamma Receptor-mediated phagocytosis in macrophages and monocytes, production of nitric oxide and reactive oxygen species in macrophages, Toll-Like receptor signaling, inflammasome pathway, G-protein coupled receptor signaling, Th2 pathway, LXR/RXR activation, role of pattern recognition receptors in recognition of bacteria and viruses, glycolysis I or the necroptosis signaling pathway.

17.-32. (canceled)

33. A method of treating a disease that results from an immune deficiency selected from the group consisting of fibromyalgia (FM), chronic fatigue, interstitial cystitis, brain fog, sleeplessness, chronic pain, mental depression, chronic anxiety, irritable bowel syndrome (IBS) and Long COVID in a subject, the method comprising:
measuring a nucleic acid expression level of a plurality of biomarkers in a sample obtained from a subject suspected of suffering from disease that results from an immune deficiency selected from the group consisting of fibromyalgia (FM), chronic fatigue, interstitial cystitis, brain fog, sleeplessness, chronic pain, mental depression, chronic anxiety, irritable bowel syndrome (IBS) and Long COVID, wherein the plurality of biomarkers is selected from Table 3, Table 4 or Table 5, wherein the nucleic acid expression level of the plurality of biomarkers indicates that the subject has disease that results from an immune deficiency selected from the group consisting of fibromyalgia (FM), chronic fatigue, interstitial cystitis, brain fog, sleeplessness, chronic pain, mental depression, chronic anxiety, irritable bowel syndrome (IBS) and Long COVID, wherein the disease that results from an immune deficiency selected from the group consisting of fibromyalgia (FM), chronic fatigue, interstitial cystitis, brain fog, sleeplessness, chronic pain, mental depression, chronic anxiety, irritable bowel syndrome (IBS) and Long COVID; and
administering a treatment for the disease that results from an immune deficiency, wherein the treatment is selected from the group consisting of a treatment that modulates the immune system, a vaccine, a nutritional supplement, a pre-biotic, a probiotic, a post-biotic and a standard of care for the disease that results from an immune deficiency to the subject.

34. The method of claim 33, wherein the measuring further comprises comparing the nucleic acid expression levels of the plurality of biomarkers from Table 3, Table 4 or Table 5 to the nucleic acid expression levels of the plurality of biomarkers from Table 3, Table 4 or Table 5 in at least one sample training set(s), wherein the at least one sample training set comprises nucleic acid expression level data of the plurality of biomarkers from Table 3, Table 4 or Table 5 from a reference the disease that results from an immune deficiency sample, nucleic acid expression level data of the plurality of biomarkers from Table 3, Table 4 or Table 5 from a reference non-disease that results from an immune deficiency FM sample or a combination thereof; and classifying the subject as having the disease that results from an immune deficiency based on the results of the comparing step.

35. The method of claim 34, wherein the disease that results from an immune deficiency is fibromyalgia (FM), and wherein the non-FM sample is obtained from an individual not known to have FM or an individual diagnosed with a disease of widespread pain selected from the group consisting of rheumatoid arthritis, systemic lupus erythematosus (SLE), Sjogren Syndrome and multiple sclerosis.

36. (canceled)

37. The method of claim 34, wherein the comparing step comprises applying a statistical algorithm which comprises determining a correlation between the expression data obtained from the sample obtained from the subject and the expression data from the at least one training set(s); and classifying the subject as having the disease that results from an immune deficiency based on the results of the statistical algorithm.

38. The method of claim 33, wherein the plurality of biomarkers selected from Table 3 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers or all the biomarkers from Table 3.

39. The method of claim 33, wherein the plurality of biomarkers selected from Table 3 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the biomarkers from Table 3, Table 4 or Table 5.

40. The method of claim 33, wherein the plurality of biomarkers selected from Table 3 comprise biomarkers associated with the GP6 signaling pathway, the wound healing signaling pathway, RHOGDI signaling, phagosome formation, the intrinsic prothrombin activation pathway, the pulmonary fibrosis idiopathic signaling pathway, synaptic long term depression, the oxytocin signaling pathway, sperm motility or cell cycle GS/M DNA damage checkpoint regulation.

41. (canceled)

42. The method of claim 33, wherein the plurality of biomarkers selected from Table 4 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers, at least 84 biomarkers, at least 86 biomarkers, at least 88 biomarkers, at least 90 biomarkers, at least 92 biomarkers, at least 94 biomarkers, at least 96 biomarkers, at least 98 biomarkers, at least 99 biomarkers or all the biomarkers from Table 4.

43. (canceled)

44. The method of claim 33, wherein the plurality of biomarkers selected from Table 4 comprise biomarkers associated with phagosome formation, the CLEAR signaling pathway, the pyroptosis signaling pathway, TREM1 signaling, the neuroinflammation signaling pathway, Th1 pathway, LPS/Il-1 mediated inhibition of RXR function, crosstalk between dendritic cells and natural killer cells, Fcgamma Receptor-mediated phagocytosis in macrophages and monocytes, production of nitric oxide and reactive oxygen species in macrophages, Toll-Like receptor signaling, inflammasome pathway, G-protein coupled receptor signaling, Th2 pathway, LXR/RXR activation, role of pattern recognition receptors in recognition of bacteria and viruses, glycolysis I or the necroptosis signaling pathway.

45.-50. (canceled)

51. A system for diagnosing a disease that results from an immune deficiency selected from the group consisting of fibromyalgia (FM), chronic fatigue, interstitial cystitis, brain fog, sleeplessness, chronic pain, mental depression, chronic anxiety, irritable bowel syndrome (IBS) and Long COVID from a sample obtained from a subject suspected of suffering from FM, the system comprising:
(a) one or more processors; and
(b) one or more memories operatively coupled to at least one of the one or more processors and having instructions stored thereon that, when executed by at least one of the one or more processors, cause the system to
(i) detect an expression level of each of a plurality of biomarkers from Table 3, Table 4 or Table 5;
(ii) compare the expression levels of each of the plurality of biomarkers from Table 3 to the expression levels of each of the plurality of biomarkers from Table 3 in a control, compare the expression levels of each of the plurality of biomarkers from Table 4 to the expression levels of each of the plurality of biomarkers from Table 4 in a control or compare the expression levels of each of the plurality of biomarkers from Table 5 to the expression levels of each of the plurality of biomarkers from Table 5 in a control; and
(iii) classifying the subject as having the disease that results from an immune deficiency selected from the group consisting of fibromyalgia (FM), chronic fatigue, interstitial cystitis, brain fog, sleeplessness, chronic pain, mental depression, chronic anxiety, irritable bowel syndrome (IBS) and Long COVID based on the results of the comparing step.

52.-76. (canceled)

77. The method of claim 1, wherein the plurality of biomarkers selected from Table 5 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers, at least 83 biomarkers or all the biomarkers from Table 5.

78. (canceled)

79. The method of claim 1, wherein the plurality of biomarkers selected from Table 5 comprise biomarkers associated with Processing of capped intron-containing pre-mRNA, the Interferon alpha/beta signaling, the Necroptosis signaling pathway, Death receptor signaling, DDX58/IFIH1-mediated induction of interferon-alpha/beta, Role of PKR in interferon induction and antiviral response, the Pyroptosis signaling pathway, ISG15 antiviral mechanism, Interferon gamma signaling, Natural killer cell signaling, Role of hyperchemokinemia in the pathogenesis of influenza, ISGylation signaling pathway, JAK/STAT signaling, Activation of IRF by cytosolic pattern recognition receptors, Interleukin-2 family signaling, RIPK1-mediated regulated necrosis or Salvage pathways of pyrimidine ribonucleotides.

80.-96. (canceled)

97. The method of claim 33, wherein the plurality of biomarkers selected from Table 5 comprises at least 2 biomarkers, at least 4 biomarkers, at least 6 biomarkers, at least 8 biomarkers, at least 10 biomarkers, at least 12 biomarkers, at least 14 biomarkers, at least 16 biomarkers, at least 18 biomarkers, at least 20 biomarkers, at least 22 biomarkers, at least 24 biomarkers, at least 26 biomarkers, at least 28 biomarkers, at least 30 biomarkers, at least 32 biomarkers, at least 34 biomarkers, at least 36 biomarkers, at least 38 biomarkers, at least 40 biomarkers, at least 42 biomarkers, at least 44 biomarkers, at least 46 biomarkers, at least 48 biomarkers, at least 50 biomarkers, at least 52 biomarkers, at least 54 biomarkers, at least 56 biomarkers, at least 58 biomarkers, at least 60 biomarkers, at least 62 biomarkers, at least 64 biomarkers, at least 66 biomarkers, at least 68 biomarkers, at least 70 biomarkers, at least 72 biomarkers, at least 74 biomarkers, at least 76 biomarkers, at least 78 biomarkers, at least 80 biomarkers, at least 82 biomarkers, at least 83 biomarkers or all the biomarkers from Table 5.

98. (canceled)

99. The method of claim 33, wherein the plurality of biomarkers selected from Table 5 comprise biomarkers associated with Processing of capped intron-containing pre-mRNA, the Interferon alpha/beta signaling, the Necroptosis signaling pathway, Death receptor signaling, DDX58/IFIH1-mediated induction of interferon-alpha/beta, Role of PKR in interferon induction and antiviral response, the Pyroptosis signaling pathway, ISG15 antiviral mechanism, Interferon gamma signaling, Natural killer cell signaling, Role of hyperchemokinemia in the pathogenesis of influenza, ISGylation signaling pathway, JAK/STAT signaling, Activation of IRF by cytosolic pattern recognition receptors, Interleukin-2 family signaling, RIPK1-mediated regulated necrosis or Salvage pathways of pyrimidine ribonucleotides.

100.-118. (canceled)
